(19) United States
(12) Patent Application Publication
(10) Pub. No.: US 2025/0319105 A1
(43) Pub. Date: Oct. 16, 2025

(54) NEUROACTIVE STEROID FOR THE TREATMENT OF ALZHEIMER'S DISEASE

(71) Applicant: SAGE THERAPEUTICS, INC., Cambridge, MA (US)

(72) Inventors: James J. Doherty, Cambridge, MA (US); Michael C. Quirk, Cambridge, MA (US); Albert Jean Robichaud, Cambridge, MA (US); Aaron Michael Koenig, Cambridge, MA (US)

(21) Appl. No.: 18/568,624

(22) PCT Filed: Jun. 10, 2022
(86) PCT No.: PCT/US2022/033122

§ 371 Date: Dec. 8, 2023

Related U.S. Application Data
(60) Provisional application No. 63/209,929, filed on Jun. 11, 2021.
(60) Provisional application No. 63/289,081, filed on Dec. 13, 2021.
(60) Provisional application No. 63/321,598, filed on Mar. 18, 2022.

(51) Int. Cl. A61K 31/575; A61K 39/395; A61K 45/06; A61P 25/28

(52) U.S. Cl. CPC A61K 31/575; A61K 39/3955; A61K 45/06; A61P 25/28

(57) ABSTRACT

The present disclosure relates to compositions, kits and methods of treating Alzheimer's disease comprising administering an NMD A receptor positive allosteric modulator or a CYP46A1 inhibitor.

### CROSS REFERENCE TO RELATED APPLICATIONS

This application is a U. S. National Stage Application under 35 U.S.C. § 371 of International Application No. PCT/US2022/033122, filed Jun. 10, 2022, which claims priority to and the benefit of U.S. Provisional Application Nos. 63/321,598, filed Mar. 18, 2022, 63/289,081, filed Dec. 13, 2021 and 63/209,929, filed Jun. 11, 2021, the disclosures of each of which, are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 7, 2024, is named 000221-0060-301_SL.txt and is 65,381 bytes in size.

### BACKGROUND

Alzheimer's disease (AD) is a progressive and multifactorial neurodegenerative disorder beginning with loss of episodic memory and cognitive function, progressing to mild cognitive impairment (MCI) and/or dementia and possibly leading to loss of the ability to carry on a conversation and respond to the environment. Moreover, patients with AD mild cognitive impairment (AD-MCI) are at a high risk for developing dementia. It is the cause of a majority of dementia cases. The most common early symptom is difficulty in remembering recent events. As the disease progresses, symptoms can include problems with language, disorientation, mood swings, loss of motivation, self-neglect, and behavioral issues. As their condition worsens, subjects with AD often withdraw from family and society. Over time, subjects lose bodily functions, ultimately leading to death.

Alzheimer's disease (AD) remains without effective and stable therapeutic strategies. Currently, available medications for AD are based on symptomatic therapy and focus on managing behavioral and psychological symptoms. Recent clinical research has investigated treatments that focus on the hallmark underlying pathology of AD, including beta-amyloid(Aβ) deposition, tau hyperphosphorylation, neuroinflammation, and vascular changes. Thus far, however, the tested agents have not shown clinical benefit with respect to improving the cognitive function and dementia that manifest in AD patients.

NMDA receptors are highly expressed in the CNS and are involved in excitatory synaptic transmission. Activating these receptors contributes to synaptic plasticity in some circumstances and excitotoxicity in others. These receptors are ligand-gated ion channels that admit Ca²⁺ after binding of the neurotransmitters, glutamate and glycine, and are fundamental to excitatory neurotransmission and normal CNS function. NMDA receptors are heteromeric complexes comprised of NR1, NR2, and/or NR3 subunits and possess distinct recognition sites for exogenous and endogenous ligands. These recognition sites include binding sites for glycine, and glutamate agonists and modulators. Positive modulators may be useful as therapeutic agents with potential clinical uses as cognitive enhancers and in the treatment of psychiatric disorders in which glutamatergic transmission is reduced or defective (see, e.g., Horak et al., J. Neuroscience, 2004, 24 (46), 10318-10325).

Therefore, there remains a need for new and improved therapeutics for treating Alzheimer's Disease. The compositions, kits and methods disclosed herein are directed toward this end.

### SUMMARY

Provided herein are methods of treating Alzheimer's Disease, including methods of treating Mild Cognitive Impairment (MCI) or mild dementia associated with Alzheimer's Disease.

In one aspect, provided herein is a method of treating Mild Cognitive Impairment (MCI) associated with Alzheimer's Disease in a subject, the method comprising administering to the subject a compound having the formula

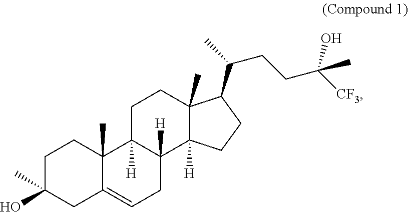

or a pharmaceutically acceptable salt thereof.

In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving executive function in a subject having Mild Cognitive Impairment (MCI) associated with Alzheimer's Disease, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving working memory in a subject having Mild Cognitive Impairment (MCI) associated with Alzheimer's Disease, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving learning in a subject having Mild Cognitive Impairment (MCI) associated with Alzheimer's Disease, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving learning and working memory in a subject having Mild Cognitive Impairment (MCI) associated with Alzheimer's Disease, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of treating mild dementia associated with Alzheimer's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving executive function in a subject having mild dementia associated with Alzheimer's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving working memory in a subject having mild dementia associated with Alzheimer's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving learning in a subject having mild dementia associated with Alzheimer's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In one aspect, provided herein is a method of improving learning and working memory in a subject having mild dementia associated with Alzheimer's Disease in a subject, the method comprising administering to the subject Compound 1 or a pharmaceutically acceptable salt thereof. In some embodiments, Compound 1 is administered to the subject daily. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, Compound 1 is administered orally.

In some embodiments, subject is an adult human. In some embodiments, Compound 1 or a pharmaceutically acceptable salt thereof is administered as a monotherapy. In some embodiments, Compound 1 or a pharmaceutically acceptable salt thereof is administered in combination with another therapeutic agent.

In one aspect, the disclosure provides a pharmaceutical composition comprising: (a) an NMDA receptor positive allosteric modulator; (b) an anti-amyloid beta antibody or antigen-binding fragment thereof; and (c) a pharmaceutically acceptable carrier.

In one aspect, the disclosure provides a pharmaceutical composition comprising: (a) a CYP46A1 inhibitor; (b) an anti-amyloid beta antibody or antigen-binding fragment thereof; and (c) a pharmaceutically acceptable carrier.

In one aspect, the disclosure provides a kit comprising a first container, a second container and a package insert, wherein: the first container comprises a composition comprising an NMDA receptor positive allosteric modulator; the second container comprises composition comprising an anti-amyloid beta antibody or antigen-binding fragment thereof; and the package insert comprises instructions for treating Alzheimer's disease in a subject.

In one aspect, the disclosure provides a kit comprising a first container, a second container and a package insert, wherein: the first container comprises a composition comprising CYP46A1 inhibitor; the second container comprises composition comprising an anti-amyloid beta antibody or antigen-binding fragment thereof; and the package insert comprises instructions for treating Alzheimer's disease in a subject.

In one aspect, the disclosure provides a method for treating or preventing Alzheimer's disease, comprising administering to a subject in need thereof (a) an NMDA receptor positive allosteric modulator, and (b) an anti-amyloid beta antibody or antigen-binding fragment thereof. In some embodiments, the disclosure provides a method for treating Alzheimer's disease, comprising administering to a subject in need thereof (a) an NMDA receptor positive allosteric modulator, and (b) an anti-amyloid beta antibody or antigen-binding fragment thereof. In some embodiments, the disclosure provides a method for preventing Alzheimer's disease, comprising administering to a subject in need thereof (a) an NMDA receptor positive allosteric modulator, and (b) an anti-amyloid beta antibody or antigen-binding fragment thereof.

In one aspect, the disclosure provides a method for treating or preventing cognitive impairment in a subject having Alzheimer's disease, comprising administering to the subject (a) an NMDA receptor positive allosteric modulator, and (b) an anti-amyloid beta antibody or antigen-binding fragment thereof. In some embodiments, the disclosure provides a method for treating cognitive impairment in a subject having Alzheimer's disease, comprising administering to the subject (a) an NMDA receptor positive allosteric modulator, and (b) an anti-amyloid beta antibody or antigen-binding fragment thereof. In some embodiments, the disclosure provides a method for preventing cognitive impairment in a subject having Alzheimer's disease, comprising administering to the subject (a) an NMDA receptor positive allosteric modulator, and (b) an anti-amyloid beta antibody or antigen-binding fragment thereof.

In one aspect, the disclosure provides a method for treating or preventing cognitive impairment due to Alzheimer's disease in a subject, comprising administering to the subject (a) an NMDA receptor positive allosteric modulator, and (b) an anti-amyloid beta antibody or antigen-binding fragment thereof. In some embodiments, the disclosure provides a method for treating cognitive impairment due to Alzheimer's disease in a subject, comprising administering to the subject (a) an NMDA receptor positive allosteric modulator, and (b) an anti-amyloid beta antibody or antigen-binding fragment thereof. In some embodiments, the disclosure provides a method for preventing cognitive impairment due to Alzheimer's disease in a subject, comprising administering to the subject (a) an NMDA receptor positive allosteric modulator, and (b) an anti-amyloid beta antibody or antigen-binding fragment thereof. In some embodiments, the cognitive impairment is mild cognitive impairment.

In one aspect, the disclosure provides a method for improving cognition in a subject having Alzheimer's disease, comprising administering to the subject (a) an NMDA receptor positive allosteric modulator, and (b) an anti-amyloid beta antibody or antigen-binding fragment thereof.

In one aspect, the disclosure provides a method for slowing cognitive decline due to Alzheimer's disease in a subject, comprising administering to the subject (a) an NMDA receptor positive allosteric modulator, and (b) an anti-amyloid beta antibody or antigen-binding fragment thereof.

In one aspect, the disclosure provides a method of treating Mild Cognitive Impairment (MCI) associated with Alzheimer's Disease in a subject, the method comprising administering to the subject (a) an NMDA receptor positive allosteric modulator, and (b) an anti-amyloid beta antibody or antigen-binding fragment thereof.

In one aspect, the disclosure provides a method of improving executive function in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving working memory in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning and working memory in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of treating mild dementia associated with Alzheimer's Disease in a subject, the method comprising administering to the subject (a) an NMDA receptor positive allosteric modulator, and (b) an anti-amyloid beta antibody or antigen-binding fragment thereof.

In one aspect, the disclosure provides a method of improving executive function in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving working memory in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning and working memory in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method for treating or preventing Alzheimer's disease, comprising administering to a subject in need thereof (a) a CYP46A1 inhibitor, and (b) an anti-amyloid beta antibody or antigen-binding fragment thereof. In some embodiments, the disclosure provides a method for treating Alzheimer's disease, comprising administering to a subject in need thereof (a) a CYP46A1 inhibitor, and (b) an anti-amyloid beta antibody or antigen-binding fragment thereof. In some embodiments, the disclosure provides a method for treating Alzheimer's disease, comprising administering to a subject in need thereof (a) a CYP46A1 inhibitor, and (b) an anti-amyloid beta antibody or antigen-binding fragment thereof.

In one aspect, the disclosure provides a method for treating or preventing cognitive impairment in a subject having Alzheimer's disease, comprising administering to the subject (a) a CYP46A1 inhibitor, and (b) an anti-amyloid beta antibody or antigen-binding fragment thereof. In some embodiments, the disclosure provides a method for treating cognitive impairment in a subject having Alzheimer's disease, comprising administering to the subject (a) a CYP46A1 inhibitor, and (b) an anti-amyloid beta antibody or antigen-binding fragment thereof. In some embodiments, the disclosure provides a method for preventing cognitive impairment in a subject having Alzheimer's disease, comprising administering to the subject (a) a CYP46A1 inhibitor, and (b) an anti-amyloid beta antibody or antigen-binding fragment thereof.

In one aspect, the disclosure provides a method for treating or preventing cognitive impairment due to Alzheimer's disease in a subject, comprising administering to the subject (a) a CYP46A1 inhibitor, and (b) an anti-amyloid beta antibody or antigen-binding fragment thereof. In some embodiments, the disclosure provides a method for treating cognitive impairment due to Alzheimer's disease in a subject, comprising administering to the subject (a) a CYP46A1 inhibitor, and (b) an anti-amyloid beta antibody or antigen-binding fragment thereof. In some embodiments, the disclosure provides a method for preventing cognitive impairment due to Alzheimer's disease in a subject, comprising administering to the subject (a) a CYP46A1 inhibitor, and (b) an anti-amyloid beta antibody or antigen-binding fragment thereof. In some embodiments, the cognitive impairment is mild cognitive impairment.

In one aspect, the disclosure provides a method for improving cognition in a subject having Alzheimer's disease, comprising administering to the subject (a) a CYP46A1 inhibitor, and (b) an anti-amyloid beta antibody or antigen-binding fragment thereof.

In one aspect, the disclosure provides a method for slowing cognitive decline due to Alzheimer's disease in a subject, comprising administering to the subject (a) a CYP46A1 inhibitor, and (b) an anti-amyloid beta antibody or antigen-binding fragment thereof.

In one aspect, the disclosure provides a method of treating Mild Cognitive Impairment (MCI) associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving executive function in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving working memory in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving learning in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving learning and working memory in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of treating mild dementia associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving executive function in a subject having mild dementia associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving working memory in a subject having mild dementia associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving learning in a subject having mild dementia associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving learning and working memory in a subject having mild dementia associated with Alzheimer's Disease in a subject, the method comprising administering to the subject (a) a CYP46A1 inhibitor, and (b) an anti-amyloid beta antibody or antigen-binding fragment thereof.

In some embodiments, the NMDA receptor positive allosteric modulator is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl)(6,7-dimethoxy-1-[(4-methoxyphenoxy)methyl]-3,4-dihydroisoquinolin-2 (1H)-yl)methanone (CIQ), NYX-458, and plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one), and pharmaceutically acceptable salts thereof.

In some embodiments, the NMDA receptor positive allosteric modulator is a neuroactive steroid, or a pharmaceutically acceptable salt thereof. In some embodiments, the neuroactive steroid is selected from any one of compounds B1-B543; and pharmaceutically acceptable salts thereof. In some embodiments, the neuroactive steroid is selected from any one of compounds B1-B140, and pharmaceutically acceptable salts thereof. In some embodiments, the neuroactive steroid is selected from any one of compounds B150-B245, and pharmaceutically acceptable salts thereof. In some embodiments, the neuroactive steroid is selected from any one of compounds B273-B349, and pharmaceutically acceptable salts thereof. In some embodiments, the neuroactive steroid is selected from any one of compounds B395-B432, and pharmaceutically acceptable salts thereof. In some embodiments, the neuroactive steroid is selected from the group consisting of any one of compounds B433-B443, and pharmaceutically acceptable salts thereof. In some embodiments, the neuroactive steroid is selected from the group consisting of any one of compounds B444-B451, and pharmaceutically acceptable salts thereof. In some embodiments, the neuroactive steroid is selected from the group consisting of any one of compounds B452-B477, and pharmaceutically acceptable salts thereof. In some embodiments, the neuroactive steroid is selected from the group consisting of any one of compounds B478-B530, and pharmaceutically acceptable salts thereof. In some embodiments, the neuroactive steroid is selected from the group consisting of any one of compounds B531-B543, and pharmaceutically acceptable salts thereof.

In some embodiments, the CYP46A1 inhibitor is selected from the group consisting of TAK-935, any one of compounds A1-A182, and pharmaceutically acceptable salts thereof. In some embodiments, the CYP46A1 inhibitor is selected from the group consisting of TAK-935, any one of compounds A1-A125, and pharmaceutically acceptable salts thereof. In some embodiments, the CYP46A1 inhibitor is TAK-935, or a pharmaceutically acceptable salt thereof. In some embodiments, the CYP46A1 inhibitor is selected from the group consisting of any one of compounds A1-A125, and pharmaceutically acceptable salts thereof. In some embodiments, the CYP46A1 inhibitor is selected from the group consisting of any one of compounds A126-A182, and pharmaceutically acceptable salts thereof.

In some embodiments, the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.

In some embodiments, the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6;
(b) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16;
(c) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26;
(d) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36;
(e) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46;
(f) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56; or
(g) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and (b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.

In some embodiments, the anti-amyloid beta antibody or antigen-binding fragment thereof comprises a heavy chain variable domain (VH) comprising: (a) the amino acid sequence of SEQ ID NO: 7; (b) the amino acid sequence of SEQ ID NO: 17; (c) the amino acid sequence of SEQ ID NO: 27; (d) the amino acid sequence of SEQ ID NO: 37; (e) the amino acid sequence of SEQ ID NO: 47; (f) the amino acid sequence of SEQ ID NO: 57; or (g) the amino acid sequence of SEQ ID NO: 67.

In some embodiments, the anti-amyloid beta antibody or antigen-binding fragment thereof comprises a light chain variable domain (VL) comprising: (a) the amino acid sequence of SEQ ID NO: 8; (b) the amino acid sequence of SEQ ID NO: 18; (c) the amino acid sequence of SEQ ID NO: 28; (d) the amino acid sequence of SEQ ID NO: 38; (e) the amino acid sequence of SEQ ID NO: 48; (f) the amino acid sequence of SEQ ID NO: 58; or (g) the amino acid sequence of SEQ ID NO: 68.

In some embodiments, the anti-amyloid beta antibody or antigen-binding fragment thereof comprises a heavy chain variable domain (VH) comprising the amino acid sequence of SEQ ID NO: 7. In some embodiments, the anti-amyloid beta antibody or antigen-binding fragment thereof comprises a light chain variable domain (VL) comprising the amino acid sequence of SEQ ID NO: 8. In some embodiments, the anti-amyloid beta antibody or antigen-binding fragment thereof comprises a heavy chain variable domain (VH) comprising the amino acid sequence of SEQ ID NO: 7; and a light chain variable domain (VL) comprising the amino acid sequence of SEQ ID NO: 8.

In some embodiments, the anti-amyloid beta antibody or antigen-binding fragment thereof comprises a human IgG1, IgG2, IgG3, or IgG4 constant domain. In some embodiments, the anti-amyloid beta antibody or antigen-binding fragment thereof comprises a human IgG1 constant region.

In some embodiments, the anti-amyloid beta antibody or antigen-binding fragment thereof is a human anti-amyloid beta antibody or antigen-binding fragment thereof.

In some embodiments, the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).

In some embodiments, the anti-amyloid beta antibody comprises a heavy chain comprising (a) the amino acid sequence of SEQ ID NO: 9; (b) the amino acid sequence of SEQ ID NO: 19; (c) the amino acid sequence of SEQ ID NO: 29; (d) the amino acid sequence of SEQ ID NO: 39; (e) the amino acid sequence of SEQ ID NO: 49; (f) the amino acid sequence of SEQ ID NO: 59; or (g) the amino acid sequence of SEQ ID NO: 69.

In some embodiments, the anti-amyloid beta antibody comprises a light chain comprising: (a) the amino acid sequence of SEQ ID NO: 10; (b) the amino acid sequence of SEQ ID NO: 20; (c) the amino acid sequence of SEQ ID NO: 30; (d) the amino acid sequence of SEQ ID NO: 40; (e) the amino acid sequence of SEQ ID NO: 50; (f) the amino acid sequence of SEQ ID NO: 60; or (g) the amino acid sequence of SEQ ID NO: 70.

In some embodiments, the anti-amyloid beta antibody comprises a heavy chain comprising the amino acid sequence of SEQ ID NO: 9.

In some embodiments, the anti-amyloid beta antibody comprises a light chain comprising the amino acid sequence of SEQ ID NO: 10.

In some embodiments, the anti-amyloid beta antibody is aducanumab. In some embodiments, the anti-amyloid beta antibody or antigen binding fragment thereof binds to the same conformational epitope of amyloid beta to which aducanumab binds.

In some embodiments, the anti-amyloid beta antibody or antigen binding fragment thereof competitively inhibits binding of aducanumab to amyloid beta. In some embodiments, the anti-amyloid beta antibody or antigen binding fragment thereof binds to amyloid beta plaques, parenchymal amyloid, cerebrovascular amyloid, or diffuse amyloid beta deposits.

In some embodiments, the NMDA receptor positive allosteric modulator and the anti-amyloid beta antibody or antigen binding fragment thereof are administered simultaneously. In some embodiments, the NMDA receptor positive allosteric modulator and the anti-amyloid beta antibody or antigen binding fragment thereof are administered sequentially. In some embodiments, the NMDA receptor positive allosteric modulator and the anti-amyloid beta antibody or antigen binding fragment thereof are administered separately. In some embodiments, the anti-amyloid beta antibody or antigen binding fragment thereof is administered before the NMDA receptor positive allosteric modulator. In some embodiments, the NMDA receptor positive allosteric modulator is administered before the anti-amyloid beta antibody or antigen binding fragment thereof. In some embodiments, the frequency of administration of the anti-amyloid beta antibody or antigen binding fragment thereof and the NMDA receptor positive allosteric modulator is the same. In some embodiments, the frequency of administration of the anti-amyloid beta antibody or antigen binding fragment thereof and the NMDA receptor positive allosteric modulator is different.

In some embodiments, the CYP46A1 inhibitor and the anti-amyloid beta antibody or antigen binding fragment thereof are administered simultaneously. In some embodiments, the CYP46A1 inhibitor and the anti-amyloid beta antibody or antigen binding fragment thereof are administered sequentially. In some embodiments, the CYP46A1 inhibitor and the anti-amyloid beta antibody or antigen binding fragment thereof are administered separately. In some embodiments, the anti-amyloid beta antibody or antigen binding fragment thereof is administered before the CYP46A1 inhibitor. In some embodiments, the CYP46A1 inhibitor is administered before the anti-amyloid beta antibody or antigen binding fragment thereof. In some embodiments, the frequency of administration of the anti-amyloid beta antibody or antigen binding fragment thereof and the CYP46A1 inhibitor is the same. In some embodiments, the frequency of administration of the anti-amyloid beta antibody or antigen binding fragment thereof and the CYP46A1 inhibitor is different.

### BRIEF DESCRIPTION OF THE DRAWINGS

FIG. 1 depicts an overview of the study design.

FIG. 2 depicts the executive functioning deficit at baseline in AD patients as measured using the Digit Symbol Substitution Test.

FIG. 3 depicts learning and memory at baseline in AD patients as measured by pattern recognition memory (PRM).

FIG. 4 depicts attention and psychomotor speed at baseline in AD patients as measured by reaction time (RT).

FIG. 5 depicts executive functioning in AD patients as measured using the 2-Back Test.

FIG. 6 depicts executive functioning in AD patients as measured using the MTT.

FIG. 7 depicts learning and memory in AD patients as measured using the VRM (Free Recall) test.

FIG. 8 depicts learning and memory in AD patients as measured using the PRM (% correct delayed) test.

### DETAILED DESCRIPTION

As generally described herein, the present invention provides compositions, kits and methods for treating various CNS diseases and disorders in a subject in need thereof.

### Definitions

The term “herein” means the entire application.

Unless otherwise defined herein, scientific and technical terms used in this application shall have the meanings that are commonly understood by those of ordinary skill in the art to which this invention belongs. Generally, nomenclature used in connection with the compounds, composition and methods described herein, are those well-known and commonly used in the art.

It should be understood that any of the embodiments described herein, including those described under different aspects of the disclosure and different parts of the specification (including embodiments described only in the Examples) can be combined with any one or more other embodiments of the invention, unless explicitly disclaimed or improper. Combination of embodiments are not limited to those specific combinations claimed via the multiple dependent claims.

All of the publications, patents and published patent applications referred to in this application are specifically incorporated by reference herein in their entirety. In case of conflict, the present specification, including its specific definitions, will control.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Standard techniques may be used for chemical syntheses and antibody production. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of subjects.

Throughout this specification, the word “comprise” or variations such as “comprises” or “comprising” will be understood to imply the inclusion of a stated integer (or components) or group of integers (or components), but not the exclusion of any other integer (or components) or group of integers (or components).

Throughout the specification, where compositions are described as having, including, or comprising (or variations thereof), specific components, it is contemplated that compositions also may consist essentially of, or consist of, the recited components. Similarly, where methods or processes are described as having, including, or comprising specific process steps, the processes also may consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

The term “including,” as used herein, means “including but not limited to.” “Including” and “including but not limited to” are used interchangeably. Thus, these terms will be understood to imply the inclusion of a stated integer (or components) or group of integers (or components), but not the exclusion of any other integer (or components) or group of integers (or components).

As used herein, “about” or “approximately” means within an acceptable error range for the particular value as determined by one of ordinary skill in the art, which will depend in part on how the value is measured or determined, i.e., the limitations of the measurement system.

Unless otherwise required by context, singular terms shall include pluralities and plural terms shall include the singular.

The use of the terms “a” and “an” and “the” and similar referents in the context of describing the elements (especially in the context of the following claims) are to be construed to cover both the singular and the plural, unless otherwise indicated herein or clearly contradicted by context.

The term “or” as used herein should be understood to mean “and/or,” unless the context clearly indicates otherwise.

Recitation of ranges of values herein are merely intended to serve as a shorthand method of referring individually to each separate value falling within the range, unless otherwise indicated herein, and each separate value is incorporated into the specification as if it were individually recited herein. Moreover, all ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The disclosure of a range should also be considered as disclosure of the endpoints of that range.

All methods described herein can be performed in any suitable order unless otherwise indicated herein or otherwise clearly contradicted by context.

The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

The terms “antibody” (Ab) and “immunoglobulin” (Ig) are used interchangeably herein and refer to a molecule (e.g., complete antibodies, antibody fragment or modified antibodies) capable of recognizing and binding to a specific target or antigen, such as a carbohydrate, polynucleotide, lipid, polypeptide, etc., through at least one antigen recognition site, located in the variable region of the molecule. An antibody may be either membrane bound or secreted. As used herein, the term encompasses not only intact, or “whole”, polyclonal or monoclonal antibodies, but also fragments thereof (such as single-variable domain (VH, VL or combination thereof) antibodies, Fab, Fab′, F(ab′)2, Fv), single chain (ScFv), synthetic variants thereof, naturally occurring variants, fusion proteins comprising an antibody portion with an antigen-binding fragment of the required specificity, humanized antibodies, chimeric antibodies, chimeric antigen receptors (CARs), and any other modified configuration of the immunoglobulin molecule that comprises an antigen-binding site or fragment (epitope recognition site) of the required specificity.

Antibody, or Ig, molecules typically comprise two heavy chains and two light chains linked together through disulfide bonds. Both heavy chains (IgH) and light chains (IgL) contain a variable (V) region or domain and a constant (C) region or domain. The portion of the IgH locus encoding the V region comprises multiple copies of variable (V), diversity (D), and joining (J) gene segments. The portion of the IgL loci encoding the V region comprises multiple copies of V and J gene segments. The V region encoding portion of the IgH and IgL loci undergo gene segment rearrangement, e.g., different combinations of a V, (D) and J gene segments arrange to form the IgH and IgL variable regions (VH and VL, respectively), to develop diverse antigen specificity in antibodies. Each variable region comprises three hypervariable complementarity-determining regions (CDRs) interspersed between the less variable framework regions (FRs). Complete variable domains, where present, comprise four framework regions (FRs) and three complementarity determining regions (CDRs), arranged, proceeding from the amino terminus, in the order FR1, CDR1, FR2, CDR2, FR3, CDR3, FR4. Visual inspection and sequence analysis can be carried out to identify the CDR boundaries. CDR sequences may be defined using the Kabat system or nomenclature (Kabat, E. A. et al., Sequences of Proteins of Immunological Interest, Fifth Edition, U.S. Department of Health and Human Services, U.S. Government Printing Office (1991)), the Chothia system or nomenclature (Chothia & Lesk, Canonical Structures for the Hypervariable Regions of Immunoglobulins, J. Mol. Biol. 196:901-917 (1987)), and/or the IMGT system or nomenclature (Dondelinger et al., Understanding the Significance and Implications of Antibody Numbering and Antigen-Binding Surface/Residue Definition, Front Immunol, 9:2278 (2018)). The heavy chain comprises HCDR1, HCDR2, and HCDR3. The light chain comprises LCDR1, LCDR2, and LCDR3. The secreted form of the IgH C region of most antibodies is made up of three C domains, CH1, CH2, CH3, and a hinge region, except for Cu, which includes a CH4 regions and lacks a hinge region. The membrane-bound form of the IgH C region also has membrane and intra-cellular domains. The IgH constant region determines the isotype of the antibody, e.g. IgM, IgD, IgG1, IgG2, IgG3, IgG4, IgA and IgE. It will be appreciated that non-human mammals, encoding multiple Ig isotypes will be able to undergo isotype class switching. There are two types of human IgL, Igκ and Igλ.

As used herein, the term “monoclonal antibody” or “mAb” refers to an antibody produced by an identical set of immune cells that is each a clone of a unique parent cell. Monoclonal antibodies have monovalent affinity (i.e., they bind to the same epitope).

The term “antigen-binding fragment” as used herein refers to a polypeptide fragment that contains at least one CDR of an immunoglobulin heavy and/or light chain that binds to amyloid beta. In this regard, an antigen-binding fragment of the antibodies may comprise 1, 2, 3, 4, 5, or all 6 CDRs of a VH and VL sequence set forth herein from anti-amyloid beta antibodies described herein. In some embodiments, the antigen-binding fragment of the anti-amyloid beta antibodies comprise all 6 CDRs of a VH and VL sequence set forth herein from an anti-amyloid beta antibody disclosed herein. An antigen-binding fragment of the amyloid beta-specific antibodies described herein is capable of binding to amyloid beta. In some embodiments, the anti-amyloid beta antibody or antigen binding fragment thereof binds to amyloid beta plaques, parenchymal amyloid, cerebrovascular amyloid, or diffuse amyloid beta deposits.

In some embodiments, antibodies and antigen-binding fragments thereof as described herein include a heavy chain and a light chain CDR set, respectively interposed between a heavy chain and a light chain framework region (FR) set that provide conformational support to the CDRs and define the spatial relationship of the CDRs relative to each other. As used herein, the term “CDR set” refers to the three hypervariable regions of a heavy or light chain V region or domain. Proceeding from the N terminus of a heavy or light chain, these regions are denoted as “CDR1,” “CDR2,” and “CDR3,” respectively. An antigen-binding site, therefore, includes six CDRs, comprising the CDR set from each of a heavy and a light chain V region or domain.

An “Fab” domain or fragment comprises the N-terminal portion of the IgH, which includes the V region and the CH1 domain of the IgH, and the entire IgL. An “F(ab′)₂” domain comprises the Fab domain and a portion of the hinge region, wherein the 2 IgH are linked together via disulfide linkage in the middle hinge region. Both the Fab and F(ab′)₂ are “antigen-binding fragments.”

The C-terminal portion of the IgH, which is the crystallizable fragment of an antibody following papain digestion and comprises the CH2 and CH3 domains, is referred to as the “Fc” domain. The Fc domain is the portion of the Ig recognized by cell receptors, such as the FcR, and to which the complement-activating protein, C1q, binds. The lower hinge region, which is encoded in the 5′ portion of the CH2 exon, provides flexibility within the antibody for binding to FcR receptors. Although the boundaries of the Fc domain may vary, the human IgG heavy chain Fc domain, as defined herein, comprises residue E216 to its carboxyl-terminus of the CH3 domain (or the CH4 domain for IgM and IgE antibodies), wherein the numbering is in the EU format as set forth in Edelman. The term “Fc domain” may refer to this sequence in isolation, or this sequence in the context of an antibody, antibody fragment, or Fc fusion protein. The amino acid sequence of a non-naturally occurring Fc domain (also referred to herein as a “variant Fc domain”) may comprise one or more amino acid modifications. Polymorphisms have been observed at a number of Fc domain positions, including but not limited to positions 270, 272, 312, 315, 356, and 358, and thus slight differences between the presented sequence and sequences in the prior art may exist.

The term “EU format as set forth in Edelman” refers to the residue numbering of the human IgG1 EU antibody as described in Edelman G M et al., (1969) Proc. Natl. Acad. USA, 63, 78-85. The human IgG2 and human IgG4 residue numbering is also in the EU format (See Dillon T M, et al., J Biol Chem. June 6; 283 (23): 16206-15 (2008); Aalberse R C et al., Immunology 105:9-19 (2002); and Scholthauer T et al., Protein Engineering, Design and Selection, 29 (10): 457-466, (2016). The EU numbering of residues may be determined by aligning an antibody at regions of homology to the sequence of the antibody with a “standard” EU numbered sequence.

An “Fv” fragment includes a non-covalent VH::VL heterodimer including an antigen-binding site. In certain embodiments, single chain Fv (scFv) antibodies are contemplated. A scFv is a covalently linked VH::VL heterodimer which is expressed from a gene fusion including VH- and VL-encoding genes linked by a peptide-encoding linker (see, e.g., Huston et al. (1988) Proc. Nat. Acad. Sci. USA 85 (16): 5879-5883, incorporated herein by reference). As used herein, the term “linker” refers to a polypeptide sequence that joins two or more antibody domains. Linkers' characteristics and their suitability for particular purposes are known in the art. See, e.g., Chen et al. Adv Drug Deliv Rev. October 15; 65 (10): 1357-1369 (2013) (disclosing various types of linkers, their properties, and associated linker designing tools and databases), which is incorporated herein by reference. Linkers may be flexible, rigid, or in vivo cleavable. Preferably, the linker is flexible. Flexible linkers typically comprise small non-polar (e.g. Gly) or polar (e.g., Ser or Thr) amino acids. The most commonly used flexible linkers have sequences consisting primarily of stretches of Gly and Ser residues (“GS” linker). Optionally, flexible linkers comprise repeats of 5 Gly and Ser residues.

Where bispecific antibodies are to be used, these may be conventional bispecific antibodies, which can be manufactured in a variety of ways (see, e.g., Holliger, P. and Winter G. Current Opinion Biotechnol. 4, 446-449 (1993)), e.g., prepared chemically or from hybrid hybridomas, or may be any of the bispecific antibody fragments mentioned above.

As used herein, the term “chimeric antibody” refers to an antibody encoded by a polynucleotide sequence containing polynucleotide sequences from two or more species, e.g., human and mouse. A chimeric antibody, as used herein, may also refer to an antibody that comprises regions from two or more different antibodies.

As used herein, the term “chimeric Ig chain” refers to an Ig heavy chain or an Ig light chain encoded by a polynucleotide sequence containing polynucleotide sequences from two or more species, e.g., human and mouse. For example, a chimeric Ig heavy chain may comprise a human VH domain, DH domain, JH domain, CH1 domain, and upper hinge region and mouse CH2 and CH3 domains. In some embodiments, the middle hinge region is mouse. In some embodiments, the middle hinge region is human. In some embodiments, the middle hinge region is chimeric.

As used herein, the term “human antibody” refers to an antibody having variable and constant regions derived from human germline immunoglobulin sequences. Human antibodies can include amino acid residues not encoded by human germline immunoglobulin sequences (e.g., mutations introduced by random or site-specific mutagenesis in vitro or by somatic mutation in vivo). The term “human antibody,” however, does not encompass antibodies in which the CDR sequences are derived from the germline of another mammalian species, such as a mouse, and have been grafted onto human framework sequences (i.e., humanized antibodies). The term encompasses antibodies with sequences derived from human genes, but which have been changed, e.g. to decrease possible immunogenicity, increase affinity, decrease effector function eliminate cysteines that might cause undesirable folding, etc. The term also encompasses such antibodies comprising human amino acid sequences produced recombinantly in non-human cells, which might impart a glycosylation pattern that is not typical of human cells.

As used herein, the terms “polypeptide,” “peptide” or “protein” are used interchangeably herein to describe a chain of amino acids that are linked together by chemical bonds. Non-limiting examples of a polypeptide or protein include an IgH, IgL, V domain, C domain, or an antibody.

As used herein, the term “amino acid modification” refers to at least one amino acid substitution, insertion, deletion or mutation in an amino acid sequence compared to a wild-type amino acid sequence. Such modifications are within the ordinary skill of an artisan. Some modifications, including amino acid deletions, substitutions and additions, of the Fc region have been shown to alter the Fc domain's binding to its ligands and/or receptors resulting in a concomitant modification of effector function (see, e.g., Shields et al., J Biol Chem 276:6591-6604 (2001); Presta et al., Biochem Soc Trans 30:487-490 (2002); Escobar-Cabrera E et al. Antibodies. 6:7 (2017); Duncan A R et al. Nature. 1988; 332:738-740 (1988); Duncan A R et al. Nature. 332:563-564 (1988); Hezareh M et al. J Virol. 75:12161-12168 (2001); Oganesyan V et al. Acta Crystallogr D Biol Crystallogr; 64:700-704 (2008); Schlothauer T et al. Protein Eng Des Sel. October; 29 (10): 457-466 (2016); Tao M H et al. J. Immunol. 143:2595-2601 (1989); Von Kreudenstein T S et al. MAbs. 5 (5): 646-654 (2013); Wang X et al. Protein Cell. 9 (1): 63-73 (2018); U.S. Patent Publications 20040132101; 20070111260; 20110287032; 20180194860; U.S. Patent No. U.S. Pat. No. 8,409,568; International Publication No. WO2017/177337, each incorporated by reference in its entirety). An amino acid deletion is indicated with a “Δ”, and an insertion is indicated with a “In”. For example, a deletion of the amino acid sequence from E216 to E222 is indicated as ΔE216-E222. An insertion of an arginine (R) between amino acid residues 234 and 235, e.g., would be indicated as InR234/235.

A “conservative amino acid substitution” replaces an amino acid residue with a different amino acid residue having similar biochemical properties (e.g., charge, hydrophobicity, or size). Generally, conservative amino acid substitutions do not substantially change the functional properties of a protein. When comparing proteins comprising conservative substitutions, the percent sequence identity or degree of similarity may be adjusted to account for the conservative nature of the substitution. Such adjustments are well-known in the art. See, e.g., Pearson, Methods Mol. Biol. 243:307-31 (1994).

The term “percent sequence identity” in the context of polypeptide (or polynucleotide) sequences is defined as the percentage of amino acid (or nucleic acid) residues in a candidate sequence that are identical with the amino acids (or nucleic acid residues) in the reference polypeptide (or polynucleotide) sequence, after aligning the sequences and introducing gaps, if necessary, to achieve the maximum percent sequence identity, and not considering any conservative substitutions as part of the sequence identity. Such conservative substitutions are considered (in addition to identical residues) in calculating the “percent sequence similarity” of two sequences. Residue positions that are not identical but are similar differ by conservative amino acid substitutions.

Sequence alignments (e.g. for determining percent amino acid sequence identity, sequence similarity or sequence homology, such as between a wild type protein and a mutein thereof) can be achieved in various ways that are within the skill in the art, for instance, using publicly available sequence analysis computer software such as BLAST, BLAST-2, ALIGN, Megalign (DNASTAR), Gap, BESTFIT®, and other programs in programs in Wisconsin Package Version 10.0 or Genetics Computer Group (GCG), Madison, Wisconsin, software. The skilled artisan can determine appropriate parameters for aligning sequences, including any algorithms needed to achieve maximal alignment over the full length of the sequences being compared. Polypeptide sequences also can be compared using FASTA using default or recommended parameters. In the context of polypeptide sequences, FASTA takes the query amino acid sequence and searches a sequence database using local sequence alignment to identify similar sequences within the database (Pearson, Methods Enzymol. 183:63-98 (1990); Pearson, Methods Mol. Biol. 132:185-219 (2000); Pearson Curr Protoc Bioinformatics. March 24; 53:3.9.1-25 (2016); each herein incorporated by reference). BLAST, especially blastp or tblastn, using default parameters may be used to compare a query sequence to a database containing sequences from different organisms. See, e.g., Altschul et al., J. Mol. Biol. 215:403-410 (1990); Altschul et al., Nucleic Acids Res. 25:3389-402 (1997); Eser et al., PLOS One. 22; 9 (12): e115445 (2014), each herein incorporated by reference.

Amino acids may be grouped based on their similar biochemical properties. Such groupings that may be used to define conservative substitutions include 1) amino acid residues with aliphatic side chains: glycine, alanine, valine, leucine, and isoleucine; 2) amino acid residues with aliphatic-hydroxyl side chains: serine and threonine; 3) amino acid residues with amide-containing side chains: asparagine and glutamine; 4) amino acid residues with aromatic side chains: phenylalanine, tyrosine, and tryptophan; 5) amino acid residues with basic side chains: lysine, arginine, and histidine; 6) amino acid residues with acidic side chains: aspartic acid and glutamic acid; and 7) amino acid residues with sulfur-containing side chains: cysteine and methionine. Non-limiting examples of preferred conservative amino acids substitution groups include: valine-leucine-isoleucine, phenylalanine-tyrosine, lysine-arginine, alanine-valine, glutamate-aspartate, and asparagine-glutamine.

The strength, or affinity of immunological binding interactions can be expressed in terms of the dissociation constant (K_{D}) of the interaction, wherein a smaller K_{D} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant, K_{D}. See, generally, Davies et al. (1990) Annual Rev. Biochem. 59:439-473. An antibody is considered to specifically bind an antigen when the K_{D} is ≤1 mM, preferably ≤100 nM. High affinity antibodies generally have a K_{D} in the low nanomolar (10⁻⁹) range, and very high affinity antibodies generally have a K_{D} in picomolar (10⁻¹²) range. A K_{D} binding affinity constant can be measured by surface plasmon resonance, e.g. using the BIACORE® system (Pharmacia Biosensor AB, Uppsala, Sweden and Piscataway, N.J.). See also, Jonsson et al., Ann. Biol. Clin. 51:19-26 (1993); Jonsson et al., Biotechniques 11:620-627 (1991); Jonsson et al., J. Mol. Recognit. 8:125-131 (1995); Johnsson et al., Anal. Biochem. 198:268-277 (1991); Hearty S et al., Methods Mol Biol. 907:411-42 (2012), each incorporated herein by reference. The K_{D} may also be measured using a KINEXA® system (Sapidyne Instruments, Hanover, Germany and Boise, ID). An antibody, or antigen-binding fragment thereof, is said to “specifically bind,” “immunologically bind,” and/or is “immunologically reactive” to amyloid beta if it reacts at a detectable level (within, for example, an ELISA assay) with amyloid beta, and does not react detectably with unrelated polypeptides under similar conditions. In some embodiments, the anti-amyloid beta antibody or antigen binding fragment thereof binds to amyloid beta plaques, parenchymal amyloid, cerebrovascular amyloid, or diffuse amyloid beta deposits.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salt” refers to a salt of a compound of the invention that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like. The term “pharmaceutically acceptable cation” refers to an acceptable cationic counter-ion of an acidic functional group. Such cations are exemplified by sodium, potassium, calcium, magnesium, ammonium, tetraalkylammonium cations, and the like. See, e.g., Berge, et al., J. Pharm. Sci. (1977) 66 (1): 1-79.

The term “administering,” as used herein, refers to any mode of transferring, delivering, introducing, or transporting a pharmaceutical composition or other agent as described herein, to a subject. Such modes include, but are not limited to, oral administration, inhalation, topical contact, intravenous, intraperitoneal, intramuscular, intranasal, or subcutaneous administration.

As used herein, the term “unit dosage form” is defined to refer to the form in which a compound as disclosed herein is administered to the subject.

As used herein, the term “daily” means a given, continuous twenty-four (24) hour period.

The terms “subject,” “patient” and “individual” are used interchangeably herein and include, but are not limited to, humans and a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human (“human subject”). In certain embodiments, the human subject is an infant, child, or adolescent (“pediatric subject”). In other embodiments, the human subject is a young adult, middle-aged adult or senior adult (“adult subject”). In certain embodiments, the subject is a non-human animal (“non-human subject”).

“Disease,” “disorder,” “condition,” and “illness” are used interchangeably herein. In some embodiments, the disease is Alzheimer's disease. In some embodiments, the disease is cognitive impairment associated with Alzheimer's disease.

As used herein, and unless otherwise specified, the terms “treat,” “treating” and “treatment” refer to an action that occurs while a subject is suffering from the specified disease, disorder, or condition, which reduces the severity of the disease, disorder, or condition, or retards or slows the progression of the disease, disorder, or condition (also, “therapeutic treatment”). The terms refer to improving at least one symptom of the subject's disease or disorder. Treating includes curing, improving, or at least partially ameliorating the disease or disorder or any symptom of the disease or disorder.

As used herein, the terms “prevent,” “preventing,” and “prevention” refer to the prevention or delay of the recurrence or onset of, or a reduction in one or more symptoms of a coronaviral infection in a subject as a result of the administration of the composition of the disclosure. For example, in the context of the administration of a therapy to a subject, “prevent,” “preventing,” and “prevention” refer to the inhibition, reduction, delay in the development, the prevention or delay of the recurrence, onset, or development of one or more symptoms associated with Alzheimer's disease.

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, to delay or minimize one or more symptoms associated with the disease, disorder or condition, or to improve cognitive function. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

A “prophylactically effective amount” refers to an amount effective, at dosages and for periods of time necessary, to achieve the desired prophylactic result. Typically, since a prophylactic dose is used in subjects prior to the disease or at an early stage of the disease, the prophylactically effective amount may be less than the therapeutically effective amount.

In some embodiments, administering a compound as described herein, or a pharmaceutically acceptable salt thereof, improves cognitive function. In some embodiments, the cognitive function refers to a collection of mental tasks and functions, including but not limited to: psychomotor performance; executive performance; emotional recognition; learning (e.g, visual, spatial); memory (e.g., semantic, episodic, procedural, priming, or working); orientation; language; problem solving; visual perception, construction, and integration; planning; organizational skills; selective attention; inhibitory control; and ability to mentally manipulate information. In some embodiments, the cognitive function is one or more selected from the group consisting of psychomotor performance; executive performance; emotional recognition; learning (e.g, visual, spatial); memory (e.g., semantic, episodic, procedural, priming, or working); orientation; language; problem solving; visual perception, construction, and integration; planning; organizational skills; selective attention; inhibitory control; and ability to mentally manipulate information. In some embodiments, the compounds disclosed herein selectively target working memory, and the improvement thereof. In some embodiments the compounds disclosed herein selectively target executive performance, and the improvement thereof. Measures of cognitive function include assessment tools designed to measure, for example: (a) general intelligence, (b) nonverbal intelligence, (c) achievement, (d) attention/executive functioning, (e) memory and learning, (f) visual-motor and motor functioning and (g) language.

Any change in cognitive function, for example, over time or through treatment, can be monitored by using one or more of these well-established tests at two or more time points and comparing the results. The phrase “improves cognitive function”, as referred to herein, means a positive change in the ability of the subject to perform a symbolic operation, for example, to perceive, remember, create a mental image, have clarity of thought, be aware, to reason, think or judge. The positive change can be measured using any of the aforementioned tests on two or more occasions, for example, a first occasion to measure baseline cognitive function and a second occasion to measure cognitive function following a period of time (in which treatment may have been administered). Such assessment tools are well-known in the art and include, for example, those assessment tools as described in Example 1 herein.

The term “memory” as used herein is defined as the biological processes of the brain that enable storage and recall of information.

The term “working memory” as used herein is defined as a combination of processes of the brain that provide temporary storage and manipulation of information necessary to perform complex cognitive tasks such as learning and reasoning.

The term “learning” as used herein refers to processes of the brain involved in the acquisition of skill, knowledge and information.

The term “executive function” or “executive performance” as used interchangeably herein, refers to an umbrella term for cognitive processes that regulate, control and manage other cognitive processes, including planning, working memory, attention, problem solving, verbal reasoning, inhibition, mental flexibility, task switching, and initiation and monitoring of actions. The prefrontal areas of the frontal lobe are necessary but not sufficient for carrying out these functions.

The term “improve,” “improving”, or “improvement” or grammatical variations thereof used in relation to working memory or executive function refer to the ability to achieve a measurable increase in performance in relation to tasks used to test working memory or executive function in subjects.

As used herein, the terms “pharmaceutically acceptable carrier” and “pharmaceutically acceptable excipient” are used interchangeably and refer to any and all solvents, dispersion media, coatings, antibacterial and antifungal agents, isotonic and absorption delaying agents, and the like that are physiologically compatible. Pharmaceutically acceptable carriers are well known in the art. See, e.g., Remington's Pharmaceutical Sciences and U.S. Pharmacopeia: National Formulary, Mack Publishing Company, Easton, PA (1984), incorporated herein by reference. Some examples of pharmaceutically acceptable carriers are water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Additional examples of pharmaceutically acceptable substances are wetting agents or minor amounts of auxiliary substances such as wetting or emulsifying agents, preservatives or buffers, which enhance the shelf life or effectiveness of the antibody. Pharmaceutical compositions may be prepared by mixing a compound, antibody or antigen-binding fragment thereof disclosed herein with acceptable carriers, excipients, or stabilizers in the form of, e.g., lyophilized powders, slurries, aqueous solutions or suspensions (see, e.g., Hardman, et al. (2001) Goodman and Gilman's The Pharmacological Basis of Therapeutics, McGraw-Hill, New York, NY; Gennaro (2000) Remington: The Science and Practice of Pharmacy, Lippincott, Williams, and Wilkins, New York, NY; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, NY; each incorporated herein by reference).

### General

Disclosed herein are pharmaceutical compositions, kits and methods of treatment comprising (a) an NMDA receptor positive allosteric modulator (NMDA PAM); (b) an anti-amyloid beta antibody or antigen-binding fragment thereof; and (c) a pharmaceutically acceptable carrier. Also disclosed herein are pharmaceutical compositions, kits and methods of treatment comprising (a) a CYP46A1 inhibitor; (b) an anti-amyloid beta antibody or antigen-binding fragment thereof; and (c) a pharmaceutically acceptable carrier.

### CYP46A1 Inhibitors

A CYP46A1 inhibitor useful in the compositions, kits and methods of the present disclosure is a compound that inhibits the enzyme cholesterol 24-hydroxylase (also known as CYP46A1 and CH24H), which converts cholesterol into 24S-hydroxycholesterol (24-HC) and exhibits a beneficial effect for treating or preventing Alzheimer's disease as disclosed herein when administered in combination with an anti-amyloid beta antibody or antigen-binding fragment thereof. CYP46A1 inhibitors are known to those skilled in the art. Therefore, any CYP46A1 inhibitor may be useful in the compositions, kits and methods of the present disclosure.

In some embodiments, the CYP46A1 inhibitor is any compound disclosed in WO2020/243027, WO2013/054822, U.S. Pat. No. 9,193,709, WO2014/092100, U.S. Pat. No. 9,643,957, WO2014/163162, U.S. Pat. No. 9,624,184, WO2014/061676, U.S. Pat. No. 9,296,746, WO2017/065287, the disclosures of each of which are incorporated herein by reference in their entirety.

In some embodiments, the CYP46A1 inhibitor is selected from the group consisting of TAK-935, and any of compounds A1-A182, and pharmaceutically acceptable salts thereof. In some embodiments, the CYP46A1 inhibitor is any one of compounds A1-A182 (see Table 1) or a pharmaceutically acceptable salt thereof.

TABLE 1

| No | Structure |
|---|---|
| A1 | 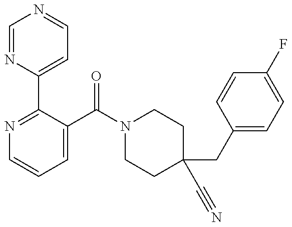 [SMILES: N#CC1(Cc2ccc(F)cc2)CCN(C(=O)c2cccnc2-c2ccncn2)CC1] |
| A2 | 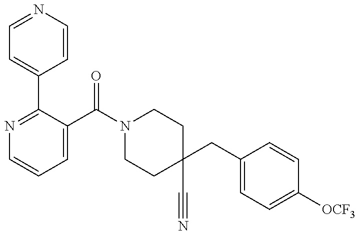 [SMILES: N#CC1(Cc2ccc(OC(F)(F)F)cc2)CCN(C(=O)c2cccnc2-c2ccncc2)CC1] |
| A3 | 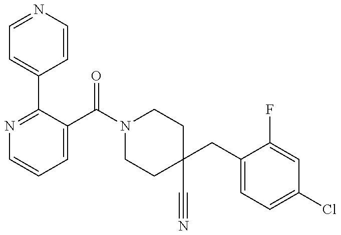 [SMILES: N#CC1(Cc2ccc(Cl)cc2F)CCN(C(=O)c2cccnc2-c2ccncc2)CC1] |
| A4 | 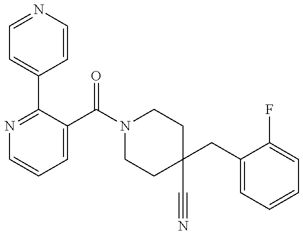 [SMILES: N#CC1(Cc2ccccc2F)CCN(C(=O)c2cccnc2-c2ccncc2)CC1] |
| A5 | 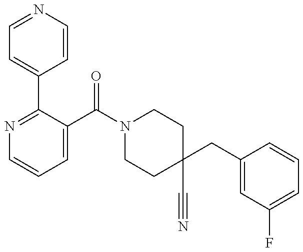 [SMILES: N#CC1(Cc2cccc(F)c2)CCN(C(=O)c2cccnc2-c2ccncc2)CC1] |
| A6 | 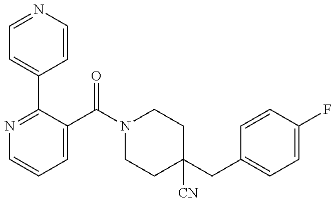 [SMILES: N#CC1(Cc2ccc(F)cc2)CCN(C(=O)c2cccnc2-c2ccncc2)CC1] |
| A7 | 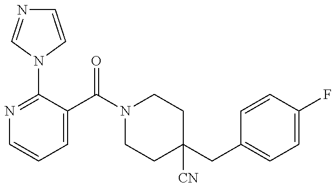 [SMILES: N#CC1(Cc2ccc(F)cc2)CCN(C(=O)c2cccnc2-n2ccnc2)CC1] |
| A8 | 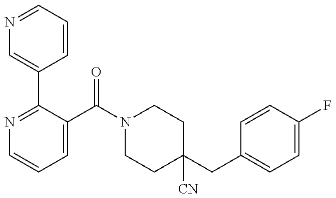 [SMILES: N#CC1(Cc2ccc(F)cc2)CCN(C(=O)c2cccnc2-c2cccnc2)CC1] |
| A9 | 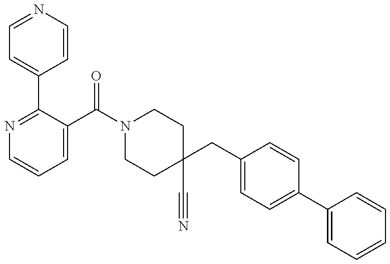 [SMILES: N#CC1(Cc2ccc(-c3ccccc3)cc2)CCN(C(=O)c2cccnc2-c2ccncc2)CC1] |
| A10 | 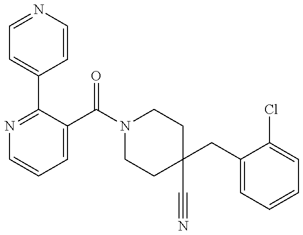 [SMILES: N#CC1(Cc2ccccc2Cl)CCN(C(=O)c2cccnc2-c2ccncc2)CC1] |
| A11 | 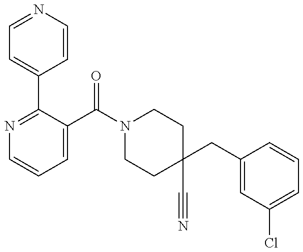 [SMILES: N#CC1(Cc2cccc(Cl)c2)CCN(C(=O)c2cccnc2-c2ccncc2)CC1] |
| A12 | 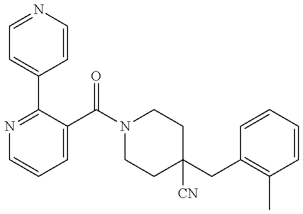 [SMILES: Cc1ccccc1CC1(C#N)CCN(C(=O)c2cccnc2-c2ccncc2)CC1] |
| A13 | 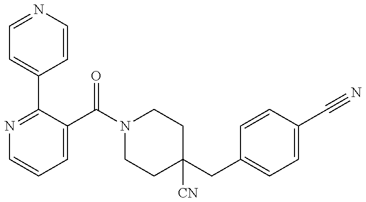 [SMILES: N#Cc1ccc(CC2(C#N)CCN(C(=O)c3cccnc3-c3ccncc3)CC2)cc1] |
| A14 | 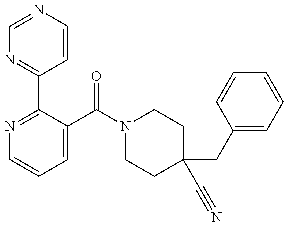 [SMILES: N#CC1(Cc2ccccc2)CCN(C(=O)c2cccnc2-c2ccncn2)CC1] |
| A15 | 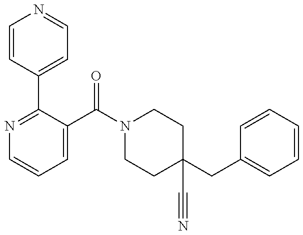 [SMILES: N#CC1(Cc2ccccc2)CCN(C(=O)c2cccnc2-c2ccncc2)CC1] |
| A16 | 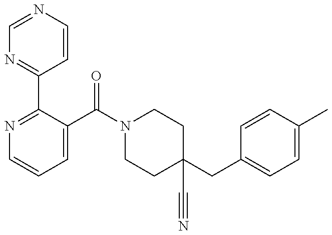 [SMILES: Cc1ccc(CC2(C#N)CCN(C(=O)c3cccnc3-c3ccncn3)CC2)cc1] |
| A17 | 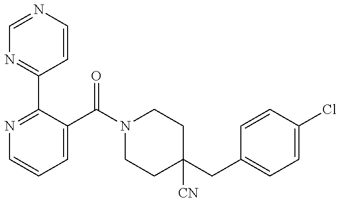 [SMILES: N#CC1(Cc2ccc(Cl)cc2)CCN(C(=O)c2cccnc2-c2ccncn2)CC1] |
| A18 | 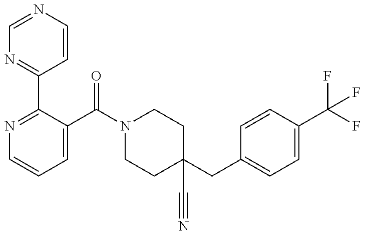 [SMILES: N#CC1(Cc2ccc(C(F)(F)F)cc2)CCN(C(=O)c2cccnc2-c2ccncn2)CC1] |
| A19 | 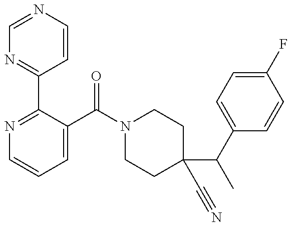 [SMILES: CC(c1ccc(F)cc1)C1(C#N)CCN(C(=O)c2cccnc2-c2ccncn2)CC1] |
| A20 | 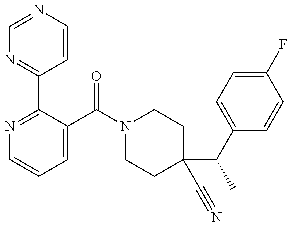 [SMILES: C[C@@H](c1ccc(F)cc1)C1(C#N)CCN(C(=O)c2cccnc2-c2ccncn2)CC1] |
| A21 | 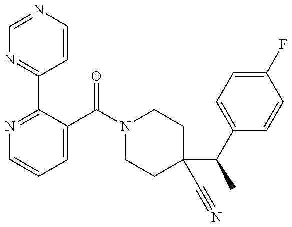 [SMILES: C[C@H](c1ccc(F)cc1)C1(C#N)CCN(C(=O)c2cccnc2-c2ccncn2)CC1] |
| A22 | 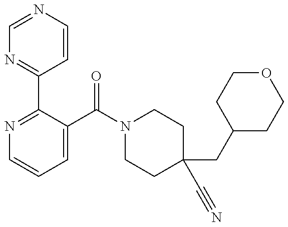 [SMILES: N#CC1(CC2CCOCC2)CCN(C(=O)c2cccnc2-c2ccncn2)CC1] |
| A23 | 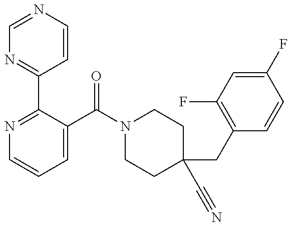 [SMILES: N#CC1(Cc2ccc(F)cc2F)CCN(C(=O)c2cccnc2-c2ccncn2)CC1] |
| A24 | 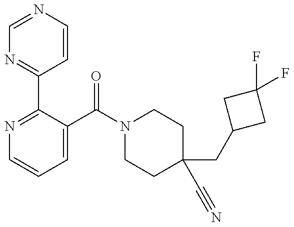 [SMILES: N#CC1(CC2CC(F)(F)C2)CCN(C(=O)c2cccnc2-c2ccncn2)CC1] |
| A25 | 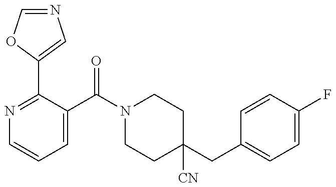 [SMILES: N#CC1(Cc2ccc(F)cc2)CCN(C(=O)c2cccnc2-c2cnco2)CC1] |
| A26 | 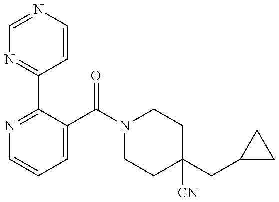 [SMILES: N#CC1(CC2CC2)CCN(C(=O)c2cccnc2-c2ccncn2)CC1] |
| A27 | 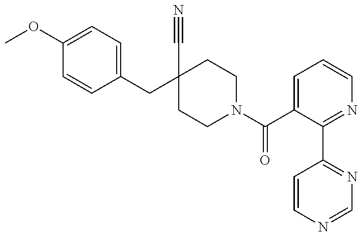 [SMILES: COc1ccc(CC2(C#N)CCN(C(=O)c3cccnc3-c3ccncn3)CC2)cc1] |
| A28 | 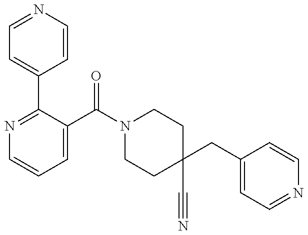 [SMILES: N#CC1(Cc2ccncc2)CCN(C(=O)c2cccnc2-c2ccncc2)CC1] |
| A29 | 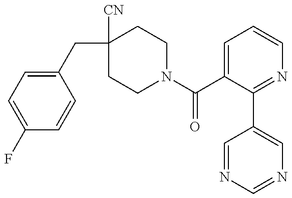 [SMILES: N#CC1(Cc2ccc(F)cc2)CCN(C(=O)c2cccnc2-c2cncnc2)CC1] |
| A30 | 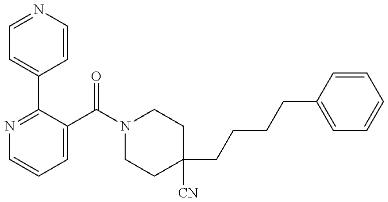 [SMILES: N#CC1(CCCCc2ccccc2)CCN(C(=O)c2cccnc2-c2ccncc2)CC1] |
| A31 | 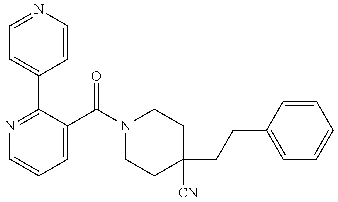 [SMILES: N#CC1(CCc2ccccc2)CCN(C(=O)c2cccnc2-c2ccncc2)CC1] |
| A32 | 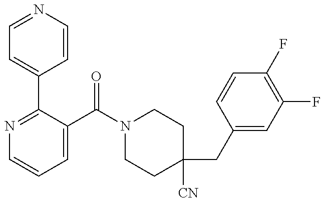 [SMILES: N#CC1(Cc2ccc(F)c(F)c2)CCN(C(=O)c2cccnc2-c2ccncc2)CC1] |
| A33 | 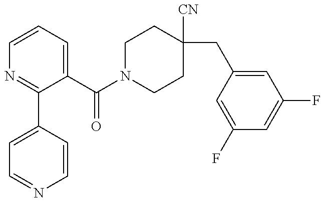 [SMILES: N#CC1(Cc2cc(F)cc(F)c2)CCN(C(=O)c2cccnc2-c2ccncc2)CC1] |
| A34 | 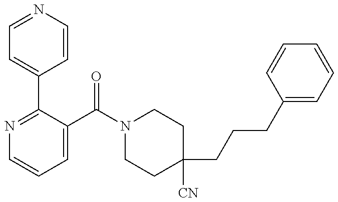 [SMILES: N#CC1(CCCc2ccccc2)CCN(C(=O)c2cccnc2-c2ccncc2)CC1] |
| A35 | 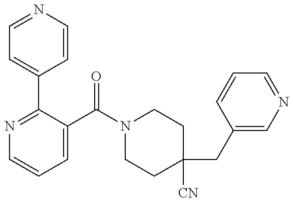 [SMILES: N#CC1(Cc2cccnc2)CCN(C(=O)c2cccnc2-c2ccncc2)CC1] |
| A36 | 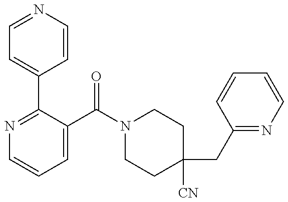 [SMILES: N#CC1(Cc2ccccn2)CCN(C(=O)c2cccnc2-c2ccncc2)CC1] |
| A37 | 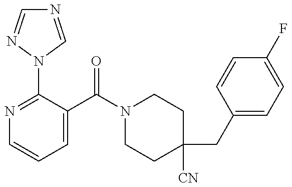 [SMILES: N#CC1(Cc2ccc(F)cc2)CCN(C(=O)c2cccnc2-n2cncn2)CC1] |
| A38 | 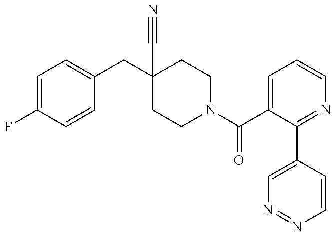 [SMILES: N#CC1(Cc2ccc(F)cc2)CCN(C(=O)c2cccnc2-c2ccnnc2)CC1] |
| A39 | 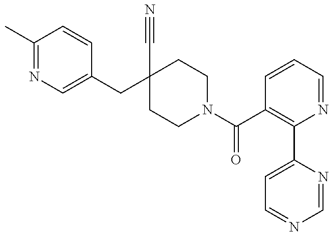 [SMILES: Cc1ccc(CC2(C#N)CCN(C(=O)c3cccnc3-c3ccncn3)CC2)cn1] |
| A40 | 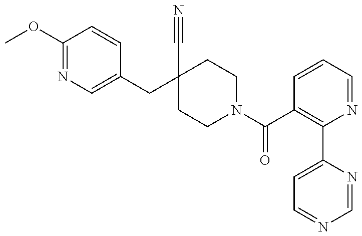 [SMILES: COc1ccc(CC2(C#N)CCN(C(=O)c3cccnc3-c3ccncn3)CC2)cn1] |
| A41 | 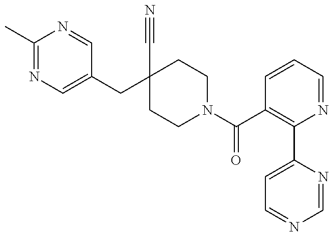 [SMILES: Cc1ncc(CC2(C#N)CCN(C(=O)c3cccnc3-c3ccncn3)CC2)cn1] |
| A42 | 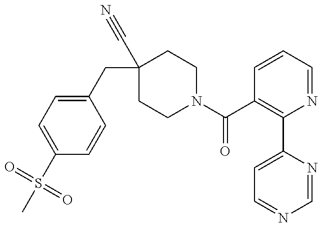 [SMILES: CS(=O)(=O)c1ccc(CC2(C#N)CCN(C(=O)c3cccnc3-c3ccncn3)CC2)cc1] |
| A43 | 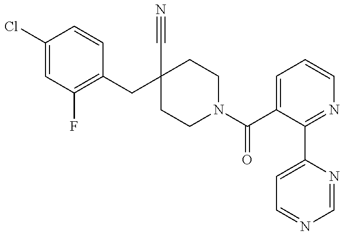 [SMILES: N#CC1(Cc2ccc(Cl)cc2F)CCN(C(=O)c2cccnc2-c2ccncn2)CC1] |
| A44 | 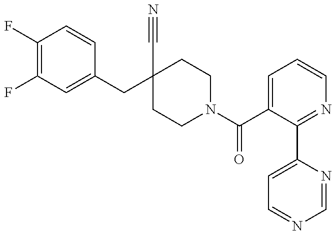 [SMILES: N#CC1(Cc2ccc(F)c(F)c2)CCN(C(=O)c2cccnc2-c2ccncn2)CC1] |
| A45 | 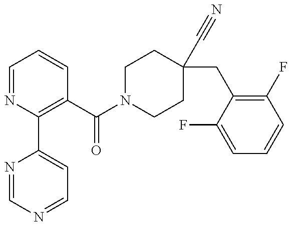 [SMILES: N#CC1(Cc2c(F)cccc2F)CCN(C(=O)c2cccnc2-c2ccncn2)CC1] |
| A46 | 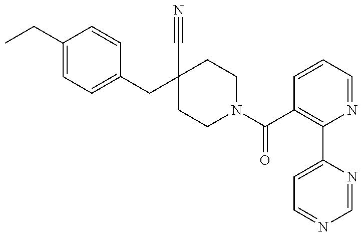 [SMILES: CCc1ccc(CC2(C#N)CCN(C(=O)c3cccnc3-c3ccncn3)CC2)cc1] |
| A47 | 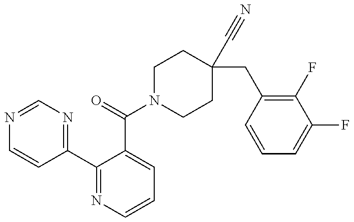 [SMILES: N#CC1(Cc2cccc(F)c2F)CCN(C(=O)c2cccnc2-c2ccncn2)CC1] |
| A48 | 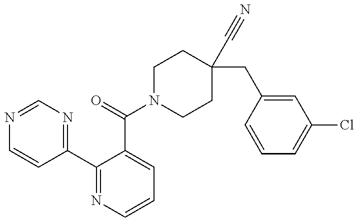 [SMILES: N#CC1(Cc2cccc(Cl)c2)CCN(C(=O)c2cccnc2-c2ccncn2)CC1] |
| A49 | 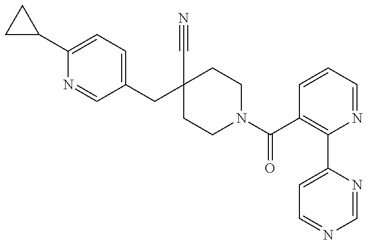 [SMILES: N#CC1(Cc2ccc(C3CC3)nc2)CCN(C(=O)c2cccnc2-c2ccncn2)CC1] |
| A50 | 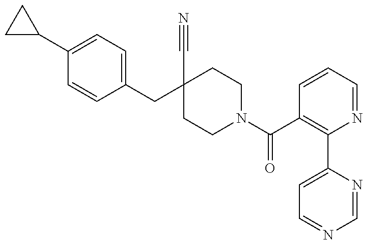 [SMILES: N#CC1(Cc2ccc(C3CC3)cc2)CCN(C(=O)c2cccnc2-c2ccncn2)CC1] |
| A51 | 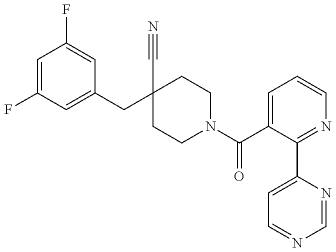 [SMILES: N#CC1(Cc2cc(F)cc(F)c2)CCN(C(=O)c2cccnc2-c2ccncn2)CC1] |
| A52 | 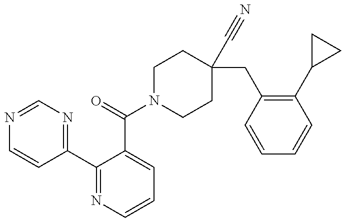 [SMILES: N#CC1(Cc2ccccc2C2CC2)CCN(C(=O)c2cccnc2-c2ccncn2)CC1] |
| A53 | 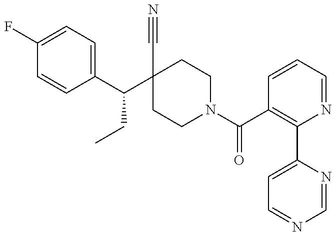 [SMILES: CC[C@H](c1ccc(F)cc1)C1(C#N)CCN(C(=O)c2cccnc2-c2ccncn2)CC1] |
| A54 | 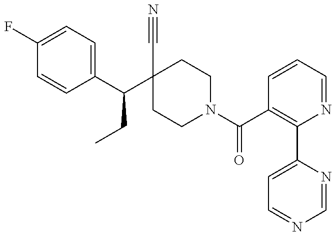 [SMILES: CC[C@@H](c1ccc(F)cc1)C1(C#N)CCN(C(=O)c2cccnc2-c2ccncn2)CC1] |
| A55 | 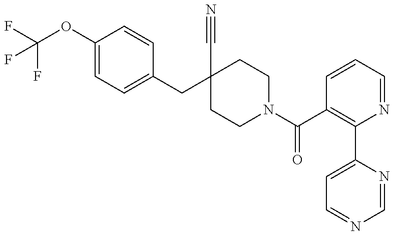 [SMILES: N#CC1(Cc2ccc(OC(F)(F)F)cc2)CCN(C(=O)c2cccnc2-c2ccncn2)CC1] |
| A56 | 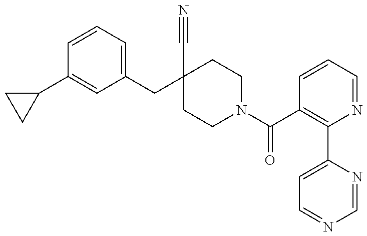 [SMILES: N#CC1(Cc2cccc(C3CC3)c2)CCN(C(=O)c2cccnc2-c2ccncn2)CC1] |
| A57 | 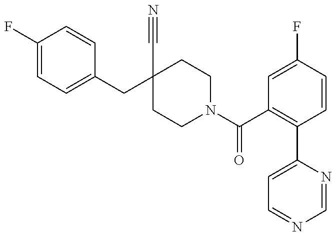 [SMILES: N#CC1(Cc2ccc(F)cc2)CCN(C(=O)c2cc(F)ccc2-c2ccncn2)CC1] |
| A58 | 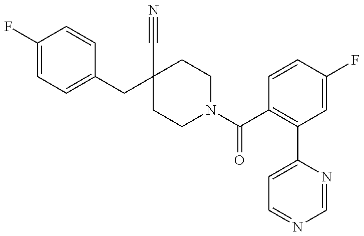 [SMILES: N#CC1(Cc2ccc(F)cc2)CCN(C(=O)c2ccc(F)cc2-c2ccncn2)CC1] |
| A59 | 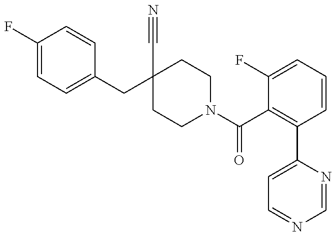 [SMILES: N#CC1(Cc2ccc(F)cc2)CCN(C(=O)c2c(F)cccc2-c2ccncn2)CC1] |
| A60 | 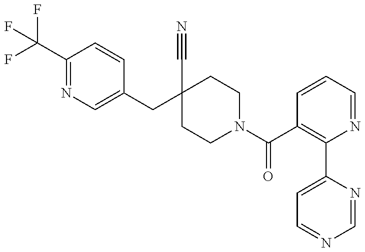 [SMILES: N#CC1(Cc2ccc(C(F)(F)F)nc2)CCN(C(=O)c2cccnc2-c2ccncn2)CC1] |
| A61 | 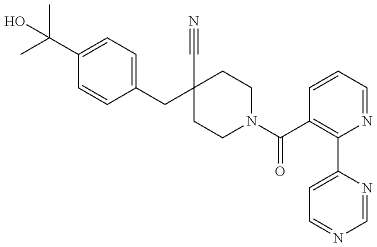 [SMILES: CC(C)(O)c1ccc(CC2(C#N)CCN(C(=O)c3cccnc3-c3ccncn3)CC2)cc1] |
| A62 | 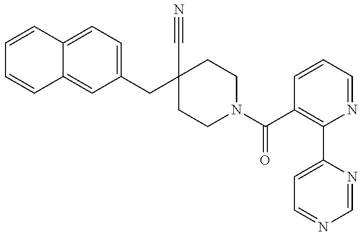 [SMILES: N#CC1(Cc2ccc3ccccc3c2)CCN(C(=O)c2cccnc2-c2ccncn2)CC1] |
| A63 | 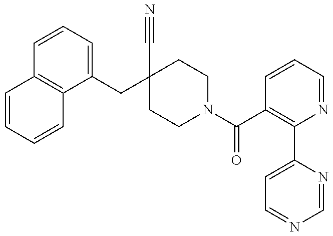 [SMILES: N#CC1(Cc2cccc3ccccc23)CCN(C(=O)c2cccnc2-c2ccncn2)CC1] |
| A64 | 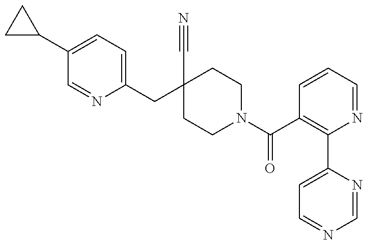 [SMILES: N#CC1(Cc2ccc(C3CC3)cn2)CCN(C(=O)c2cccnc2-c2ccncn2)CC1] |
| A65 | 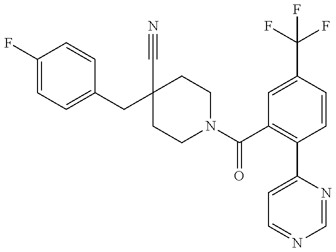 [SMILES: N#CC1(Cc2ccc(F)cc2)CCN(C(=O)c2cc(C(F)(F)F)ccc2-c2ccncn2)CC1] |
| A66 | 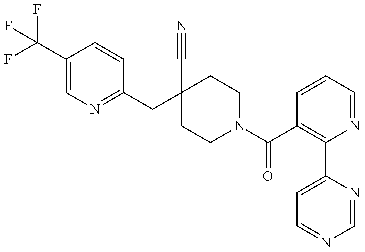 [SMILES: N#CC1(Cc2ccc(C(F)(F)F)cn2)CCN(C(=O)c2cccnc2-c2ccncn2)CC1] |
| A67 | 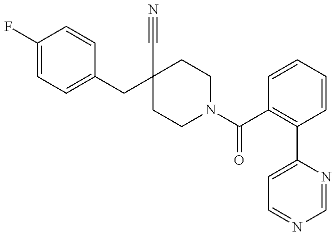 [SMILES: N#CC1(Cc2ccc(F)cc2)CCN(C(=O)c2ccccc2-c2ccncn2)CC1] |
| A68 | 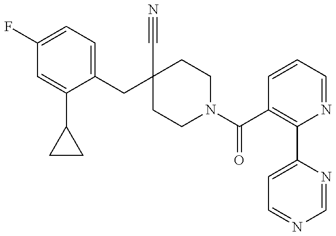 [SMILES: N#CC1(Cc2ccc(F)cc2C2CC2)CCN(C(=O)c2cccnc2-c2ccncn2)CC1] |
| A69 | 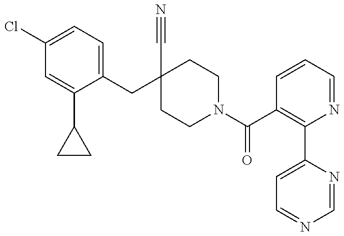 [SMILES: N#CC1(Cc2ccc(Cl)cc2C2CC2)CCN(C(=O)c2cccnc2-c2ccncn2)CC1] |
| A70 | 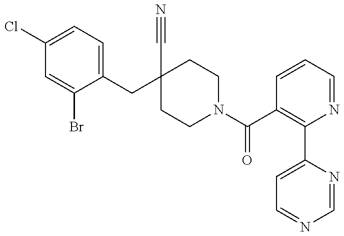 [SMILES: N#CC1(Cc2ccc(Cl)cc2Br)CCN(C(=O)c2cccnc2-c2ccncn2)CC1] |
| A71 | 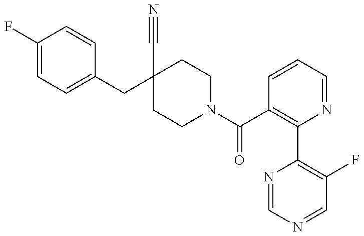 [SMILES: N#CC1(Cc2ccc(F)cc2)CCN(C(=O)c2cccnc2-c2ncncc2F)CC1] |
| A72 | 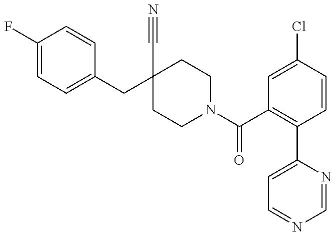 [SMILES: N#CC1(Cc2ccc(F)cc2)CCN(C(=O)c2cc(Cl)ccc2-c2ccncn2)CC1] |
| A73 | 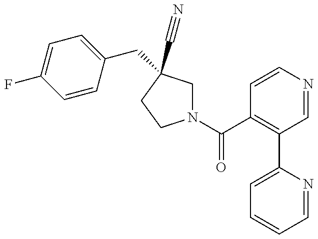 [SMILES: N#C[C@]1(Cc2ccc(F)cc2)CCN(C(=O)c2ccncc2-c2ccccn2)C1] |
| A74 | 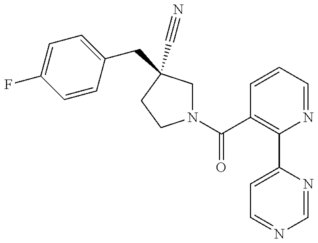 [SMILES: N#C[C@@]1(Cc2ccc(F)cc2)CCN(C(=O)c2cccnc2-c2ccncn2)C1] |
| A75 | 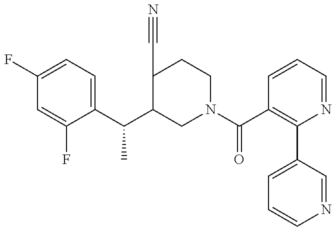 [SMILES: C[C@H](c1ccc(F)cc1F)C1CN(C(=O)c2cccnc2-c2cccnc2)CCC1C#N] |
| A76 | 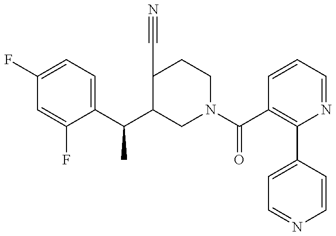 [SMILES: C[C@@H](c1ccc(F)cc1F)C1CN(C(=O)c2cccnc2-c2ccncc2)CCC1C#N] |
| A77 | 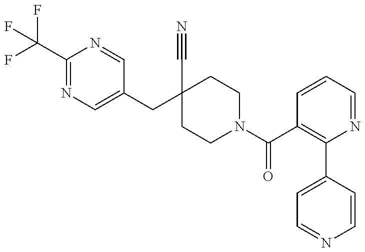 [SMILES: N#CC1(Cc2cnc(C(F)(F)F)nc2)CCN(C(=O)c2cccnc2-c2ccncc2)CC1] |
| A78 | 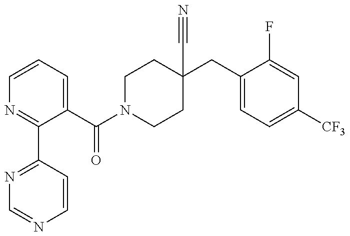 [SMILES: N#CC1(Cc2ccc(C(F)(F)F)cc2F)CCN(C(=O)c2cccnc2-c2ccncn2)CC1] |
| A79 | 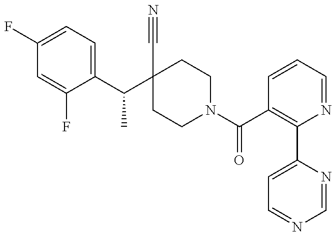 [SMILES: C[C@H](c1ccc(F)cc1F)C1(C#N)CCN(C(=O)c2cccnc2-c2ccncn2)CC1] |
| A80 | 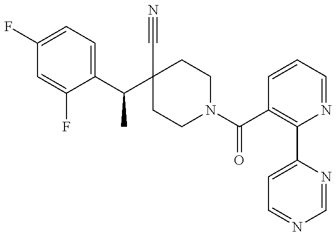 [SMILES: C[C@@H](c1ccc(F)cc1F)C1(C#N)CCN(C(=O)c2cccnc2-c2ccncn2)CC1] |
| A81 | 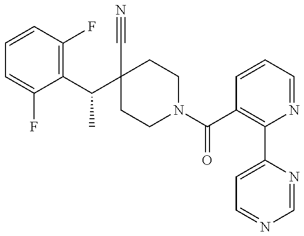 [SMILES: C[C@H](c1c(F)cccc1F)C1(C#N)CCN(C(=O)c2cccnc2-c2ccncn2)CC1] |
| A82 | 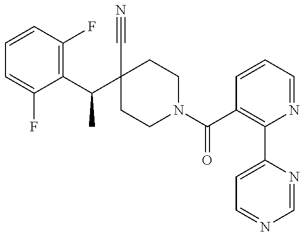 [SMILES: C[C@@H](c1c(F)cccc1F)C1(C#N)CCN(C(=O)c2cccnc2-c2ccncn2)CC1] |
| A83 | 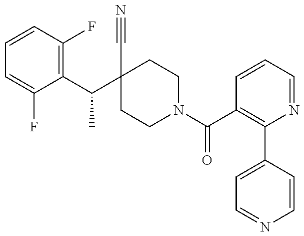 [SMILES: C[C@H](c1c(F)cccc1F)C1(C#N)CCN(C(=O)c2cccnc2-c2ccncc2)CC1] |
| A84 | 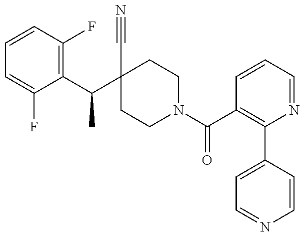 [SMILES: C[C@@H](c1c(F)cccc1F)C1(C#N)CCN(C(=O)c2cccnc2-c2ccncc2)CC1] |
| A85 | 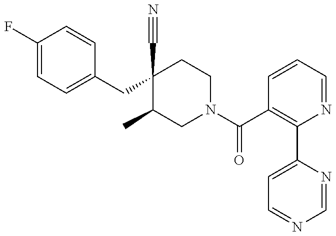 [SMILES: C[C@@H]1CN(C(=O)c2cccnc2-c2ccncn2)CC[C@@]1(C#N)Cc1ccc(F)cc1] |
| A86 | 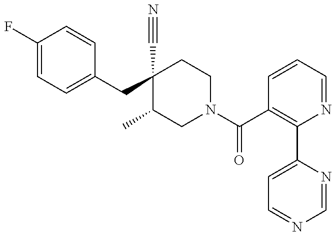 [SMILES: C[C@H]1CN(C(=O)c2cccnc2-c2ccncn2)CC[C@]1(C#N)Cc1ccc(F)cc1] |
| A87 | 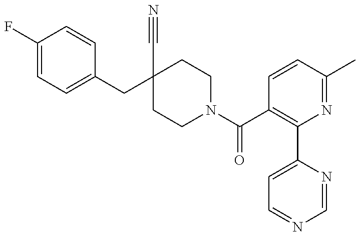 [SMILES: Cc1ccc(C(=O)N2CCC(C#N)(Cc3ccc(F)cc3)CC2)c(-c2ccncn2)n1] |
| A88 | 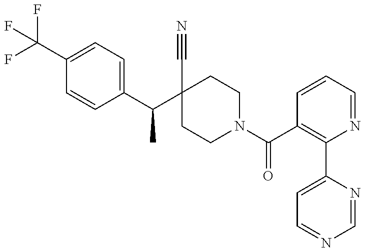 [SMILES: C[C@@H](c1ccc(C(F)(F)F)cc1)C1(C#N)CCN(C(=O)c2cccnc2-c2ccncn2)CC1] |
| A89 | 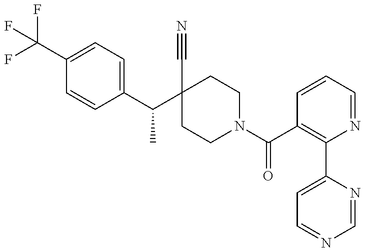 [SMILES: C[C@H](c1ccc(C(F)(F)F)cc1)C1(C#N)CCN(C(=O)c2cccnc2-c2ccncn2)CC1] |
| A90 | 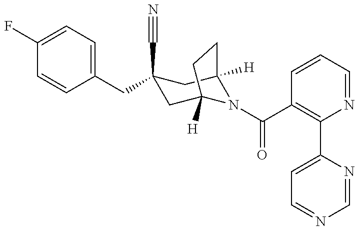 [SMILES: N#C[C@@]1(Cc2ccc(F)cc2)C[C@H]2CC[C@@H](C1)N2C(=O)c1cccnc1-c1ccncn1] |
| A91 | 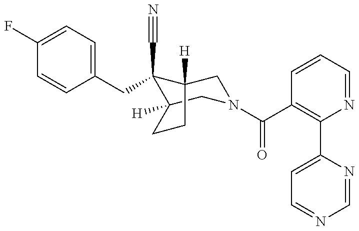 [SMILES: N#C[C@]1(Cc2ccc(F)cc2)[C@@H]2CC[C@H]1CN(C(=O)c1cccnc1-c1ccncn1)C2] |
| A92 | 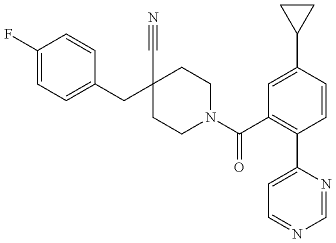 [SMILES: N#CC1(Cc2ccc(F)cc2)CCN(C(=O)c2cc(C3CC3)ccc2-c2ccncn2)CC1] |
| A93 | 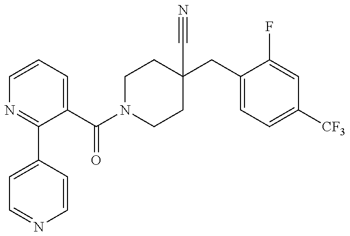 [SMILES: N#CC1(Cc2ccc(C(F)(F)F)cc2F)CCN(C(=O)c2cccnc2-c2ccncc2)CC1] |
| A94 | 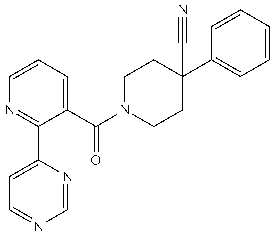 [SMILES: N#CC1(c2ccccc2)CCN(C(=O)c2cccnc2-c2ccncn2)CC1] |
| A95 | 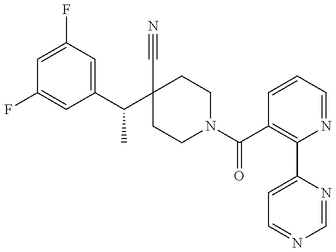 [SMILES: C[C@H](c1cc(F)cc(F)c1)C1(C#N)CCN(C(=O)c2cccnc2-c2ccncn2)CC1] |
| A96 | 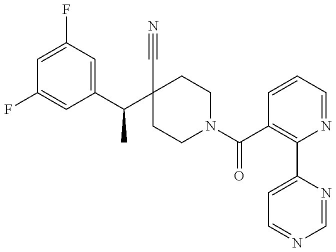 [SMILES: C[C@@H](c1cc(F)cc(F)c1)C1(C#N)CCN(C(=O)c2cccnc2-c2ccncn2)CC1] |
| A97 | 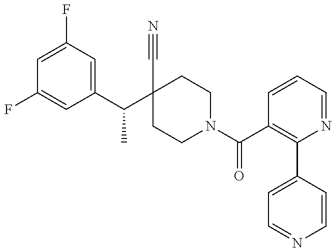 [SMILES: C[C@H](c1cc(F)cc(F)c1)C1(C#N)CCN(C(=O)c2cccnc2-c2ccncc2)CC1] |
| A98 | 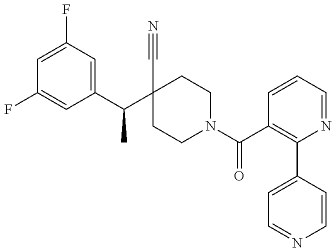 [SMILES: C[C@@H](c1cc(F)cc(F)c1)C1(C#N)CCN(C(=O)c2cccnc2-c2ccncc2)CC1] |
| A99 | 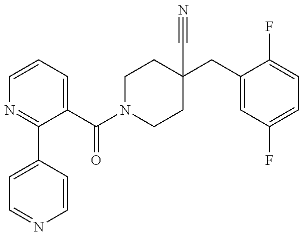 [SMILES: N#CC1(Cc2cc(F)ccc2F)CCN(C(=O)c2cccnc2-c2ccncc2)CC1] |
| A100 | 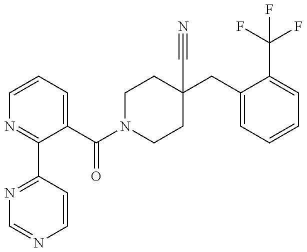 [SMILES: N#CC1(Cc2ccccc2C(F)(F)F)CCN(C(=O)c2cccnc2-c2ccncn2)CC1] |
| A101 | 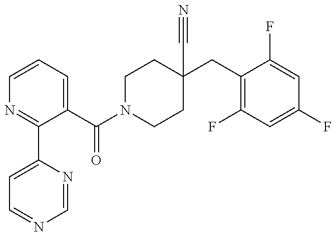 [SMILES: N#CC1(Cc2c(F)cc(F)cc2F)CCN(C(=O)c2cccnc2-c2ccncn2)CC1] |
| A102 | 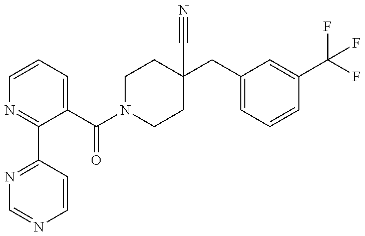 [SMILES: N#CC1(Cc2cccc(C(F)(F)F)c2)CCN(C(=O)c2cccnc2-c2ccncn2)CC1] |
| A103 | 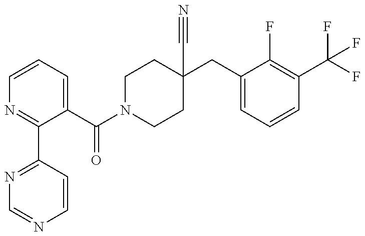 [SMILES: N#CC1(Cc2cccc(C(F)(F)F)c2F)CCN(C(=O)c2cccnc2-c2ccncn2)CC1] |
| A104 | 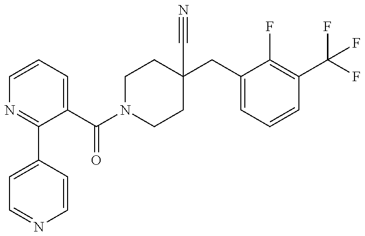 [SMILES: N#CC1(Cc2cccc(C(F)(F)F)c2F)CCN(C(=O)c2cccnc2-c2ccncc2)CC1] |
| A105 | 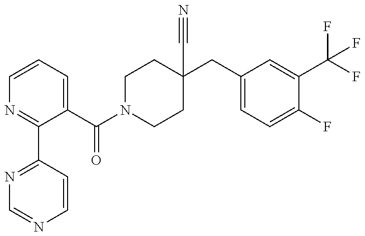 [SMILES: N#CC1(Cc2ccc(F)c(C(F)(F)F)c2)CCN(C(=O)c2cccnc2-c2ccncn2)CC1] |
| A106 | 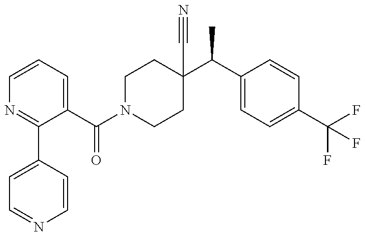 [SMILES: C[C@@H](c1ccc(C(F)(F)F)cc1)C1(C#N)CCN(C(=O)c2cccnc2-c2ccncc2)CC1] |
| A107 | 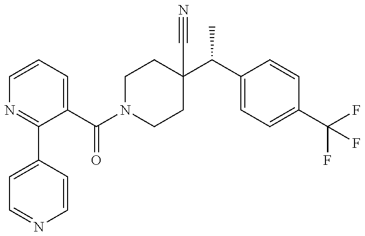 [SMILES: C[C@H](c1ccc(C(F)(F)F)cc1)C1(C#N)CCN(C(=O)c2cccnc2-c2ccncc2)CC1] |
| A108 | 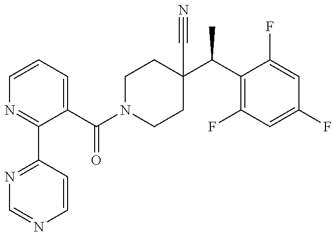 [SMILES: C[C@@H](c1c(F)cc(F)cc1F)C1(C#N)CCN(C(=O)c2cccnc2-c2ccncn2)CC1] |
| A109 | 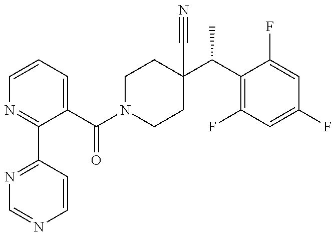 [SMILES: C[C@H](c1c(F)cc(F)cc1F)C1(C#N)CCN(C(=O)c2cccnc2-c2ccncn2)CC1] |
| A110 | 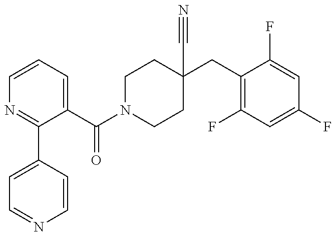 [SMILES: N#CC1(Cc2c(F)cc(F)cc2F)CCN(C(=O)c2cccnc2-c2ccncc2)CC1] |
| A111 | 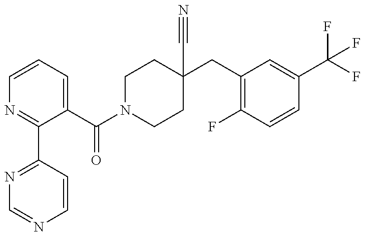 [SMILES: N#CC1(Cc2cc(C(F)(F)F)ccc2F)CCN(C(=O)c2cccnc2-c2ccncn2)CC1] |
| A112 | 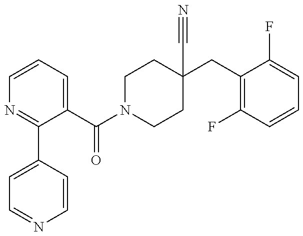 [SMILES: N#CC1(Cc2c(F)cccc2F)CCN(C(=O)c2cccnc2-c2ccncc2)CC1] |
| A113 | 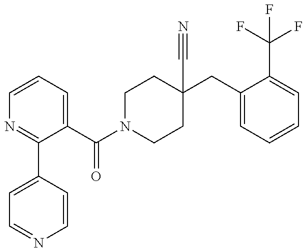 [SMILES: N#CC1(Cc2ccccc2C(F)(F)F)CCN(C(=O)c2cccnc2-c2ccncc2)CC1] |
| A114 | 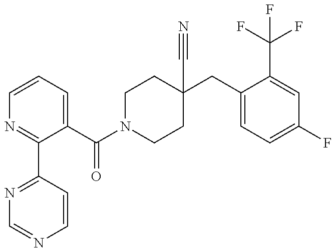 [SMILES: N#CC1(Cc2ccc(F)cc2C(F)(F)F)CCN(C(=O)c2cccnc2-c2ccncn2)CC1] |
| A115 | 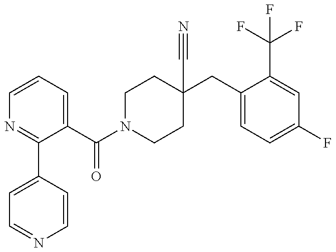 [SMILES: N#CC1(Cc2ccc(F)cc2C(F)(F)F)CCN(C(=O)c2cccnc2-c2ccncc2)CC1] |
| A116 | 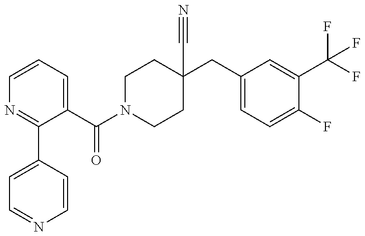 [SMILES: N#CC1(Cc2ccc(F)c(C(F)(F)F)c2)CCN(C(=O)c2cccnc2-c2ccncc2)CC1] |
| A117 | 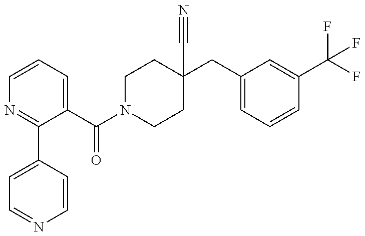 [SMILES: N#CC1(Cc2cccc(C(F)(F)F)c2)CCN(C(=O)c2cccnc2-c2ccncc2)CC1] |
| A118 | 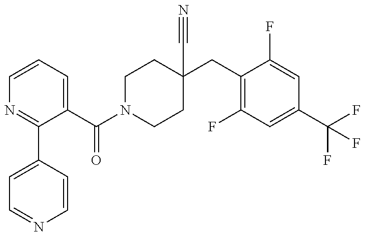 [SMILES: N#CC1(Cc2c(F)cc(C(F)(F)F)cc2F)CCN(C(=O)c2cccnc2-c2ccncc2)CC1] |
| A119 | 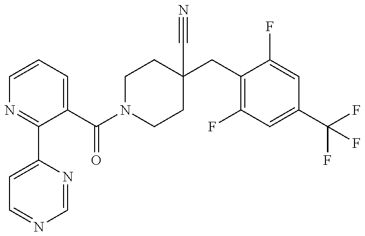 [SMILES: N#CC1(Cc2c(F)cc(C(F)(F)F)cc2F)CCN(C(=O)c2cccnc2-c2ccncn2)CC1] |
| A120 | 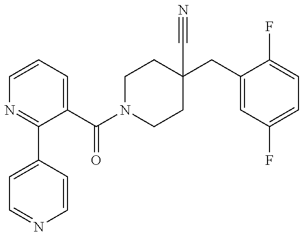 [SMILES: N#CC1(Cc2cc(F)ccc2F)CCN(C(=O)c2cccnc2-c2ccncc2)CC1] |
| A121 | 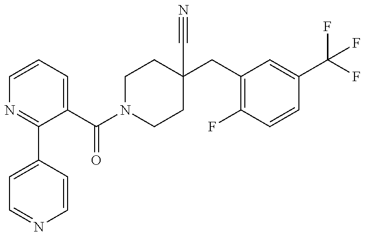 [SMILES: N#CC1(Cc2cc(C(F)(F)F)ccc2F)CCN(C(=O)c2cccnc2-c2ccncc2)CC1] |
| A122 | 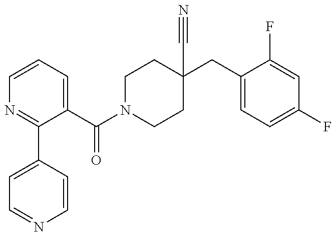 [SMILES: N#CC1(Cc2ccc(F)cc2F)CCN(C(=O)c2cccnc2-c2ccncc2)CC1] |
| A123 | 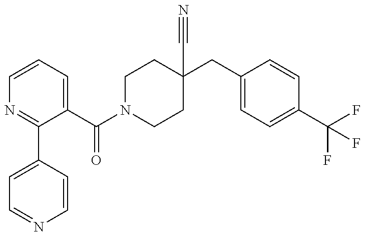 [SMILES: N#CC1(Cc2ccc(C(F)(F)F)cc2)CCN(C(=O)c2cccnc2-c2ccncc2)CC1] |
| A124 | 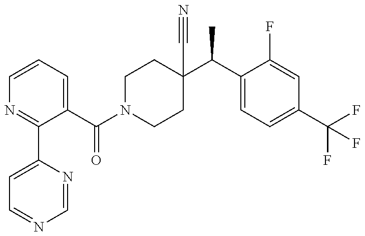 [SMILES: C[C@@H](c1ccc(C(F)(F)F)cc1F)C1(C#N)CCN(C(=O)c2cccnc2-c2ccncn2)CC1] |
| A125 | 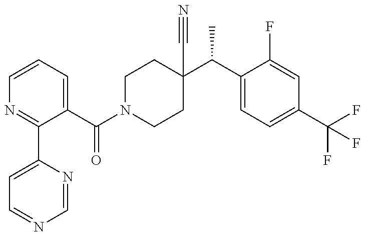 [SMILES: C[C@H](c1ccc(C(F)(F)F)cc1F)C1(C#N)CCN(C(=O)c2cccnc2-c2ccncn2)CC1] |
| A126 | 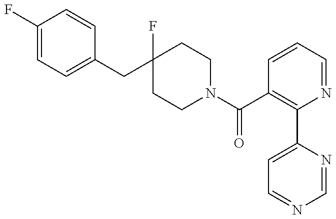 [SMILES: O=C(c1cccnc1-c1ccncn1)N1CCC(F)(Cc2ccc(F)cc2)CC1] |
| A127 | 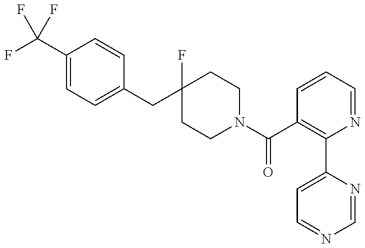 [SMILES: O=C(c1cccnc1-c1ccncn1)N1CCC(F)(Cc2ccc(C(F)(F)F)cc2)CC1] |
| A128 | 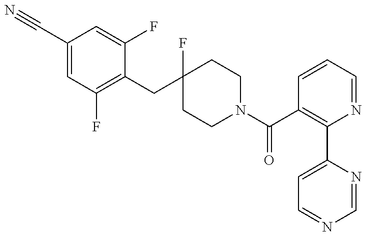 [SMILES: N#Cc1cc(F)c(CC2(F)CCN(C(=O)c3cccnc3-c3ccncn3)CC2)c(F)c1] |
| A129 | 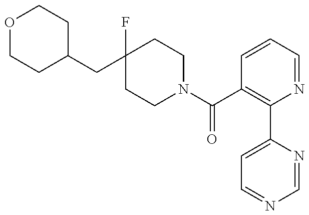 [SMILES: O=C(c1cccnc1-c1ccncn1)N1CCC(F)(CC2CCOCC2)CC1] |
| A130 | 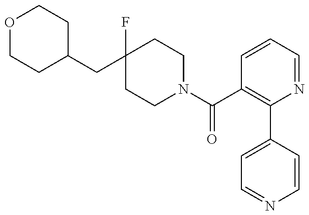 [SMILES: O=C(c1cccnc1-c1ccncc1)N1CCC(F)(CC2CCOCC2)CC1] |
| A131 | 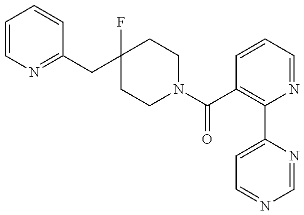 [SMILES: O=C(c1cccnc1-c1ccncn1)N1CCC(F)(Cc2ccccn2)CC1] |
| A132 | 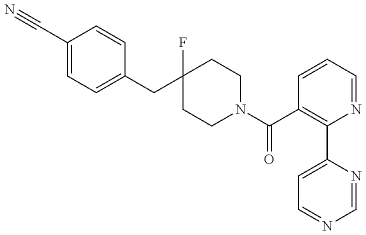 [SMILES: N#Cc1ccc(CC2(F)CCN(C(=O)c3cccnc3-c3ccncn3)CC2)cc1] |
| A133 | 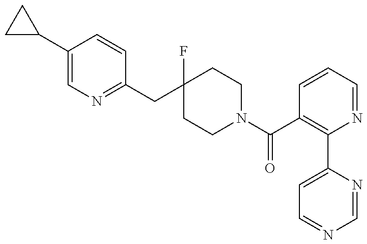 [SMILES: O=C(c1cccnc1-c1ccncn1)N1CCC(F)(Cc2ccc(C3CC3)cn2)CC1] |
| A134 | 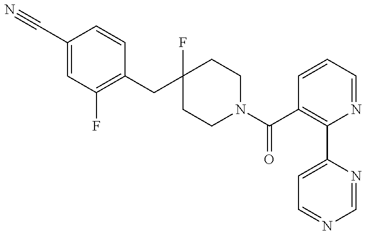 [SMILES: N#Cc1ccc(CC2(F)CCN(C(=O)c3cccnc3-c3ccncn3)CC2)c(F)c1] |
| A135 | 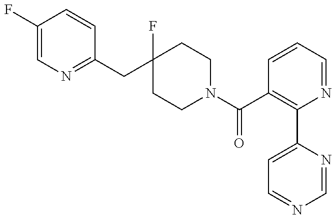 [SMILES: O=C(c1cccnc1-c1ccncn1)N1CCC(F)(Cc2ccc(F)cn2)CC1] |
| A136 | 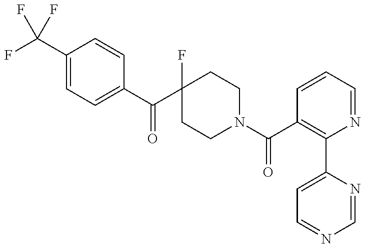 [SMILES: O=C(c1cccnc1-c1ccncn1)N1CCC(F)(C(=O)c2ccc(C(F)(F)F)cc2)CC1] |
| A137 | 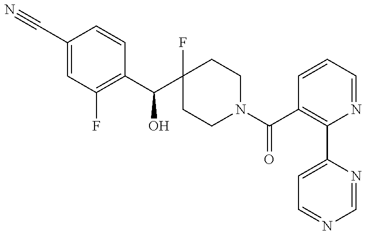 [SMILES: N#Cc1ccc([C@H](O)C2(F)CCN(C(=O)c3cccnc3-c3ccncn3)CC2)c(F)c1] |
| A138 | 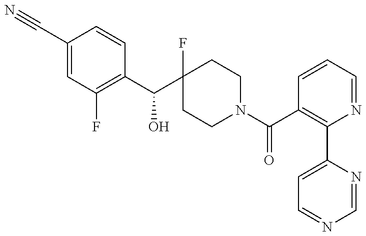 [SMILES: N#Cc1ccc([C@@H](O)C2(F)CCN(C(=O)c3cccnc3-c3ccncn3)CC2)c(F)c1] |
| A139 | 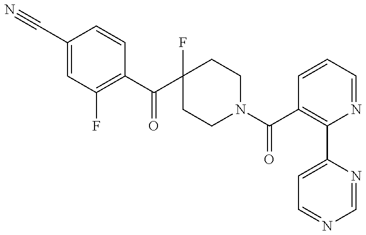 [SMILES: N#Cc1ccc(C(=O)C2(F)CCN(C(=O)c3cccnc3-c3ccncn3)CC2)c(F)c1] |
| A140 | 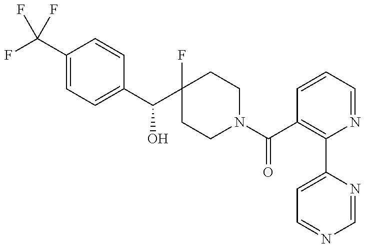 [SMILES: O=C(c1cccnc1-c1ccncn1)N1CCC(F)([C@H](O)c2ccc(C(F)(F)F)cc2)CC1] |
| A141 | 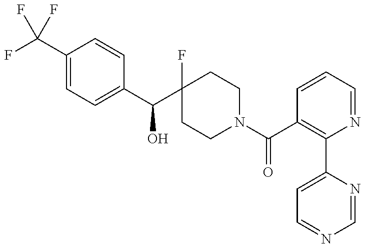 [SMILES: O=C(c1cccnc1-c1ccncn1)N1CCC(F)([C@@H](O)c2ccc(C(F)(F)F)cc2)CC1] |
| A142 | 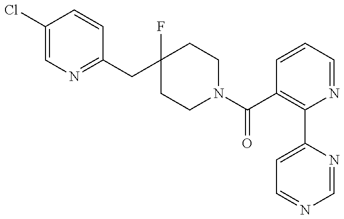 [SMILES: O=C(c1cccnc1-c1ccncn1)N1CCC(F)(Cc2ccc(Cl)cn2)CC1] |
| A143 | 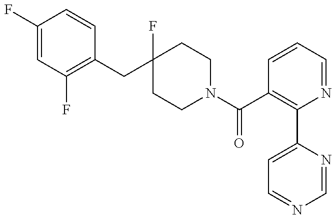 [SMILES: O=C(c1cccnc1-c1ccncn1)N1CCC(F)(Cc2ccc(F)cc2F)CC1] |
| A144 | 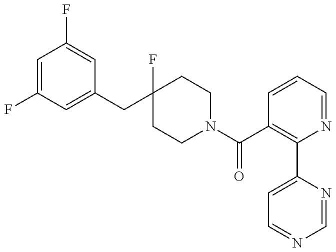 [SMILES: O=C(c1cccnc1-c1ccncn1)N1CCC(F)(Cc2cc(F)cc(F)c2)CC1] |
| A145 | 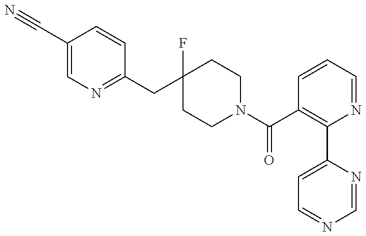 [SMILES: N#Cc1ccc(CC2(F)CCN(C(=O)c3cccnc3-c3ccncn3)CC2)nc1] |
| A146 | 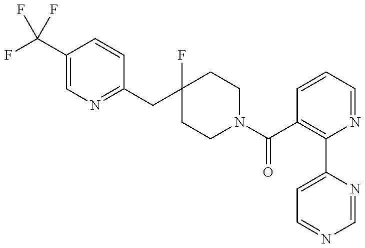 [SMILES: O=C(c1cccnc1-c1ccncn1)N1CCC(F)(Cc2ccc(C(F)(F)F)cn2)CC1] |
| A147 | 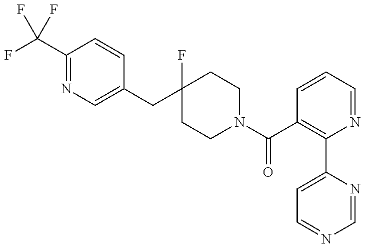 [SMILES: O=C(c1cccnc1-c1ccncn1)N1CCC(F)(Cc2ccc(C(F)(F)F)nc2)CC1] |
| A148 | 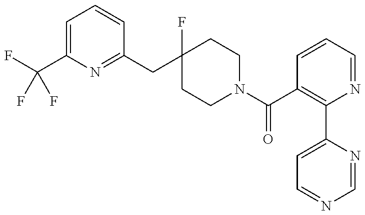 [SMILES: O=C(c1cccnc1-c1ccncn1)N1CCC(F)(Cc2cccc(C(F)(F)F)n2)CC1] |
| A149 | 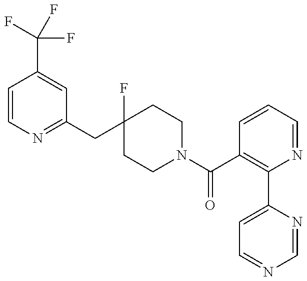 [SMILES: O=C(c1cccnc1-c1ccncn1)N1CCC(F)(Cc2cc(C(F)(F)F)ccn2)CC1] |
| A150 | 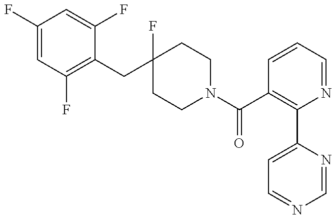 [SMILES: O=C(c1cccnc1-c1ccncn1)N1CCC(F)(Cc2c(F)cc(F)cc2F)CC1] |
| A151 | 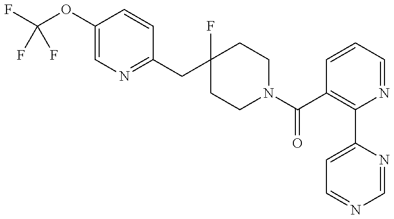 [SMILES: O=C(c1cccnc1-c1ccncn1)N1CCC(F)(Cc2ccc(OC(F)(F)F)cn2)CC1] |
| A152 | 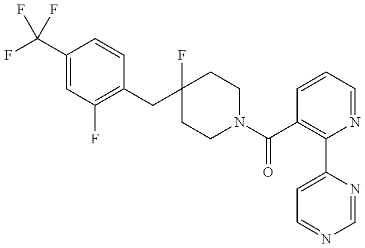 [SMILES: O=C(c1cccnc1-c1ccncn1)N1CCC(F)(Cc2ccc(C(F)(F)F)cc2F)CC1] |
| A153 | 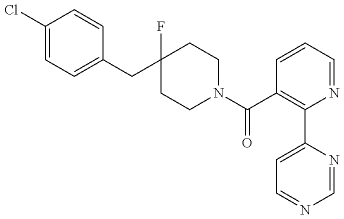 [SMILES: O=C(c1cccnc1-c1ccncn1)N1CCC(F)(Cc2ccc(Cl)cc2)CC1] |
| A154 | 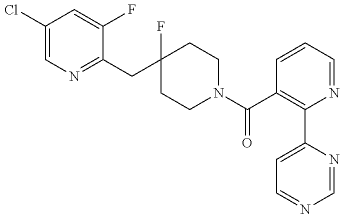 [SMILES: O=C(c1cccnc1-c1ccncn1)N1CCC(F)(Cc2ncc(Cl)cc2F)CC1] |
| A155 | 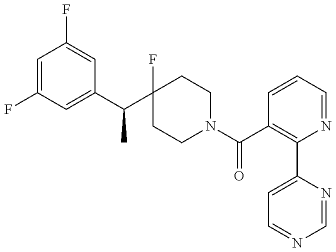 [SMILES: C[C@@H](c1cc(F)cc(F)c1)C1(F)CCN(C(=O)c2cccnc2-c2ccncn2)CC1] |
| A156 | 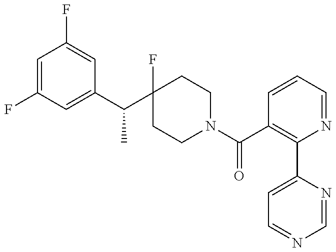 [SMILES: C[C@H](c1cc(F)cc(F)c1)C1(F)CCN(C(=O)c2cccnc2-c2ccncn2)CC1] |
| A157 | 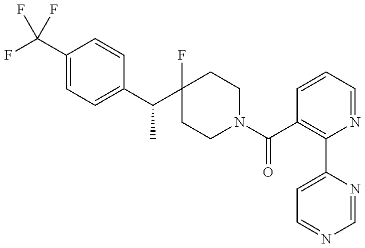 [SMILES: C[C@H](c1ccc(C(F)(F)F)cc1)C1(F)CCN(C(=O)c2cccnc2-c2ccncn2)CC1] |
| A158 | 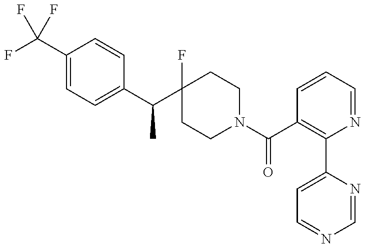 [SMILES: C[C@@H](c1ccc(C(F)(F)F)cc1)C1(F)CCN(C(=O)c2cccnc2-c2ccncn2)CC1] |
| A159 | 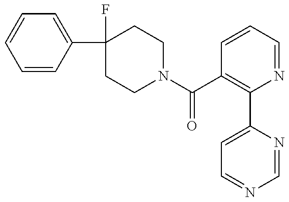 [SMILES: O=C(c1cccnc1-c1ccncn1)N1CCC(F)(c2ccccc2)CC1] |
| A160 | 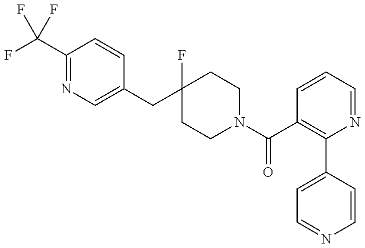 [SMILES: O=C(c1cccnc1-c1ccncc1)N1CCC(F)(Cc2ccc(C(F)(F)F)nc2)CC1] |
| A161 | 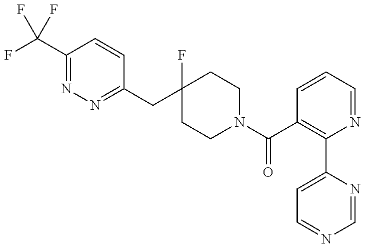 [SMILES: O=C(c1cccnc1-c1ccncn1)N1CCC(F)(Cc2ccc(C(F)(F)F)nn2)CC1] |
| A162 | 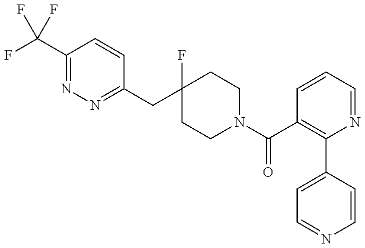 [SMILES: O=C(c1cccnc1-c1ccncc1)N1CCC(F)(Cc2ccc(C(F)(F)F)nn2)CC1] |
| A163 | 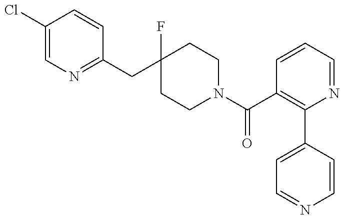 [SMILES: O=C(c1cccnc1-c1ccncc1)N1CCC(F)(Cc2ccc(Cl)cn2)CC1] |
| A164 | 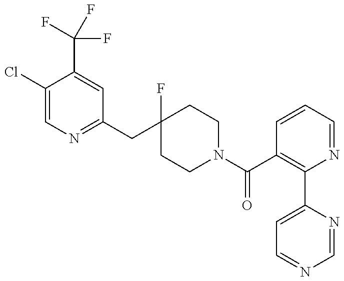 [SMILES: O=C(c1cccnc1-c1ccncn1)N1CCC(F)(Cc2cc(C(F)(F)F)c(Cl)cn2)CC1] |
| A165 | 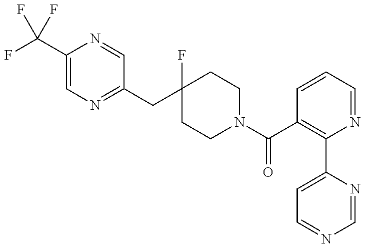 [SMILES: O=C(c1cccnc1-c1ccncn1)N1CCC(F)(Cc2cnc(C(F)(F)F)cn2)CC1] |
| A166 | 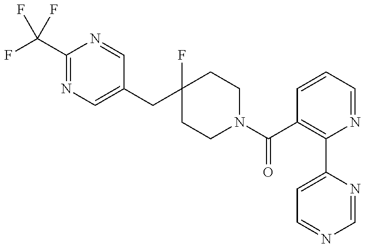 [SMILES: O=C(c1cccnc1-c1ccncn1)N1CCC(F)(Cc2cnc(C(F)(F)F)nc2)CC1] |
| A167 | 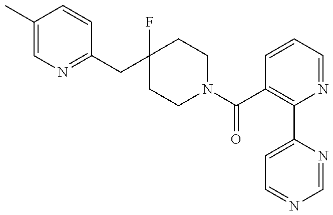 [SMILES: Cc1ccc(CC2(F)CCN(C(=O)c3cccnc3-c3ccncn3)CC2)nc1] |
| A168 | 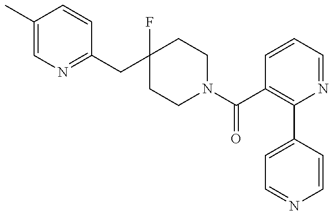 [SMILES: Cc1ccc(CC2(F)CCN(C(=O)c3cccnc3-c3ccncc3)CC2)nc1] |
| A169 | 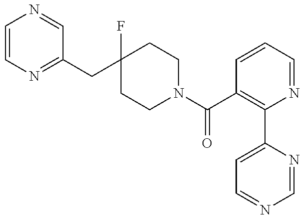 [SMILES: O=C(c1cccnc1-c1ccncn1)N1CCC(F)(Cc2cnccn2)CC1] |
| A170 | 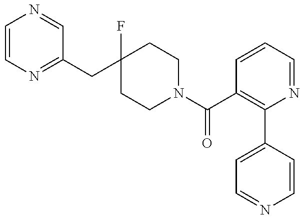 [SMILES: O=C(c1cccnc1-c1ccncc1)N1CCC(F)(Cc2cnccn2)CC1] |
| A171 | 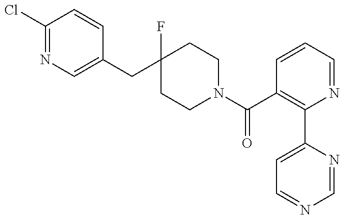 [SMILES: O=C(c1cccnc1-c1ccncn1)N1CCC(F)(Cc2ccc(Cl)nc2)CC1] |
| A172 | 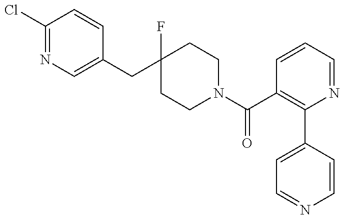 [SMILES: O=C(c1cccnc1-c1ccncc1)N1CCC(F)(Cc2ccc(Cl)nc2)CC1] |
| A173 | 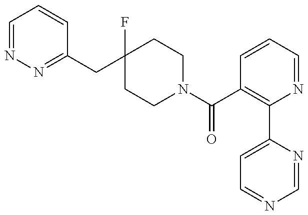 [SMILES: O=C(c1cccnc1-c1ccncn1)N1CCC(F)(Cc2cccnn2)CC1] |
| A174 | 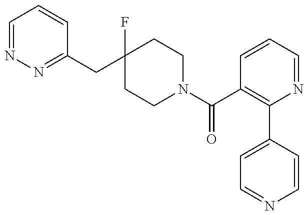 [SMILES: O=C(c1cccnc1-c1ccncc1)N1CCC(F)(Cc2cccnn2)CC1] |
| A175 | 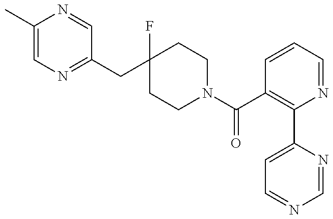 [SMILES: Cc1cnc(CC2(F)CCN(C(=O)c3cccnc3-c3ccncn3)CC2)cn1] |
| A176 | 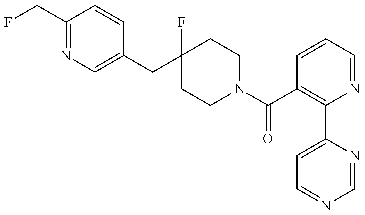 [SMILES: O=C(c1cccnc1-c1ccncn1)N1CCC(F)(Cc2ccc(CF)nc2)CC1] |
| A177 | 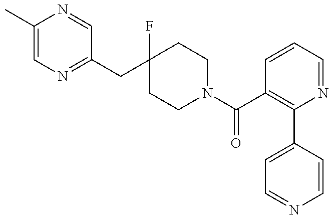 [SMILES: Cc1cnc(CC2(F)CCN(C(=O)c3cccnc3-c3ccncc3)CC2)cn1] |
| A178 | 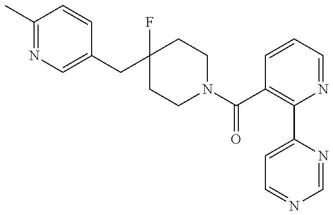 [SMILES: Cc1ccc(CC2(F)CCN(C(=O)c3cccnc3-c3ccncn3)CC2)cn1] |
| A179 | 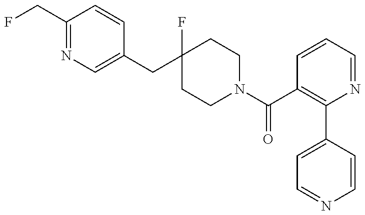 [SMILES: O=C(c1cccnc1-c1ccncc1)N1CCC(F)(Cc2ccc(CF)nc2)CC1] |
| A180 | 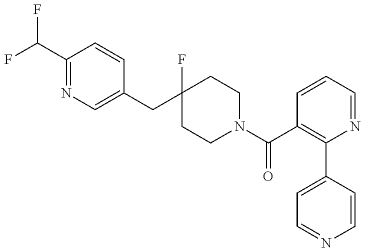 [SMILES: O=C(c1cccnc1-c1ccncc1)N1CCC(F)(Cc2ccc(C(F)F)nc2)CC1] |
| A181 | 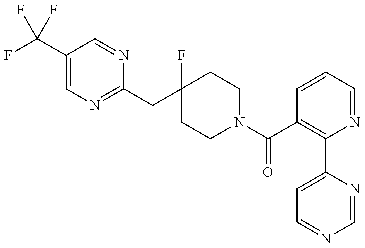 [SMILES: O=C(c1cccnc1-c1ccncn1)N1CCC(F)(Cc2ncc(C(F)(F)F)cn2)CC1] |
| A182 | 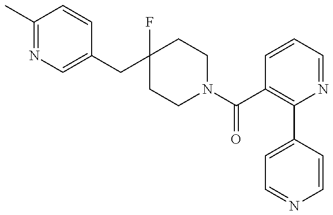 [SMILES: Cc1ccc(CC2(F)CCN(C(=O)c3cccnc3-c3ccncc3)CC2)cn1] |

In some embodiments, the CYP46A1 inhibitor is any single compound set forth in Table 1 or a pharmaceutically acceptable salt thereof.

The synthesis of compounds A1-A125 is described in WO2020/243027, and the synthesis of compounds A126-A182 is described in WO 2022/115620. The disclosures of each of the foregoing applications is incorporated by reference herein in their entirety.

### NMDA Receptor Positive Allosteric Modulators

An NMDA PAM useful in the compositions, kits and methods of the present disclosure is a compound that is a modulator of synaptic and/or extrasynaptic NMDA receptors, and exhibits a beneficial effect for treating or preventing Alzheimer's disease as disclosed herein when administered in combination with an anti-amyloid beta antibody or antigen-binding fragment thereof. NMDA PAMs are known to those skilled in the art. Therefore, any NDMA PAM may be useful in the compositions, kits and methods of the present disclosure.

In some embodiments, the NDMA PAM is a neuroactive steroid. In some embodiments, the neuroactive steroid is any one of compounds B1-B543, or pharmaceutically acceptable salts thereof. In some embodiments, the neuroactive steroid is any one of compounds B1-B543 (See Table 2):

TABLE 2

| No | Structure |
|---|---|
| B1 | 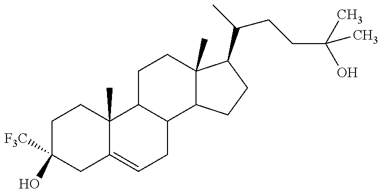 [SMILES: CC(CCC(C)(C)O)[C@H]1CCC2C3CC=C4C[C@](O)(C(F)(F)F)CC[C@]4(C)C3CC[C@@]21C] |
| B2 | 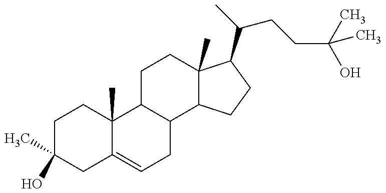 [SMILES: CC(CCC(C)(C)O)[C@H]1CCC2C3CC=C4C[C@@](C)(O)CC[C@]4(C)C3CC[C@@]21C] |
| B3 | 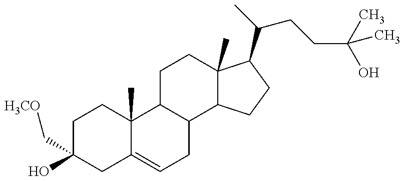 [SMILES: COC[C@]1(O)CC[C@@]2(C)C(=CCC3C2CC[C@@]2(C)C3CC[C@@H]2C(C)CCC(C)(C)O)C1] |
| B4 | 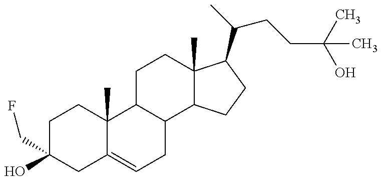 [SMILES: CC(CCC(C)(C)O)[C@H]1CCC2C3CC=C4C[C@](O)(CF)CC[C@]4(C)C3CC[C@@]21C] |
| B5 | 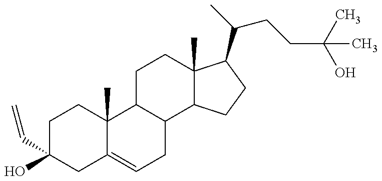 [SMILES: C=C[C@]1(O)CC[C@@]2(C)C(=CCC3C2CC[C@@]2(C)C3CC[C@@H]2C(C)CCC(C)(C)O)C1] |
| B6 | 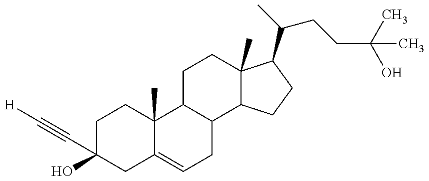 [SMILES: C#C[C@]1(O)CC[C@@]2(C)C(=CCC3C2CC[C@@]2(C)C3CC[C@@H]2C(C)CCC(C)(C)O)C1] |
| B7 | 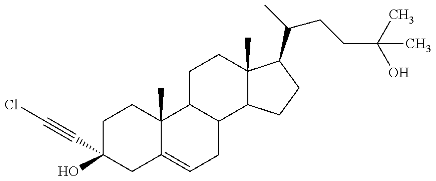 [SMILES: CC(CCC(C)(C)O)[C@H]1CCC2C3CC=C4C[C@](O)(C#CCl)CC[C@]4(C)C3CC[C@@]21C] |
| B8 | 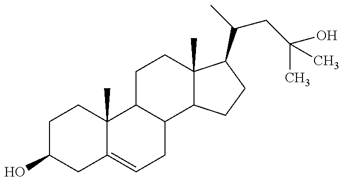 [SMILES: CC(CC(C)(C)O)[C@H]1CCC2C3CC=C4C[C@@H](O)CC[C@]4(C)C3CC[C@@]21C] |
| B9 | 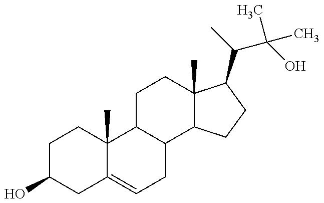 [SMILES: CC([C@H]1CCC2C3CC=C4C[C@@H](O)CC[C@]4(C)C3CC[C@@]21C)C(C)(C)O] |
| B10 | 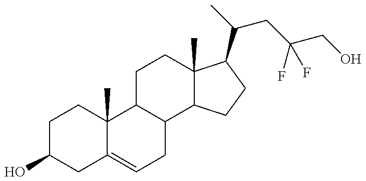 [SMILES: CC(CC(F)(F)CO)[C@H]1CCC2C3CC=C4C[C@@H](O)CC[C@]4(C)C3CC[C@@]21C] |
| B11 | 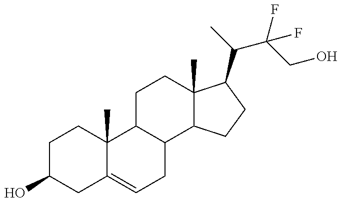 [SMILES: CC([C@H]1CCC2C3CC=C4C[C@@H](O)CC[C@]4(C)C3CC[C@@]21C)C(F)(F)CO] |
| B12 | 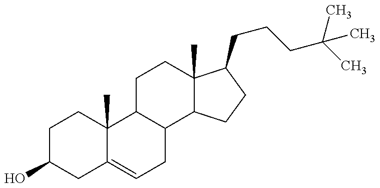 [SMILES: CC(C)(C)CCC[C@H]1CCC2C3CC=C4C[C@@H](O)CC[C@]4(C)C3CC[C@@]21C] |
| B13 | 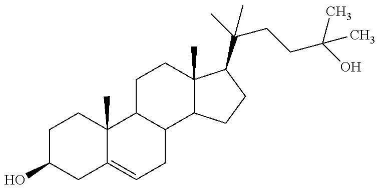 [SMILES: CC(C)(O)CCC(C)(C)[C@H]1CCC2C3CC=C4C[C@@H](O)CC[C@]4(C)C3CC[C@@]21C] |
| B14 | 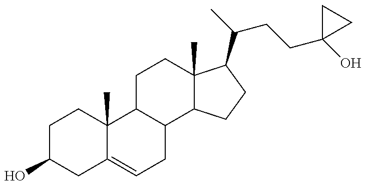 [SMILES: CC(CCC1(O)CC1)[C@H]1CCC2C3CC=C4C[C@@H](O)CC[C@]4(C)C3CC[C@@]21C] |
| B15 | 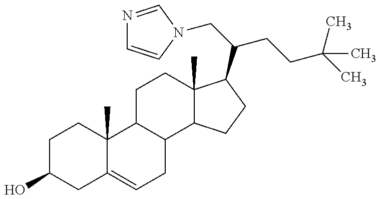 [SMILES: CC(C)(C)CCC(Cn1ccnc1)[C@H]1CCC2C3CC=C4C[C@@H](O)CC[C@]4(C)C3CC[C@@]21C] |
| B16 | 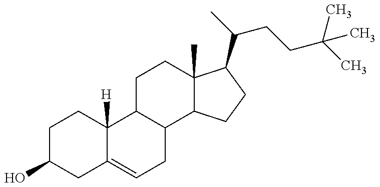 [SMILES: CC(CCC(C)(C)C)[C@H]1CCC2C3CC=C4C[C@@H](O)CC[C@@H]4C3CC[C@@]21C] |
| B17 | 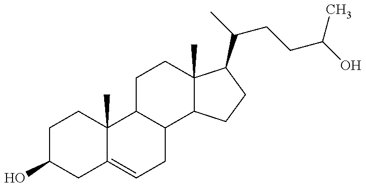 [SMILES: CC(O)CCC(C)[C@H]1CCC2C3CC=C4C[C@@H](O)CC[C@]4(C)C3CC[C@@]21C] |
| B18 | 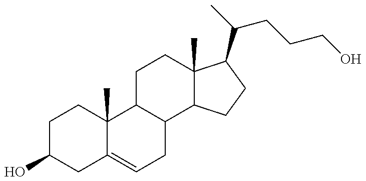 [SMILES: CC(CCCO)[C@H]1CCC2C3CC=C4C[C@@H](O)CC[C@]4(C)C3CC[C@@]21C] |
| B19 | 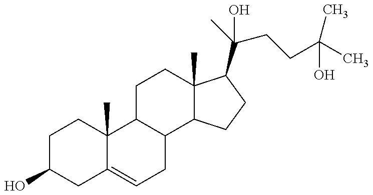 [SMILES: CC(C)(O)CCC(C)(O)[C@H]1CCC2C3CC=C4C[C@@H](O)CC[C@]4(C)C3CC[C@@]21C] |
| B20 | 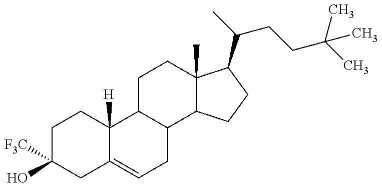 [SMILES: CC(CCC(C)(C)C)[C@H]1CCC2C3CC=C4C[C@](O)(C(F)(F)F)CC[C@@H]4C3CC[C@@]21C] |
| B21 | 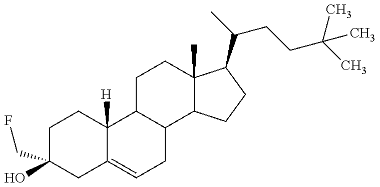 [SMILES: CC(CCC(C)(C)C)[C@H]1CCC2C3CC=C4C[C@](O)(CF)CC[C@@H]4C3CC[C@@]21C] |
| B22 | 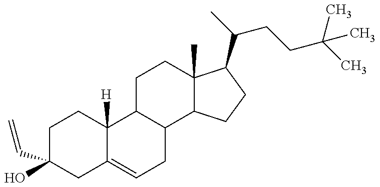 [SMILES: C=C[C@]1(O)CC[C@H]2C(=CCC3C2CC[C@@]2(C)C3CC[C@@H]2C(C)CCC(C)(C)C)C1] |
| B23 | 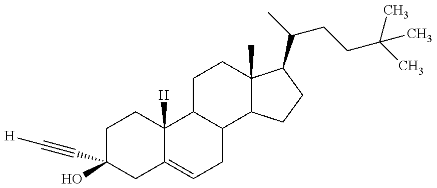 [SMILES: C#C[C@]1(O)CC[C@H]2C(=CCC3C2CC[C@@]2(C)C3CC[C@@H]2C(C)CCC(C)(C)C)C1] |
| B24 | 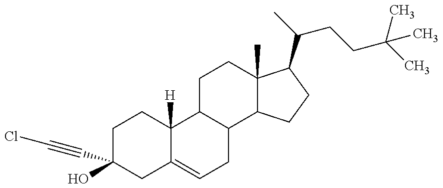 [SMILES: CC(CCC(C)(C)C)[C@H]1CCC2C3CC=C4C[C@](O)(C#CCl)CC[C@@H]4C3CC[C@@]21C] |
| B25 | 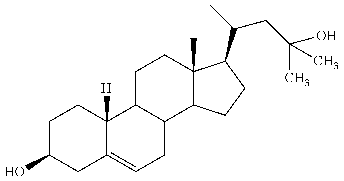 [SMILES: CC(CC(C)(C)O)[C@H]1CCC2C3CC=C4C[C@@H](O)CC[C@@H]4C3CC[C@@]21C] |
| B26 | 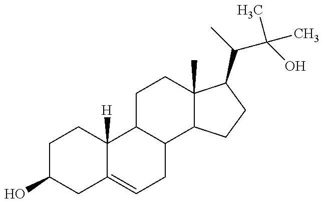 [SMILES: CC([C@H]1CCC2C3CC=C4C[C@@H](O)CC[C@@H]4C3CC[C@@]21C)C(C)(C)O] |
| B27 | 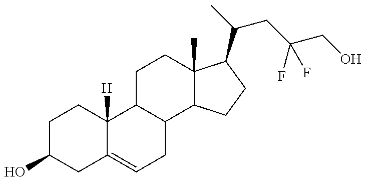 [SMILES: CC(CC(F)(F)CO)[C@H]1CCC2C3CC=C4C[C@@H](O)CC[C@@H]4C3CC[C@@]21C] |
| B28 | 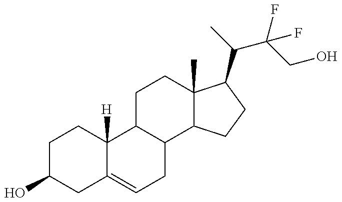 [SMILES: CC([C@H]1CCC2C3CC=C4C[C@@H](O)CC[C@@H]4C3CC[C@@]21C)C(F)(F)CO] |
| B29 | 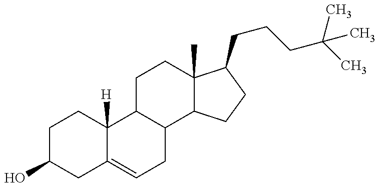 [SMILES: CC(C)(C)CCC[C@H]1CCC2C3CC=C4C[C@@H](O)CC[C@@H]4C3CC[C@@]21C] |
| B30 | 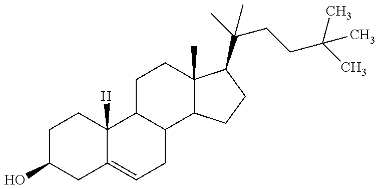 [SMILES: CC(C)(C)CCC(C)(C)[C@H]1CCC2C3CC=C4C[C@@H](O)CC[C@@H]4C3CC[C@@]21C] |
| B31 | 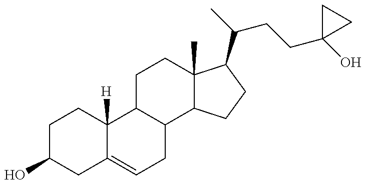 [SMILES: CC(CCC1(O)CC1)[C@H]1CCC2C3CC=C4C[C@@H](O)CC[C@@H]4C3CC[C@@]21C] |
| B32 | 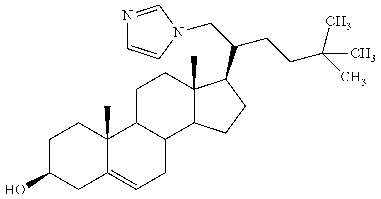 [SMILES: CC(C)(C)CCC(Cn1ccnc1)[C@H]1CCC2C3CC=C4C[C@@H](O)CC[C@]4(C)C3CC[C@@]21C] |
| B33 | 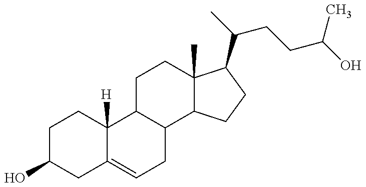 [SMILES: CC(O)CCC(C)[C@H]1CCC2C3CC=C4C[C@@H](O)CC[C@@H]4C3CC[C@@]21C] |
| B34 | 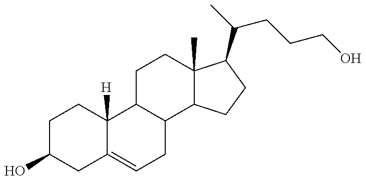 [SMILES: CC(CCCO)[C@H]1CCC2C3CC=C4C[C@@H](O)CC[C@@H]4C3CC[C@@]21C] |
| B35 | 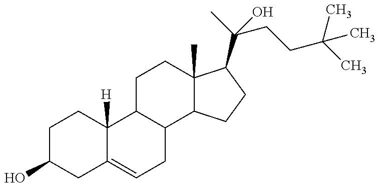 [SMILES: CC(C)(C)CCC(C)(O)[C@H]1CCC2C3CC=C4C[C@@H](O)CC[C@@H]4C3CC[C@@]21C] |
| B36 | 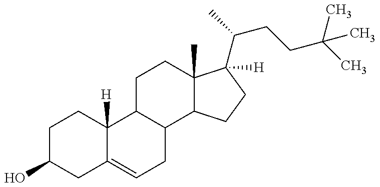 [SMILES: C[C@H](CCC(C)(C)C)[C@H]1CCC2C3CC=C4C[C@@H](O)CC[C@@H]4C3CC[C@@]21C] |
| B37 | 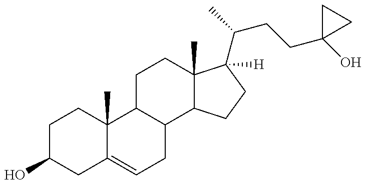 [SMILES: C[C@H](CCC1(O)CC1)[C@H]1CCC2C3CC=C4C[C@@H](O)CC[C@]4(C)C3CC[C@@]21C] |
| B38 | 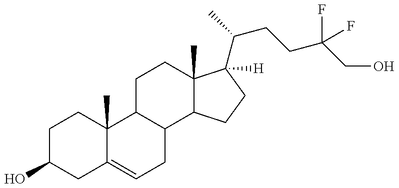 [SMILES: C[C@H](CCC(F)(F)CO)[C@H]1CCC2C3CC=C4C[C@@H](O)CC[C@]4(C)C3CC[C@@]21C] |
| B39 | 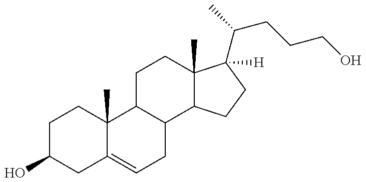 [SMILES: C[C@H](CCCO)[C@H]1CCC2C3CC=C4C[C@@H](O)CC[C@]4(C)C3CC[C@@]21C] |
| B40 | 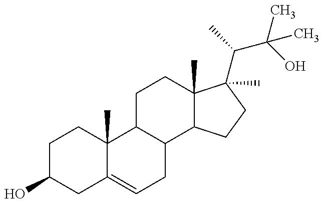 [SMILES: C[C@H](C(C)(C)O)[C@@]1(C)CCC2C3CC=C4C[C@@H](O)CC[C@]4(C)C3CC[C@@]21C] |
| B41 | 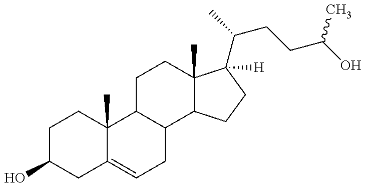 [SMILES: CC(O)CC[C@@H](C)[C@H]1CCC2C3CC=C4C[C@@H](O)CC[C@]4(C)C3CC[C@@]21C] |
| B42 | 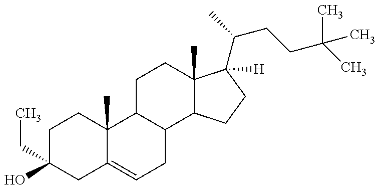 [SMILES: CC[C@]1(O)CC[C@@]2(C)C(=CCC3C2CC[C@@]2(C)C3CC[C@@H]2[C@H](C)CCC(C)(C)C)C1] |
| B43 | 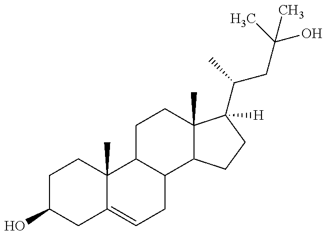 [SMILES: C[C@H](CC(C)(C)O)[C@H]1CCC2C3CC=C4C[C@@H](O)CC[C@]4(C)C3CC[C@@]21C] |
| B44 | 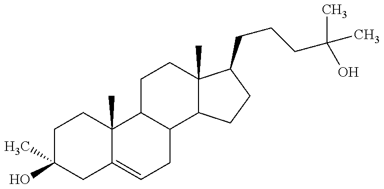 [SMILES: CC(C)(O)CCC[C@H]1CCC2C3CC=C4C[C@@](C)(O)CC[C@]4(C)C3CC[C@@]21C] |
| B45 | 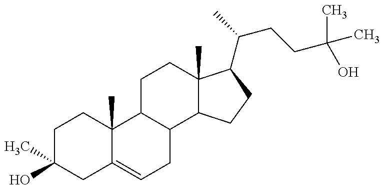 |
| B46 | 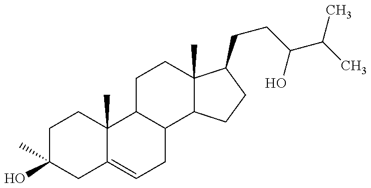 [SMILES: CC(C)C(O)CC[C@H]1CCC2C3CC=C4C[C@@](C)(O)CC[C@]4(C)C3CC[C@@]21C] |
| B47 | 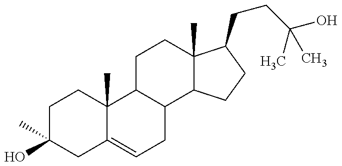 [SMILES: CC(C)(O)CC[C@H]1CCC2C3CC=C4C[C@@](C)(O)CC[C@]4(C)C3CC[C@@]21C] |
| B48 | 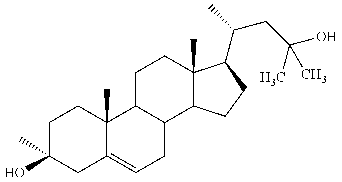 |
| B49 | 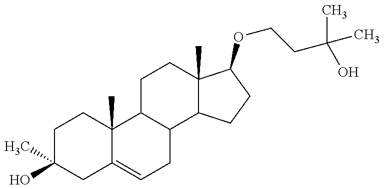 [SMILES: CC(C)(O)CCO[C@H]1CCC2C3CC=C4C[C@@](C)(O)CC[C@]4(C)C3CC[C@@]21C] |
| B50 | 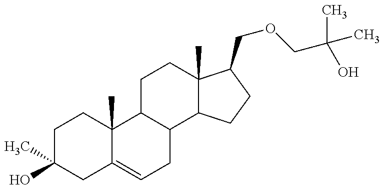 [SMILES: CC(C)(O)COC[C@H]1CCC2C3CC=C4C[C@@](C)(O)CC[C@]4(C)C3CC[C@@]21C] |
| B51 | 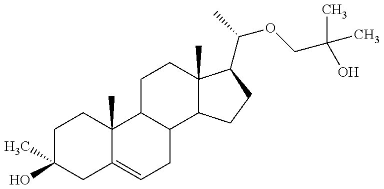 |
| B52 | 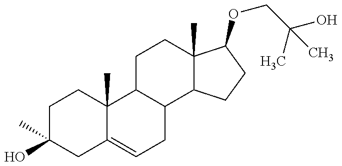 [SMILES: CC(C)(O)CO[C@H]1CCC2C3CC=C4C[C@@](C)(O)CC[C@]4(C)C3CC[C@@]21C] |
| B53 | 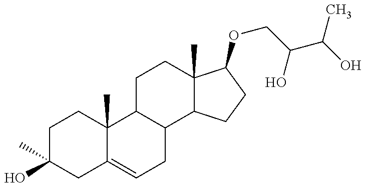 [SMILES: CC(O)C(O)CO[C@H]1CCC2C3CC=C4C[C@@](C)(O)CC[C@]4(C)C3CC[C@@]21C] |
| B54 | 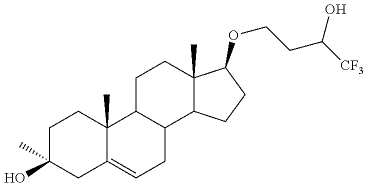 [SMILES: C[C@]1(O)CC[C@@]2(C)C(=CCC3C2CC[C@@]2(C)C3CC[C@@H]2OCCC(O)C(F)(F)F)C1] |
| B55 | 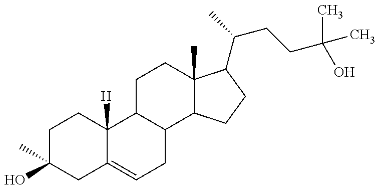 [SMILES: C[C@H](CCC(C)(C)O)C1CCC2C3CC=C4C[C@@](C)(O)CC[C@@H]4C3CC[C@@]21C] |
| B56 | 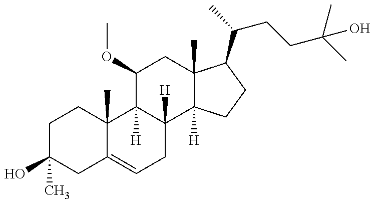 |
| B57 | 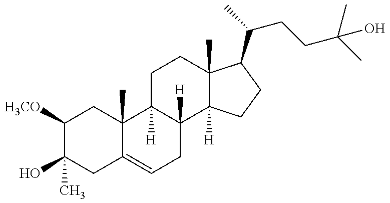 |
| B58 | 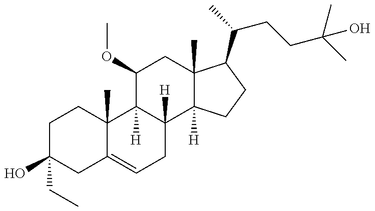 |
| B59 | 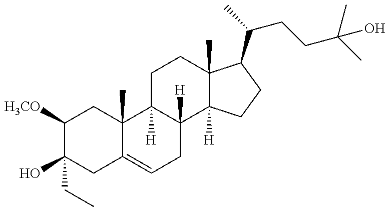 |
| B60 | 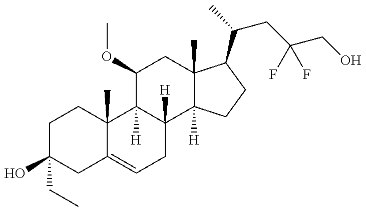 |
| B61 | 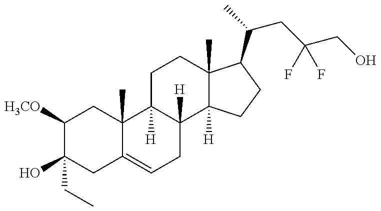 |
| B62 | 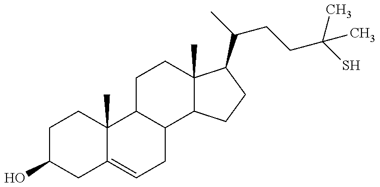 [SMILES: CC(CCC(C)(C)S)[C@H]1CCC2C3CC=C4C[C@@H](O)CC[C@]4(C)C3CC[C@@]21C] |
| B63 | 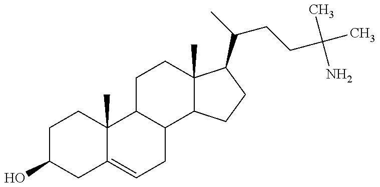 [SMILES: CC(CCC(C)(C)N)[C@H]1CCC2C3CC=C4C[C@@H](O)CC[C@]4(C)C3CC[C@@]21C] |
| B64 | 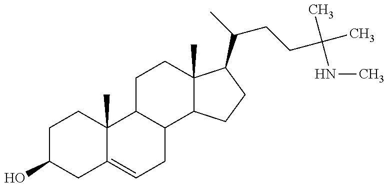 [SMILES: CNC(C)(C)CCC(C)[C@H]1CCC2C3CC=C4C[C@@H](O)CC[C@]4(C)C3CC[C@@]21C] |
| B65 | 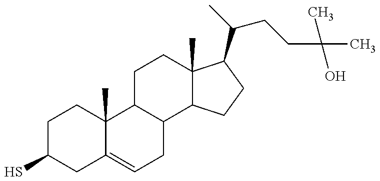 [SMILES: CC(CCC(C)(C)O)[C@H]1CCC2C3CC=C4C[C@@H](S)CC[C@]4(C)C3CC[C@@]21C] |
| B66 | 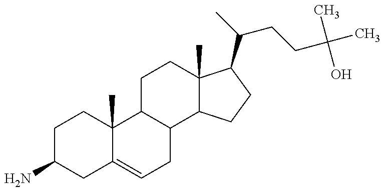 [SMILES: CC(CCC(C)(C)O)[C@H]1CCC2C3CC=C4C[C@@H](N)CC[C@]4(C)C3CC[C@@]21C] |
| B67 | 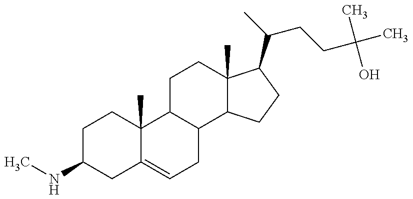 [SMILES: CN[C@H]1CC[C@@]2(C)C(=CCC3C2CC[C@@]2(C)C3CC[C@@H]2C(C)CCC(C)(C)O)C1] |
| B68 | 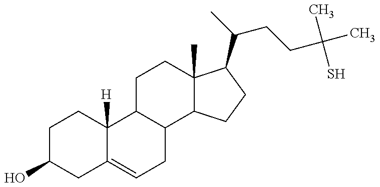 [SMILES: CC(CCC(C)(C)S)[C@H]1CCC2C3CC=C4C[C@@H](O)CC[C@@H]4C3CC[C@@]21C] |
| B69 | 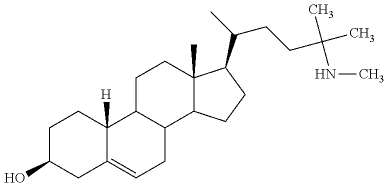 [SMILES: CNC(C)(C)CCC(C)[C@H]1CCC2C3CC=C4C[C@@H](O)CC[C@@H]4C3CC[C@@]21C] |
| B70 | 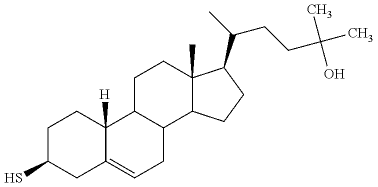 [SMILES: CC(CCC(C)(C)O)[C@H]1CCC2C3CC=C4C[C@@H](S)CC[C@@H]4C3CC[C@@]21C] |
| B71 | 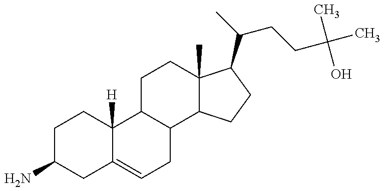 [SMILES: CC(CCC(C)(C)O)[C@H]1CCC2C3CC=C4C[C@@H](N)CC[C@@H]4C3CC[C@@]21C] |
| B72 | 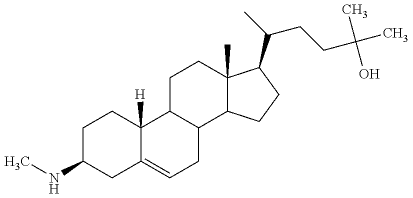 [SMILES: CN[C@H]1CC[C@H]2C(=CCC3C2CC[C@@]2(C)C3CC[C@@H]2C(C)CCC(C)(C)O)C1] |
| B73 | 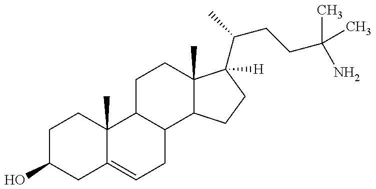 [SMILES: C[C@H](CCC(C)(C)N)[C@H]1CCC2C3CC=C4C[C@@H](O)CC[C@]4(C)C3CC[C@@]21C] |
| B74 | 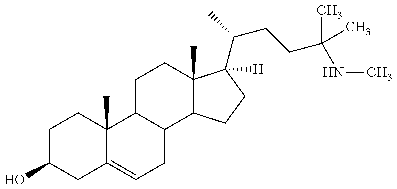 [SMILES: CNC(C)(C)CC[C@@H](C)[C@H]1CCC2C3CC=C4C[C@@H](O)CC[C@]4(C)C3CC[C@@]21C] |
| B75 | 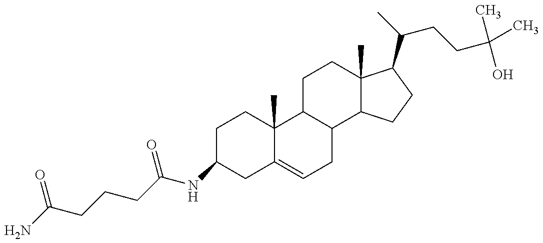 [SMILES: CC(CCC(C)(C)O)[C@H]1CCC2C3CC=C4C[C@@H](NC(=O)CCCC(N)=O)CC[C@]4(C)C3CC[C@@]21C] |
| B76 | 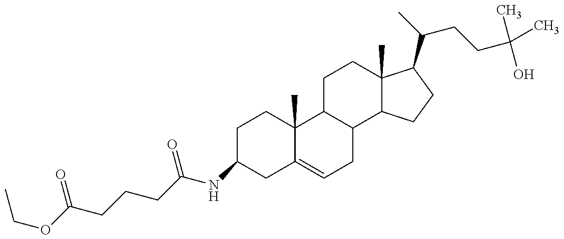 [SMILES: CCOC(=O)CCCC(=O)N[C@H]1CC[C@@]2(C)C(=CCC3C2CC[C@@]2(C)C3CC[C@@H]2C(C)CCC(C)(C)O)C1] |
| B77 | 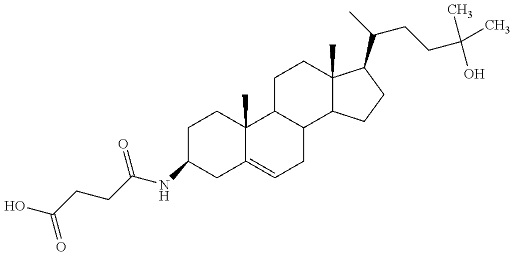 [SMILES: CC(CCC(C)(C)O)[C@H]1CCC2C3CC=C4C[C@@H](NC(=O)CCC(=O)O)CC[C@]4(C)C3CC[C@@]21C] |
| B78 | 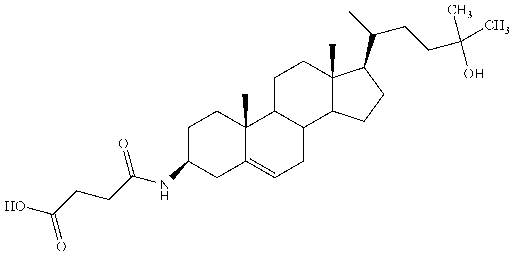 [SMILES: CC(CCC(C)(C)O)[C@H]1CCC2C3CC=C4C[C@@H](NC(=O)CCC(=O)O)CC[C@]4(C)C3CC[C@@]21C] |
| B79 | 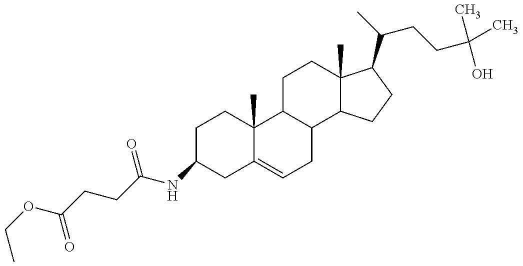 [SMILES: CCOC(=O)CCC(=O)N[C@H]1CC[C@@]2(C)C(=CCC3C2CC[C@@]2(C)C3CC[C@@H]2C(C)CCC(C)(C)O)C1] |
| B80 | 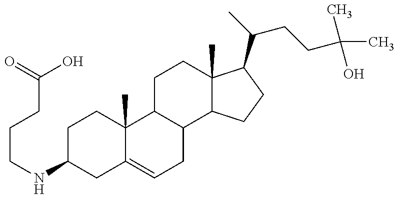 [SMILES: CC(CCC(C)(C)O)[C@H]1CCC2C3CC=C4C[C@@H](NCCCC(=O)O)CC[C@]4(C)C3CC[C@@]21C] |
| B81 | 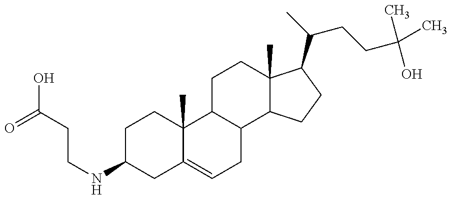 [SMILES: CC(CCC(C)(C)O)[C@H]1CCC2C3CC=C4C[C@@H](NCCC(=O)O)CC[C@]4(C)C3CC[C@@]21C] |
| B82 | 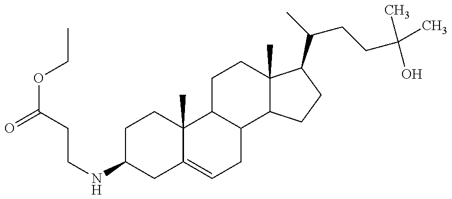 [SMILES: CCOC(=O)CCN[C@H]1CC[C@@]2(C)C(=CCC3C2CC[C@@]2(C)C3CC[C@@H]2C(C)CCC(C)(C)O)C1] |
| B83 | 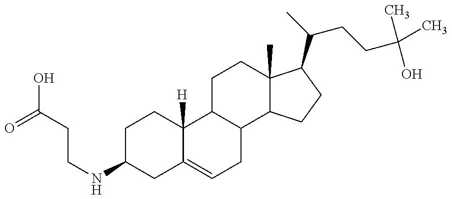 [SMILES: CC(CCC(C)(C)O)[C@H]1CCC2C3CC=C4C[C@@H](NCCC(=O)O)CC[C@@H]4C3CC[C@@]21C] |
| B84 | 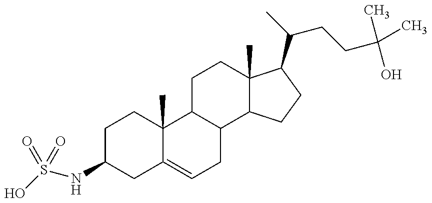 [SMILES: CC(CCC(C)(C)O)[C@H]1CCC2C3CC=C4C[C@@H](NS(=O)(=O)O)CC[C@]4(C)C3CC[C@@]21C] |
| B85 | 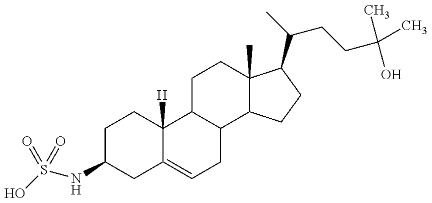 [SMILES: CC(CCC(C)(C)O)[C@H]1CCC2C3CC=C4C[C@@H](NS(=O)(=O)O)CC[C@@H]4C3CC[C@@]21C] |
| B86 | 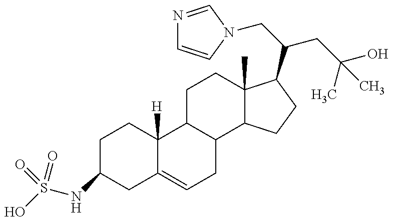 [SMILES: CC(C)(O)CC(Cn1ccnc1)[C@H]1CCC2C3CC=C4C[C@@H](NS(=O)(=O)O)CC[C@@H]4C3CC[C@@]21C] |
| B87 | 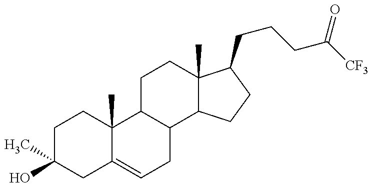 [SMILES: C[C@]1(O)CC[C@@]2(C)C(=CCC3C2CC[C@@]2(C)C3CC[C@@H]2CCCC(=O)C(F)(F)F)C1] |
| B88 | 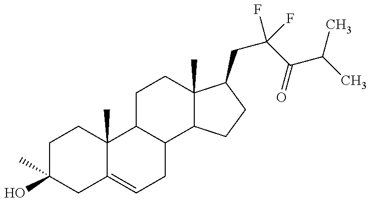 [SMILES: CC(C)C(=O)C(F)(F)C[C@H]1CCC2C3CC=C4C[C@@](C)(O)CC[C@]4(C)C3CC[C@@]21C] |
| B89 | 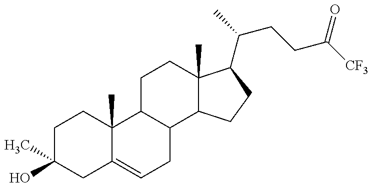 |
| B90 | 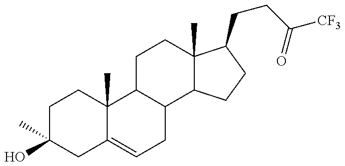 [SMILES: C[C@]1(O)CC[C@@]2(C)C(=CCC3C2CC[C@@]2(C)C3CC[C@@H]2CCC(=O)C(F)(F)F)C1] |
| B91 | 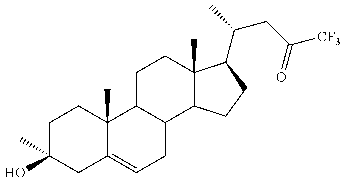 |
| B92 | 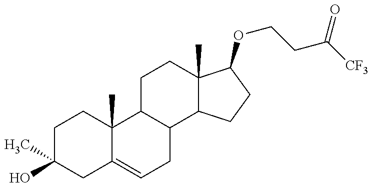 [SMILES: C[C@]1(O)CC[C@@]2(C)C(=CCC3C2CC[C@@]2(C)C3CC[C@@H]2OCCC(=O)C(F)(F)F)C1] |
| B93 | 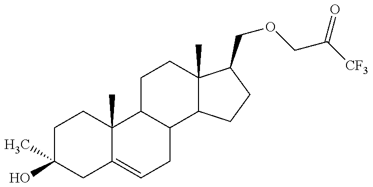 [SMILES: C[C@]1(O)CC[C@@]2(C)C(=CCC3C2CC[C@@]2(C)C3CC[C@@H]2COCC(=O)C(F)(F)F)C1] |
| B94 | 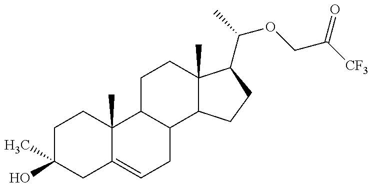 |
| B95 | 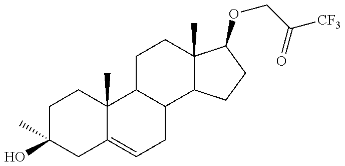 [SMILES: C[C@]1(O)CC[C@@]2(C)C(=CCC3C2CC[C@@]2(C)C3CC[C@@H]2OCC(=O)C(F)(F)F)C1] |
| B96 | 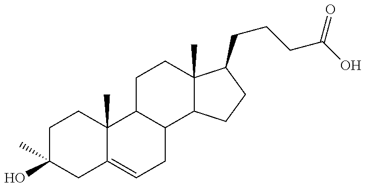 [SMILES: C[C@]1(O)CC[C@@]2(C)C(=CCC3C2CC[C@@]2(C)C3CC[C@@H]2CCCC(=O)O)C1] |
| B97 | 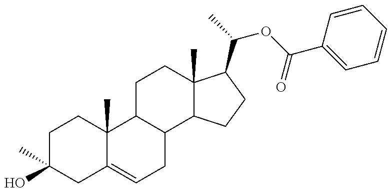 [SMILES: C[C@H](OC(=O)c1ccccc1)[C@H]1CCC2C3CC=C4C[C@@](C)(O)CC[C@]4(C)C3CC[C@@]21C] |
| B98 | 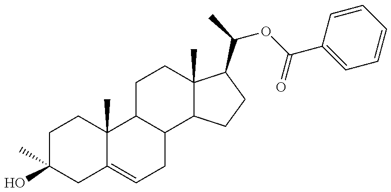 [SMILES: C[C@@H](OC(=O)c1ccccc1)[C@H]1CCC2C3CC=C4C[C@@](C)(O)CC[C@]4(C)C3CC[C@@]21C] |
| B99 | 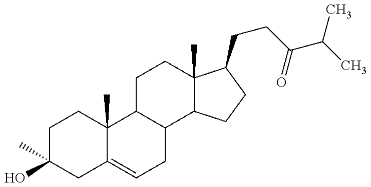 [SMILES: CC(C)C(=O)CC[C@H]1CCC2C3CC=C4C[C@@](C)(O)CC[C@]4(C)C3CC[C@@]21C] |
| B100 | 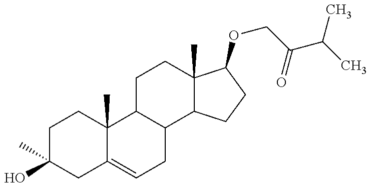 [SMILES: CC(C)C(=O)CO[C@H]1CCC2C3CC=C4C[C@@](C)(O)CC[C@]4(C)C3CC[C@@]21C] |
| B101 | 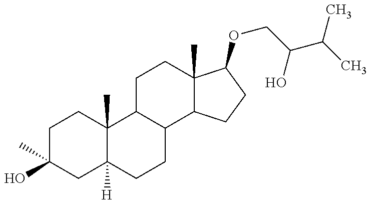 [SMILES: CC(C)C(O)CO[C@H]1CCC2C3CC[C@H]4C[C@@](C)(O)CC[C@]4(C)C3CC[C@@]21C] |
| B102 | 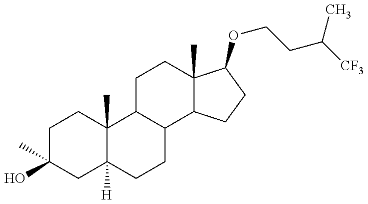 [SMILES: CC(CCO[C@H]1CCC2C3CC[C@H]4C[C@@](C)(O)CC[C@]4(C)C3CC[C@@]21C)C(F)(F)F] |
| B103 | 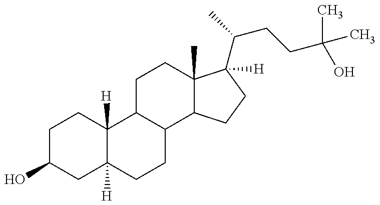 [SMILES: C[C@H](CCC(C)(C)O)[C@H]1CCC2C3CC[C@H]4C[C@@H](O)CC[C@@H]4C3CC[C@@]21C] |
| B104 | 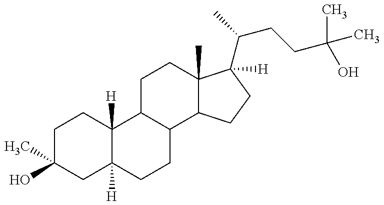 [SMILES: C[C@H](CCC(C)(C)O)[C@H]1CCC2C3CC[C@H]4C[C@@](C)(O)CC[C@@H]4C3CC[C@@]21C] |
| B105 | 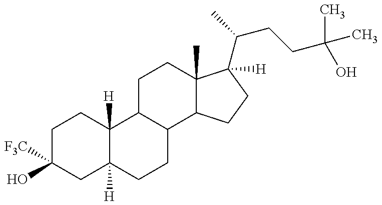 [SMILES: C[C@H](CCC(C)(C)O)[C@H]1CCC2C3CC[C@H]4C[C@](O)(C(F)(F)F)CC[C@@H]4C3CC[C@@]21C] |
| B106 | 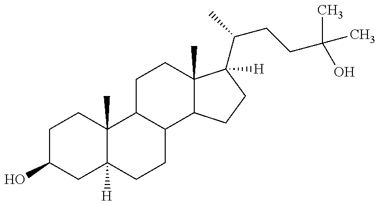 [SMILES: C[C@H](CCC(C)(C)O)[C@H]1CCC2C3CC[C@H]4C[C@@H](O)CC[C@]4(C)C3CC[C@@]21C] |
| B107 | 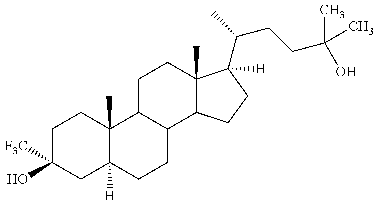 [SMILES: C[C@H](CCC(C)(C)O)[C@H]1CCC2C3CC[C@H]4C[C@](O)(C(F)(F)F)CC[C@]4(C)C3CC[C@@]21C] |
| B108 | 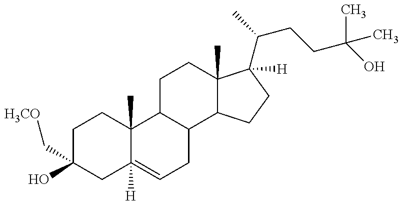 |
| B109 | 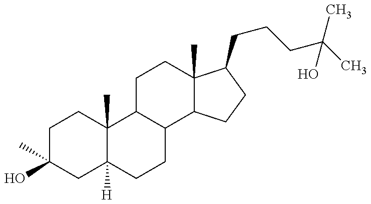 [SMILES: CC(C)(O)CCC[C@H]1CCC2C3CC[C@H]4C[C@@](C)(O)CC[C@]4(C)C3CC[C@@]21C] |
| B110 | 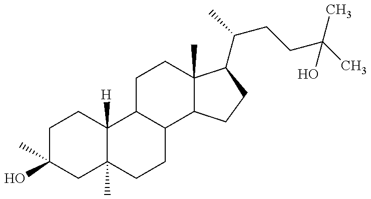 |
| B111 | 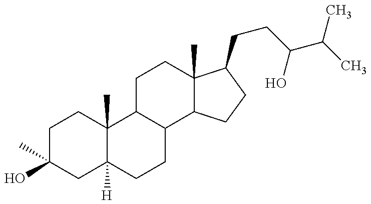 [SMILES: CC(C)C(O)CC[C@H]1CCC2C3CC[C@H]4C[C@@](C)(O)CC[C@]4(C)C3CC[C@@]21C] |
| B112 | 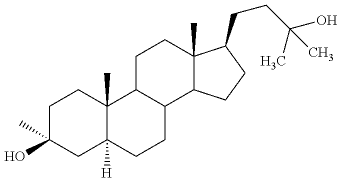 [SMILES: CC(C)(O)CC[C@H]1CCC2C3CC[C@H]4C[C@@](C)(O)CC[C@]4(C)C3CC[C@@]21C] |
| B113 | 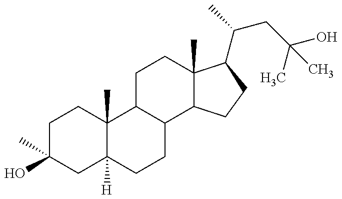 |
| B114 | 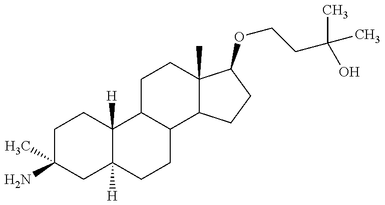 [SMILES: CC(C)(O)CCO[C@H]1CCC2C3CC[C@H]4C[C@@](C)(N)CC[C@@H]4C3CC[C@@]21C] |
| B115 | 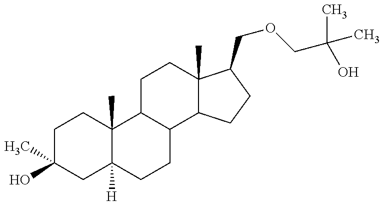 [SMILES: CC(C)(O)COC[C@H]1CCC2C3CC[C@H]4C[C@@](C)(O)CC[C@]4(C)C3CC[C@@]21C] |
| B116 | 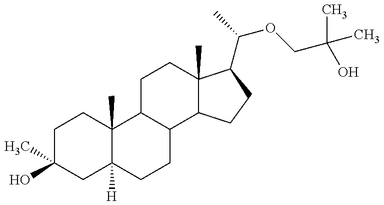 |
| B117 | 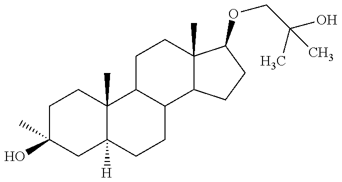 [SMILES: CC(C)(O)CO[C@H]1CCC2C3CC[C@H]4C[C@@](C)(O)CC[C@]4(C)C3CC[C@@]21C] |
| B118 | 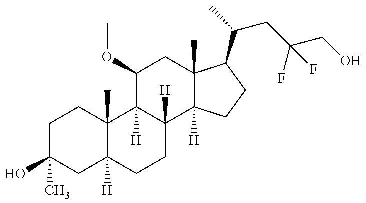 |
| B119 | 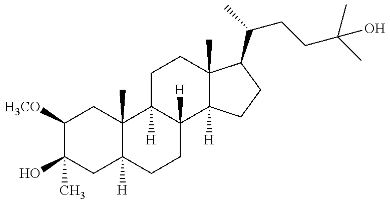 |
| B120 | 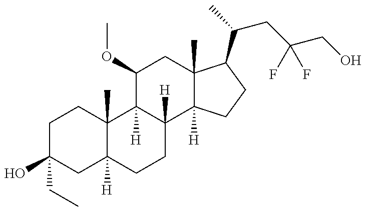 |
| B121 | 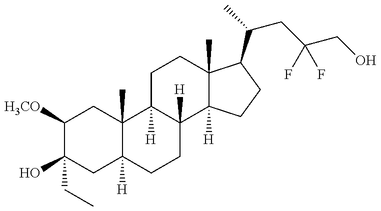 |
| B122 | 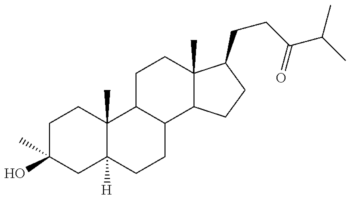 [SMILES: CC(C)C(=O)CC[C@H]1CCC2C3CC[C@H]4C[C@@](C)(O)CC[C@]4(C)C3CC[C@@]21C] |
| B123 | 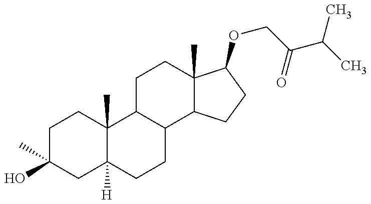 [SMILES: CC(C)C(=O)CO[C@H]1CCC2C3CC[C@H]4C[C@@](C)(O)CC[C@]4(C)C3CC[C@@]21C] |
| B124 | 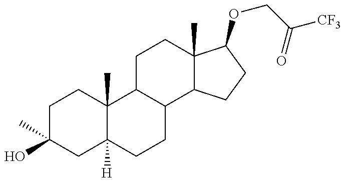 [SMILES: C[C@]1(O)CC[C@]2(C)C3CC[C@@]4(C)C(CC[C@@H]4OCC(=O)C(F)(F)F)C3CC[C@H]2C1] |
| B125 | 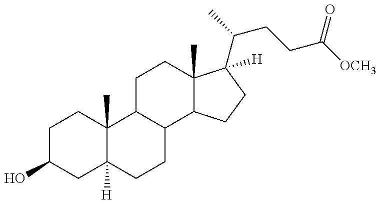 [SMILES: COC(=O)CC[C@@H](C)[C@H]1CCC2C3CC[C@H]4C[C@@H](O)CC[C@]4(C)C3CC[C@@]21C] |
| B126 | 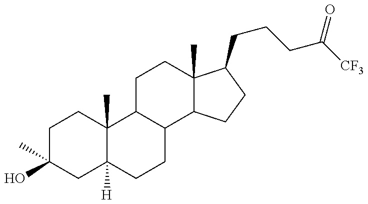 [SMILES: C[C@]1(O)CC[C@]2(C)C3CC[C@@]4(C)C(CC[C@@H]4CCCC(=O)C(F)(F)F)C3CC[C@H]2C1] |
| B127 | 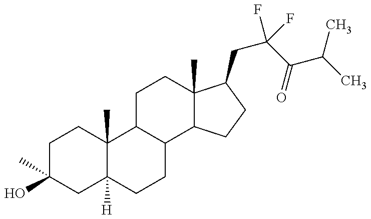 [SMILES: CC(C)C(=O)C(F)(F)C[C@H]1CCC2C3CC[C@H]4C[C@@](C)(O)CC[C@]4(C)C3CC[C@@]21C] |
| B128 | 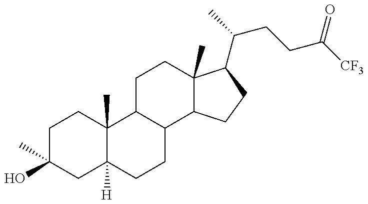 |
| B129 | 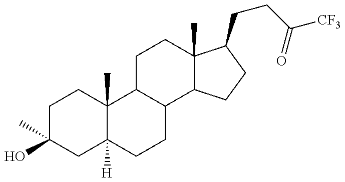 [SMILES: C[C@]1(O)CC[C@]2(C)C3CC[C@@]4(C)C(CC[C@@H]4CCC(=O)C(F)(F)F)C3CC[C@H]2C1] |
| B130 | 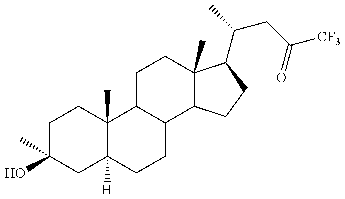 |
| B131 | 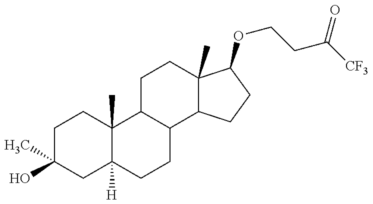 [SMILES: C[C@]1(O)CC[C@]2(C)C3CC[C@@]4(C)C(CC[C@@H]4OCCC(=O)C(F)(F)F)C3CC[C@H]2C1] |
| B132 | 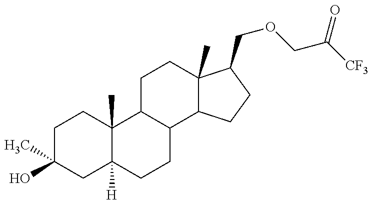 [SMILES: C[C@]1(O)CC[C@]2(C)C3CC[C@@]4(C)C(CC[C@@H]4COCC(=O)C(F)(F)F)C3CC[C@H]2C1] |
| B133 | 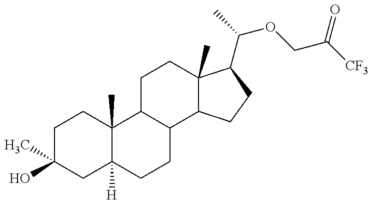 |
| B134 | 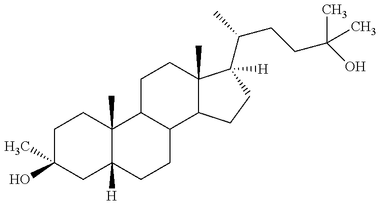 |
| B135 | 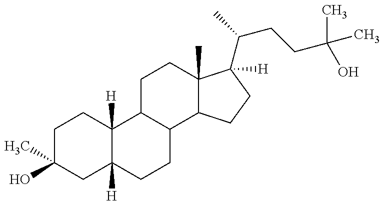 [SMILES: C[C@H](CCC(C)(C)O)[C@H]1CCC2C3CC[C@@H]4C[C@@](C)(O)CC[C@@H]4C3CC[C@@]21C] |
| B136 | 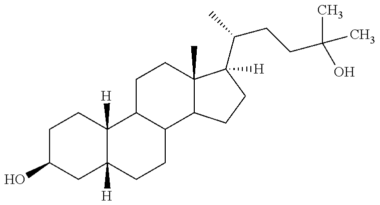 [SMILES: C[C@H](CCC(C)(C)O)[C@H]1CCC2C3CC[C@@H]4C[C@@H](O)CC[C@@H]4C3CC[C@@]21C] |
| B137 | 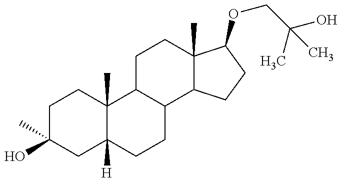 [SMILES: CC(C)(O)CO[C@H]1CCC2C3CC[C@@H]4C[C@@](C)(O)CC[C@]4(C)C3CC[C@@]21C] |
| B138 | 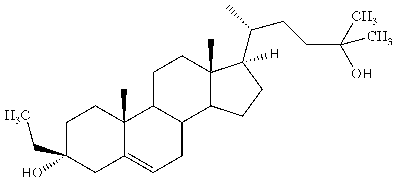 [SMILES: CC[C@@]1(O)CC[C@@]2(C)C(=CCC3C2CC[C@@]2(C)C3CC[C@@H]2[C@H](C)CCC(C)(C)O)C1] |
| B139 | 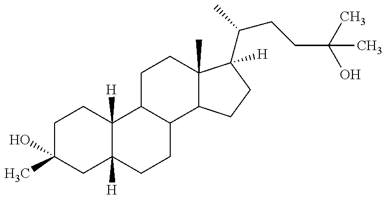 [SMILES: C[C@H](CCC(C)(C)O)[C@H]1CCC2C3CC[C@@H]4C[C@](C)(O)CC[C@@H]4C3CC[C@@]21C] |
| B140 | 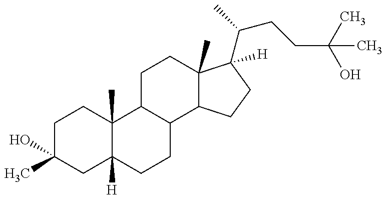 [SMILES: C[C@H](CCC(C)(C)O)[C@H]1CCC2C3CC[C@@H]4C[C@](C)(O)CC[C@]4(C)C3CC[C@@]21C] |
| B141 | 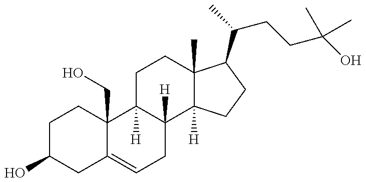 |
| B142 | 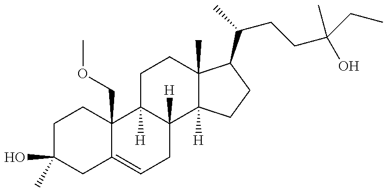 |
| B143 | 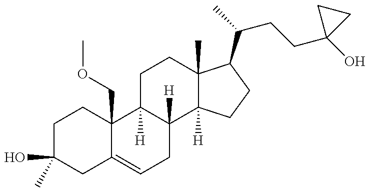 |
| B144 | 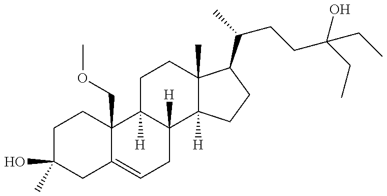 |
| B145 | 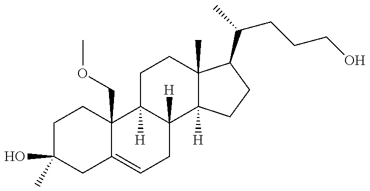 |
| B146 | 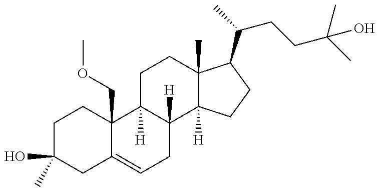 |
| B147 | 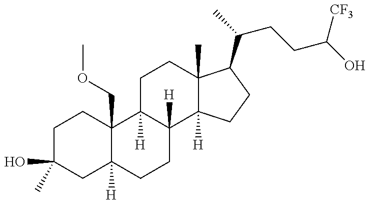 |
| B148 | 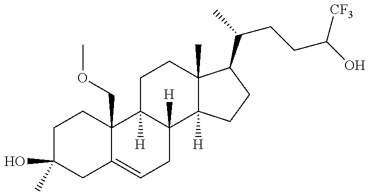 |
| B149 | 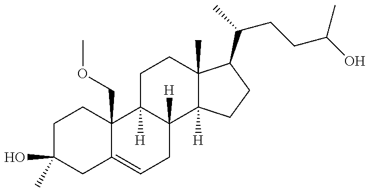 |
| B150 | 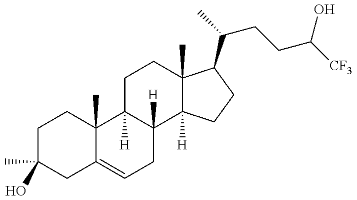 |
| B151 | 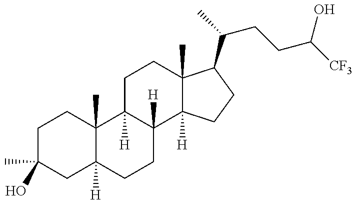 |
| B152 | 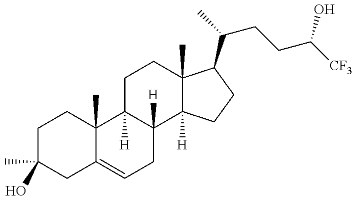 |
| B153 | 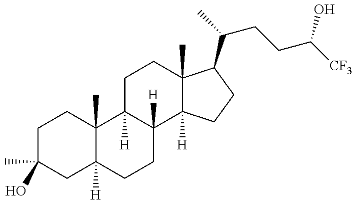 |
| B154 | 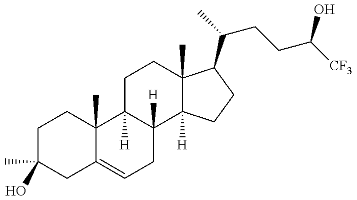 |
| B155 | 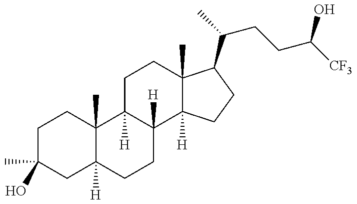 |
| B156 | 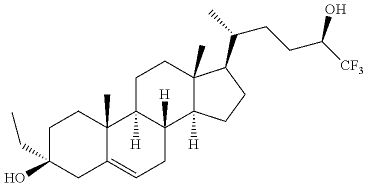 |
| B157 | 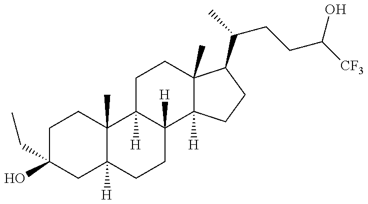 |
| B158 | 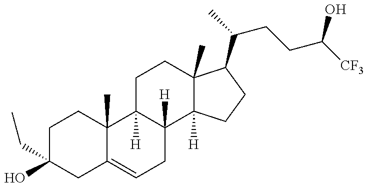 |
| B159 | 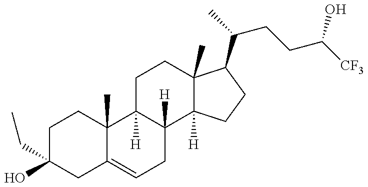 |
| B160 | 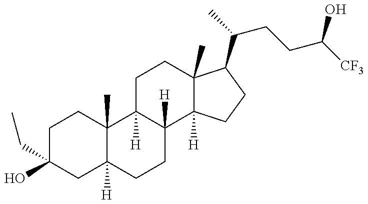 |
| B161 | 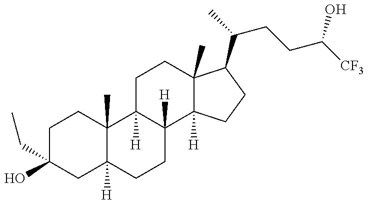 |
| B162 | 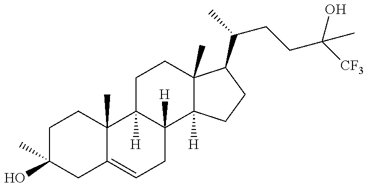 |
| B163 | 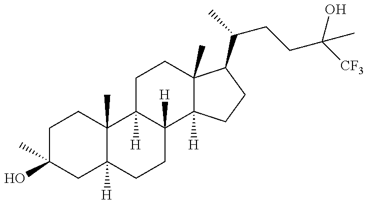 |
| B164 | 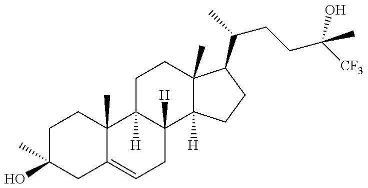 |
| B165 | 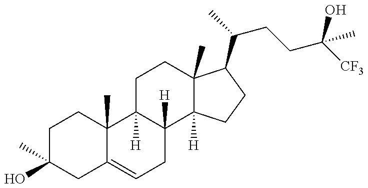 |
| B166 | 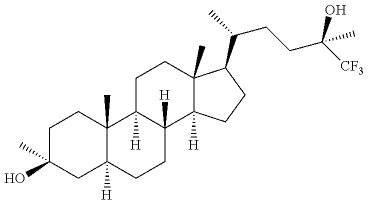 |
| B167 | 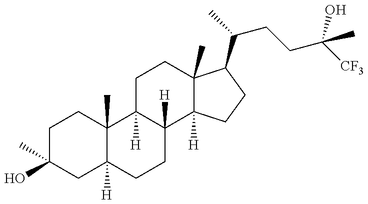 |
| B168 | 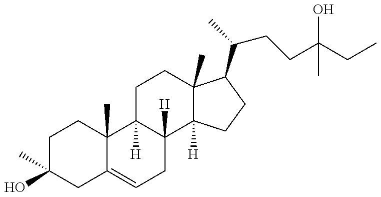 |
| B169 | 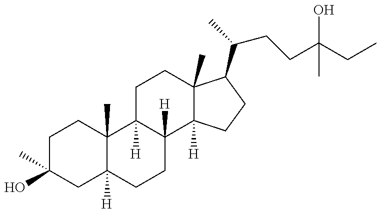 |
| B170 | 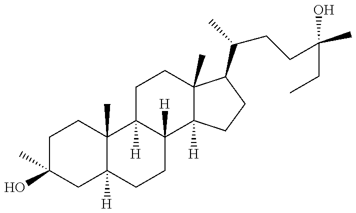 |
| B171 | 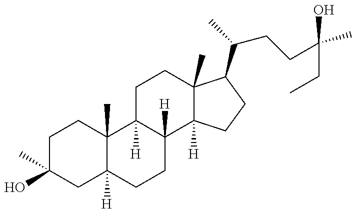 |
| B172 | 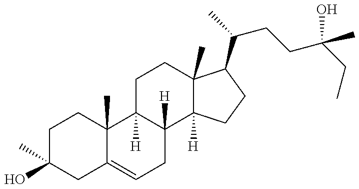 |
| B173 | 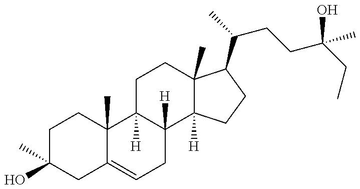 |
| B174 | 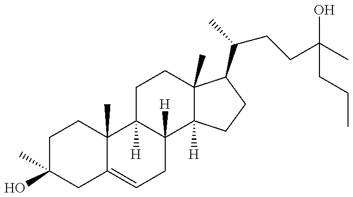 |
| B175 | 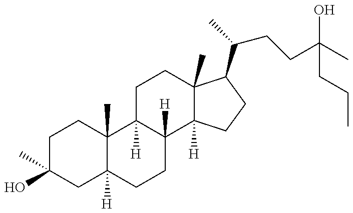 |
| B176 | 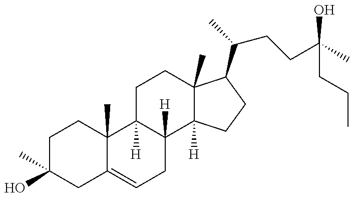 |
| B177 | 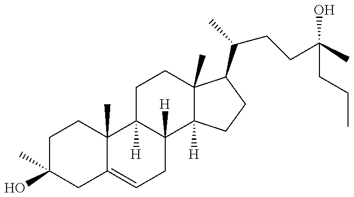 |
| B178 | 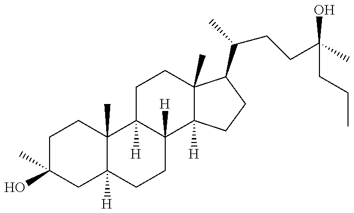 |
| B179 | 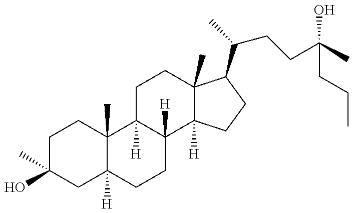 |
| B180 | 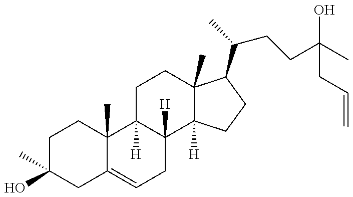 |
| B181 | 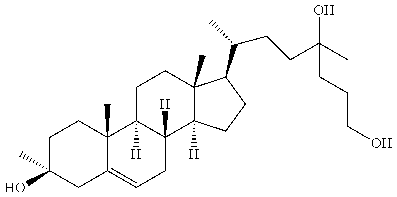 |
| B182 | 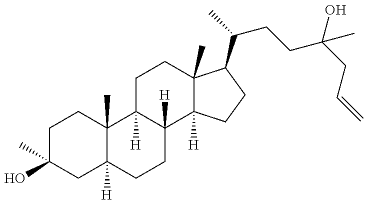 |
| B183 | 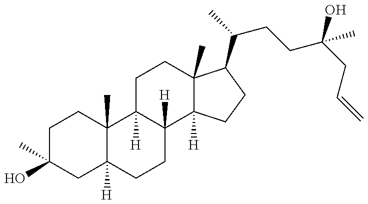 |
| B184 | 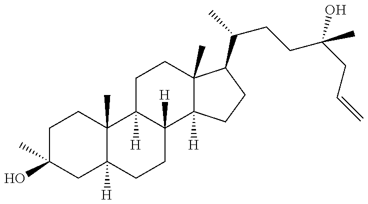 |
| B185 | 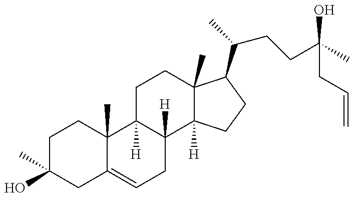 |
| B186 | 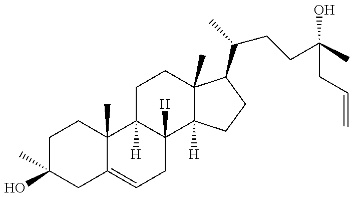 |
| B187 | 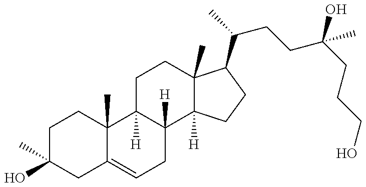 |
| B188 | 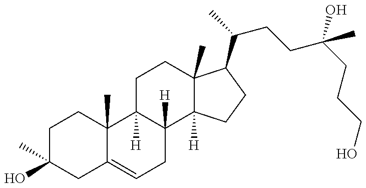 |
| B189 | 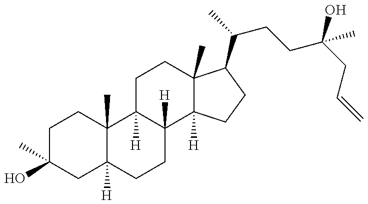 |
| B190 | 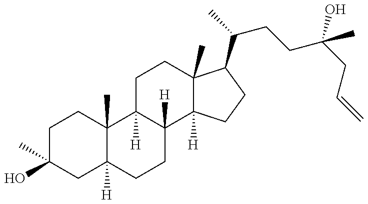 |
| B191 | 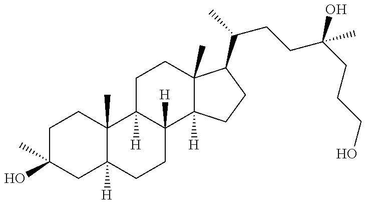 |
| B192 | 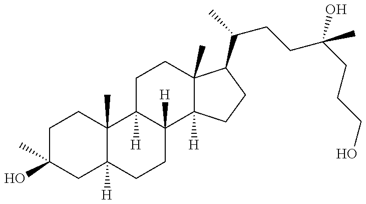 |
| B193 | 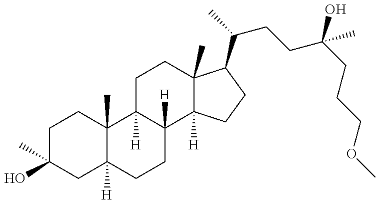 |
| B194 | 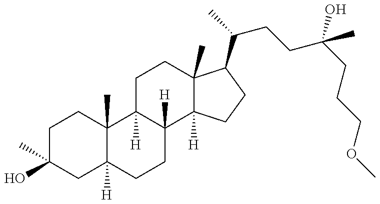 |
| B195 | 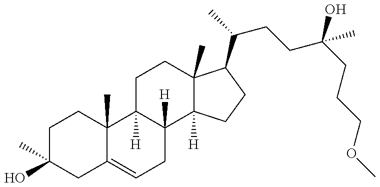 |
| B196 | 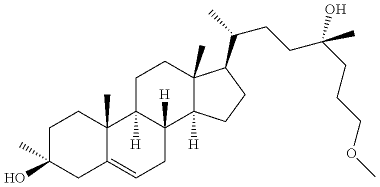 |
| B197 | 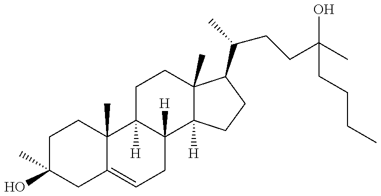 |
| B198 | 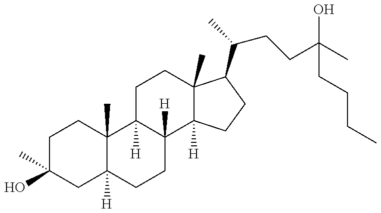 |
| B199 | 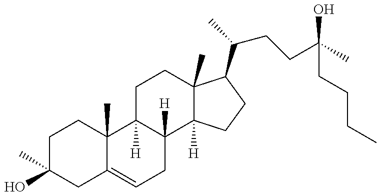 |
| B200 | 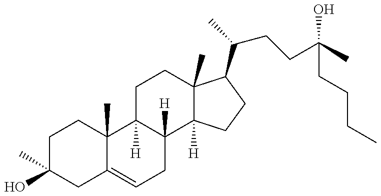 |
| B201 | 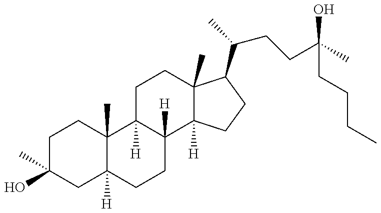 |
| B202 | 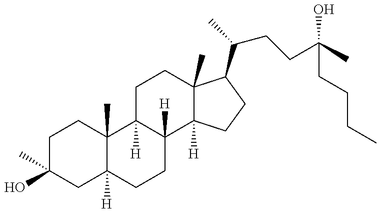 |
| B203 | 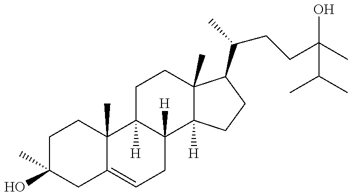 |
| B204 | 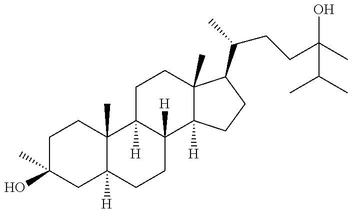 |
| B205 | 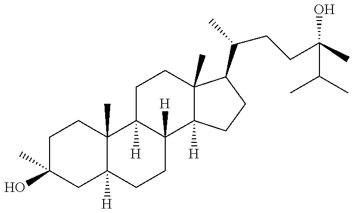 |
| B206 | 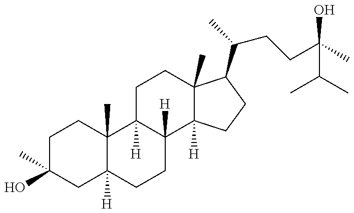 |
| B207 | 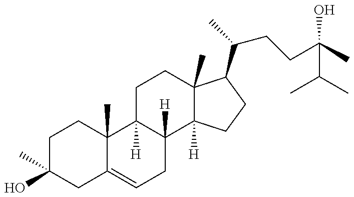 |
| B208 | 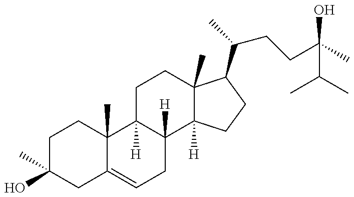 |
| B209 | 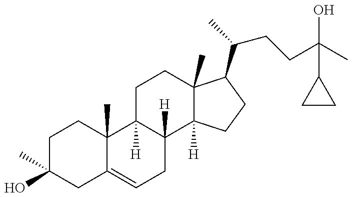 |
| B210 | 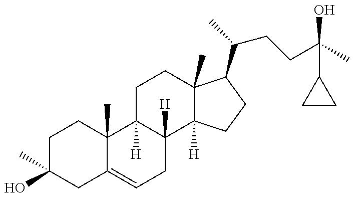 |
| B211 | 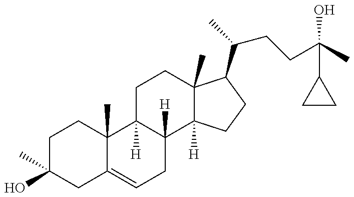 |
| B212 | 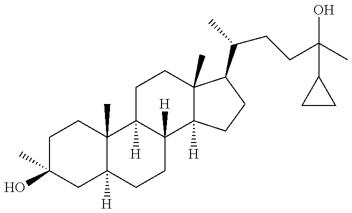 |
| B213 | 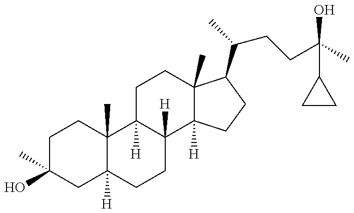 |
| B214 | 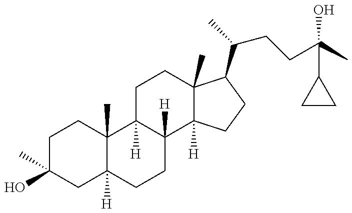 |
| B215 | 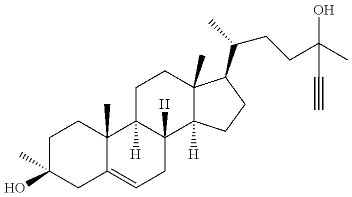 |
| B216 | 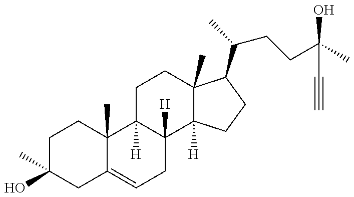 |
| B217 | 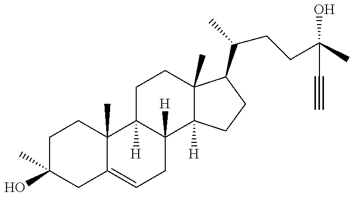 |
| B218 | 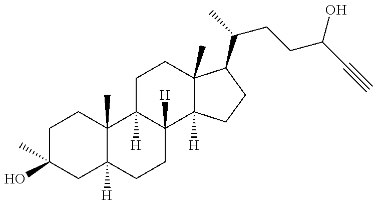 |
| B219 | 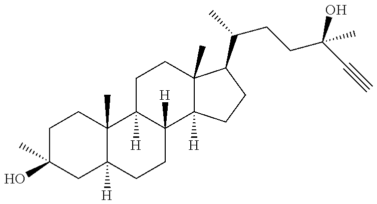 |
| B220 | 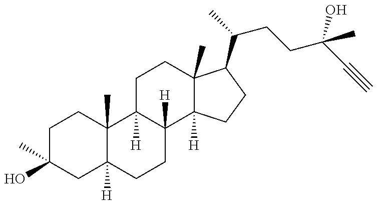 |
| B221 | 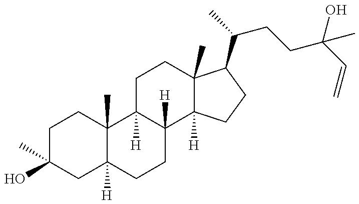 |
| B222 | 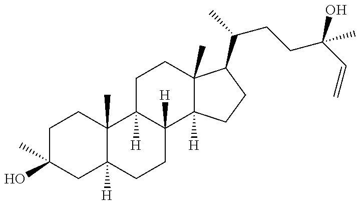 |
| B223 | 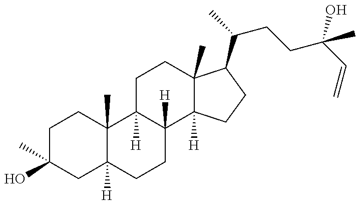 |
| B224 | 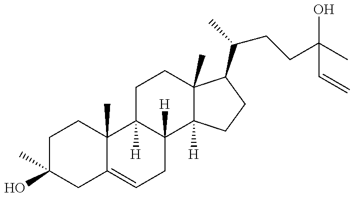 |
| B225 | 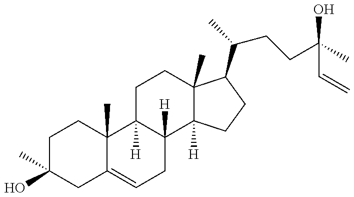 |
| B226 | 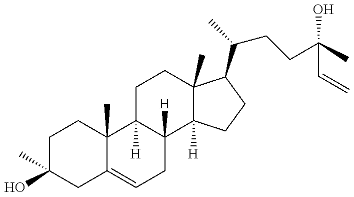 |
| B227 | 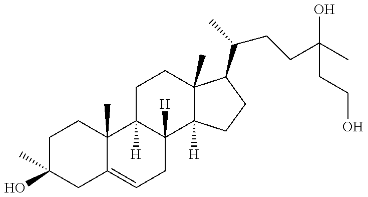 |
| B228 | 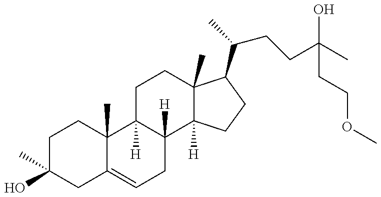 |
| B229 | 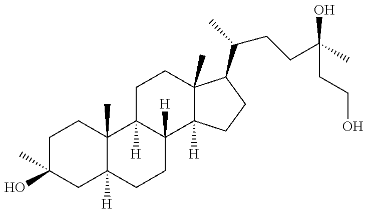 |
| B230 | 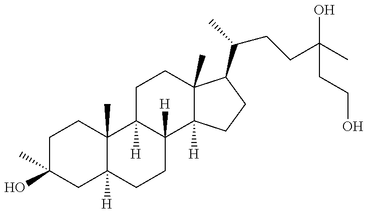 |
| B231 | 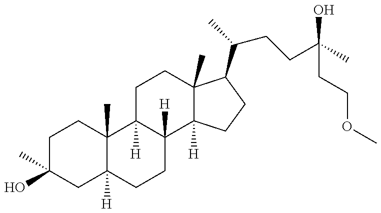 |
| B232 | 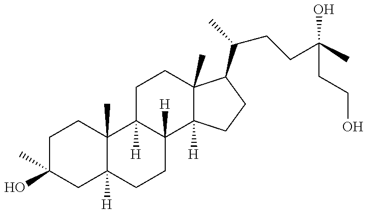 |
| B233 | 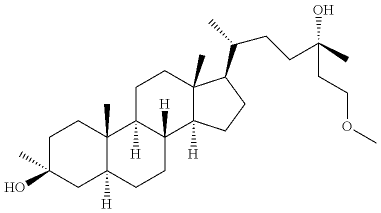 |
| B234 | 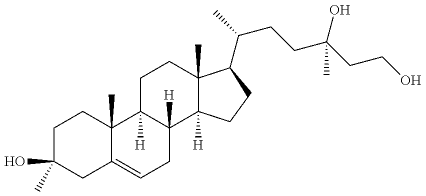 |
| B235 | 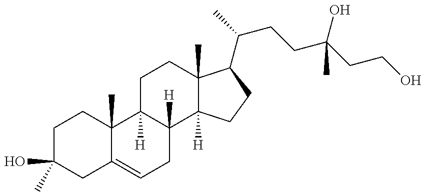 |
| B236 | 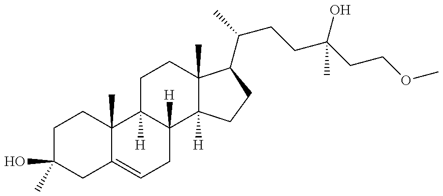 |
| B237 | 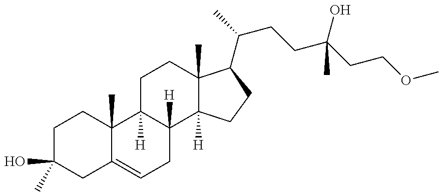 |
| B238 | 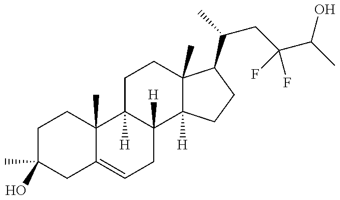 |
| B239 | 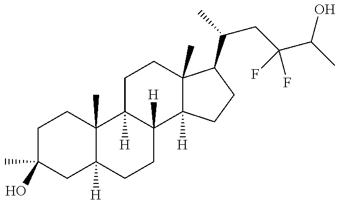 |
| B240 | 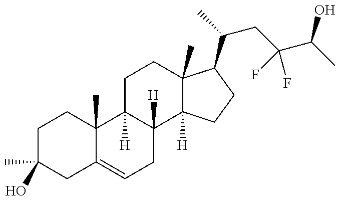 |
| B241 | 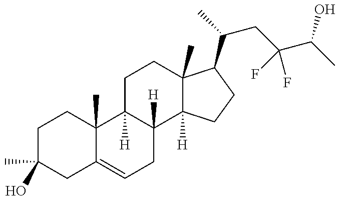 |
| B242 | 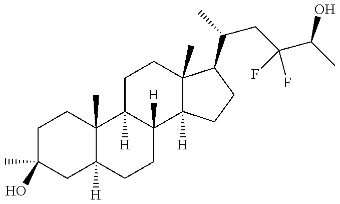 |
| B243 | 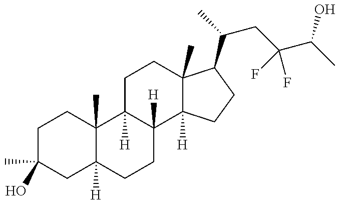 |
| B244 | 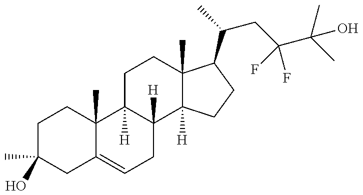 |
| B245 | 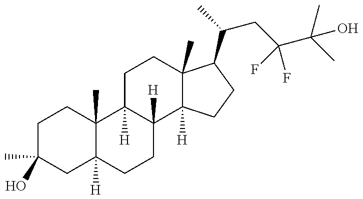 |
| B246 | 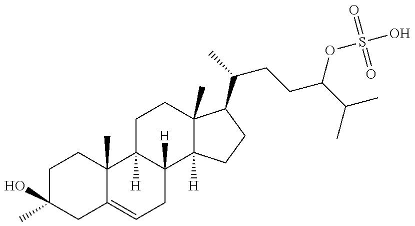 |
| B247 | 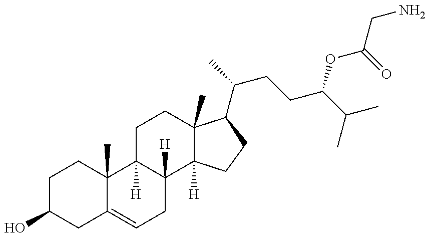 |
| B248 | 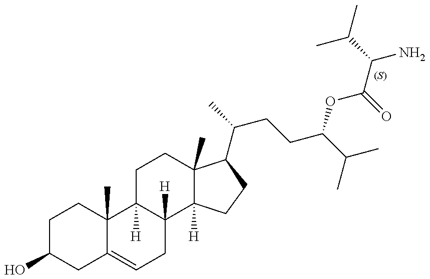 |
| B249 | 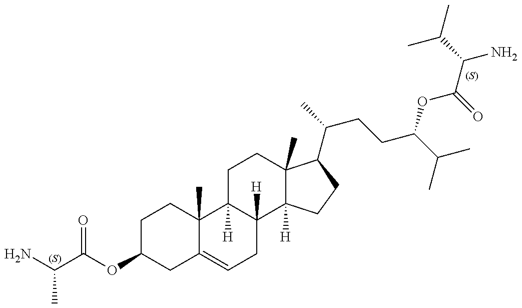 |
| B250 | 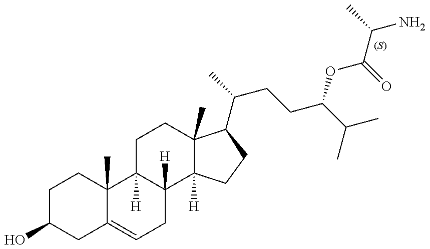 |
| B251 | 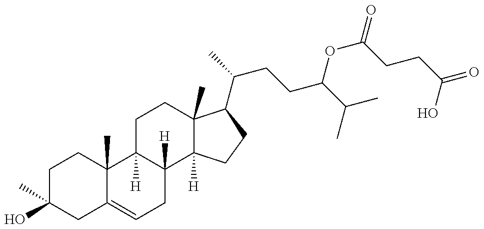 |
| B252 | 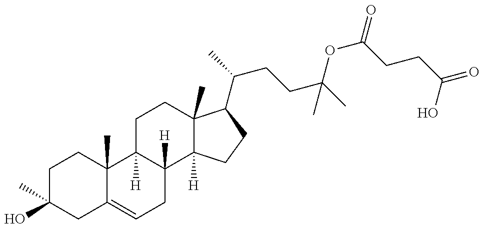 |
| B253 | 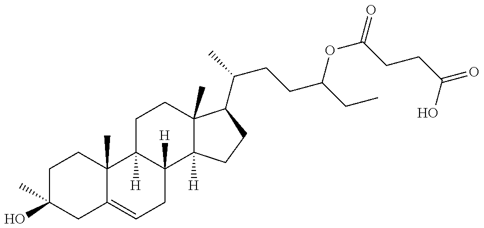 |
| B254 | 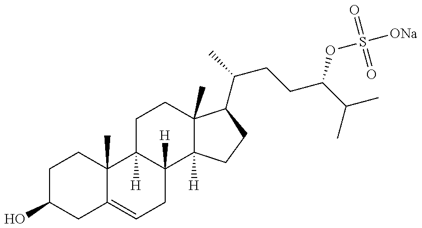 |
| B255 | 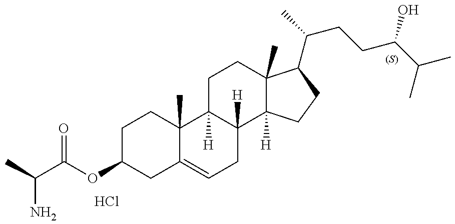 |
| B256 | 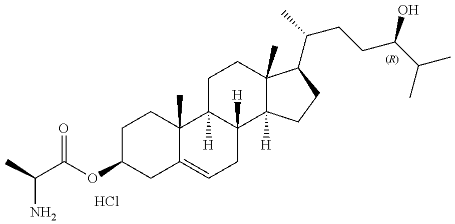 |
| B257 | 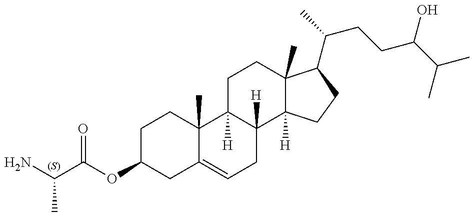 |
| B258 | 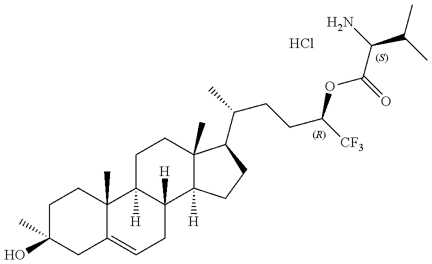 |
| B259 | 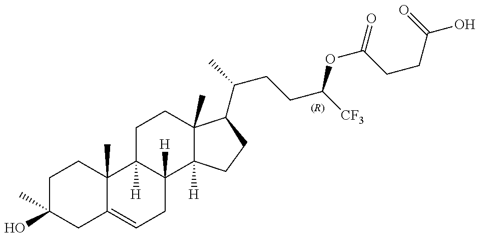 |
| B260 | 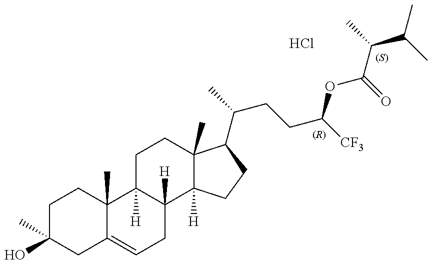 |
| B261 | 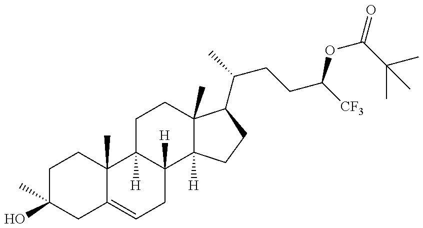 |
| B262 | 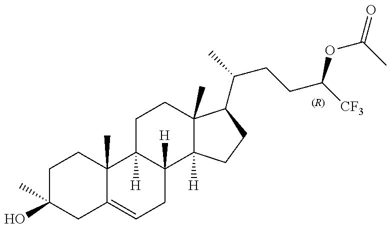 |
| B263 | 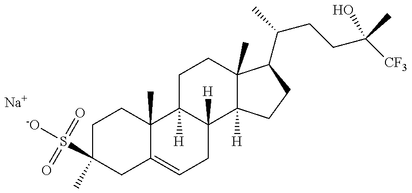 |
| B264 | 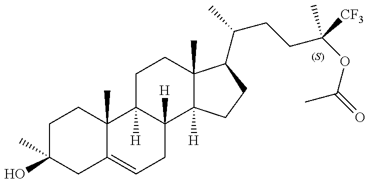 |
| B265 | 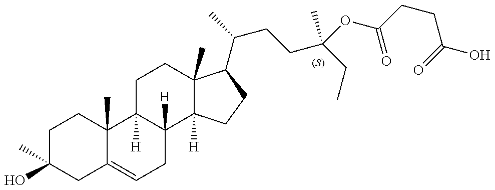 |
| B266 | 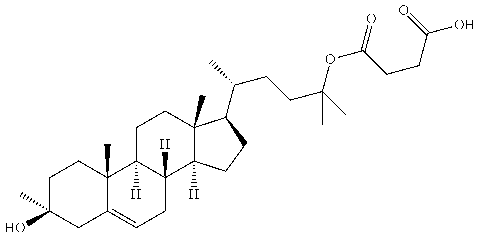 |
| B267 | 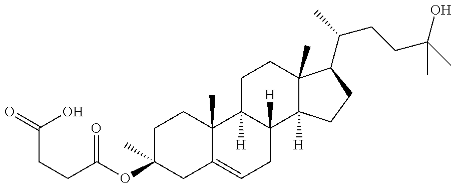 |
| B268 | 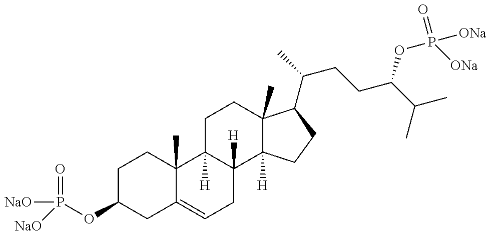 |
| B269 | 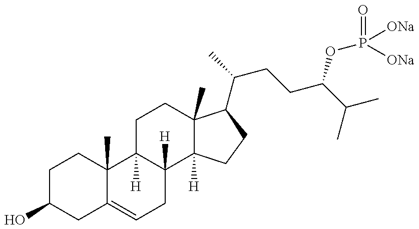 |
| B270 | 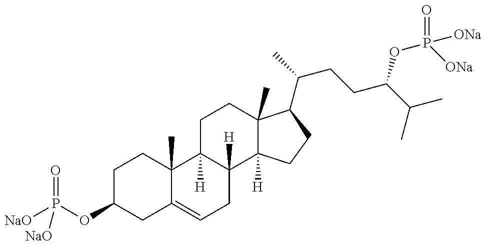 |
| B271 | 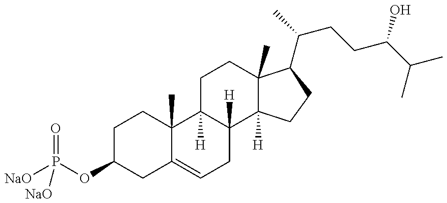 |
| B272 | 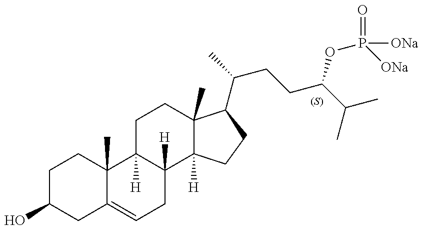 |
| B273 | 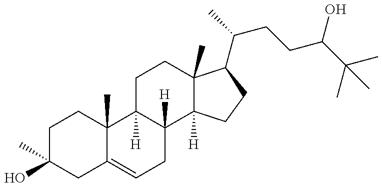 |
| B274 | 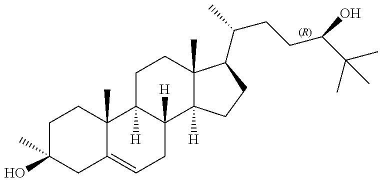 |
| B275 | 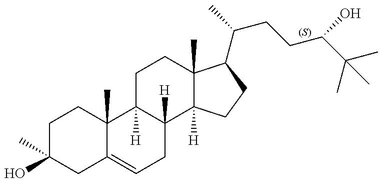 |
| B276 | 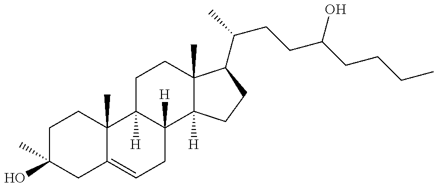 |
| B277 | 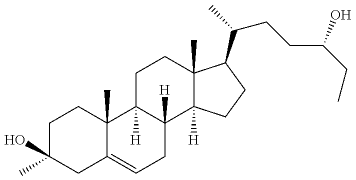 |
| B278 | 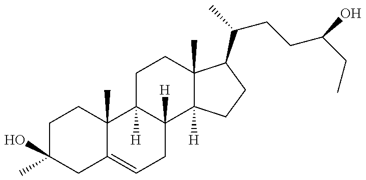 |
| B279 | 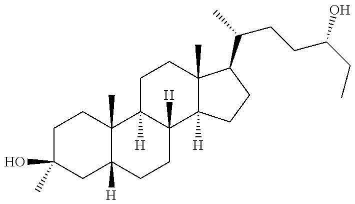 |
| B280 | 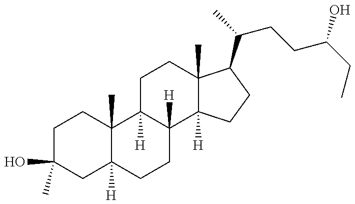 |
| B281 | 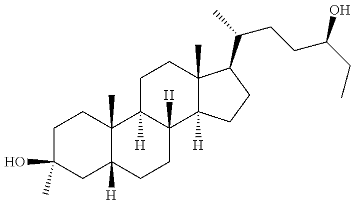 |
| B282 | 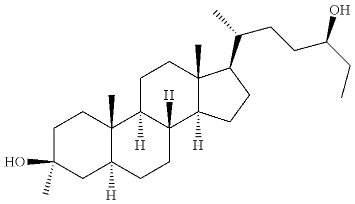 |
| B283 | 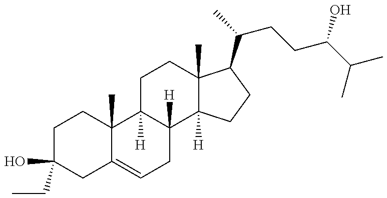 |
| B284 | 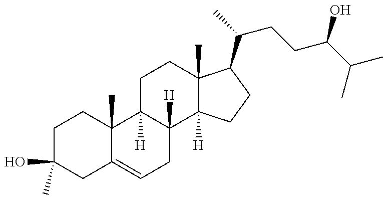 |
| B285 | 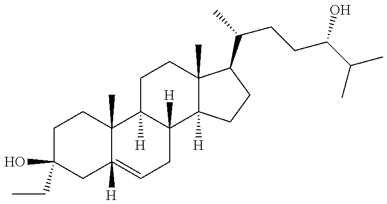 |
| B286 | 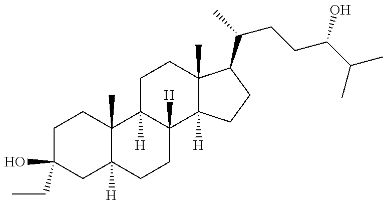 |
| B287 | 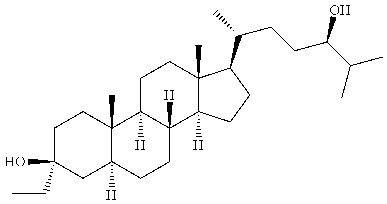 |
| B288 | 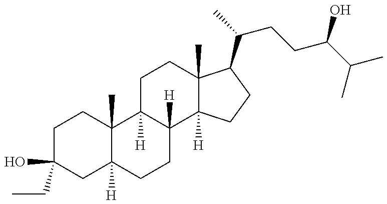 |
| B289 | 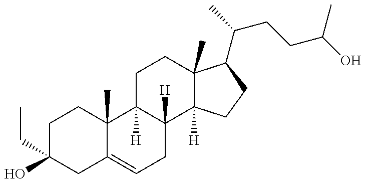 |
| B290 | 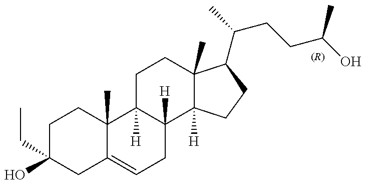 |
| B291 | 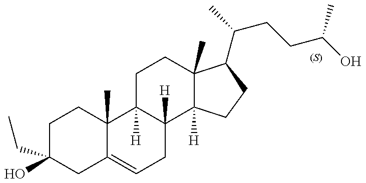 |
| B292 | 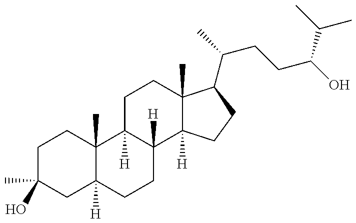 |
| B293 | 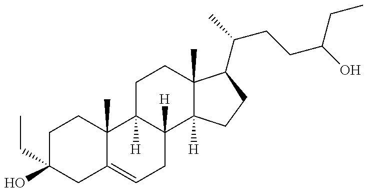 |
| B294 | 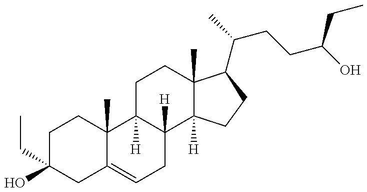 |
| B295 | 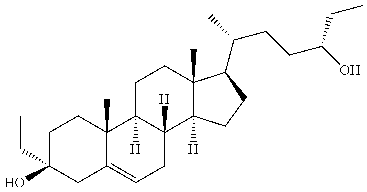 |
| B296 | 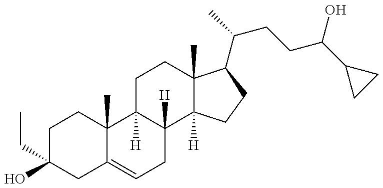 |
| B297 | 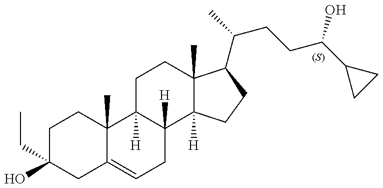 |
| B298 | 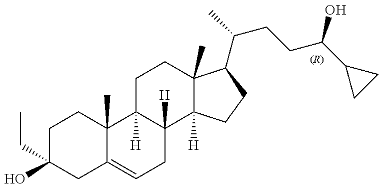 |
| B299 | 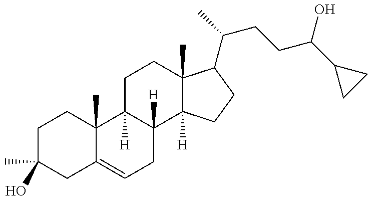 |
| B300 | 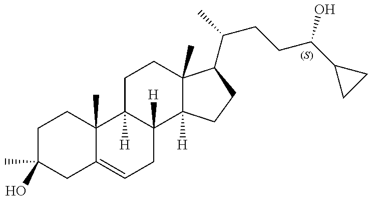 |
| B301 | 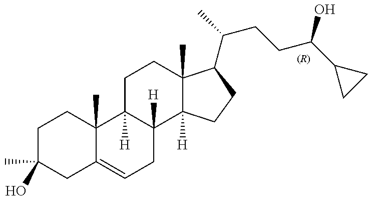 |
| B302 | 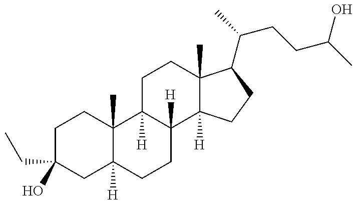 |
| B303 | 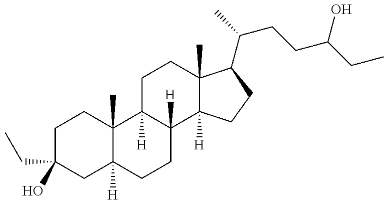 |
| B304 | 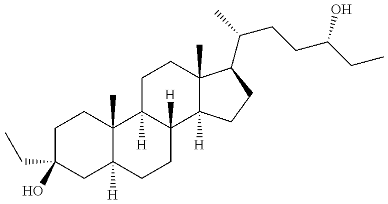 |
| B305 | 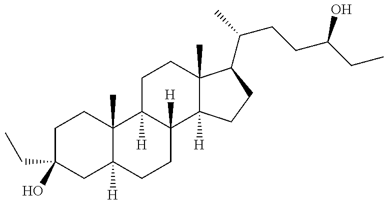 |
| B306 | 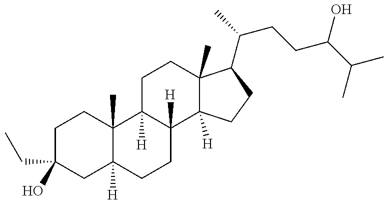 |
| B307 | 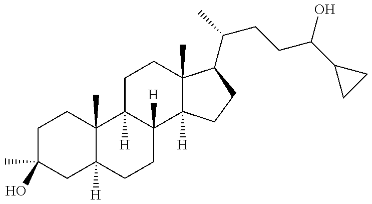 |
| B308 | 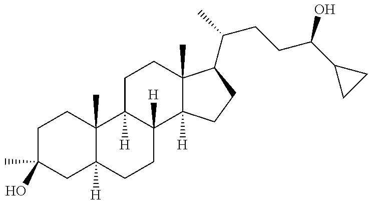 |
| B309 | 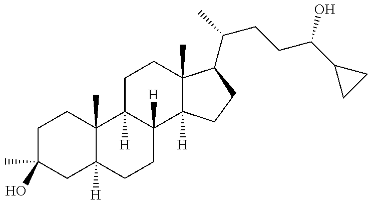 |
| B310 | 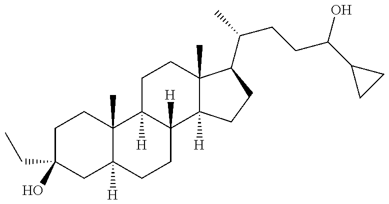 |
| B311 | 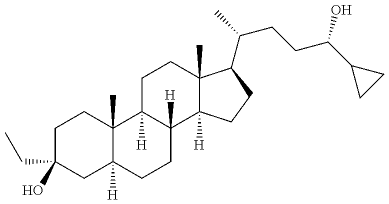 |
| B312 | 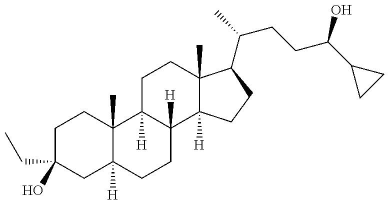 |
| B313 | 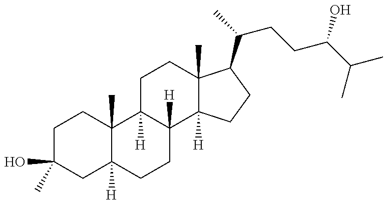 |
| B314 | 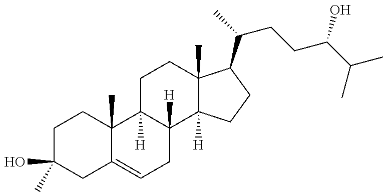 |
| B315 | 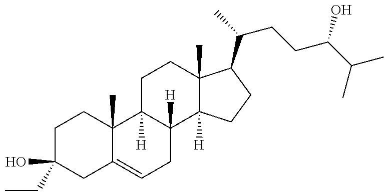 |
| B316 | 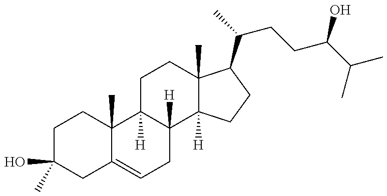 |
| B317 | 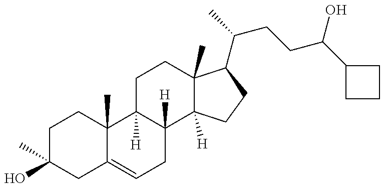 |
| B318 | 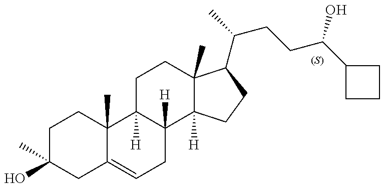 |
| B319 | 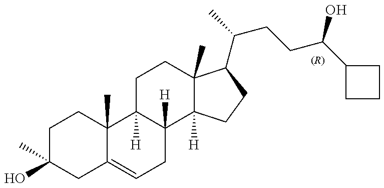 |
| B320 | 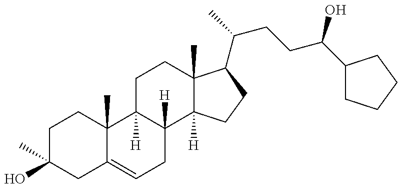 |
| B321 | 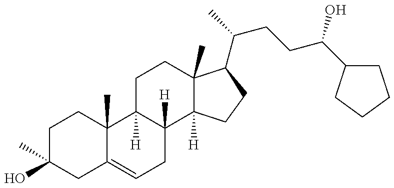 |
| B322 | 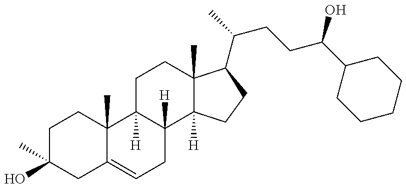 |
| B323 | 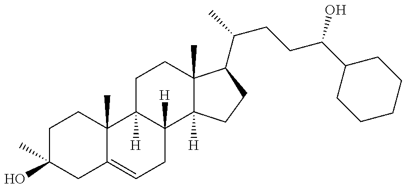 |
| B324 | 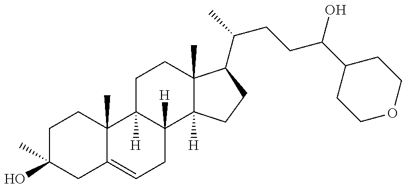 |
| B325 | 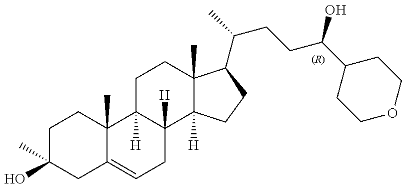 |
| B326 | 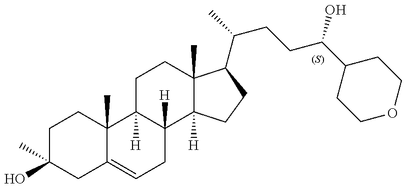 |
| B327 | 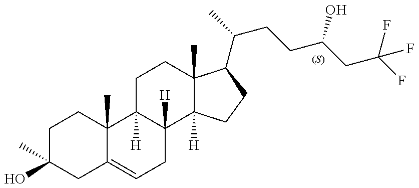 |
| B328 | 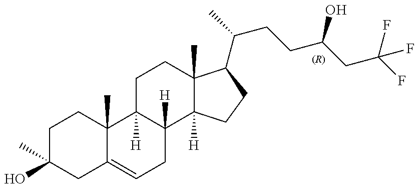 |
| B329 | 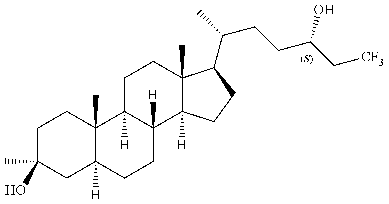 |
| B330 | 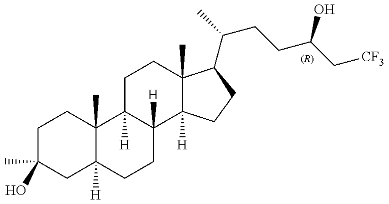 |
| B331 | 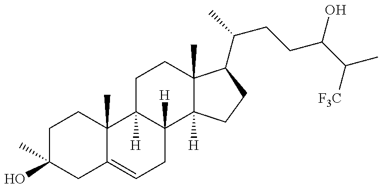 |
| B332 | 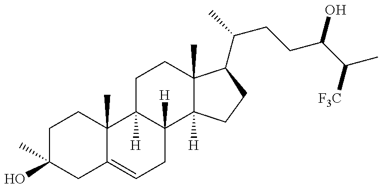 |
| B333 | 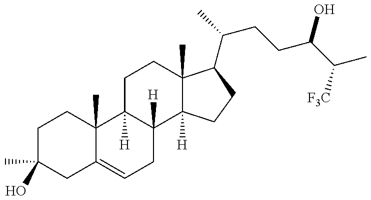 |
| B334 | 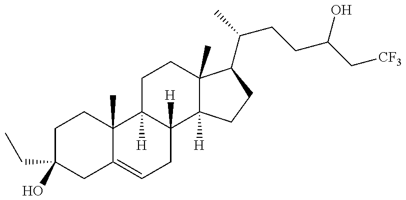 |
| B335 | 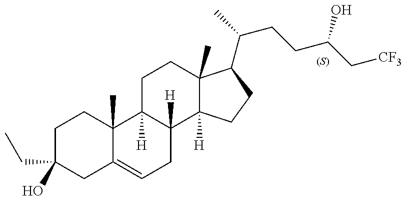 |
| B336 | 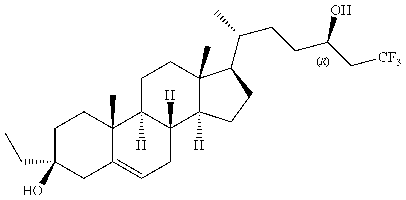 |
| B337 | 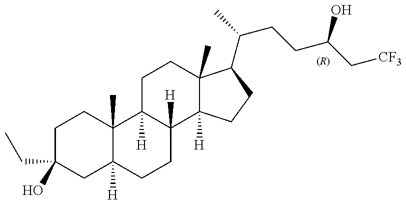 |
| B338 | 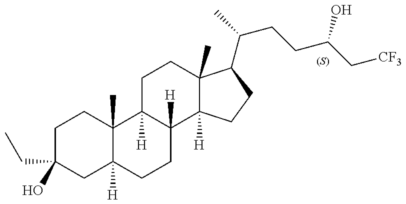 |
| B339 | 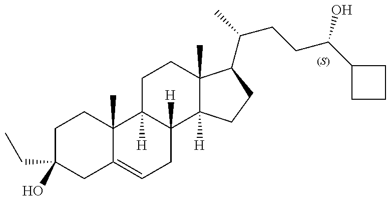 |
| B340 | 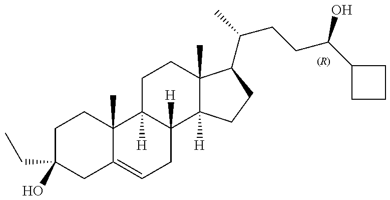 |
| B341 | 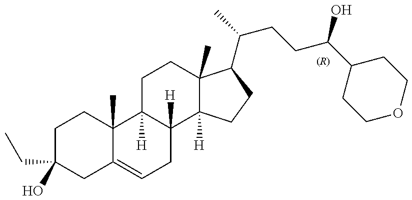 |
| B342 | 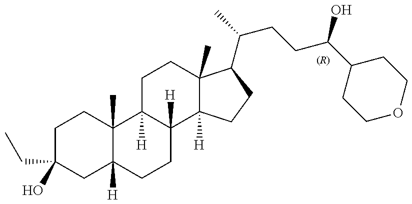 |
| B343 | 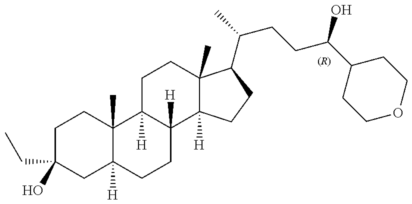 |
| B344 | 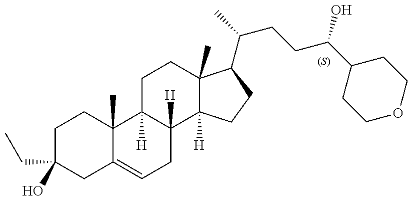 |
| B345 | 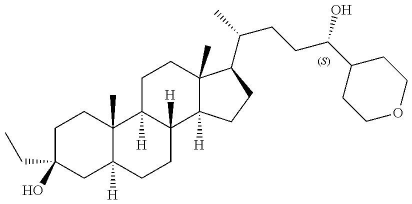 |
| B346 | 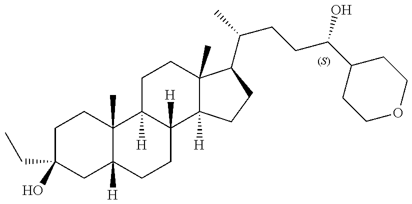 |
| B347 | 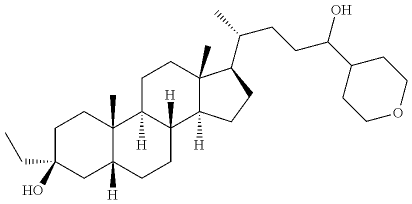 |
| B348 | 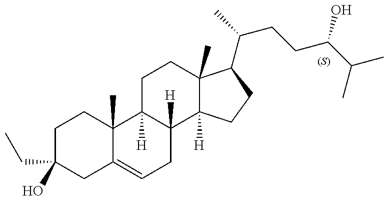 |
| B349 | 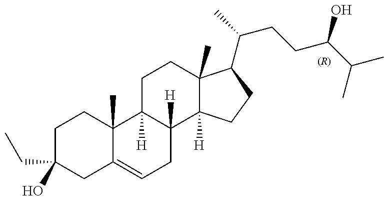 |
| B350 | 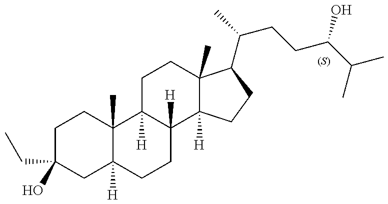 |
| B351 | 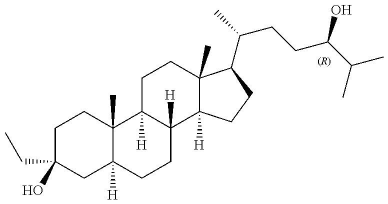 |
| B352 | 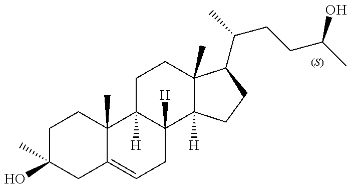 |
| B353 | 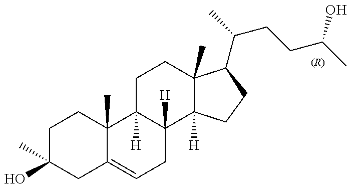 |
| B354 | 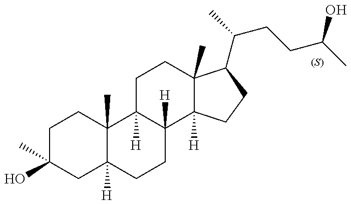 |
| B355 | 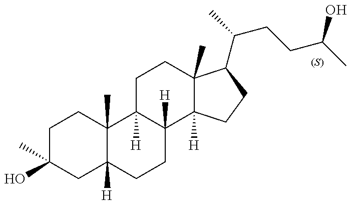 |
| B356 | 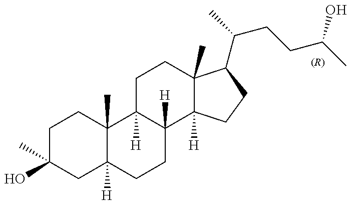 |
| B357 | 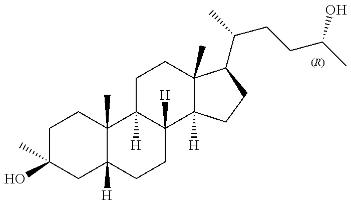 |
| B358 | 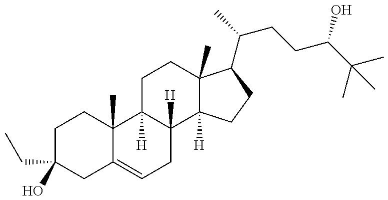 |
| B359 | 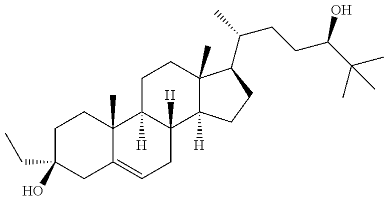 |
| B360 | 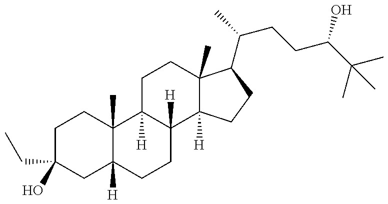 |
| B361 | 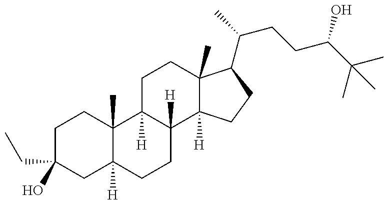 |
| B362 | 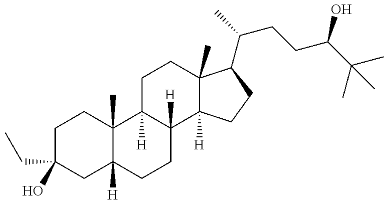 |
| B363 | 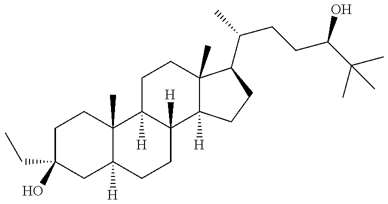 |
| B364 | 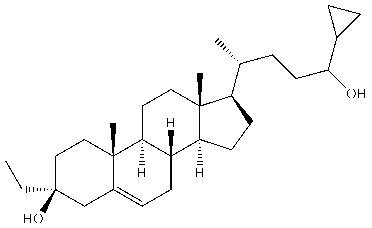 |
| B365 | 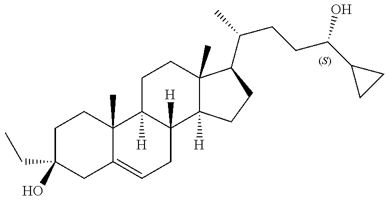 |
| B366 | 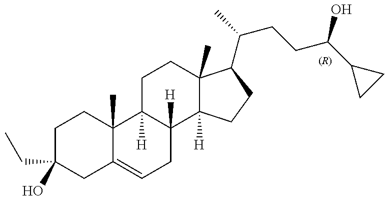 |
| B367 | 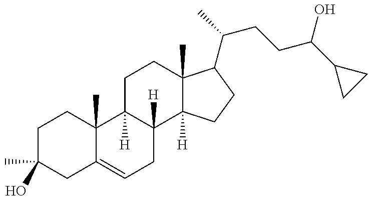 |
| B368 | 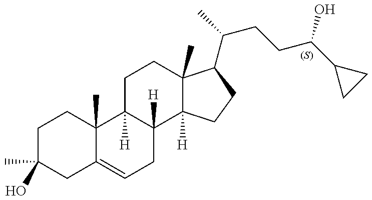 |
| B369 | 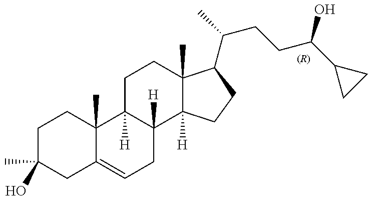 |
| B370 | 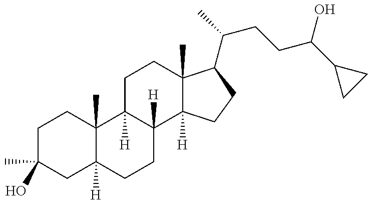 |
| B371 | 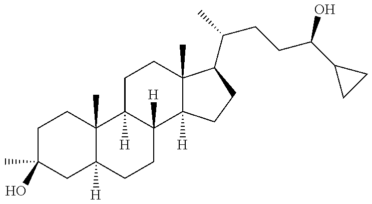 |
| B372 | 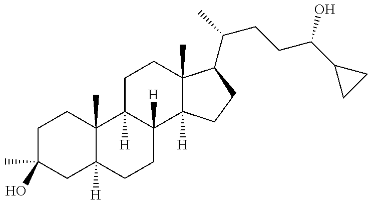 |
| B373 | 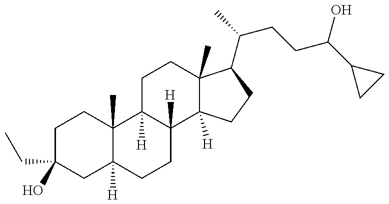 |
| B374 | 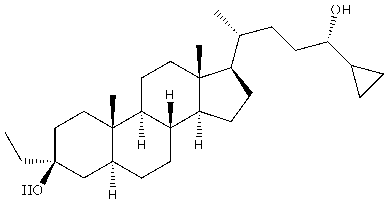 |
| B375 | 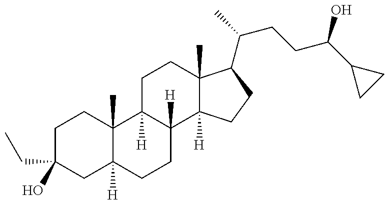 |
| B376 | 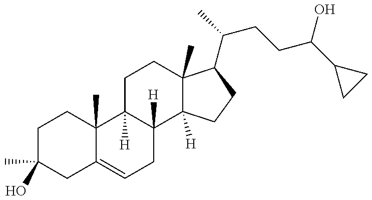 |
| B377 | 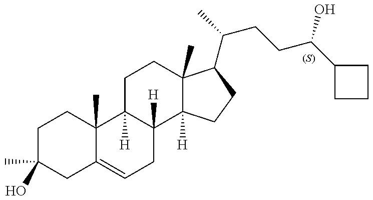 |
| B378 | 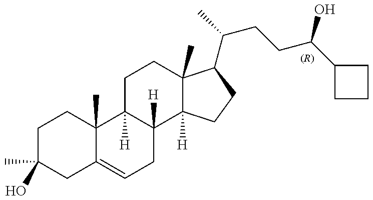 |
| B379 | 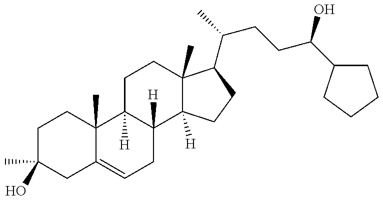 |
| B380 | 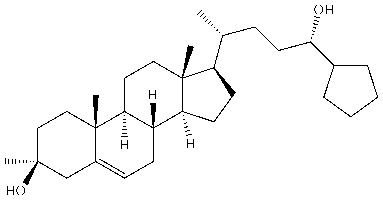 |
| B381 | 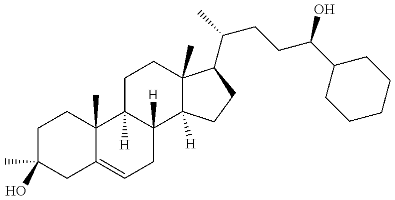 |
| B382 | 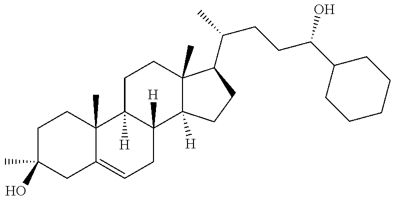 |
| B383 | 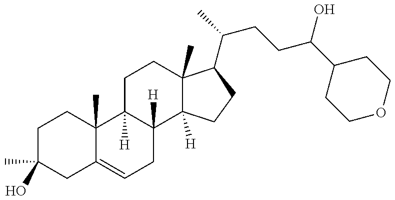 |
| B384 | 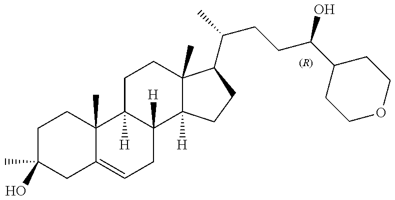 |
| B385 | 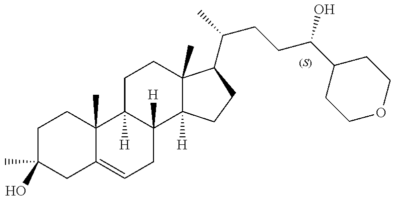 |
| B386 | 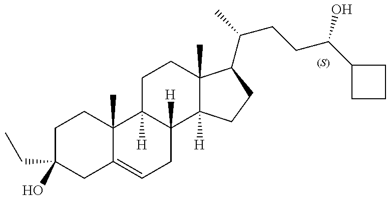 |
| B387 | 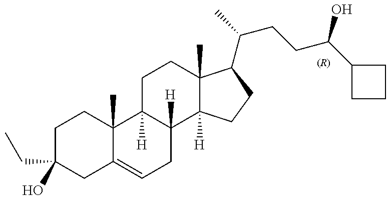 |
| B388 | 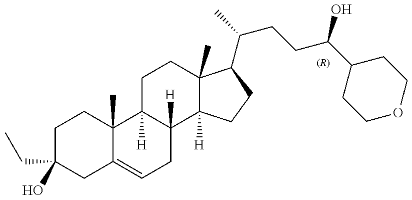 |
| B389 | 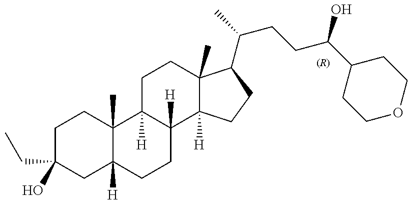 |
| B390 | 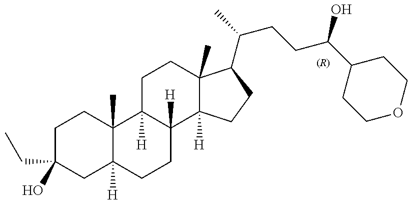 |
| B391 | 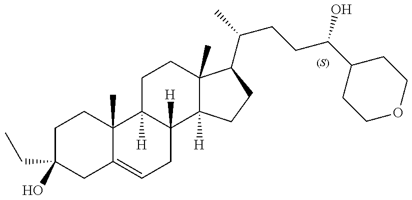 |
| B392 | 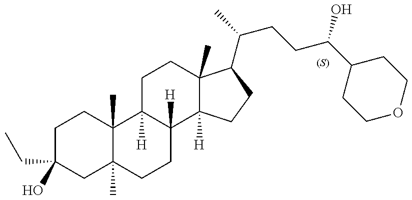 |
| B393 | 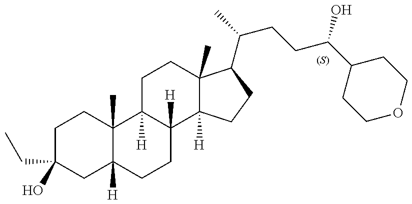 |
| B394 | 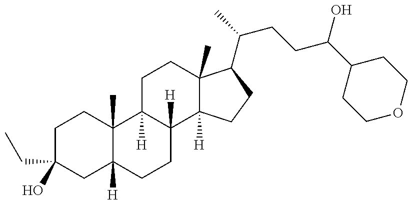 |
| B395 | 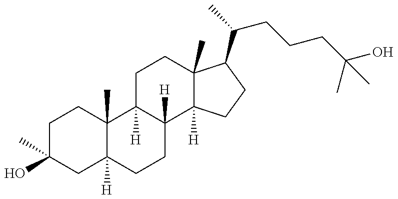 |
| B396 | 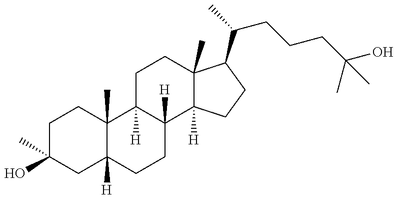 |
| B397 | 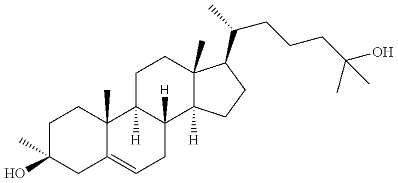 |
| B398 | 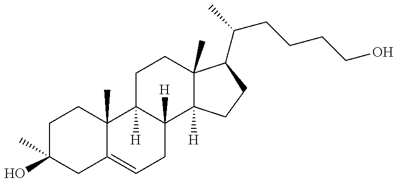 |
| B399 | 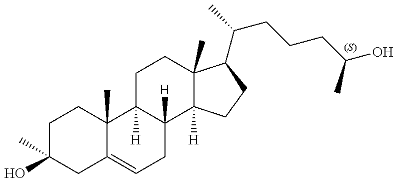 |
| B400 | 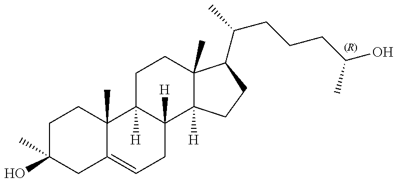 |
| B401 | 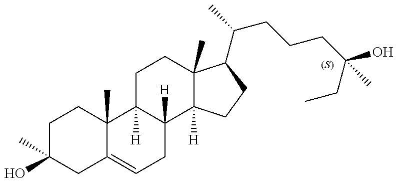 |
| B402 | 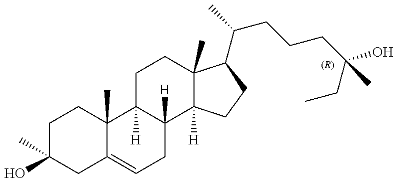 |
| B403 | 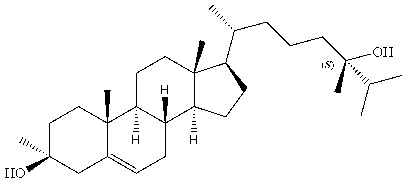 |
| B404 | 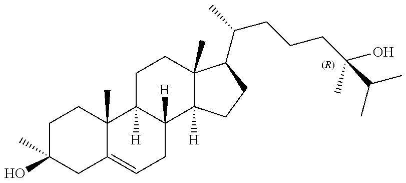 |
| B405 | 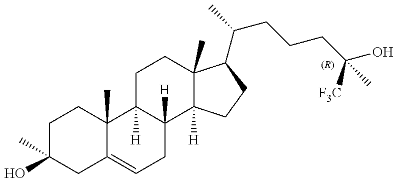 |
| B406 | 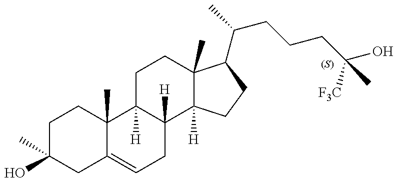 |
| B407 | 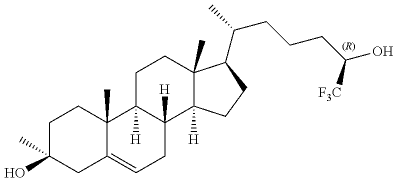 |
| B408 | 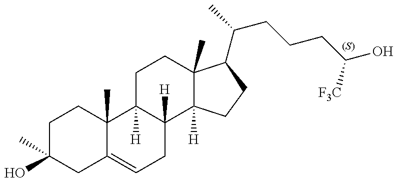 |
| B409 | 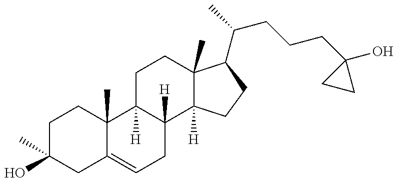 |
| B410 | 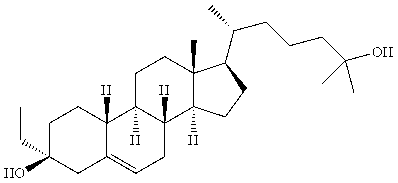 |
| B411 | 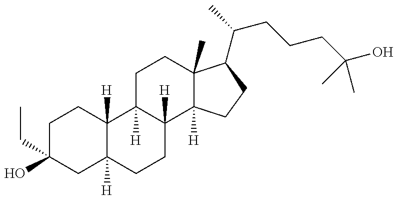 |
| B412 | 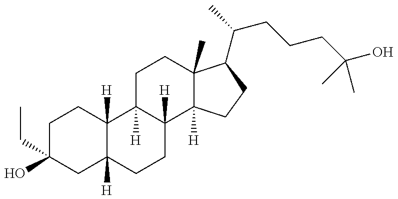 |
| B413 | 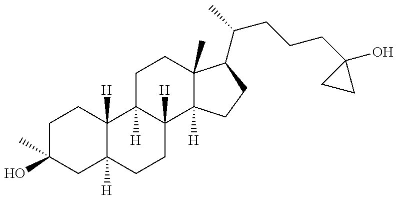 |
| B414 | 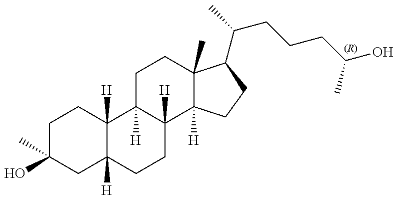 |
| B415 | 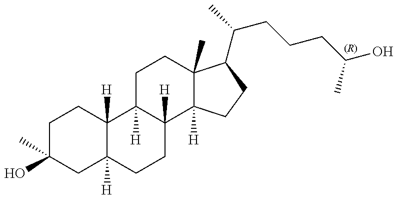 |
| B416 | 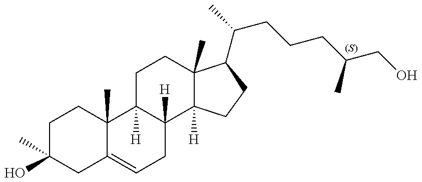 |
| B417 | 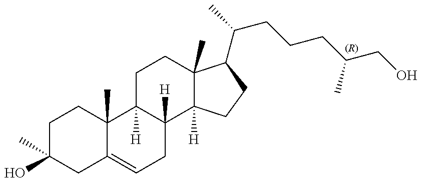 |
| B418 | 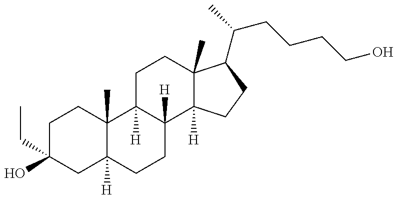 |
| B419 | 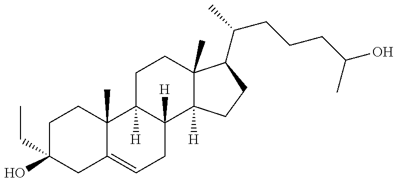 |
| B420 | 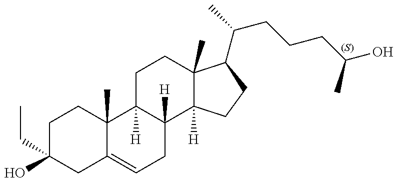 |
| B421 | 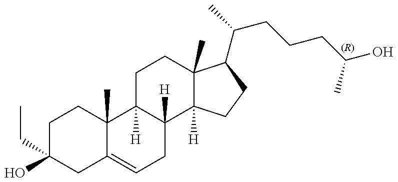 |
| B422 | 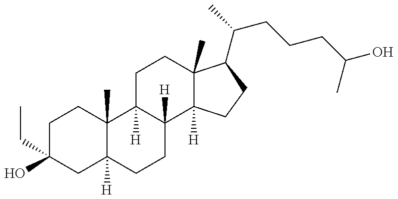 |
| B423 | 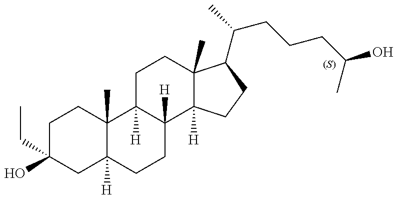 |
| B424 | 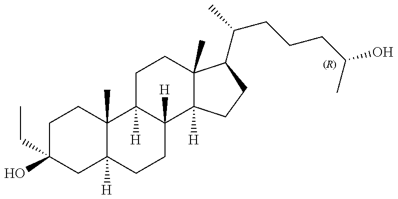 |
| B425 | 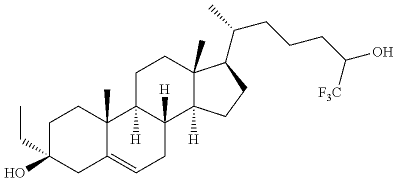 |
| B426 | 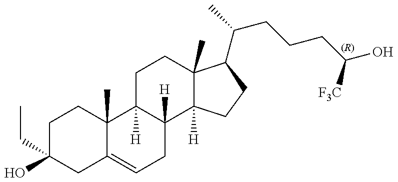 |
| B427 | 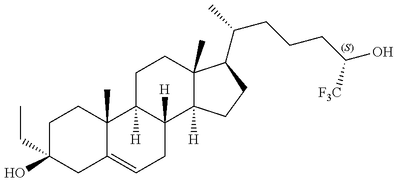 |
| B428 | 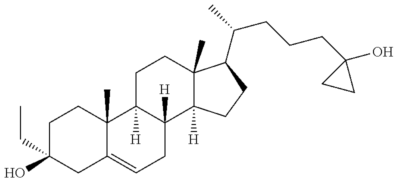 |
| B429 | 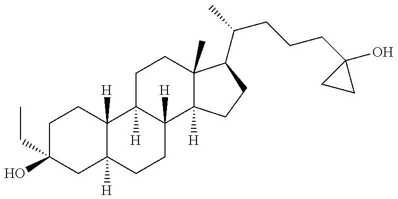 |
| B430 | 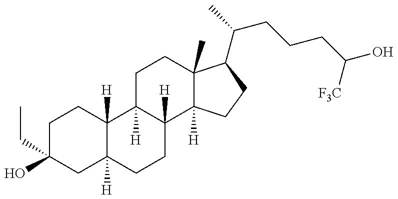 |
| B431 | 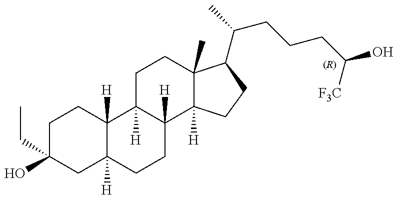 |
| B432 | 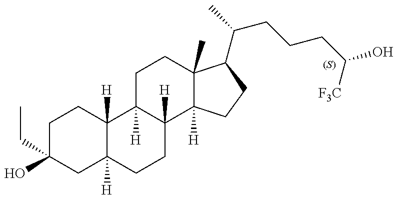 |
| B433 | 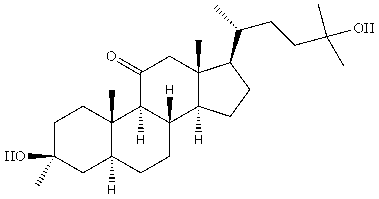 |
| B434 | 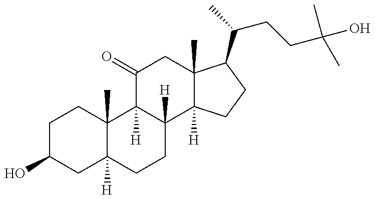 |
| B435 | 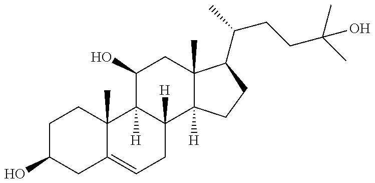 |
| B436 | 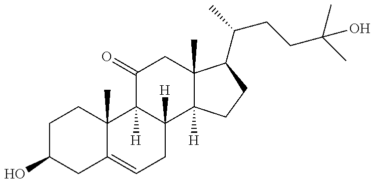 |
| B437 | 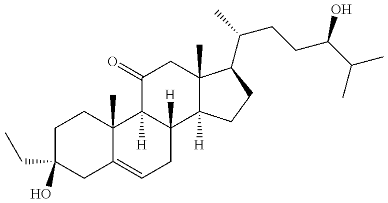 |
| B438 | 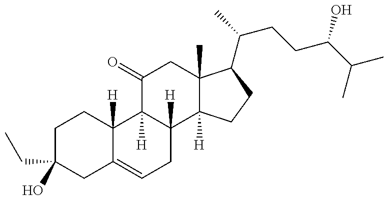 |
| B439 | 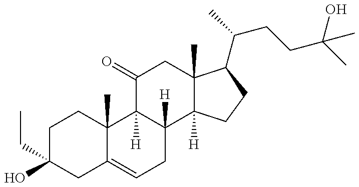 |
| B440 | 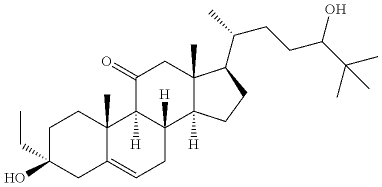 |
| B441 | 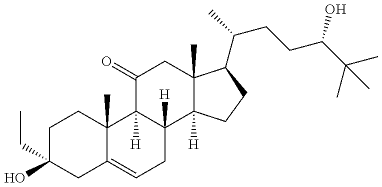 |
| B442 | 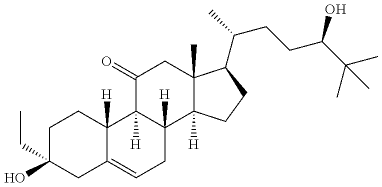 |
| B443 | 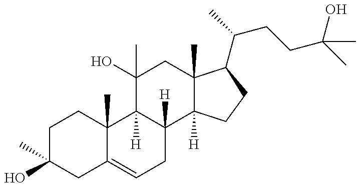 |
| B444 | 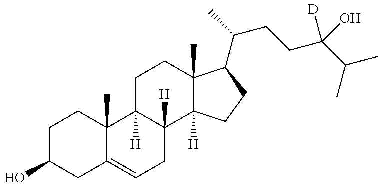 |
| B445 | 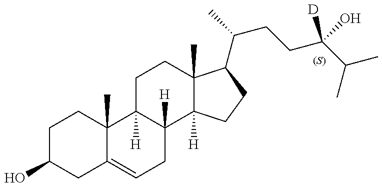 |
| B446 | 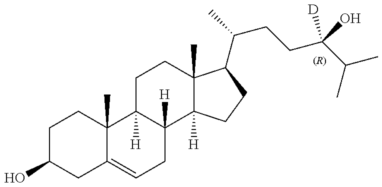 |
| B447 | 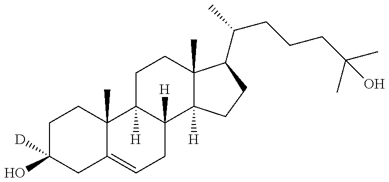 |
| B448 | 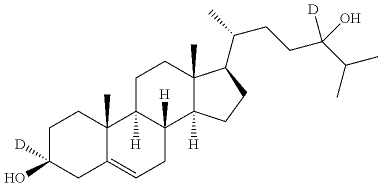 |
| B449 | 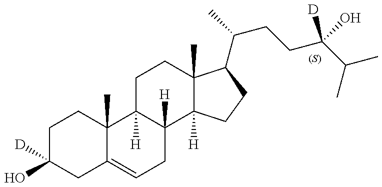 |
| B450 | 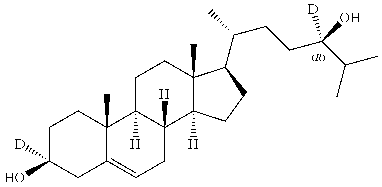 |
| B451 | 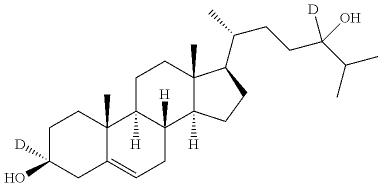 |
| B452 | 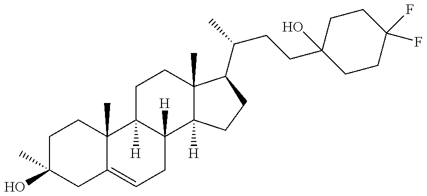 |
| B453 | 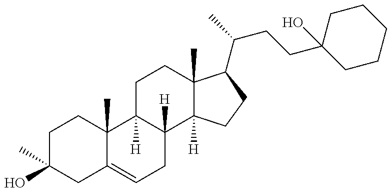 |
| B454 | 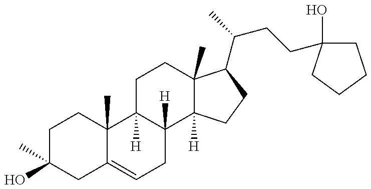 |
| B455 | 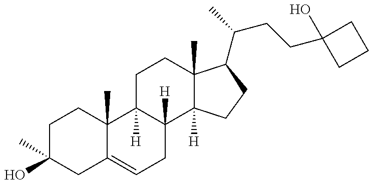 |
| B456 | 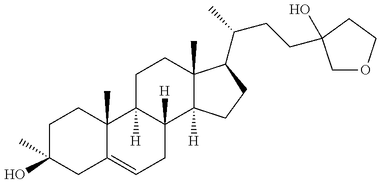 |
| B457 | 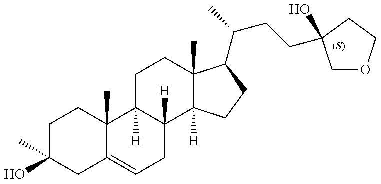 |
| B458 | 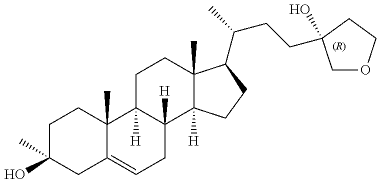 |
| B459 | 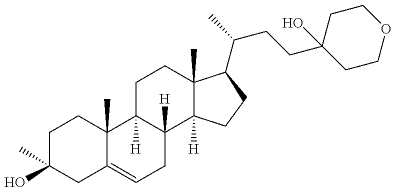 |
| B460 | 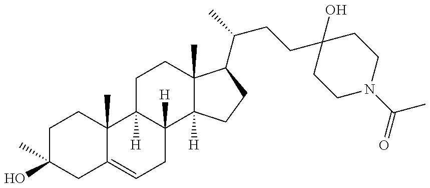 |
| B461 | 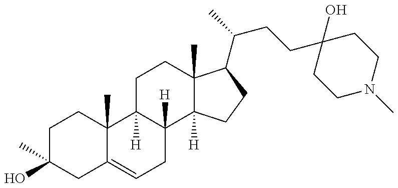 |
| B462 | 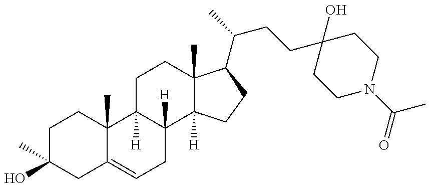 |
| B463 | 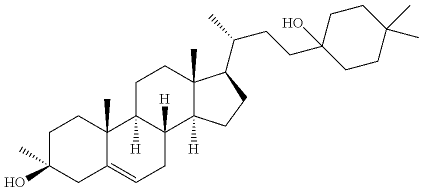 |
| B464 | 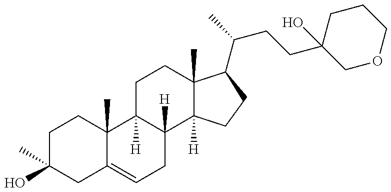 |
| B465 | 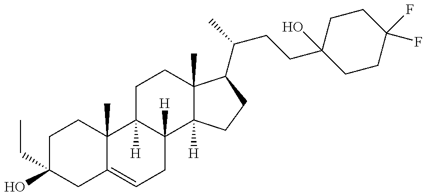 |
| B466 | 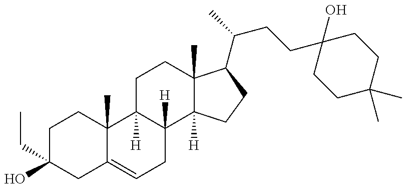 |
| B467 | 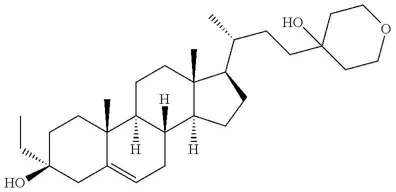 |
| B468 | 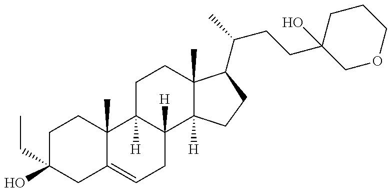 |
| B469 | 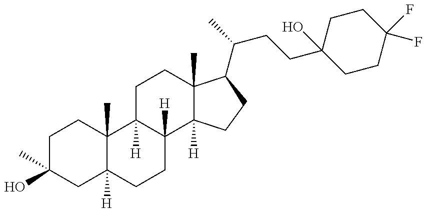 |
| B470 | 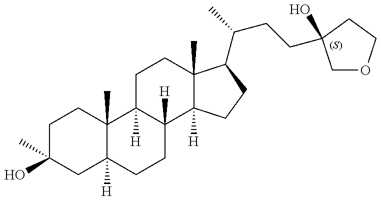 |
| B471 | 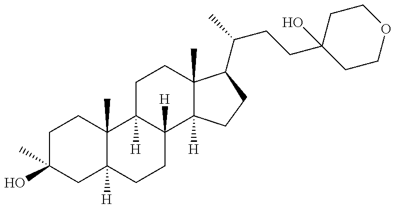 |
| B472 | 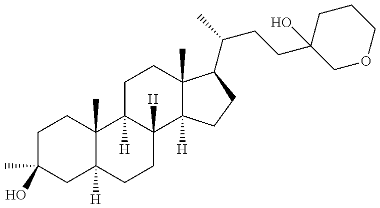 |
| B473 | 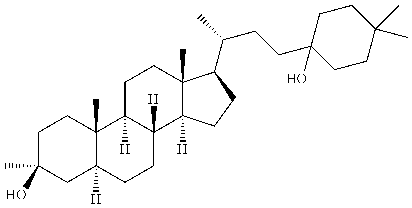 |
| B474 | 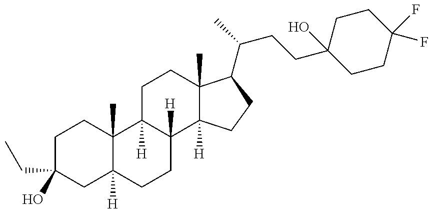 |
| B475 | 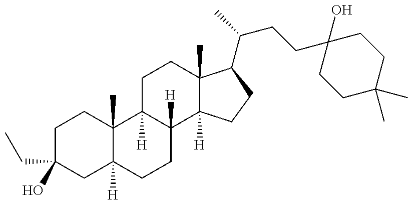 |
| B476 | 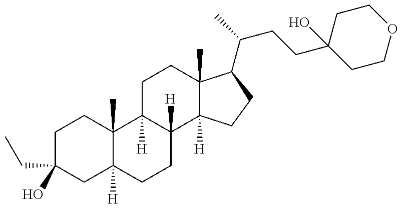 |
| B477 | 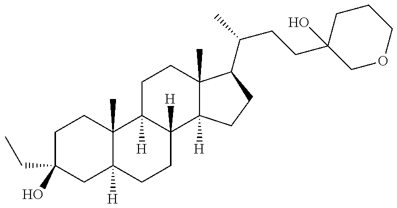 |
| B478 | 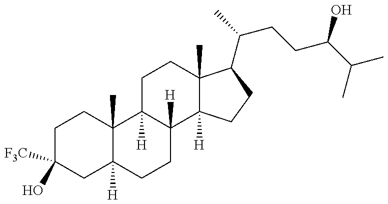 |
| B479 | 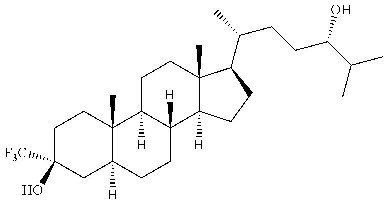 |
| B480 | 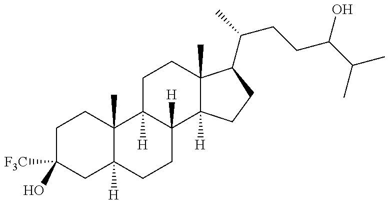 |
| B481 | 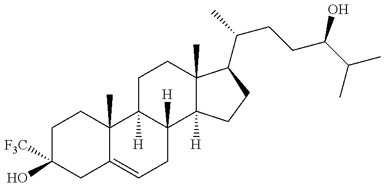 |
| B482 | 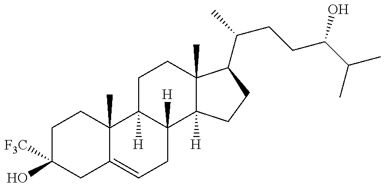 |
| B483 | 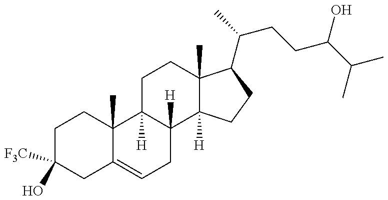 |
| B484 | 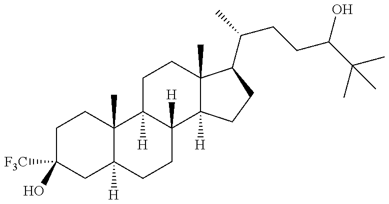 |
| B485 | 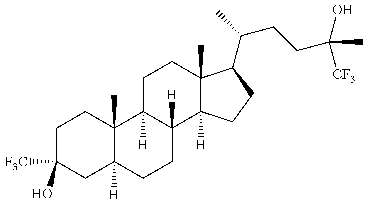 |
| B486 | 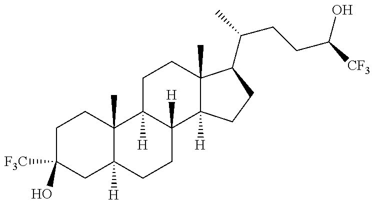 |
| B487 | 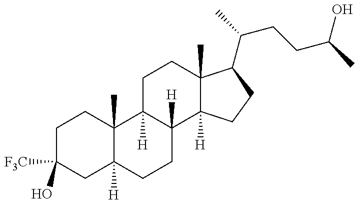 |
| B488 | 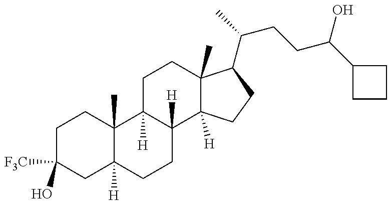 |
| B489 | 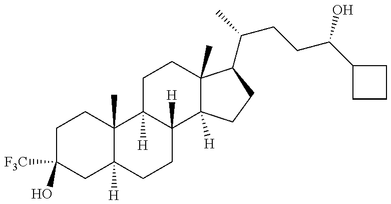 |
| B490 | 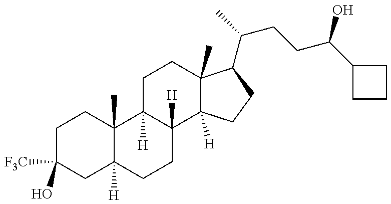 |
| B491 | 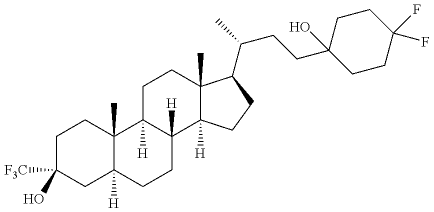 |
| B492 | 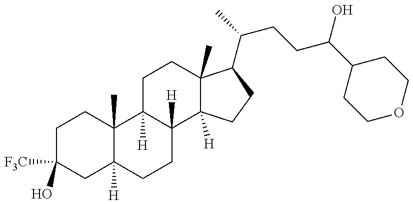 |
| B493 | 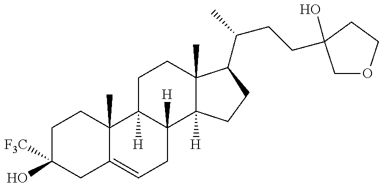 |
| B494 | 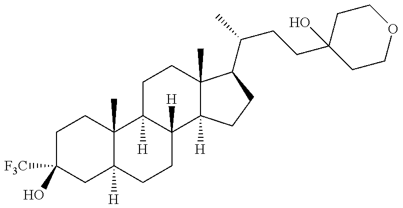 |
| B495 | 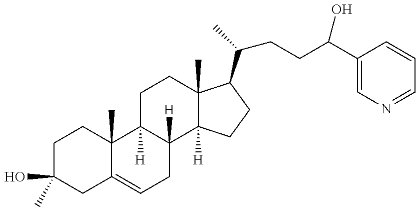 |
| B496 | 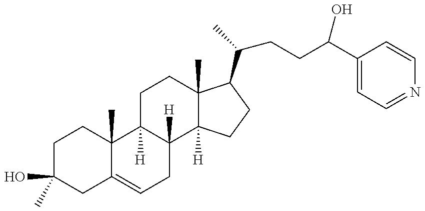 |
| B497 | 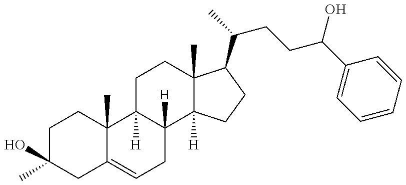 |
| B498 | 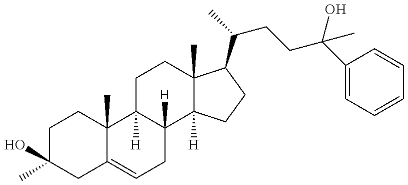 |
| B499 | 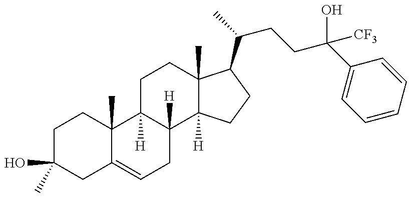 |
| B500 | 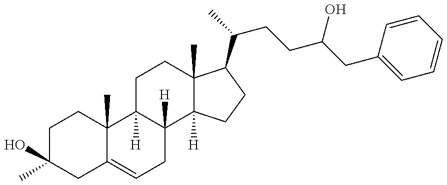 |
| B501 | 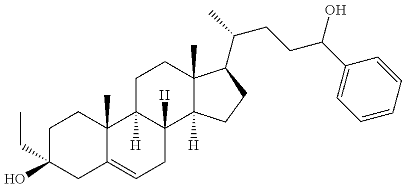 |
| B502 | 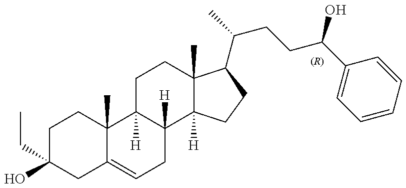 |
| B503 | 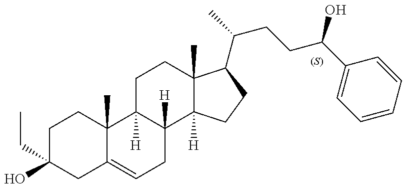 |
| B504 | 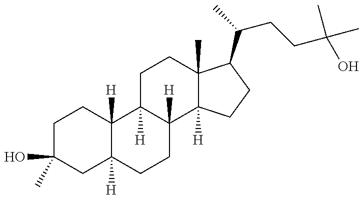 |
| B505 | 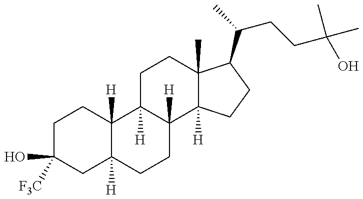 |
| B506 | 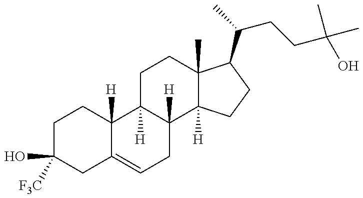 |
| B507 | 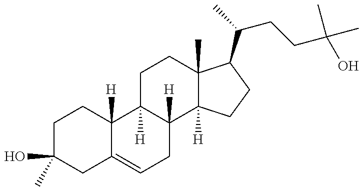 |
| B508 | 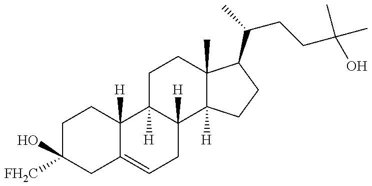 |
| B509 | 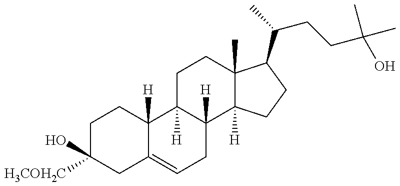 |
| B510 | 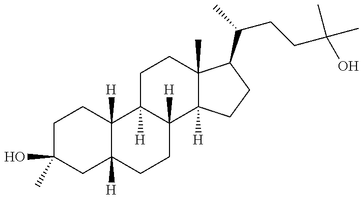 |
| B511 | 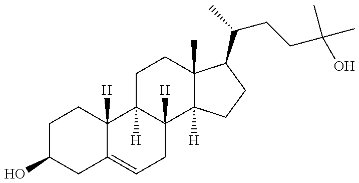 |
| B512 | 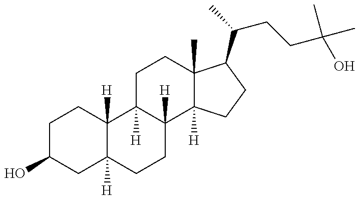 |
| B513 | 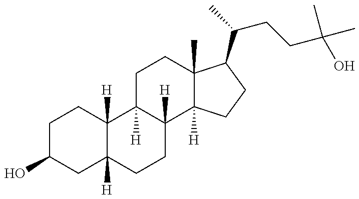 |
| B514 | 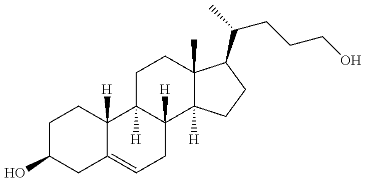 |
| B515 | 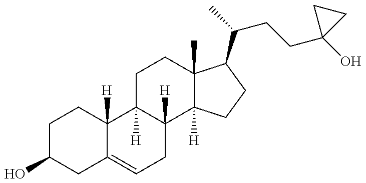 |
| B516 | 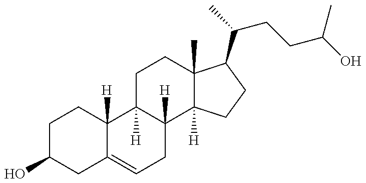 |
| B517 | 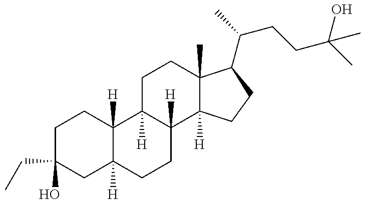 |
| B518 | 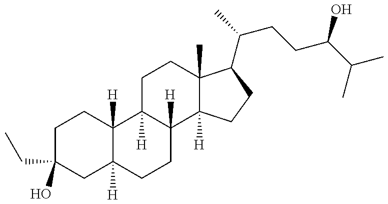 |
| B519 | 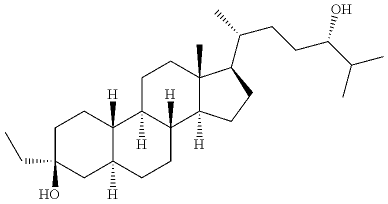 |
| B520 | 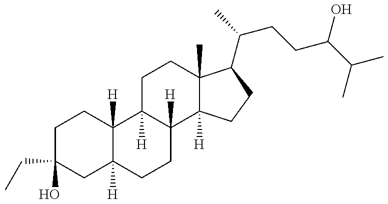 |
| B521 | 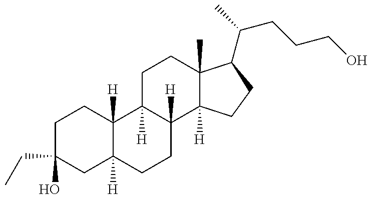 |
| B522 | 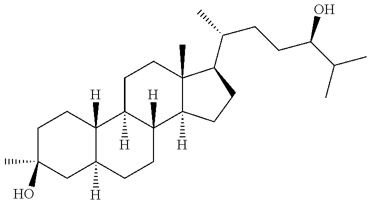 |
| B523 | 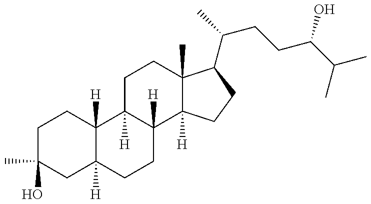 |
| B524 | 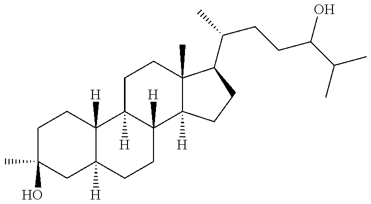 |
| B525 | 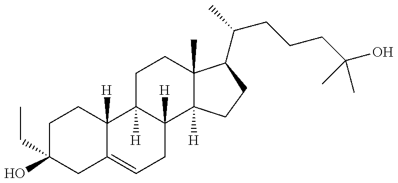 |
| B526 | 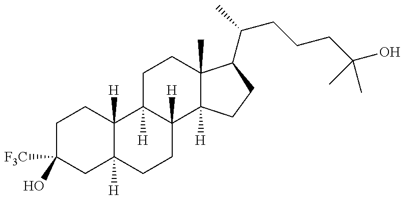 |
| B527 | 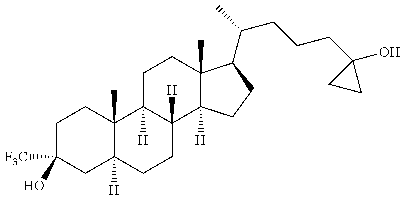 |
| B528 | 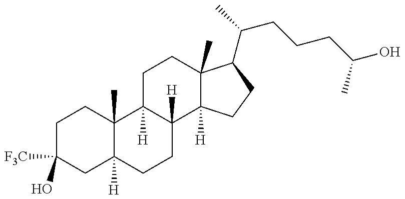 |
| B529 | 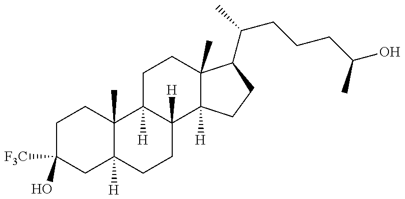 |
| B530 | 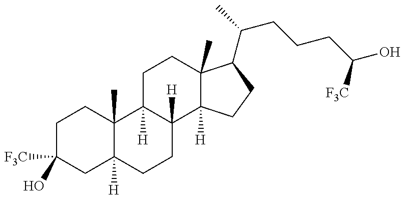 |
| B531 | 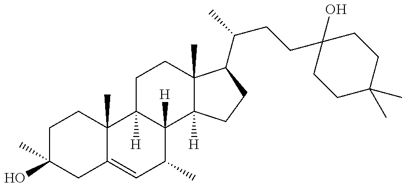 |
| B532 | 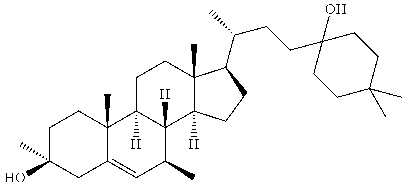 |
| B533 | 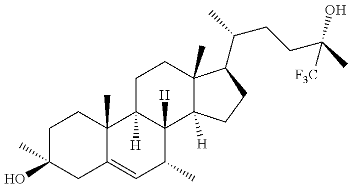 |
| B534 | 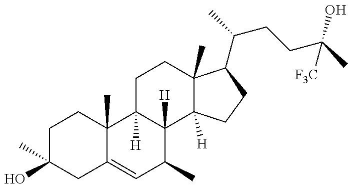 |
| B535 | 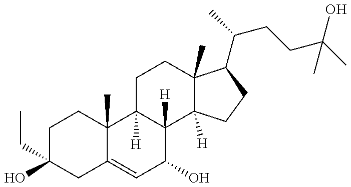 |
| B536 | 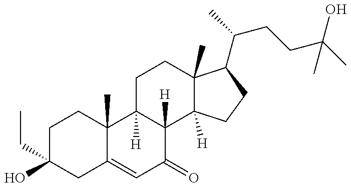 |
| B537 | 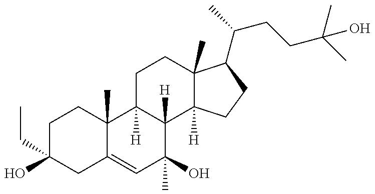 |
| B538 | 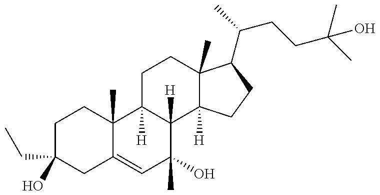 |
| B539 | 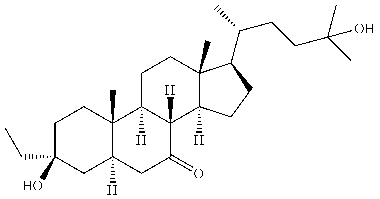 |
| B540 | 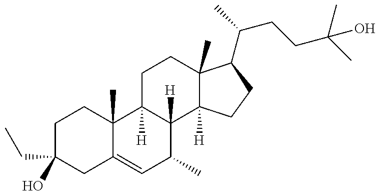 |
| B541 | 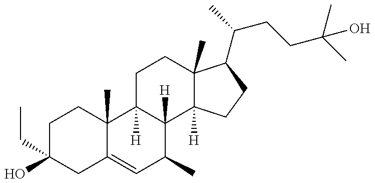 |
| B542 | 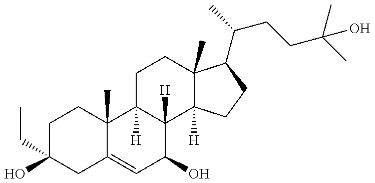 |
| B543 | 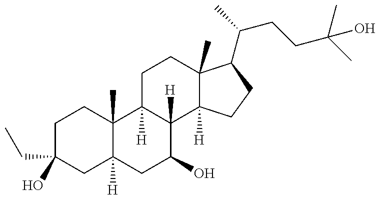 |

In some embodiments, the neuroactive steroid is selected from the group consisting of any one of compounds B1-B140, and pharmaceutically acceptable salts thereof. In some embodiments, the neuroactive steroid is selected from the group consisting of any one of compounds B141-B149, and pharmaceutically acceptable salts thereof. In some embodiments, the neuroactive steroid is selected from the group consisting of any one of compounds B150-B245, and pharmaceutically acceptable salts thereof. In some embodiments, the neuroactive steroid is selected from the group consisting of any one of compounds B246-B272, and pharmaceutically acceptable salts thereof. In some embodiments, the neuroactive steroid is selected from the group consisting of any one of compounds B273-B349, and pharmaceutically acceptable salts thereof. In some embodiments, the neuroactive steroid is selected from the group consisting of any one of compounds B395-B432, and pharmaceutically acceptable salts thereof. In some embodiments, the neuroactive steroid is selected from the group consisting of any one of compounds B433-B443, and pharmaceutically acceptable salts thereof. In some embodiments, the neuroactive steroid is selected from the group consisting of any one of compounds B444-B451, and pharmaceutically acceptable salts thereof. In some embodiments, the neuroactive steroid is selected from the group consisting of any one of compounds B452-B477, and pharmaceutically acceptable salts thereof. In some embodiments, the neuroactive steroid is selected from the group consisting of any one of compounds B478-B530, and pharmaceutically acceptable salts thereof. In some embodiments, the neuroactive steroid is selected from the group consisting of any one of compounds B531-B543, and pharmaceutically acceptable salts thereof.

In some embodiments, the neuroactive in any single compound set forth in Table 2 or a pharmaceutically acceptable salt thereof.

The synthesis of compounds B1-B140 is described in WO 2013/036835, B141-B149 in WO 2015/195967; the synthesis of compounds B150-B245 is described in WO 2014/160480, B246-B272 in WO2017/007832, B273-B394 in WO 2017/007840, B395-B432 in WO2017/007836, B433-B443 in WO 2018/009867, B444-B451 in WO 2017/193046, B452-B477 in WO 2017/173358, B478-B530 in WO 2018/075699, and B531-B543 in WO 2018/064659. The disclosure of each of the foregoing applications is incorporated by reference herein in their entirety.

In some embodiments, the NMDA PAM is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl) (6,7-dimethoxy-1-[(4-methoxyphenoxy)methyl]-3,4-dihydroisoquinolin-2 (1H)-yl)methanone (CIQ), NYX-458, plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one), and pharmaceutically acceptable salts thereof. In some embodiments, the NMDA PAM is BP512 or pharmaceutically acceptable salts thereof. In some embodiments, the NMDA PAM is UBP684 or a pharmaceutically acceptable salt thereof. In some embodiments, the NMDA PAM is GNE-9278 or a pharmaceutically acceptable salt thereof. In some embodiments, the NMDA PAM is GNE-3476 or a pharmaceutically acceptable salt thereof. In some embodiments, the NMDA PAM is PYD-111 or a pharmaceutically acceptable salt thereof. In some embodiments, the NMDA PAM is PYD-106, or a pharmaceutically acceptable salt thereof. In some embodiments, the NMDA PAM is CIQ or a pharmaceutically acceptable salt thereof. In some embodiments, the NMDA PAM is NYX-458, or a pharmaceutically acceptable salt thereof. In some embodiments, the NMDA PAM is plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one) or a pharmaceutically acceptable salt thereof.

### Anti-Amyloid Beta Antibodies

An anti-amyloid beta antibody or antigen-binding fragment thereof useful in the compositions, kits and methods of the present disclosure is an antibody or fragment thereof that binds to amyloid beta and exhibits a beneficial effect for treating or preventing Alzheimer's disease as disclosed herein when administered in combination with an NMDA PAM or a CYP inhibitor. Any anti-amyloid beta antibody or antigen binding fragment thereof, including those disclosed in U.S. Pat. Nos. 10,842,871, 10,131,708, 9,828,420, and 8,906,367, and US patent publication 2015/013155267, the disclosures of all of which are incorporated by reference herein, may be used in the pharmaceutical compositions, kits and methods disclosed herein.

More specifically, the pharmaceutical compositions, kits and methods disclosed herein comprise an anti-amyloid beta antibody, antigen-binding fragment (or portion) thereof, the light chain of the antibody, the heavy chain of the antibody, and fragments of these light chains or heavy chains. The anti-amyloid beta antibodies and antigen-binding fragments thereof disclosed herein also included antibodies lacking the heavy and/or light chain signal sequences and glycosylated antibodies. The anti-amyloid beta antibodies and antigen-binding fragments thereof disclosed herein also include precursor antibodies, nonglycosylated antibodies, and antibodies whose heavy and/or light chains comprise signal sequences.

The antibodies and antigen-binding fragments thereof described herein can be purified and/or isolated using known techniques. Antibodies or portions that are “purified” or “isolated” have been at least partially separated away from molecules (e.g., peptides) of their source of origin (e.g., the supernatant of cells; in a mixture such as in a mixture of antibodies in a library; etc.), and include antibodies obtained by any other suitable methods. Isolated antibodies include substantially pure (e.g., essentially pure) antibodies, as well as antibodies produced by chemical synthesis, recombinant techniques and a combination thereof.

An anti-amyloid beta antibody or antigen-binding fragment thereof, may be human. In some embodiments, an anti-amyloid beta antibody or antigen-binding fragment thereof, of the disclosure is chimeric. In some embodiments, the chimeric anti-amyloid beta antibody or antigen-binding fragment thereof, comprises a chimeric IgH chain and a human Igκ chain. In some embodiments, the chimeric anti-amyloid beta antibody or antigen-binding fragment thereof, comprises a chimeric IgH chain and a human Igλ chain. In some embodiments, the chimeric anti-S1 antibody comprises human and mouse sequences.

In some embodiments, the anti-amyloid beta antibody or antigen-binding fragment thereof comprises one to six complementarity determining regions (CDRs) disclosed herein.

In some embodiments, the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof. In some embodiments, the anti-amyloid beta antibody or antigen-binding fragment thereof is bapineuzumab or an antigen-binding fragments thereof. In some embodiments, the anti-amyloid beta antibody or antigen-binding fragment thereof is solanezumab, or an antigen-binding fragments thereof. In some embodiments, the anti-amyloid beta antibody or antigen-binding fragment thereof is gantenerumab or an antigen-binding fragments thereof. In some embodiments, the anti-amyloid beta antibody or antigen-binding fragment thereof is crenezumab or an antigen-binding fragments thereof. In some embodiments, the anti-amyloid beta antibody or antigen-binding fragment thereof is ponezumab or an antigen-binding fragments thereof. In some embodiments, the anti-amyloid beta antibody or antigen-binding fragment thereof is lecanemab (BAN2401) or an antigen-binding fragments thereof. In some embodiments, the anti-amyloid beta antibody or antigen-binding fragment thereof is aducanumab or an antigen-binding fragments thereof.

In some embodiments, the anti-amyloid beta antibody or antigen binding fragment thereof binds to the same conformational epitope of amyloid beta to which any one of bapineuzumab, solanezumab, gantenerumab, crenezumab, ponezumab, lecanemab (BAN2401), or aducanumab binds. In some embodiments, the anti-amyloid beta antibody or antigen binding fragment thereof binds to the same conformational epitope of amyloid beta to which bapineuzumab binds. In some embodiments, the anti-amyloid beta antibody or antigen binding fragment thereof binds to the same conformational epitope of amyloid beta to which solanezumab binds. In some embodiments, the anti-amyloid beta antibody or antigen binding fragment thereof binds to the same conformational epitope of amyloid beta to which gantenerumab binds. In some embodiments, the anti-amyloid beta antibody or antigen binding fragment thereof binds to the same conformational epitope of amyloid beta to which crenezumab binds. In some embodiments, the anti-amyloid beta antibody or antigen binding fragment thereof binds to the same conformational epitope of amyloid beta to which ponezumab binds. In some embodiments, the anti-amyloid beta antibody or antigen binding fragment thereof binds to the same conformational epitope of amyloid beta to which lecanemab (BAN2401) binds. In some embodiments, the anti-amyloid beta antibody or antigen binding fragment thereof binds to the same conformational epitope of amyloid beta to which aducanumab binds.

In some embodiments, the anti-amyloid beta antibody or antigen binding fragment thereof competitively inhibits binding of any one of bapineuzumab, solanezumab, gantenerumab, crenezumab, ponezumab, lecanemab (BAN2401), or aducanumab to amyloid beta. In some embodiments, the anti-amyloid beta antibody or antigen binding fragment thereof competitively inhibits binding of bapineuzumab to amyloid beta. In some embodiments, the anti-amyloid beta antibody or antigen binding fragment thereof competitively inhibits binding of solanezumab to amyloid beta. In some embodiments, the anti-amyloid beta antibody or antigen binding fragment thereof competitively inhibits binding of gantenerumab to amyloid beta. In some embodiments, the anti-amyloid beta antibody or antigen binding fragment thereof competitively inhibits binding of ponezumab to amyloid beta. In some embodiments, the anti-amyloid beta antibody or antigen binding fragment thereof competitively inhibits binding of lecanemab (BAN2401) to amyloid beta. In some embodiments, the anti-amyloid beta antibody or antigen binding fragment thereof competitively inhibits binding of aducanumab to amyloid beta.

In some embodiments, the anti-amyloid beta antibody or antigen-binding fragment thereof comprises the 6 CDRs of any one of the anti-amyloid beta antibodies disclosed herein as determined by Kabat, Chothia or IMTG nomenclature. In some embodiments, the anti-amyloid beta antibody or antigen-binding fragment thereof comprises the 6 CDRs of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the 6 CDRs of bapineuzumab. In some embodiments, the anti-amyloid beta antibody or antigen-binding fragment thereof comprises the 6 CDRs of solanezumab. In some embodiments, the anti-amyloid beta antibody or antigen-binding fragment thereof comprises the 6 CDRs of gantenerumab. In some embodiments, the anti-amyloid beta antibody or antigen-binding fragment thereof comprises the 6 CDRs of crenezumab. In some embodiments, the anti-amyloid beta antibody or antigen-binding fragment thereof comprises the 6 CDRs of ponezumab. In some embodiments, the anti-amyloid beta antibody or antigen-binding fragment thereof comprises the 6 CDRs of lecanemab (BAN2401). In some embodiments, the anti-amyloid beta antibody or antigen-binding fragment thereof comprises the 6 CDRs of aducanumab.

In some embodiments, the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6;
(b) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16;
(c) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26;
(d) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36;
(e) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46;
(f) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56; or
(g) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and (b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and (b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and (b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and (b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and (b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and (b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.

In some embodiments, the anti-amyloid beta antibody or antigen-binding fragment thereof comprises: (a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and (b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.

In some embodiments, the anti-amyloid beta antibody or antigen-binding fragment thereof comprises the heavy chain variable domain (VH) and/or the light chain variable domain (VL) domain of any one of the anti-amyloid beta antibodies disclosed herein. In some embodiments, the anti-amyloid beta antibody or antigen-binding fragment thereof comprises the VH domain of any one of bapineuzumab, solanezumab, gantenerumab, crenezumab, ponezumab, lecanemab (BAN2401), aducanumab.

In some embodiments, the anti-amyloid beta antibody or antigen-binding fragment thereof comprises the VH domain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the VH domain of bapineuzumab. In some embodiments, the anti-amyloid beta antibody or antigen-binding fragment thereof comprises the VH domain of solanezumab. In some embodiments, the anti-amyloid beta antibody or antigen-binding fragment thereof comprises the VH domain of gantenerumab. In some embodiments, the anti-amyloid beta antibody or antigen-binding fragment thereof comprises the VH domain of crenezumab. In some embodiments, the anti-amyloid beta antibody or antigen-binding fragment thereof comprises the VH domain of ponezumab. In some embodiments, the anti-amyloid beta antibody or antigen-binding fragment thereof comprises the VH domain of lecanemab (BAN2401). In some embodiments, the anti-amyloid beta antibody or antigen-binding fragment thereof comprises the VH domain of aducanumab.

In some embodiments, the anti-amyloid beta antibody or antigen-binding fragment thereof comprises the VL domain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the VL domain of bapineuzumab. In some embodiments, the anti-amyloid beta antibody or antigen-binding fragment thereof comprises the VL domain of solanezumab. In some embodiments, the anti-amyloid beta antibody or antigen-binding fragment thereof comprises the VL domain of gantenerumab. In some embodiments, the anti-amyloid beta antibody or antigen-binding fragment thereof comprises the VL domain of crenezumab. In some embodiments, the anti-amyloid beta antibody or antigen-binding fragment thereof comprises the VL domain of ponezumab. In some embodiments, the anti-amyloid beta antibody or antigen-binding fragment thereof comprises the VL domain of lecanemab (BAN2401). In some embodiments, the anti-amyloid beta antibody or antigen-binding fragment thereof comprises the VL domain of aducanumab.

In some embodiments, the anti-amyloid beta antibody or antigen-binding fragment thereof comprises the VH and VH domains of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the VH and VL domains of bapineuzumab. In some embodiments, the anti-amyloid beta antibody or antigen-binding fragment thereof comprises the VH and VL domains of solanezumab. In some embodiments, the anti-amyloid beta antibody or antigen-binding fragment thereof comprises the VH and VL domains of gantenerumab. In some embodiments, the anti-amyloid beta antibody or antigen-binding fragment thereof comprises the VH and VL domains of crenezumab. In some embodiments, the anti-amyloid beta antibody or antigen-binding fragment thereof comprises the VH and VL domains of ponezumab. In some embodiments, the anti-amyloid beta antibody or antigen-binding fragment thereof comprises the VH and VL domains of lecanemab (BAN2401). In some embodiments, the anti-amyloid beta antibody or antigen-binding fragment thereof comprises the VH and VL domains of aducanumab.

In some embodiments, the anti-amyloid beta antibody or antigen-binding fragment thereof comprises a heavy chain variable domain (VH) comprising: (a) the amino acid sequence of SEQ ID NO: 7; (b) the amino acid sequence of SEQ ID NO: 17; (c) the amino acid sequence of SEQ ID NO: 27; (d) the amino acid sequence of SEQ ID NO: 37; (e) the amino acid sequence of SEQ ID NO: 47; (f) the amino acid sequence of SEQ ID NO: 57; or (g) the amino acid sequence of SEQ ID NO: 67.

In some embodiments, the anti-amyloid beta antibody or antigen-binding fragment thereof comprises a light chain variable domain (VL) comprising: (a) the amino acid sequence of SEQ ID NO: 8; (b) the amino acid sequence of SEQ ID NO: 18; (c) the amino acid sequence of SEQ ID NO: 28; (d) the amino acid sequence of SEQ ID NO: 38; (e) the amino acid sequence of SEQ ID NO: 48; (f) the amino acid sequence of SEQ ID NO: 58; or (g) the amino acid sequence of SEQ ID NO: 68.

In some embodiments, the anti-amyloid beta antibody or antigen-binding fragment thereof comprises a heavy chain variable domain (VH) comprising the amino acid sequence of SEQ ID NO: 7. In some embodiments, the anti-amyloid beta antibody or antigen-binding fragment thereof comprises a light chain variable domain (VL) comprising the amino acid sequence of SEQ ID NO: 8. In some embodiments, the anti-amyloid beta antibody or antigen-binding fragment thereof comprises a heavy chain variable domain (VH) comprising the amino acid sequence of SEQ ID NO: 7; and a light chain variable domain (VL) comprising the amino acid sequence of SEQ ID NO: 8.

In some embodiments, the anti-amyloid beta antibody or antigen-binding fragment thereof comprises a heavy chain variable domain (VH) comprising the amino acid sequence of SEQ ID NO: 17. In some embodiments, the anti-amyloid beta antibody or antigen-binding fragment thereof comprises a light chain variable domain (VL) comprising the amino acid sequence of SEQ ID NO: 18. In some embodiments, the anti-amyloid beta antibody or antigen-binding fragment thereof comprises a heavy chain variable domain (VH) comprising the amino acid sequence of SEQ ID NO: 17; and a light chain variable domain (VL) comprising the amino acid sequence of SEQ ID NO: 18.

In some embodiments, the anti-amyloid beta antibody or antigen-binding fragment thereof comprises a heavy chain variable domain (VH) comprising the amino acid sequence of SEQ ID NO: 27. In some embodiments, the anti-amyloid beta antibody or antigen-binding fragment thereof comprises a light chain variable domain (VL) comprising the amino acid sequence of SEQ ID NO: 28. In some embodiments, the anti-amyloid beta antibody or antigen-binding fragment thereof comprises a heavy chain variable domain (VH) comprising the amino acid sequence of SEQ ID NO: 27; and a light chain variable domain (VL) comprising the amino acid sequence of SEQ ID NO: 28.

In some embodiments, the anti-amyloid beta antibody or antigen-binding fragment thereof comprises a heavy chain variable domain (VH) comprising the amino acid sequence of SEQ ID NO: 37. In some embodiments, the anti-amyloid beta antibody or antigen-binding fragment thereof comprises a light chain variable domain (VL) comprising the amino acid sequence of SEQ ID NO: 38. In some embodiments, the anti-amyloid beta antibody or antigen-binding fragment thereof comprises a heavy chain variable domain (VH) comprising the amino acid sequence of SEQ ID NO: 37; and a light chain variable domain (VL) comprising the amino acid sequence of SEQ ID NO: 38.

In some embodiments, the anti-amyloid beta antibody or antigen-binding fragment thereof comprises a heavy chain variable domain (VH) comprising the amino acid sequence of SEQ ID NO: 47. In some embodiments, the anti-amyloid beta antibody or antigen-binding fragment thereof comprises a light chain variable domain (VL) comprising the amino acid sequence of SEQ ID NO: 48. In some embodiments, the anti-amyloid beta antibody or antigen-binding fragment thereof comprises a heavy chain variable domain (VH) comprising the amino acid sequence of SEQ ID NO: 47; and a light chain variable domain (VL) comprising the amino acid sequence of SEQ ID NO: 48.

In some embodiments, the anti-amyloid beta antibody or antigen-binding fragment thereof comprises a heavy chain variable domain (VH) comprising the amino acid sequence of SEQ ID NO: 57. In some embodiments, the anti-amyloid beta antibody or antigen-binding fragment thereof comprises a light chain variable domain (VL) comprising the amino acid sequence of SEQ ID NO: 58. In some embodiments, the anti-amyloid beta antibody or antigen-binding fragment thereof comprises a heavy chain variable domain (VH) comprising the amino acid sequence of SEQ ID NO: 57; and a light chain variable domain (VL) comprising the amino acid sequence of SEQ ID NO: 58.

In some embodiments, the anti-amyloid beta antibody or antigen-binding fragment thereof comprises a heavy chain variable domain (VH) comprising the amino acid sequence of SEQ ID NO: 67. In some embodiments, the anti-amyloid beta antibody or antigen-binding fragment thereof comprises a light chain variable domain (VL) comprising the amino acid sequence of SEQ ID NO: 68. In some embodiments, the anti-amyloid beta antibody or antigen-binding fragment thereof comprises a heavy chain variable domain (VH) comprising the amino acid sequence of SEQ ID NO: 67; and a light chain variable domain (VL) comprising the amino acid sequence of SEQ ID NO: 68.

In some embodiments, the anti-amyloid beta antibody or antigen-binding fragment thereof comprises the heavy chain and/or the light chain of any one of the anti-amyloid beta antibodies disclosed herein.

In some embodiments, the anti-amyloid beta antibody or antigen-binding fragment thereof comprises the heavy chain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the heavy chain of bapineuzumab. In some embodiments, the anti-amyloid beta antibody or antigen-binding fragment thereof comprises the heavy chain of solanezumab. In some embodiments, the anti-amyloid beta antibody or antigen-binding fragment thereof comprises the heavy chain of gantenerumab. In some embodiments, the anti-amyloid beta antibody or antigen-binding fragment thereof comprises the heavy chain of crenezumab. In some embodiments, the anti-amyloid beta antibody or antigen-binding fragment thereof comprises the heavy chain of ponezumab. In some embodiments, the anti-amyloid beta antibody or antigen-binding fragment thereof comprises the heavy chain of lecanemab (BAN2401). In some embodiments, the anti-amyloid beta antibody or antigen-binding fragment thereof comprises the heavy chain of aducanumab.

In some embodiments, the anti-amyloid beta antibody or antigen-binding fragment thereof comprises the light chain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the light chain of bapineuzumab. In some embodiments, the anti-amyloid beta antibody or antigen-binding fragment thereof comprises the light chain of solanezumab. In some embodiments, the anti-amyloid beta antibody or antigen-binding fragment thereof comprises the light chain of gantenerumab. In some embodiments, the anti-amyloid beta antibody or antigen-binding fragment thereof comprises the light chain of crenezumab. In some embodiments, the anti-amyloid beta antibody or antigen-binding fragment thereof comprises the light chain of ponezumab. In some embodiments, the anti-amyloid beta antibody or antigen-binding fragment thereof comprises the light chain of lecanemab (BAN2401). In some embodiments, the anti-amyloid beta antibody or antigen-binding fragment thereof comprises the light chain of aducanumab.

In some embodiments, the anti-amyloid beta antibody or antigen-binding fragment thereof comprises the heavy chain and light chain of any one of bapineuzumab, solanezumab, gantenerumab, crenezumab, ponezumab, lecanemab (BAN2401), aducanumab. In some embodiments, the anti-amyloid beta antibody or antigen-binding fragment thereof comprises the heavy chain and light chain of bapineuzumab. In some embodiments, the anti-amyloid beta antibody or antigen-binding fragment thereof comprises the heavy chain and light chain of solanezumab. In some embodiments, the anti-amyloid beta antibody or antigen-binding fragment thereof comprises the heavy chain and light chain of gantenerumab. In some embodiments, the anti-amyloid beta antibody or antigen-binding fragment thereof comprises the heavy chain and light chain domain of crenezumab. In some embodiments, the anti-amyloid beta antibody or antigen-binding fragment thereof comprises the heavy chain and light chain of ponezumab. In some embodiments, the anti-amyloid beta antibody or antigen-binding fragment thereof comprises the heavy chain and light chain of lecanemab (BAN2401). In some embodiments, the anti-amyloid beta antibody or antigen-binding fragment thereof comprises the heavy chain and light chain domain of aducanumab.

In some embodiments, the anti-amyloid beta antibody comprises a heavy chain comprising the amino acid sequence of SEQ ID NO: 9. In some embodiments, the anti-amyloid beta antibody comprises a light chain comprising the amino acid sequence of SEQ ID NO: 10. In some embodiments, the anti-amyloid beta antibody comprises a heavy chain comprising the amino acid sequence of SEQ ID NO: 9, and a light chain comprising the amino acid sequence of SEQ ID NO: 10.

In some embodiments, the anti-amyloid beta antibody comprises a heavy chain comprising the amino acid sequence of SEQ ID NO: 19. In some embodiments, the anti-amyloid beta antibody comprises a light chain comprising the amino acid sequence of SEQ ID NO: 20. In some embodiments, the anti-amyloid beta antibody comprises a heavy chain comprising the amino acid sequence of SEQ ID NO: 19, and a light chain comprising the amino acid sequence of SEQ ID NO: 20.

In some embodiments, the anti-amyloid beta antibody comprises a heavy chain comprising the amino acid sequence of SEQ ID NO: 29. In some embodiments, the anti-amyloid beta antibody comprises a light chain comprising the amino acid sequence of SEQ ID NO: 30. In some embodiments, the anti-amyloid beta antibody comprises a heavy chain comprising the amino acid sequence of SEQ ID NO: 29, and a light chain comprising the amino acid sequence of SEQ ID NO: 30.

In some embodiments, the anti-amyloid beta antibody comprises a heavy chain comprising the amino acid sequence of SEQ ID NO: 39. In some embodiments, the anti-amyloid beta antibody comprises a light chain comprising the amino acid sequence of SEQ ID NO: 40. In some embodiments, the anti-amyloid beta antibody comprises a heavy chain comprising the amino acid sequence of SEQ ID NO: 39, and a light chain comprising the amino acid sequence of SEQ ID NO: 40.

In some embodiments, the anti-amyloid beta antibody comprises a heavy chain comprising the amino acid sequence of SEQ ID NO: 49. In some embodiments, the anti-amyloid beta antibody comprises a light chain comprising the amino acid sequence of SEQ ID NO: 50. In some embodiments, the anti-amyloid beta antibody comprises a heavy chain comprising the amino acid sequence of SEQ ID NO: 49, and a light chain comprising the amino acid sequence of SEQ ID NO: 50.

In some embodiments, the anti-amyloid beta antibody comprises a heavy chain comprising the amino acid sequence of SEQ ID NO: 59. In some embodiments, the anti-amyloid beta antibody comprises a light chain comprising the amino acid sequence of SEQ ID NO: 60. In some embodiments, the anti-amyloid beta antibody comprises a heavy chain comprising the amino acid sequence of SEQ ID NO: 59, and a light chain comprising the amino acid sequence of SEQ ID NO: 60.

In some embodiments, the anti-amyloid beta antibody comprises a heavy chain comprising the amino acid sequence of SEQ ID NO: 69. In some embodiments, the anti-amyloid beta antibody comprises a light chain comprising the amino acid sequence of SEQ ID NO: 70. In some embodiments, the anti-amyloid beta antibody comprises a heavy chain comprising the amino acid sequence of SEQ ID NO: 69, and a light chain comprising the amino acid sequence of SEQ ID NO: 70.

The anti-amyloid beta antibody or antigen-binding fragment thereof may comprise any of the complementarity determining regions (CDRs) (i.e., HCDRs, LCDRs), VH, VL, heavy chain or light chain sequences set forth in Tables 3-9. Tables 3-9 provide the details of the VH, VL and of the various anti-amyloid beta antibodies.

TABLE 3
Aducanumab (HCDR1-3 and LCDR1-3 based on Kabat nomenclature)

| SEQ ID NO | Description | Amino Acid Sequence |
|---|---|---|
| SEQ ID NO: 1 | HCDR1 | SYGMH |
| SEQ ID NO: 2 | HCDR2 | VIWEDGMKYYTDSVKG |
| SEQ ID NO: 3 | HCDR3 | DRGIGARRGPYYMDV |
| SEQ ID NO: 4 | LCDR1 | RASQSISSYLN |
| SEQ ID NO: 5 | LCDR2 | AASSLQS |
| SEQ ID NO: 6 | LCDR3 | QQSYSTPLI |
| SEQ ID NO: 7 | VH | QVQLVESGGGVVQPGRSLRLSCAASGFAFSS |
| | | YGMHWVRQAPGKGLEWVAVIWEDGTKKYYTD |
| | | SVKGRFTISRDNSKNTLYLQMNTLRAEDTAV |
| | | YYCARDRGIGARRGPYYMDVWGKGTTVYVSS |
| SEQ ID NO: 8 | VL | DIQMTQSPSSLSASVGDRVTITCRASQSISS |
| | | YLNWYQQKPGKAPKLLIYAASSLQSGVPSRF |
| | | SGSGSGTDFTLTISSLQPEDFATYYCQQSYS |
| | | TPLTFGGGTKVEIKR |
| SEQ ID NO: 9 | Heavy chain | QVQLVESGGGVVQPGRSLRLSCAASGFAFSS |
| | (H-Gamma-1) | YGMHWVRQAPGKGLEWVAVIWFDGTKKYYTD |
| | | SVKGRFTISRDNSKNTLYLQMNTLRAEDTAV |
| | | YYCARDRGIGARRGPYYMDVWGKGTTVTVSS |
| | | ASTKGPSVFPLAPSSKSTSGGTAALGCLVKD |
| | | YFPEPVTVSWNSGALTSGVHTFPAVLQSSGL |
| | | YSLSSVVTVPSSSLGTQTYICNVNHKPSNTK |
| | | VDKRVEPKSCDKTHTCPPCPAPELLGGPSVF |
| | | LFPPKPKDTLMISRTPEVTCVVVDVSHEDPE |
| | | VKFNWYVDGVEVHNAKTKPREEQYNSTYRVV |
| | | SVLTVLHQDWLNGKEYKCKVSNKALPAPIEK |
| | | TISKAKGQPREPQVYTLPPSREEMTKNQVSL |
| | | TCLVKGFYPSDIAVEWESNGQPENNYKTTPP |
| | | VLDSDGSFFLYSKLTVDKSRWQQGNVFSCSV |
| | | MHEALHNHYTQKSLSLSPG |
| SEQ ID NO: 10 | Light chain | DIQMTQSPSSLSASVGDRVTITCRASQSISS |
| | (L-Kappa) | YLNWYQQKPGKAPKLLIYAASSLQSGVPSRF |
| | | SGSGSGTDFTLTISSLQPEDFATYYCQQSYS |
| | | TPLTFGGGTKVEIKRTVAAPSVFIFPPSDEQ |
| | | LKSGTASVVCLLNNFYPREAKVQWKVDNALQ |
| | | SGNSQESVTEQDSKDSTYSLSSTLTLSKADY |
| | | EKHKVYACEVTHQGLSSPVTKSFNRGEC |

TABLE 4
Solanezumab (HCDR1-3 and LCDR1-3 based on IMGT nomenclature)

| SEQ ID NO | Description | Amino Acid Sequence |
|---|---|---|
| SEQ ID NO: 11 | HCDR1 | GFTFSRYS |
| SEQ ID NO: 12 | HCDR2 | INSVGNST |
| SEQ ID NO: 13 | HCDR3 | ASGDY |
| SEQ ID NO: 14 | LCDR1 | QSLIYSDGNAY |
| SEQ ID NO: 15 | LCDR2 | KVS |
| SEQ ID NO: 16 | LCDR3 | SQSTHVPWT |
| SEQ ID NO: 17 | VH | EVQLVESGGGLVQPGGSLRLSCAASGFTF |
| | | SRYSMSWVRQAPGKGLELVAQINSVGNST |
| | | YYPDTVKGRFTISRDNAKNTLYLQMNSLR |
| | | AEDTAVYYCA |
| SEQ ID NO: 18 | VL | DVVMTQSPLSLPVTLGQPASISCRSSQSL |
| | | IYSDGNAYLHWFLQKPGQSPRLLIYKVSN |
| | | RESGVPDRFSGSGSGTDFTLKISRVEAED |
| | | VGVYYCSQSTHVP |
| SEQ ID NO: 19 | Heavy chain | EVQLVESGGGLVQPGGSLRLSCAASGFTF |
| | (H-Gamma-1) | SRYSMSWVRQAPGKGLELVAQINSVGNST |
| | | YYPDTVKGRFTISRDNAKNTLYLQMNSLR |
| | | AEDTAVYYCASGDYWGQGTLVTVSSASTK |
| | | GPSVFPLAPSSKSTSGGTAALGCLVKDYF |
| | | PEPVTVSWNSGALTSGVHTFPAVLQSSGL |
| | | YSLSSVVTVPSSSLGTQTYICNVNHKPSN |
| | | TKVDKKVEPKSCDKTHTCPPCPAPELLGG |
| | | PSVFLFPPKPKDTLMISRTPEVTCVVVDV |
| | | SHEDPEVKFNWYVDGVEVHNAKTKPREEQ |
| | | YNSTYRVVSVLTVLHQDWLNGKEYKCKVS |
| | | NKALPAPIEKTISKAKGQPREPQVYTLPP |
| | | SRDELTKNQVSLTCLVKGFYPSDIAVEWE |
| | | SNGQPENNYKTTPPVLDSDGSFFLYSKLT |
| | | VDKSRWQQGNVFSCSVMHEALHNHYTQKS |
| | | LSLSPGK |
| SEQ ID NO: 20 | Light chain | DVVMTQSPLSLPVTLGQPASISCRSSQSL |
| | (L-Kappa) | IYSDGNAYLHWFLQKPGQSPRLLIYKVSN |
| | | RFSGVPDRFSGSGSGTDETLKISRVEAED |
| | | VGVYYCSQSTHVPWTFGQGTKVEIKRTVA |
| | | APSVEIFPPSDEQLKSGTASVVCLLNNFY |
| | | PREAKVQWKVDNALQSGNSQESVTEQDSK |
| | | DSTYSLSSTLTLSKADYEKHKVYACEVTH |
| | | QGLSSPVTKSFNRGEC |

TABLE 5
Crenezumab (HCDR1-3 and LCDR1-3 based on IMGT nomenclature)

| SEQ ID NO | Description | Amino Acid Sequence |
|---|---|---|
| SEQ ID NO: 21 | HCDR1 | GFTFSSYG |
| SEQ ID NO: 22 | HCDR2 | INSNGGST |
| SEQ ID NO: 23 | HCDR3 | ASGDY |
| SEQ ID NO: 24 | LCDR1 | QSLVYSNGDTY |
| SEQ ID NO: 25 | LCDR2 | KVS |
| SEQ ID NO: 26 | LCDR3 | SQSTHVPWT |
| SEQ ID NO: 27 | VH | EVQLVESGGGLVQPGGSLRLSCAASGFTFS |
| | | SYGMSWVRQAPGKGLELVASINSNGGSTYY |
| | | PDSVKGRFTISRDNAKNSLYLQMNSLRAED |
| | | TAVYYCA |
| SEQ ID NO: 28 | VL | DIVMTQSPLSLPVTPGEPASISCRSSQSLV |
| | | YSNGDTYLHWYLQKPGQSPQLLIYKVSNRF |
| | | SGVPDRFSGSGSGTDFTLKISRVEAEDVGV |
| | | YYCSQSTHV |
| SEQ ID NO: 29 | Heavy chain | EVQLVESGGGLVQPGGSLRLSCAASGFTFS |
| | H-Gamma-4 | SYGMSWVRQAPGKGLELVASINSNGGSTYY |
| | | PDSVKGRFTISRDNAKNSLYLQMNSLRAED |
| | | TAVYYCASGDYWGQGTTVTVSSASTKGPSV |
| | | FPLAPCSRSTSESTAALGCLVKDYFPEPVT |
| | | VSWNSGALTSGVHTFPAVLQSSGLYSLSSV |
| | | CHVTVPSSSLGTKTYTCNVDHKPSNTKVDK |
| | | RVESKYGPPCPPCPAPEFLGGPSVFLFPPK |
| | | PKDTLMISRTPEVTCVVVDVSQEDPEVQFN |
| | | WYVDGVEVHNAKTKPREEQFNSTYRVVSVL |
| | | TVLHQDWLNGKEYKCKVSNKGLPSSIEKTI |
| | | SKAKGQPREPQVYTLPPSQEEMTKNQVSLT |
| | | CLVKGFYPSDIAVEWESNGQPENNYKTTPP |
| | | VLDSDGSFFLYSRLTVDKSRWQEGNVFSCS |
| | | VMHEALHNHYTQKSLSLSLG |
| SEQ ID NO: 30 | Light chain | DIVMTQSPLSLPVTPGEPASISCRSSQSLV |
| | L-Kappa | YSNGDTYLHWYLQKPGQSPQLLIYKVSNRF |
| | | SGVPDRFSGSGSGTDFTLKISRVEAEDVGV |
| | | YYCSQSTHVPWTFGQGTKVEIKRTVAAPSV |
| | | FIFPPSDEQLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |

TABLE 6
Gantenerumab (HCDR1-3 and LCDR1-3 based on IMGT nomenclature)

| SEQ ID NO | Description | Amino Acid Sequence |
|---|---|---|
| SEQ ID NO: 31 | HCDR1 | GFTFSSYA |
| SEQ ID NO: 32 | HCDR2 | INASGTRT |
| SEQ ID NO: 33 | HCDR3 | ARGKGNTHKPYGYVRYFDV |
| SEQ ID NO: 34 | LCDR1 | QSVSSSY |
| SEQ ID NO: 35 | LCDR2 | GAS |
| SEQ ID NO: 36 | LCDR3 | LQIYNMPIT |
| SEQ ID NO: 37 | VH | QVELVESGGGLVQPGGSLRLSCAASGFTF |
| | | SSYAMSWVRQAPGKGLEWVSAINASGTRT |
| | | YYADSVKGRFTISRDNSKNTLYLQMNSLR |
| | | AEDTAV |
| SEQ ID NO: 38 | VL | IVLTQSPATLSLSPGERATLSCRASQSVS |
| | | SSYLAWYQQKPGQAPRLLIYGASSRATGV |
| | | PARFSGSGSGTDFTLTISSLEPEDFATYY |
| | | CLQIYNMP |
| SEQ ID NO: 39 | Heavy chain | QVELVESGGGLVQPGGSLRLSCAASGFTF |
| | H-Gamma-1 | SSYAMSWVRQAPGKGLEWVSAINASGTRT |
| | | YYADSVKGRFTISRDNSKNTLYLQMNSLR |
| | | AEDTAVYYCARGKGNTHKPYGYVRYFDVW |
| | | GQGTLVTVSSASTKGPSVFPLAPSSKSTS |
| | | GGTAALGCLVKDYFPEPVTVSWNSGALTS |
| | | GVHTFPAVLQSSGLYSLSSVVTVPSSSLG |
| | | TQTYICNVNHKPSNTKVDKKVEPKSCDKT |
| | | HTCPPCPAPELLGGPSVFLFPPKPKDTLM |
| | | ISRTPEVTCVVVDVSHEDPEVKFNWYVDG |
| | | VEVHNAKTKPREEQYNSTYRVVSVLTVLH |
| | | QDWLNGKEYKCKVSNKALPAPIEKTISKA |
| | | KGQPREPQVYTLPPSRDELTKNQVSLTCL |
| | | VKGFYPSDIAVEWESNGQPENNYKTTPPV |
| | | LDSDGSFFLYSKLTVDKSRWQQGNVFSCS |
| | | VMHEALHNHYTQKSLSLSPGK |
| SEQ ID NO: 40 | Light chain | DIVLTQSPATLSLSPGERATLSCRASQSV |
| | L-Kappa | SSSYLAWYQQKPGQAPRLLIYGASSRATG |
| | | VPARFSGSGSGTDFTLTISSLEPEDFATY |
| | | YCLQIYNMPITFGQGTKVEIKRTVAAPSV |
| | | FIFPPSDEQLKSGTASVVCLLNNFYPREA |
| | | KVQWKVDNALQSGNSQESVTEQDSKDSTY |
| | | SLSSTLTLSKADYEKHKVYACEVTHQGLS |
| | | SPVTKSFNRGEC |

TABLE 7
Ponezumab (HCDR1-3 and LCDR1-3 based on IMGT nomenclature)

| SEQ ID NO | Description | Amino Acid Sequence |
|---|---|---|
| SEQ ID NO: 41 | HCDR1 | GYYTEAYY |
| SEQ ID NO: 42 | HCDR2 | IDPATGNT |
| SEQ ID NO: 43 | HCDR3 | ASLYSLPVY |
| SEQ ID NO: 44 | LCDR1 | QSLLYSDAKTY |
| SEQ ID NO: 45 | LCDR2 | QIS |
| SEQ ID NO: 46 | LCDR3 | LQGTHYPVL |
| SEQ ID NO: 47 | VH | QVQLVQSGAEVKKPGASVKVSCKASGYYT |
| | | EAYYIHWVRQAPGQGLEWMGRIDPATGNT |
| | | KYAPRLQDRVTMTRDTSTSTVYMELSSLR |
| | | SEDTAVYYCA |
| SEQ ID NO: 48 | VL | DVVMTQSPLSLPVTLGQPASISCKSSQSL |
| | | LYSDAKTYLNWFQQRPGQSPRRLIYQISR |
| | | LDPGVPDRFSGSGSGTDFTLKISRVEAED |
| | | VGVYYCLQGTHYP |
| SEQ ID NO: 49 | Heavy chain | QVQLVQSGAEVKKPGASVKVSCKASGYYT |
| | H-Gamma-2 | EAYYIHWVRQAPGQGLEWMGRIDPATGNT |
| | | KYAPRLQDRVTMTRDTSTSTVYMELSSLR |
| | | SEDTAVYYCASLYSLPVYWGQGTTVTVSS |
| | | ASTKGPSVFPLAPCSRSTSESTAALGCLV |
| | | KDYFPEPVTVSWNSGALTSGVHTFPAVLQ |
| | | SSGLYSLSSVVTVPSSNFGTQTYTCNVDH |
| | | KPSNTKVDKTVERKCCVECPPCPAPPVAG |
| | | PSVFLFPPKPKDTLMISRTPEVTCVVVDV |
| | | SHEDPEVQFNWYVDGVEVHNAKTKPREEQ |
| | | FNSTFRVVSVLTVVHQDWLNGKEYKCKVS |
| | | NKGLPSSIEKTISKTKGQPREPQVYTLPP |
| | | SREEMTKNQVSLTCLVKGFYPSDIAVEWE |
| | | SNGQPENNYKTTPPMLDSDGSFFLYSKLT |
| | | VDKSRWQQGNVFSCSVMHEALHNHYTQKS |
| | | LSLSPGK |
| SEQ ID NO: 50 | Light chain | DVVMTQSPLSLPVTLGQPASISCKSSQSL |
| | L-Kappa | LYSDAKTYLNWFQQRPGQSPRRLIYQISR |
| | | LDPGVPDRFSGSGSGTDFTLKISRVEAED |
| | | VGVYYCLQGTHYPVLFGQGTRLEIKRTVA |
| | | APSVFIFPPSDEQLKSGTASVVCLLNNFY |
| | | PREAKVQWKVDNALQSGNSQESVTEQDSK |
| | | DSTYSLSSTLTLSKADYEKHKVYACEVTH |
| | | QGLSSPVTKSFNRGEC |

TABLE 8
Bapineuzumab (HCDR1-3 and LCDR1-3 based on IMGT nomenclature)

| SEQ ID NO | Description | Amino Acid Sequence |
|---|---|---|
| SEQ ID NO: 51 | HCDR1 | GFTFSNYG |
| SEQ ID NO: 52 | HCDR2 | ASIRSGGGRT |
| SEQ ID NO: 53 | HCDR3 | VRYDHYSGSSDY |
| SEQ ID NO: 54 | LCDR1 | QSLLDSDGKTY |
| SEQ ID NO: 55 | LCDR2 | LVS |
| SEQ ID NO: 56 | LCDR3 | WQGTHEPRT |
| SEQ ID NO: 57 | VH | EVQLLESGGGLVQPGGSLRLSCAASGFTFS |
| | | NYGMSWVRQAPGKGLEWVASIRSGGGRTYY |
| | | SDNVKGRFTISRDNSKNTLYLQMNSLRAED |
| | | TAVYYCVR |
| SEQ ID NO: 58 | VL | DVVMTQSPLSLPVTPGEPASISCKSSQSLL |
| | | DSDGKTYLNWLLQKPGQSPQRLIYLVSKLD |
| | | SGVPDRFSGSGSGTDFTLKISRVEAEDVGV |
| | | YYCWQGTHEP |
| SEQ ID NO: 59 | Heavy chain | EVQLLESGGGLVQPGGSLRLSCAASGFTFS |
| | H-Gamma-1 | NYGMSWVRQAPGKGLEWVASIRSGGGRTYY |
| | | SDNVKGRFTISRDNSKNTLYLQMNSLRAED |
| | | TAVYYCVRYDHYSGSSDYWGQGTLVTVSSA |
| | | STKGPSVFPLAPSSKSTSGGTAALGCLVKD |
| | | YFPEPVTVSWNSGALTSGVHTFPAVLQSSG |
| | | LYSLSSVVTVPSSSLGTQTYICNVNHKPSN |
| | | TKVDKKVEPKSCKTHTCPPCPAPELLGGPS |
| | | VFLFPPKPKDTLMISRTPEVTCVVVDVSHE |
| | | DPEVKFNWYVDGVEVHNAKTKPREEQYNST |
| | | YRVVSVLTVLHQDWLNGKEYKCKVSNKALP |
| | | APIEKTISKAKGQPREPQVYTLPPSREEMT |
| | | KNQVSLTCLVKGFYPSDIAVEWESNGQPEN |
| | | NYKTTPPVLDSDGSFFLYSKLTVDKSRWQQ |
| | | GNVFSCSVMHEALHNKYTQKSLSLSPG |
| SEQ ID NO: 60 | Light chain | DVVMTQSPLSLPVTPGEPASISCKSSQSLL |
| | L-Kappa | DSDGKTYLNWLLQKPGQSPQRLIYLVSKLD |
| | | SGVPDRFSGSGSGTDFTLKISRVEAEDVGV |
| | | YYCWQGTHFPRTFGQGTKVEIKRTVAAPSV |
| | | FIFPPSDEQLKSGTASVVCLLNNFYPREAK |
| | | VQWKVDNALQSGNSQESVTEQDSKDSTYSL |

TABLE 9
Lecanemab (HCDR1-3 and LCDR1-3 based on IMGT nomenclature)

| SEQ ID NO | Description | Amino Acid Sequence |
|---|---|---|
| SEQ ID NO: 61 | HCDR1 | GFTFSSFG |
| SEQ ID NO: 62 | HCDR2 | ISSGSSTI |
| SEQ ID NO: 63 | HCDR3 | AREGGYYYGRSYYTMDY |
| SEQ ID NO: 64 | LCDR1 | QSIVHSNGNTY |
| SEQ ID NO: 65 | LCDR2 | KVS |
| SEQ ID NO: 66 | LCDR3 | FQGSHVPPT |
| SEQ ID NO: 67 | VH | EVQLVESGGGLVQPGGSLRLSCSASGFTFS |
| | | SFGMHWVRQAPGKGLEWVAYISSGSSTIYY |
| | | GDTVKGRFTISRDNAKNSLFLQMSSLRAED |
| | | TAVYYCAR |
| SEQ ID NO: 68 | VL | DVVMTQSPLSLPVTPGAPASISCRSSQSIV |
| | | HSNGNTYLEWYLQKPGQSPKLLIYKVSNRF |
| | | SGVPDRFSGSGSGTDFTLRISRVEAEDVGI |
| | | YYCFQGSHVP |
| SEQ ID NO: 69 | Heavy chain | EVQLVESGGGLVQPGGSLRLSCSASGFTFS |
| | H-Gamma-1 | SFGMHWVRQAPGKGLEWVAYISSGSSTIYY |
| | | GDTVKGRFTISRDNAKNSLFLQMSSLRAED |
| | | TAVYYCAREGGYYYGRSYYTMDYWGQGTTV |
| | | TVSSASTKGPSVFPLAPSSKSTSGGTAALG |
| | | CLVKDYFPEPVTVSWNSGALTSGVHTFPAV |
| | | LQSSGLYSLSSVVTVPSSSLGTQTYICNVN |
| | | HKPSNTKVDKRVEPKSCDKTHTCPPCPAPE |
| | | LLGGPSVFLFPPKPKDTLMISRTPEVTCVV |
| | | VDVSHEDPEVKFNWYVDGVEVHNAKTKPRE |
| | | EQYNSTYRVVSVLTVLHQDWLNGKEYKCKV |
| | | SNKALPAPIEKTISKAKGQPREPQVYTLPP |
| | | SREEMTKNQVSLTCLVKGFYPSDIAVEWES |
| | | NGQPENNYKTTPPVLDSDGSFFLYSKLTVD |
| | | KSRWQQGNVFSCSVMHEALHNHYTQKSLSL |
| | | SPGK |
| SEQ ID NO: 70 | Light chain | DVVMTQSPLSLPVTPGAPASISCRSSQSIV |
| | L-Kappa | HSNGNTYLEWYLQKPGQSPKLLIYKVSNRF |
| | | SGVPDRFSGSGSGTDFTLRISRVEAEDVGI |
| | | YYCFQGSHVPPTFGPGTKLEIKRTVAAPSV |
| | | FIFPPSDEQLKSGTASVVCLLNNFYPREAK |
| | | VQWKVDNALQSGNSQESVTEQDSKDSTYSL |
| | | SSTLTLSKADYEKHKVYACEVTHQGLSSPV |
| | | TKSFNRGEC |

In some embodiments, the anti-amyloid beta antibody or antigen-binding fragment thereof comprises a human IgG1, IgG2, IgG3, or IgG4 constant domain. In some embodiments, the anti-amyloid beta antibody or antigen-binding fragment thereof comprises a human IgG1 constant region. In some embodiments, the anti-amyloid beta antibody or antigen-binding fragment thereof comprises a human IgG2 constant region. In some embodiments, the anti-amyloid beta antibody or antigen-binding fragment thereof comprises a human IgG3 constant region. In some embodiments, the anti-amyloid beta antibody or antigen-binding fragment thereof comprises a human IgG4 constant region.

In some embodiments, the anti-amyloid beta antibody or antigen-binding fragment thereof is a human anti-amyloid beta antibody or antigen-binding fragment thereof.

In some embodiments, the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb). In some embodiments, the anti-amyloid beta antibody is a whole antibody. In some embodiments, an anti-amyloid beta antibody is a single chain antibody. In some embodiments, an anti-amyloid beta antibody is a scFv. In some embodiments, an anti-amyloid beta antibody is a Fab. In some embodiments, an anti-amyloid beta antibody is a F(ab′)₂. In some embodiments, an anti-amyloid beta antibody is a Fv. In some embodiments, an anti-amyloid beta antibody is a Fd. In some embodiments, an anti-amyloid beta antibody is a bispecific single chain Fv dimer. In some embodiments, an anti-amyloid beta antibody is a diabody. In some embodiments, an anti-amyloid beta antibody is a single domain antibody (dAb). In some embodiments, an anti-amyloid beta antibody is a bispecific antibody.

In some embodiments, an anti-amyloid beta antibody, or an antigen-binding fragment thereof, of the disclosure is human. In some embodiments, the human anti-amyloid beta, or an antigen-binding fragment thereof, comprises a human IgH chain and a human Igκ chain. In some embodiments, the human anti-amyloid beta, or an antigen-binding fragment thereof, comprises a human IgH chain and a human Igλ chain. In some embodiments, the isotype of the anti-amyloid beta antibody is selected from IgM, IgD, IgG (such as IgG1, IgG2, IgG3, and IgG4), IgA and IgE. In some embodiments, the isotype of the anti-amyloid beta antibody is selected from IgG1, IgG2, IgG3, and IgG4.

In some embodiments, the anti-amyloid beta antibody binds an Fc receptor (FcR) selected from an FcγR, an FcεR, and an FcαR. In some embodiments, the anti-amyloid beta antibody binds an FcγR selected from FcγRI (CD64), FcγRII (CD32), and FcγRIII (CD16), including isoforms thereof. In some embodiments, the Fc region of the anti-amyloid beta antibody comprises a mutation so that it preferentially binds a particular FcγR.

In some embodiments, the anti-amyloid beta antibody or antigen binding fragment thereof binds to amyloid beta plaques, parenchymal amyloid, cerebrovascular amyloid, or diffuse amyloid beta deposits.

### Pharmaceutical Compositions

In one aspect, the disclosure provides a pharmaceutical composition comprising a Compound 1 or a pharmaceutically acceptable salt thereof (also referred to as the “active ingredient”), and a pharmaceutically acceptable excipient. In certain embodiments, the pharmaceutical composition comprises an effective amount of Compound 1 or a pharmaceutically acceptable salt thereof. In certain embodiments, the pharmaceutical composition comprises a therapeutically effective amount of Compound 1 or a pharmaceutically acceptable salt thereof. In certain embodiments, the pharmaceutical composition comprises a prophylactically effective amount of Compound 1 or a pharmaceutically acceptable salt thereof.

In some embodiment, the pharmaceutical composition comprises about 0.1-about 1.0 mg of Compound 1 or a pharmaceutically acceptable salt thereof. In some embodiment, the pharmaceutical composition comprises about 0.1-about 1.0 mg of Compound 1. In some embodiment, the pharmaceutical composition comprises about 0.1-about 1.0 mg of a pharmaceutically acceptable salt of Compound 1. In some embodiment, the pharmaceutical composition comprises about 0.5 mg of Compound 1 or a pharmaceutically acceptable salt thereof. In some embodiment, the pharmaceutical composition comprises about 0.5 mg of Compound 1. In some embodiment, the pharmaceutical composition comprises about 0.5 mg of a pharmaceutically acceptable salt of Compound 1.

In some embodiment, the pharmaceutical composition comprises 0.1-1.0 mg of Compound 1 or a pharmaceutically acceptable salt thereof. In some embodiment, the pharmaceutical composition comprises 0.1-1.0 mg of Compound 1. In some embodiment, the pharmaceutical composition comprises 0.1-1.0 mg of a pharmaceutically acceptable salt of Compound 1. In some embodiment, the pharmaceutical composition comprises 0.5 mg of Compound 1 or a pharmaceutically acceptable salt thereof. In some embodiment, the pharmaceutical composition comprises 0.5 mg of Compound 1. In some embodiment, the pharmaceutical composition comprises 0.5 mg of a pharmaceutically acceptable salt of Compound 1. In some embodiment, the pharmaceutical composition comprises 1.0 mg of Compound 1 or a pharmaceutically acceptable salt thereof. In some embodiment, the pharmaceutical composition comprises 1.0 mg of Compound 1. In some embodiment, the pharmaceutical composition comprises 1.0 mg of a pharmaceutically acceptable salt of Compound 1.

In one aspect, provided herein is a pharmaceutical composition comprising: (a) an NMDA receptor positive allosteric modulator; (b) an anti-amyloid beta antibody or antigen-binding fragment thereof; and (c) a pharmaceutically acceptable carrier. In some embodiments, the pharmaceutical composition comprises (a) an effective amount of an NMDA receptor positive allosteric modulator; (b) an effective amount of an anti-amyloid beta or antigen binding fragment thereof, and (c) a pharmaceutically acceptable carrier. In some embodiments, the pharmaceutical composition comprises (a) a therapeutically effective amount of an NMDA receptor positive allosteric modulator; (b) a therapeutically effective amount of an anti-amyloid beta or antigen binding fragment thereof, and (c) a pharmaceutically acceptable carrier.

In one aspect, provided herein is a pharmaceutical composition comprising: (a) a CYP46A1 inhibitor; (b) an anti-amyloid beta antibody or antigen-binding fragment thereof; and (c) a pharmaceutically acceptable carrier. In some embodiments, the pharmaceutical composition comprises (a) an effective amount of a CYP46A1; (b) an effective amount of an anti-amyloid beta or antigen binding fragment thereof, and (c) a pharmaceutically acceptable carrier. In some embodiments, the pharmaceutical composition comprises (a) a therapeutically effective amount of a CYP46A1; (b) a therapeutically effective amount of an anti-amyloid beta or antigen binding fragment thereof, and (c) a pharmaceutically acceptable carrier.

The pharmaceutical compositions provided herein can be administered by a variety of routes including, but not limited to, oral (enteral) administration, parenteral (by injection) administration, rectal administration, transdermal administration, intradermal administration, intrathecal administration, subcutaneous (SC) administration, intravenous (IV) administration, intramuscular (IM) administration, and intranasal administration. In some embodiments, the compound or pharmaceutical composition is administered to a subject orally. In some embodiments, the compound or pharmaceutical composition is administered to a subject parenterally. In some embodiments, the compound or pharmaceutical composition is administered to a subject rectally. In some embodiments, the compound or pharmaceutical composition is administered to a subject transdermally. In some embodiments, the compound or pharmaceutical composition is administered to a subject intradermally. In some embodiments, the compound or pharmaceutical composition is administered to a subject intrathecally. In some embodiments, the compound or pharmaceutical composition is administered to a subject subcutaneously. In some embodiments, the compound or pharmaceutical composition is administered to a subject intravenously. In some embodiments, the compound or pharmaceutical composition is administered to a subject intramuscularly. In some embodiments, the compound or pharmaceutical composition is administered to a subject intranasally.

Generally, the compounds (i.e., Compound 1, the CYP46A1 inhibitors, the NMDA PAMs and the anti-amyloid beta or antigen binding fragment thereof) provided herein are administered in an effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of the present invention may be further delivered using a variety of dosing methods. For example, in certain embodiments, the pharmaceutical composition may be given as a bolus, e.g., in order to raise the concentration of the compound in the blood to an effective level. The placement of the bolus dose depends on the systemic levels of the active ingredient desired throughout the body, e.g., an intramuscular or subcutaneous bolus dose allows a slow release of the active ingredient, while a bolus delivered directly to the veins (e.g., through an IV drip) allows a much faster delivery which quickly raises the concentration of the active ingredient in the blood to an effective level. In other embodiments, the pharmaceutical composition may be administered as a continuous infusion, e.g., by IV drip, to provide maintenance of a steady-state concentration of the active ingredient in the subject's body. Furthermore, in still yet other embodiments, the pharmaceutical composition may be administered as first as a bolus dose, followed by continuous infusion.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, or the like in the case of solid compositions. In such compositions, the compound is usually a minor component (from about 0.1 to about 50% by weight or preferably from about 1 to about 40% by weight) with the remainder being various vehicles or excipients and processing aids helpful for forming the desired dosing form. The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pennsylvania, which is incorporated herein by reference.

The compositions may be in sustained release forms or from sustained release drug delivery systems. A description of representative sustained release materials can be found in Remington's Pharmaceutical Sciences.

The present invention also relates to the pharmaceutically acceptable acid addition salt of the compounds (i.e., Compound 1, CYP46A1 inhibitors and the NMDA PAMs) of the disclosure. The acid which may be used to prepare the pharmaceutically acceptable salt is that which forms a non-toxic acid addition salt, i.e., a salt containing pharmacologically acceptable anions such as the hydrochloride, hydroiodide, hydrobromide, nitrate, sulfate, bisulfate, phosphate, acetate, lactate, citrate, tartrate, succinate, maleate, fumarate, benzoate, para-toluenesulfonate, and the like.

When used to prevent the onset of a CNS-disorder, the compounds provided herein will be administered to a subject at risk for developing the condition, typically on the advice and under the supervision of a physician, at the dosage levels described herein. Subjects at risk for developing a particular condition generally include those that have a family history of the condition, or those who have been identified by genetic testing or screening to be particularly susceptible to developing the condition.

### Kits

In one aspect, provided herein is a kit comprising a composition comprising an NMDA receptor positive allosteric modulator as disclosed herein; a composition comprising an anti-amyloid beta antibody or antigen-binding fragment thereof as disclosed herein; and a package insert. In some embodiments, provided herein is a kit comprising a first container, a second container and a package insert, wherein the first container comprises a composition comprising an NMDA receptor positive allosteric modulator as disclosed herein; the second container comprises composition comprising an anti-amyloid beta antibody or antigen-binding fragment thereof as disclosed herein; and the package insert comprises instructions for treating or preventing Alzheimer's disease in a subject. It is understood that the kit may comprise one distinct composition or two or more distinct compositions wherein the first composition comprises an NMDA PAM and second composition comprises an anti-amyloid beta antibody or antigen-binding fragment thereof.

It is understood that the NMDA PAM compositions and anti-amyloid beta antibody or antigen-binding fragment thereof compositions may independently have any dosage form. For example, the anti-amyloid beta antibody or antigen-binding fragment thereof composition may be a liquid dosage form, whereas the NMDA PAM composition may be a solid dosage form.

In one aspect, provided herein is a kit comprising a composition comprising a CYP46A1 inhibitor as disclosed herein; a composition comprising an anti-amyloid beta antibody or antigen-binding fragment thereof as disclosed herein; and a package insert. In some embodiments, provided herein is a kit comprising a first container, a second container and a package insert, wherein the first container comprises a composition comprising a CYP46A1 inhibitor as disclosed herein; the second container comprises composition comprising an anti-amyloid beta antibody or antigen-binding fragment thereof as disclosed herein; and the package insert comprises instructions for treating or preventing Alzheimer's disease in a subject. It is understood that the kit may comprise one distinct composition or two or more distinct compositions wherein the first composition comprises a CYP46A1 inhibitor, and second composition comprises an anti-amyloid beta antibody or antigen-binding fragment thereof.

It is understood that the CYP46A1 inhibitor compositions and anti-amyloid beta antibody or antigen-binding fragment thereof compositions may independently have any dosage form. For example, the anti-amyloid beta antibody or antigen-binding fragment thereof composition may be a liquid dosage form, whereas the CYP46A1 inhibitor composition may be a solid dosage form.

It is understood that the package insert contains instructions for treating any of conditions disclosed herein. In some embodiments, the instructions are for treating Alzheimer's disease. In some embodiments, the instructions are for preventing Alzheimer's disease. In some embodiments, the instructions are for treating cognitive impairment in a subject having Alzheimer's disease. In some embodiments, the instructions are for preventing Alzheimer's disease. In some embodiments, the instructions are for preventing cognitive impairment in a subject having Alzheimer's disease. In some embodiments, the instructions are for treating cognitive impairment due to Alzheimer's disease in a subject. In some embodiments, the instructions are for preventing cognitive impairment due to Alzheimer's disease in a subject. In some embodiments, the instructions are for treating mild cognitive impairment due to Alzheimer's disease in a subject. In some embodiments, the instructions are for preventing mild cognitive impairment due to Alzheimer's disease in a subject. In some embodiments, the instructions are for improving cognition in a subject having Alzheimer's disease. In some embodiments, the instructions are for slowing cognitive decline due to Alzheimer's disease in a subject. In some embodiments, the instructions are for decreasing amyloid beta plaques in the brain of a subject having Alzheimer's. In some embodiments, the instructions are for decreasing the deposition of amyloid beta plaques in the brain of a subject having Alzheimer's disease. In some embodiments, the instructions are for treating Mild Cognitive Impairment (MCI) associated with Alzheimer's Disease. In some embodiments, the instructions are for improving executive function in a subject having Mild Cognitive Impairment (MCI) associated with Alzheimer's Disease. In some embodiments, the instructions are for improving working memory in a subject having Mild Cognitive Impairment (MCI) associated with Alzheimer's Disease. In some embodiments, the instructions are for improving learning in a subject having Mild Cognitive Impairment (MCI) associated with Alzheimer's Disease. In some embodiments, the instructions are for improving learning and working memory in a subject having Mild Cognitive Impairment (MCI) associated with Alzheimer's Disease. In some embodiments, the instructions are for treating mild dementia associated with Alzheimer's Disease in a subject. In some embodiments, the instructions are for improving executive function in a subject having mild dementia associated with Alzheimer's Disease. In some embodiments, the instructions are for improving working memory in a subject having mild dementia associated with Alzheimer's Disease. In some embodiments, the instructions are for improving learning in a subject having mild dementia associated with Alzheimer's Disease. In some embodiments, the instructions are for improving learning and working memory in a subject having mild dementia associated with Alzheimer's Disease.

In some embodiments, the kit comprises instructions for administering the anti-amyloid beta antibody or antigen-binding fragment thereof and the NMDA PAM simultaneously. In some embodiments, the kit comprises instructions for administering anti-amyloid beta antibody or antigen-binding fragment thereof and the NMDA PAM sequentially. In some embodiments, the kit comprises instructions for administering the anti-amyloid beta antibody or antigen-binding fragment thereof and the NMDA PAM separately. In some embodiments, the kit comprises instructions for administering the anti-amyloid beta antibody or antigen-binding fragment thereof before the NMDA PAM. In some embodiments, the kit comprises instructions for administering the NMDA PAM before anti-amyloid beta antibody or antigen-binding fragment thereof. In some embodiments, the kit comprises instructions for administering the anti-amyloid beta antibody or antigen-binding fragment thereof and the NMDA PAM with the same frequency. In some embodiments, the kit comprises instructions for administering the anti-amyloid beta antibody or antigen-binding fragment thereof and the NMDA PAM with different frequencies.

In some embodiments, the kit comprises instructions for administering the anti-amyloid beta antibody or antigen-binding fragment thereof and the CYP46A1 inhibitor simultaneously. In some embodiments, the kit comprises instructions for administering anti-amyloid beta antibody or antigen-binding fragment thereof and the CYP46A1 inhibitor sequentially. In some embodiments, the kit comprises instructions for administering the anti-amyloid beta antibody or antigen-binding fragment thereof and the CYP46A1 inhibitor separately. In some embodiments, the kit comprises instructions for administering the anti-amyloid beta antibody or antigen-binding fragment thereof before the CYP46A1 inhibitor. In some embodiments, the kit comprises instructions for administering the CYP46A1 inhibitor before anti-amyloid beta antibody or antigen-binding fragment thereof. In some embodiments, the kit comprises instructions for administering the anti-amyloid beta antibody or antigen-binding fragment thereof and the CYP46A1 inhibitor with the same frequency. In some embodiments, the kit comprises instructions for administering the anti-amyloid beta antibody or antigen-binding fragment thereof and the CYP46A1 inhibitor with different frequencies.

The kits of the disclosure may be in any suitable packaging. Suitable packaging includes, but is not limited to, vials, bottles, ampoules, jars, syringes, flexible packaging (e.g., sealed Mylar or plastic bags), and the like. Kits may optionally provide additional components such as buffers and interpretive information.

The first and second containers may be of the same or different shape (e.g., vials, syringes, and bottles) and/or material (e.g., plastic or glass). The kit may further comprise other materials that may be useful in administering the medicaments, such as diluents, filters, IV bags and lines, needles and syringes.

In some embodiments, the invention provides articles of manufacture comprising contents of the kits described herein.

### Methods of Treatment and Use

The compounds, pharmaceutical compositions and kits disclosed herein are useful in treating or preventing Alzheimer's disease.

Alzheimer's disease (AD) includes dementia which is primarily identified by clinical diagnosis and established by markers of the disease. It is continuum having certain operationally defined stages of disease progression. AD pathology begins prior to the onset of clinical symptoms. For example, amyloid plaques, one marker of AD pathology, form 10-20 years prior to the onset of AD dementia. The currently recognized stages of AD include preclinical, prodromal, mild, moderate, and severe. These stages may be further divided into subcategories based on the severity of symptoms and measures of AD progression.

Those skilled in the art will recognize that the differences between patient groups may not be distinct in a particular clinical setting. Nevertheless, the clinical disease stage can be characterized by measuring various characteristics or markers, and changes in these characteristics or markers over time, such as amyloid-beta accumulation (CSF/PET), synaptic dysfunction (FDG-PET/fMRI), tau-mediated neuronal injury (CSF), brain structure (volumetric MRI), cognition, and clinical function. Clifford Jack et al. Lancet. Neurol. 2010 January; 9 (1): 119.

Current core clinical criteria for all dementia, referred to as the NINCDS-ADRDA criteria (McKhann G M, V. diagnosis of dementia due to Alzheimer's disease: Recommendations from the National Inst. on Aging-Alzheimer's Association workgroups on diagnostic guidelines for Alzheimer's disease. Alzheimer's & Dementia 7 (2011) 263-269), are known in the art and can be employed in practicing this invention. They include cognitive or behavioral impairment involving impaired ability to acquire and remember new information, impaired reasoning and handling of complex tasks, impaired visuospatial abilities, impaired language functions (speaking, reading, writing), and changes in personality, behavior, or comportment. Id. Alzheimer's disease is currently diagnosed using the core criteria and is typically characterized by symptoms which have a gradual onset over months to years, not sudden over hours or days (insidious onset). There is usually a clear-cut history of worsening of cognition by report or observation in Alzheimer's disease subjects. Id. Other diagnostic classification systems have evolved as new information on AD has become available. These systems include the International Working Group (IWG) new research criteria for diagnosis of AD (Dubois B et al. Lancet Neurol 2007; 6 (8): 734-736), IWG research criteria, (Dubois et al. Lancet Neurol 2010; 9 (11): 1118-27), NIA/AA Criteria (Jack C R et al. Alzheimer's Dement 2011; 7 (3): 257-62), and DSM-5 criteria (American Psychiatric Association, DSM-5, 2013). These classification systems can also be employed in diagnosing AD subjects for treatment according to the methods of this invention.

The subject having Alzheimer's disease referred to in the methods herein include without limitation, patients with preclinical, prodromal, mild, moderate, or severe Alzheimer's disease.

The subjects having Alzheimer's disease in need of treatment range from subjects with amyloid pathology and early neuronal degeneration to subjects with widespread neurodegeneration and irreversible neuronal loss with progressive cognitive and functional impairment to subjects with dementia. Subjects with preclinical Alzheimer's disease can be identified by asymptomatic stages with or without memory complaints and emerging episodic memory and executive function deficits. This stage is typically characterized by the appearance of in vivo molecular biomarkers of Alzheimer's disease and the absence clinical symptoms.

Prodromal Alzheimer's disease patients are pre-dementia stage characterized predominantly by cognitive deficits and emerging functional impairment with disease progression. Prodromal AD patients typically have MMSE scores between 24-30 (inclusive), a spontaneous memory complaint, objective memory loss defined as a free recall score of ≤27 on the FCSRT, a global CDR score of 0.5, absence of significant levels of impairment in other cognitive domains, and essentially preserved activities of daily living, and an absence of dementia.

Patients with mild AD typically have MMSE scores between 20-26 (inclusive), a global CDR of 0.5 or 1.0, and meet the National Institute on Aging-Alzheimer's Association core clinical criteria for probable AD (see Section 22).

Basing AD diagnosis on clinical symptoms, mild stage AD patients will exhibit conspicuous behavior at work, forgetfulness, mood swings, and attention disturbances. Moderate stage AD patients will exhibit cognitive deficits, restricted everyday activities, orientation disturbance, apraxia, agnosia, aphasia, and behavioral abnormalities. Severe stage AD patients are characterized by loss of independence, decay of memory and speech, and incontinence.

In some embodiments, the disclosure provides treatment of earlier-stage patients who are amyloid positive as assessed by ¹⁸F-AV-45 PET scans. The patient may be asymptomatic for, or exhibit only transient symptoms of, headache, confusion, gait difficulties, or visual disturbances. The patient may or may not be an ApoE4 carrier as determined by ApoE genotyping. In other embodiments, the disclosure provides treatment of patients having any medical or neurological condition (other than AD) that might be a contributing cause of the subject's cognitive impairment, such as stroke or other cerebrovascular condition, other neurodegenerative disease, a history of clinically significant psychiatric illness, acute or sub-acute micro- or macro-hemorrhage, prior macro-hemorrhage, or superficial siderosis, but even these patients can be treated following screening and selection by a qualified clinician.

Thus, in one aspect, provided herein is a method of treating Mild Cognitive Impairment (MCI) associated with Alzheimer's Disease in a subject, the method comprising administering to the subject Compound 1

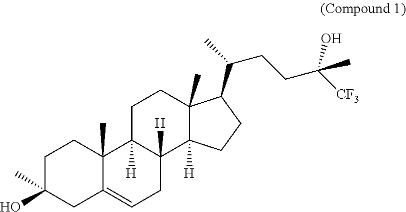

or a pharmaceutically acceptable salt thereof. In some embodiments, provided herein is a method of treating Mild Cognitive Impairment (MCI) associated with Alzheimer's Disease in a subject, the method comprising administering to the subject Compound 1. In some embodiments, provided herein is a method of treating Mild Cognitive Impairment (MCI) associated with Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of treating mild dementia associated with Alzheimer's Disease in a subject, the method comprising administering to the subject Compound 1, or a pharmaceutically acceptable salt thereof. In some embodiments, provided herein is a method of treating mild dementia associated with Alzheimer's Disease in a subject, the method comprising administering to the subject Compound 1. In some embodiments, provided herein is a method of treating mild dementia associated with Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of treating Mild Cognitive Impairment (MCI) resulting from Alzheimer's Disease in a subject, the method comprising administering to the subject Compound 1, or a pharmaceutically acceptable salt thereof. In some embodiments, provided herein is a method of treating Mild Cognitive Impairment (MCI) resulting from Alzheimer's Disease in a subject, the method comprising administering to the subject Compound 1. In some embodiments, provided herein is a method of treating Mild Cognitive Impairment (MCI) resulting from Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of treating mild dementia resulting from Alzheimer's Disease in a subject, the method comprising administering to the subject Compound 1, or a pharmaceutically acceptable salt thereof. In some embodiments, provided herein is a method of treating mild dementia resulting from Alzheimer's Disease in a subject, the method comprising administering to the subject Compound 1. In some embodiments, provided herein is a method of treating mild dementia resulting from Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of treating Mild Cognitive Impairment (MCI) in a subject having Alzheimer's Disease, the method comprising administering to the subject Compound 1, or a pharmaceutically acceptable salt thereof. In some embodiments, provided herein is a method of treating Mild Cognitive Impairment (MCI) in a subject having Alzheimer's Disease, the method comprising administering to the subject Compound 1. In some embodiments, provided herein is a method of treating Mild Cognitive Impairment (MCI) in a subject having Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of treating mild dementia in a subject having Alzheimer's Disease, the method comprising administering to the subject Compound 1, or a pharmaceutically acceptable salt thereof. In some embodiments, provided herein is a method of treating mild dementia in a subject having Alzheimer's Disease, the method comprising administering to the subject Compound 1. In some embodiments, provided herein is a method of treating mild dementia in a subject having Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving executive function in a subject having Mild Cognitive Impairment (MCI) associated with Alzheimer's Disease, the method comprising administering to the subject Compound 1, or a pharmaceutically acceptable salt thereof. In some embodiments, provided herein is a method of improving executive function in a subject having Mild Cognitive Impairment (MCI) associated with Alzheimer's Disease, the method comprising administering to the subject Compound 1. In some embodiments, provided herein is a method of improving executive function in a subject having Mild Cognitive Impairment (MCI) associated with Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving executive function in a subject having mild dementia associated with Alzheimer's Disease, the method comprising administering to the subject Compound 1, or a pharmaceutically acceptable salt thereof. In some embodiments, provided herein is a method of improving executive function in a subject having mild dementia associated with Alzheimer's Disease in a subject, the method comprising administering to the subject Compound 1. In some embodiments, provided herein is a method of improving executive function in a subject having mild dementia associated with Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving executive function in a subject having Mild Cognitive Impairment (MCI) resulting from Alzheimer's Disease, the method comprising administering to the subject Compound 1, or a pharmaceutically acceptable salt thereof. In some embodiments, provided herein is a method of improving executive function in a subject having Mild Cognitive Impairment (MCI) resulting from Alzheimer's Disease, the method comprising administering to the subject Compound 1. In some embodiments, provided herein is a method of improving executive function in a subject having Mild Cognitive Impairment (MCI) resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving executive function in a subject having mild dementia resulting from Alzheimer's Disease, the method comprising administering to the subject Compound 1, or a pharmaceutically acceptable salt thereof. In some embodiments, provided herein is a method of improving executive function in a subject having mild dementia resulting from Alzheimer's Disease, the method comprising administering to the subject Compound 1. In some embodiments, provided herein is a method of improving executive function in a subject having mild dementia resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving executive function in an Alzheimer's Disease subject having Mild Cognitive Impairment (MCI), the method comprising administering to the subject Compound 1, or a pharmaceutically acceptable salt thereof. In some embodiments, provided herein is a method of improving executive function, in an Alzheimer's Disease subject having Mild Cognitive Impairment (MCI), the method comprising administering to the subject Compound 1. In some embodiments, provided herein is a method of improving executive function in an Alzheimer's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving executive function in an Alzheimer's Disease subject having mild dementia, the method comprising administering to the subject Compound 1, or a pharmaceutically acceptable salt thereof. In some embodiments, provided herein is a method of improving executive function, in an Alzheimer's Disease subject having mild dementia, the method comprising administering to the subject Compound 1. In some embodiments, provided herein is a method of improving executive function in an Alzheimer's Disease subject having mild dementia, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving working memory in a subject having Mild Cognitive Impairment (MCI) associated with Alzheimer's Disease, the method comprising administering to the subject Compound 1, or a pharmaceutically acceptable salt thereof. In some embodiments, provided herein is a method of improving working memory in a subject having Mild Cognitive Impairment (MCI) associated with Alzheimer's Disease in a subject, the method comprising administering to the subject Compound 1. In some embodiments, provided herein is a method of improving working memory in a subject having Mild Cognitive Impairment (MCI) associated with Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving working memory in a subject having mild dementia associated with Alzheimer's Disease, the method comprising administering to the subject Compound 1, or a pharmaceutically acceptable salt thereof. In some embodiments, provided herein is a method of improving working memory in a subject having mild dementia associated with Alzheimer's Disease in a subject, the method comprising administering to the subject Compound 1. In some embodiments, provided herein is a method of improving working memory in a subject having mild dementia associated with Alzheimer's Disease in a subject, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving working memory in a subject having Mild Cognitive Impairment (MCI) resulting from Alzheimer's Disease, the method comprising administering to the subject Compound 1, or a pharmaceutically acceptable salt thereof. In some embodiments, provided herein is a method of improving working memory in a subject having Mild Cognitive Impairment (MCI) resulting from Alzheimer's Disease, the method comprising administering to the subject Compound 1. In some embodiments, provided herein is a method of improving working memory in a subject having Mild Cognitive Impairment (MCI) resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving working memory in a subject having mild dementia resulting from Alzheimer's Disease, the method comprising administering to the subject Compound 1, or a pharmaceutically acceptable salt thereof. In some embodiments, provided herein is a method of improving working memory in a subject having mild dementia resulting from Alzheimer's Disease, the method comprising administering to the subject Compound 1. In some embodiments, provided herein is a method of improving working memory in a subject having mild dementia resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving working memory in an Alzheimer's Disease subject having Mild Cognitive Impairment (MCI), the method comprising administering to the subject Compound 1, or a pharmaceutically acceptable salt thereof. In some embodiments, provided herein is a method of improving working memory, in an Alzheimer's Disease subject having Mild Cognitive Impairment (MCI), the method comprising administering to the subject Compound 1. In some embodiments, provided herein is a method of improving working memory in an Alzheimer's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving working memory in an Alzheimer's Disease subject having mild dementia, the method comprising administering to the subject Compound 1, or a pharmaceutically acceptable salt thereof. In some embodiments, provided herein is a method of improving working memory, in an Alzheimer's Disease subject having mild dementia, the method comprising administering to the subject Compound 1. In some embodiments, provided herein is a method of improving working memory in an Alzheimer's Disease subject having mild dementia, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving learning in a subject having Mild Cognitive Impairment (MCI) associated with Alzheimer's Disease, the method comprising administering to the subject Compound 1, or a pharmaceutically acceptable salt thereof. In some embodiments, provided herein is a method of improving learning in a subject having Mild Cognitive Impairment (MCI) associated with Alzheimer's Disease, the method comprising administering to the subject Compound 1. In some embodiments, provided herein is a method of improving learning in a subject having Mild Cognitive Impairment (MCI) associated with Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving learning in a subject having mild dementia associated with Alzheimer's Disease, the method comprising administering to the subject Compound 1, or a pharmaceutically acceptable salt thereof. In some embodiments, provided herein is a method of improving learning in a subject having mild dementia associated with Alzheimer's Disease, the method comprising administering to the subject Compound 1. In some embodiments, provided herein is a method of improving learning in a subject having mild dementia associated with Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning in a subject having Mild Cognitive Impairment (MCI) resulting from Alzheimer's Disease, the method comprising administering to the subject Compound 1, or a pharmaceutically acceptable salt thereof. In some embodiments, provided herein is a method of improving learning in a subject having Mild Cognitive Impairment (MCI) resulting from Alzheimer's Disease, the method comprising administering to the subject Compound 1. In some embodiments, provided herein is a method of improving learning in a subject having Mild Cognitive Impairment (MCI) resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning in a subject having mild dementia resulting from Alzheimer's Disease, the method comprising administering to the subject Compound 1, or a pharmaceutically acceptable salt thereof. In some embodiments, provided herein is a method of improving learning in a subject having mild dementia resulting from Alzheimer's Disease, the method comprising administering to the subject Compound 1. In some embodiments, provided herein is a method of improving learning in a subject having mild dementia resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning in an Alzheimer's Disease subject having Mild Cognitive Impairment (MCI), the method comprising administering to the subject Compound 1, or a pharmaceutically acceptable salt thereof. In some embodiments, provided herein is a method of improving learning in an Alzheimer's Disease subject having Mild Cognitive Impairment (MCI), the method comprising administering to the subject Compound 1. In some embodiments, provided herein is a method of improving learning in an Alzheimer's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning in an Alzheimer's Disease subject having mild dementia, the method comprising administering to the subject Compound 1, or a pharmaceutically acceptable salt thereof. In some embodiments, provided herein is a method of improving learning in an Alzheimer's Disease subject having mild dementia, the method comprising administering to the subject Compound 1. In some embodiments, provided herein is a method of improving learning in an Alzheimer's Disease subject having mild dementia, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving learning and working memory in a subject having Mild Cognitive Impairment (MCI) associated with Alzheimer's Disease, the method comprising administering to the subject Compound 1, or a pharmaceutically acceptable salt thereof. In some embodiments, provided herein is a method of improving learning and working memory in a subject having Mild Cognitive Impairment (MCI) associated with Alzheimer's Disease, the method comprising administering to the subject Compound 1. In some embodiments, provided herein is a method of improving learning and working memory in a subject having Mild Cognitive Impairment (MCI) associated with Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is a method of improving learning and working memory in a subject having mild dementia associated with Alzheimer's Disease, the method comprising administering to the subject Compound 1, or a pharmaceutically acceptable salt thereof. In some embodiments, provided herein is a method of improving learning and working memory in a subject having mild dementia associated with Alzheimer's Disease, the method comprising administering to the subject Compound 1. In some embodiments, provided herein is a method of improving learning and working memory in a subject having mild dementia associated with Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning and working memory in a subject having Mild Cognitive Impairment (MCI) resulting from Alzheimer's Disease, the method comprising administering to the subject Compound 1, or a pharmaceutically acceptable salt thereof. In some embodiments, provided herein is a method of improving learning and working memory in a subject having Mild Cognitive Impairment (MCI) resulting from Alzheimer's Disease, the method comprising administering to the subject Compound 1. In some embodiments, provided herein is a method of improving learning and working memory in a subject having Mild Cognitive Impairment (MCI) resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning and working memory in a subject having mild dementia resulting from Alzheimer's Disease, the method comprising administering to the subject Compound 1, or a pharmaceutically acceptable salt thereof. In some embodiments, provided herein is a method of improving learning and working memory in a subject having mild dementia resulting from Alzheimer's Disease, the method comprising administering to the subject Compound 1. In some embodiments, provided herein is a method of improving learning and working memory in a subject having mild dementia resulting from Alzheimer's Disease, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning and working memory in an Alzheimer's Disease subject having Mild Cognitive Impairment (MCI), the method comprising administering to the subject Compound 1, or a pharmaceutically acceptable salt thereof. In some embodiments, provided herein is a method of improving learning and working memory in an Alzheimer's Disease subject having Mild Cognitive Impairment (MCI), the method comprising administering to the subject Compound 1. In some embodiments, provided herein is a method of improving learning and working memory in an Alzheimer's Disease subject having Mild Cognitive Impairment (MCI), the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In another aspect, provided herein is a method of improving learning and working memory in an Alzheimer's Disease subject having mild dementia, the method comprising administering to the subject Compound 1, or a pharmaceutically acceptable salt thereof. In some embodiments, provided herein is a method of improving learning and working memory in an Alzheimer's Disease subject having mild dementia, the method comprising administering to the subject Compound 1. In some embodiments, provided herein is a method of improving learning and working memory in an Alzheimer's Disease subject having mild dementia, the method comprising administering to the subject a pharmaceutically acceptable salt of Compound 1. In some embodiments, Compound 1 is administered to the subject daily. In some embodiments, a pharmaceutically acceptable salt of Compound 1 is administered to the subject daily. In some embodiments, Compound 1 or a pharmaceutically acceptable salt thereof is administered to the subject at a dose of about 0.3-6 mg/day. In some embodiments, Compound 1 is administered to the subject at a dose of about 0.3-6 mg/day. In some embodiments, a pharmaceutically acceptable salt of Compound 1 is administered to the subject at a dose of about 0.3-6 mg/day. In some embodiments, Compound 1 or a pharmaceutically acceptable salt thereof is administered to the subject at a dose of about 3 mg/day. In some embodiments, Compound 1 is administered to the subject at a dose of about 3 mg/day. In some embodiments, a pharmaceutically acceptable salt of Compound 1 is administered to the subject at a dose of about 3 mg/day. In some embodiments, Compound 1 or a pharmaceutically acceptable salt thereof is administered to the subject at a dose of 0.3-6 mg/day. In some embodiments, Compound 1 is administered to the subject at a dose of 0.3-6 mg/day. In some embodiments, a pharmaceutically acceptable salt of Compound 1 is administered to the subject at a dose of 0.3-6 mg/day. In some embodiments, Compound 1 or a pharmaceutically acceptable salt thereof is administered to the subject at a dose of 3 mg/day. In some embodiments, Compound 1 is administered to the subject at a dose of 3 mg/day. In some embodiments, a pharmaceutically acceptable salt of Compound 1 is administered to the subject at a dose of 3 mg/day. In some embodiments, Compound 1 is administered orally. In some embodiments, a pharmaceutically acceptable salt of Compound 1 is administered orally.

In one aspect, provided herein is the use of Compound 1, or a pharmaceutically acceptable salt thereof for the manufacture of a medicament for treating Mild Cognitive Impairment (MCI) associated with Alzheimer's Disease in a subject. In some embodiments, provided herein is the use of Compound 1 for the manufacture of a medicament for treating Mild Cognitive Impairment (MCI) associated with Alzheimer's Disease in a subject. In some embodiments, provided herein is the use of a pharmaceutically acceptable salt of Compound 1 for the manufacture of a medicament for treating Mild Cognitive Impairment (MCI) associated with Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In some embodiments, provided herein is the use of Compound 1, or a pharmaceutically acceptable salt thereof for the manufacture of a medicament for treating mild dementia associated with Alzheimer's Disease in a subject. In some embodiments, provided herein is the use of Compound 1 for the manufacture of a medicament for treating mild dementia associated with Alzheimer's Disease in a subject. In some embodiments, provided herein is the use of a pharmaceutically acceptable salt of Compound 1 for the manufacture of a medicament for treating mild dementia associated with Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for treating Mild Cognitive Impairment (MCI) resulting from Alzheimer's Disease in a subject. In some embodiments, provided herein is the use of Compound 1 for the manufacture of a medicament for treating Mild Cognitive Impairment (MCI) resulting from Alzheimer's Disease in a subject. In some embodiments, provided herein is the use of a pharmaceutically acceptable salt of Compound 1 for the manufacture of a medicament for treating Mild Cognitive Impairment (MCI) resulting from Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for treating mild dementia resulting from Alzheimer's Disease in a subject. In some embodiments, provided herein is the use of Compound 1 for the manufacture of a medicament for treating mild dementia resulting from Alzheimer's Disease in a subject. In some embodiments, provided herein is the use of a pharmaceutically acceptable salt of Compound 1 for the manufacture of a medicament for treating mild dementia resulting from Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for treating Mild Cognitive Impairment (MCI) in a subject having Alzheimer's Disease. In some embodiments, provided herein is the use of Compound 1 for the manufacture of a medicament for treating Mild Cognitive Impairment (MCI) in a subject having Alzheimer's Disease. In some embodiments, provided herein is the use of a pharmaceutically acceptable salt of Compound 1 for the manufacture of a medicament for treating Mild Cognitive Impairment (MCI) in a subject having Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for treating mild dementia in a subject having Alzheimer's Disease. In some embodiments, provided herein is the use of Compound 1 for the manufacture of a medicament for treating mild dementia in a subject having Alzheimer's Disease. In some embodiments, provided herein is the use of a pharmaceutically acceptable salt of Compound 1 for the manufacture of a medicament for treating mild dementia in a subject having Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving executive function in a subject having Mild Cognitive Impairment (MCI) associated with Alzheimer's Disease. In some embodiments, provided herein is the use of Compound 1 for the manufacture of a medicament for improving executive function in a subject having Mild Cognitive Impairment (MCI) associated with Alzheimer's Disease. In some embodiments, provided herein is the use of a pharmaceutically acceptable salt of Compound 1 for the manufacture of a medicament for improving executive function in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving executive function in a subject having mild dementia associated with Alzheimer's Disease. In some embodiments, provided herein is the use of Compound 1 for the manufacture of a medicament for improving executive function in a subject having mild dementia associated with Alzheimer's Disease. In some embodiments, provided herein is the use of a pharmaceutically acceptable salt of Compound 1 for the manufacture of a medicament for improving executive function in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving executive function in a subject having Mild Cognitive Impairment (MCI) resulting from Alzheimer's Disease. In some embodiments, provided herein is the use of Compound 1 for the manufacture of a medicament for improving executive function in a subject having Mild Cognitive Impairment (MCI) resulting from Alzheimer's Disease. In some embodiments, provided herein is the use of a pharmaceutically acceptable salt of Compound 1 for the manufacture of a medicament for improving executive function in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving executive function in a subject having mild dementia resulting from Alzheimer's Disease. In some embodiments, provided herein is the use of Compound 1 for the manufacture of a medicament for improving executive function in a subject having mild dementia resulting from Alzheimer's Disease. In some embodiments, provided herein is the use of a pharmaceutically acceptable salt of Compound 1 for the manufacture of a medicament for improving executive function in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving executive function in an Alzheimer's Disease subject having Mild Cognitive Impairment (MCI). In some embodiments, provided herein is the use of Compound 1 for the manufacture of a medicament for improving executive function in an Alzheimer's Disease subject having Mild Cognitive Impairment (MCI). In some embodiments, provided herein is the use of a pharmaceutically acceptable salt of Compound 1 for the manufacture of a medicament for improving executive function in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving executive function in an Alzheimer's Disease subject having mild dementia. In some embodiments, provided herein is the use of Compound 1 for the manufacture of a medicament for improving executive function in an Alzheimer's Disease subject having mild dementia. In some embodiments, provided herein is the use of a pharmaceutically acceptable salt of Compound 1 for the manufacture of a medicament for improving executive function in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving working memory in a subject having Mild Cognitive Impairment (MCI) associated with Alzheimer's Disease. In some embodiments, provided herein is the use of Compound 1 for the manufacture of a medicament for improving working memory in a subject having Mild Cognitive Impairment (MCI) associated with Alzheimer's Disease. In some embodiments, provided herein is the use of a pharmaceutically acceptable salt of Compound 1 for the manufacture of a medicament for improving working memory in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving working memory in a subject having mild dementia associated with Alzheimer's Disease. In some embodiments, provided herein is the use of Compound 1 for the manufacture of a medicament for improving working memory in a subject having mild dementia associated with Alzheimer's Disease. In some embodiments, provided herein is the use of a pharmaceutically acceptable salt of Compound 1 for the manufacture of a medicament for improving working memory in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving working memory in a subject having Mild Cognitive Impairment (MCI) resulting from Alzheimer's Disease. In some embodiments, provided herein is the use of Compound 1 for the manufacture of a medicament for method of improving working memory in a subject having Mild Cognitive Impairment (MCI) resulting from Alzheimer's Disease. In some embodiments, provided herein is the use of a pharmaceutically acceptable salt of Compound 1 for the manufacture of a medicament for method of improving working memory in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving working memory in a subject having mild dementia resulting from Alzheimer's Disease. In some embodiments, provided herein is the use of Compound 1 for the manufacture of a medicament for method of improving working memory in a subject having mild dementia resulting from Alzheimer's Disease. In some embodiments, provided herein is the use of a pharmaceutically acceptable salt of Compound 1 for the manufacture of a medicament for method of improving working memory in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, the medicament comprises about 0.1-about 1.0 mg of Compound 1 or a pharmaceutically acceptable salt thereof. In some embodiments, the medicament comprises about 0.1-about 1.0 mg of Compound 1. In some embodiments, the medicament comprises about 0.1-about 1.0 mg of a pharmaceutically acceptable salt of Compound 1. In some embodiments, the medicament comprises about 0.5 mg of Compound 1 or a pharmaceutically acceptable salt thereof. In some embodiments, the medicament comprises about 0.5 mg of Compound 1. In some embodiments, the medicament comprises about 0.5 mg of a pharmaceutically acceptable salt of Compound 1. In some embodiments, the medicament comprises about 1.0 mg of Compound 1 or a pharmaceutically acceptable salt thereof. In some embodiments, the medicament comprises about 1.0 mg of Compound 1. In some embodiments, the medicament comprises about 1.0 mg of a pharmaceutically acceptable salt of Compound 1. In some embodiments, the medicament comprises 0.1-1.0 mg of Compound 1 or a pharmaceutically acceptable salt thereof. In some embodiments, the medicament comprises 0.1-1.0 mg of Compound 1. In some embodiments, the medicament comprises 0.1-1.0 mg of a pharmaceutically acceptable salt of Compound 1. In some embodiments, the medicament comprises 0.5 mg of Compound 1 or a pharmaceutically acceptable salt thereof. In some embodiments, the medicament comprises 0.5 mg of Compound 1. In some embodiments, the medicament comprises 0.5 mg of a pharmaceutically acceptable salt of Compound 1. In some embodiments, the medicament comprises 1.0 mg of Compound 1 or a pharmaceutically acceptable salt thereof. In some embodiments, the medicament comprises 1.0 mg of Compound 1. In some embodiments, the medicament comprises 1.0 mg of a pharmaceutically acceptable salt of Compound 1. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving working memory in an Alzheimer's Disease subject having Mild Cognitive Impairment (MCI). In some embodiments, provided herein is the use of Compound 1 for the manufacture of a medicament for improving working memory in an Alzheimer's Disease subject having Mild Cognitive Impairment (MCI). In some embodiments, provided herein is the use of a pharmaceutically acceptable salt of Compound 1 for the manufacture of a medicament for improving working memory in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day.

In one aspect, provided herein is the use of Compound 1, or a pharmaceutically acceptable salt thereof for the manufacture of a medicament for improving working memory in an Alzheimer's Disease subject having mild dementia. In some embodiments, provided herein is the use of Compound 1 for the manufacture of a medicament for improving working memory in an Alzheimer's Disease subject having mild dementia. In some embodiments, provided herein is the use of a pharmaceutically acceptable salt of Compound 1 for the manufacture of a medicament for improving working memory in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning in a subject having Mild Cognitive Impairment (MCI) associated with Alzheimer's Disease. In some embodiments, provided herein is the use of Compound 1 for the manufacture of a medicament for improving learning in a subject having Mild Cognitive Impairment (MCI) associated with Alzheimer's Disease. In some embodiments, provided herein is the use of a pharmaceutically acceptable salt of Compound 1 for the manufacture of a medicament for improving learning in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning in a subject having mild dementia associated with Alzheimer's Disease. In some embodiments, provided herein is the use of Compound 1 for the manufacture of a medicament for improving learning in a subject having mild dementia associated with Alzheimer's Disease. In some embodiments, provided herein is the use of a pharmaceutically acceptable salt of Compound 1 for the manufacture of a medicament for improving learning in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning in a subject having Mild Cognitive Impairment (MCI) resulting from Alzheimer's Disease. In some embodiments, provided herein is the use of Compound 1 for the manufacture of a medicament for improving learning in a subject having Mild Cognitive Impairment (MCI) resulting from Alzheimer's Disease. In some embodiments, provided herein is the use of a pharmaceutically acceptable salt of Compound 1 for the manufacture of a medicament for improving learning in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning in a subject having mild dementia resulting from Alzheimer's Disease. In some embodiments, provided herein is the use of Compound 1 for the manufacture of a medicament for improving learning in a subject having mild dementia resulting from Alzheimer's Disease. In some embodiments, provided herein is the use of a pharmaceutically acceptable salt of Compound 1 for the manufacture of a medicament for improving learning in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning in an Alzheimer's Disease subject having Mild Cognitive Impairment (MCI). In some embodiments, provided herein is the use of Compound 1 for the manufacture of a medicament for improving learning in an Alzheimer's Disease subject having Mild Cognitive Impairment (MCI). In some embodiments, provided herein is the use of a pharmaceutically acceptable salt of Compound 1 for the manufacture of a medicament for improving learning in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning in an Alzheimer's Disease subject having mild dementia. In some embodiments, provided herein is the use of Compound 1 for the manufacture of a medicament for improving learning in an Alzheimer's Disease subject having mild dementia. In some embodiments, provided herein is the use of a pharmaceutically acceptable salt of Compound 1 for the manufacture of a medicament for improving learning in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning and working memory in a subject having Mild Cognitive Impairment (MCI) associated with Alzheimer's Disease. In some embodiments, provided herein is the use of Compound 1 for the manufacture of a medicament for improving learning and working memory in a subject having Mild Cognitive Impairment (MCI) associated with Alzheimer's Disease. In some embodiments, provided herein is the use of a pharmaceutically acceptable salt of Compound 1 for the manufacture of a medicament for improving learning and working memory in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning and working memory in a subject having mild dementia associated with Alzheimer's Disease. In some embodiments, provided herein is the use of Compound 1 for the manufacture of a medicament for improving learning and working memory in a subject having mild dementia associated with Alzheimer's Disease. In some embodiments, provided herein is the use of a pharmaceutically acceptable salt of Compound 1 for the manufacture of a medicament for improving learning and working memory in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning and working memory in a subject having Mild Cognitive Impairment (MCI) resulting from Alzheimer's Disease. In some embodiments, provided herein is the use of Compound 1 for the manufacture of a medicament for improving learning and working memory in a subject having Mild Cognitive Impairment (MCI) resulting from Alzheimer's Disease. In some embodiments, provided herein is the use of a pharmaceutically acceptable salt of Compound 1 for the manufacture of a medicament for improving learning and working memory in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning and working memory in a subject having mild dementia resulting from Alzheimer's Disease. In some embodiments, provided herein is the use of Compound 1 for the manufacture of a medicament for improving learning and working memory in a subject having mild dementia resulting from Alzheimer's Disease. In some embodiments, provided herein is the use of a pharmaceutically acceptable salt of Compound 1 for the manufacture of a medicament for improving learning and working memory in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning and working memory in an Alzheimer's Disease subject having Mild Cognitive Impairment (MCI). In some embodiments, provided herein is the use of Compound 1 for the manufacture of a medicament for improving learning and working memory in an Alzheimer's Disease subject having Mild Cognitive Impairment (MCI). In some embodiments, provided herein is the use of a pharmaceutically acceptable salt of Compound 1 for the manufacture of a medicament for improving learning and working memory in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is the use of Compound 1, or a pharmaceutically acceptable salt thereof for the manufacture of a medicament for improving learning and working memory in an Alzheimer's Disease subject having mild dementia. In some embodiments, provided herein is the use of Compound 1 for the manufacture of a medicament for improving learning and working memory in an Alzheimer's Disease subject having mild dementia. In some embodiments, provided herein is the use of a pharmaceutically acceptable salt of Compound 1 for the manufacture of a medicament for improving learning and working memory in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in treating Mild Cognitive Impairment (MCI) associated with Alzheimer's Disease in a subject. In some embodiments, provided herein is Compound 1 for use in treating Mild Cognitive Impairment (MCI) associated with Alzheimer's Disease in a subject. In some embodiments, provided herein is a pharmaceutically acceptable salt of Compound 1 for use in treating Mild Cognitive Impairment (MCI) associated with Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in treating mild dementia associated with Alzheimer's Disease in a subject. In some embodiments, provided herein is Compound 1 for use in treating mild dementia associated with Alzheimer's Disease in a subject. In some embodiments, provided herein is a pharmaceutically acceptable salt of Compound 1 for use in treating mild dementia associated with Alzheimer's Disease in a subject.

In one aspect, provided herein is Compound 1, or a pharmaceutically acceptable salt thereof for use in treating Mild Cognitive Impairment (MCI) resulting from Alzheimer's Disease in a subject. In some embodiments, provided herein is Compound 1 for use in treating Mild Cognitive Impairment (MCI) resulting from Alzheimer's Disease in a subject. In some embodiments, provided herein is a pharmaceutically acceptable salt of Compound 1 for use in treating Mild Cognitive Impairment (MCI) resulting from Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in treating mild dementia resulting from Alzheimer's Disease in a subject. In some embodiments, provided herein is Compound 1 for use in treating mild dementia resulting from Alzheimer's Disease in a subject. In some embodiments, provided herein is a pharmaceutically acceptable salt of Compound 1 for use in treating mild dementia resulting from Alzheimer's Disease in a subject. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in treating an Alzheimer's Disease subject having Mild Cognitive Impairment (MCI). In some embodiments, provided herein is Compound 1 for use in treating an Alzheimer's Disease subject having Mild Cognitive Impairment (MCI). In some embodiments, provided herein is a pharmaceutically acceptable salt of Compound 1 for use in treating an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in treating an Alzheimer's Disease subject having mild dementia. In some embodiments, provided herein is Compound 1 for use in treating an Alzheimer's Disease subject having mild dementia. In some embodiments, provided herein is a pharmaceutically acceptable salt of Compound 1 for use in treating an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving executive function in a subject having Mild Cognitive Impairment (MCI) associated with Alzheimer's Disease. In some embodiments, provided herein is Compound 1 for use in improving executive function in a subject having Mild Cognitive Impairment (MCI) associated with Alzheimer's Disease. In some embodiments, provided herein is a pharmaceutically acceptable salt of Compound 1 for use in improving executive function in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving executive function in a subject having mild dementia associated with Alzheimer's Disease. In some embodiments, provided herein is Compound 1 for use in improving executive function in a subject having mild dementia associated with Alzheimer's Disease. In some embodiments, provided herein is a pharmaceutically acceptable salt of Compound 1 for use in improving executive function in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving executive function in a subject having Mild Cognitive Impairment (MCI) resulting from Alzheimer's Disease. In some embodiments, provided herein is Compound 1 for use in improving executive function in a subject having Mild Cognitive Impairment (MCI) resulting from Alzheimer's Disease. In some embodiments, provided herein is a pharmaceutically acceptable salt of Compound 1 for use in improving executive function in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving executive function in a subject having mild dementia resulting from Alzheimer's Disease. In some embodiments, provided herein is Compound 1 for use in improving executive function in a subject having mild dementia resulting from Alzheimer's Disease. In some embodiments, provided herein is a pharmaceutically acceptable salt of Compound 1 for use in improving executive function in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving executive function in an Alzheimer's Disease subject having Mild Cognitive Impairment (MCI). In some embodiments, provided herein is Compound 1 for use in improving executive function in an Alzheimer's Disease subject having Mild Cognitive Impairment (MCI). In some embodiments, provided herein is a pharmaceutically acceptable salt of Compound 1 for use in improving executive function in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving executive function in an Alzheimer's Disease subject having mild dementia. In some embodiments, provided herein is Compound 1 for use in improving executive function in an Alzheimer's Disease subject having mild dementia. In some embodiments, provided herein is a pharmaceutically acceptable salt of Compound 1 for use in improving executive function in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving working memory in a subject having Mild Cognitive Impairment (MCI) associated with Alzheimer's Disease. In some embodiments, provided herein is Compound 1 for use in improving working memory in a subject having Mild Cognitive Impairment (MCI) associated with Alzheimer's Disease. In some embodiments, provided herein is a pharmaceutically acceptable salt of Compound 1 for use in improving working memory in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving working memory in a subject having mild dementia associated with Alzheimer's Disease. In some embodiments, provided herein is Compound 1 for use in improving working memory in a subject having mild dementia associated with Alzheimer's Disease. In some embodiments, provided herein is a pharmaceutically acceptable salt of Compound 1 for use in improving working memory in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving working memory in a subject having Mild Cognitive Impairment (MCI) resulting from Alzheimer's Disease. In some embodiments, provided herein is Compound 1 for use in improving working memory in a subject having Mild Cognitive Impairment (MCI) resulting from Alzheimer's Disease. In some embodiments, provided herein is a pharmaceutically acceptable salt of Compound 1 for use in improving working memory in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving working memory in a subject having mild dementia resulting from Alzheimer's Disease. In some embodiments, provided herein is Compound 1 for use in improving working memory in a subject having mild dementia resulting from Alzheimer's Disease. In some embodiments, provided herein is a pharmaceutically acceptable salt of Compound 1 for use in improving working memory in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving working memory in an Alzheimer's Disease subject having Mild Cognitive Impairment (MCI). In some embodiments, provided herein is Compound 1 for use in improving working memory in an Alzheimer's Disease subject having Mild Cognitive Impairment (MCI). In some embodiments, provided herein is a pharmaceutically acceptable salt of Compound 1 for use in improving working memory in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving working memory in an Alzheimer's Disease subject having mild dementia. In some embodiments, provided herein is Compound 1 for use in improving working memory in an Alzheimer's Disease subject having mild dementia. In some embodiments, provided herein is a pharmaceutically acceptable salt of Compound 1 for use in improving working memory in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning in a subject having Mild Cognitive Impairment (MCI) associated with Alzheimer's Disease. In some embodiments, provided herein is Compound 1 for use in improving learning in a subject having Mild Cognitive Impairment (MCI) associated with Alzheimer's Disease. In some embodiments, provided herein is a pharmaceutically acceptable salt of Compound 1 for use in improving learning in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning in a subject having mild dementia associated with Alzheimer's Disease. In some embodiments, provided herein is Compound 1 for use in improving learning in a subject having mild dementia associated with Alzheimer's Disease. In some embodiments, provided herein is a pharmaceutically acceptable salt of Compound 1 for use in improving learning in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning in a subject having Mild Cognitive Impairment (MCI) resulting from Alzheimer's Disease. In some embodiments, provided herein is Compound 1 for use in improving learning in a subject having Mild Cognitive Impairment (MCI) resulting from Alzheimer's Disease. In some embodiments, provided herein is a pharmaceutically acceptable salt of Compound 1 for use in improving learning in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning in a subject having mild dementia resulting from Alzheimer's Disease. In some embodiments, provided herein is Compound 1 for use in improving learning in a subject having mild dementia resulting from Alzheimer's Disease. In some embodiments, provided herein is a pharmaceutically acceptable salt of Compound 1 for use in improving learning in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning in an Alzheimer's Disease subject having Mild Cognitive Impairment (MCI). In some embodiments, provided herein is Compound 1 for use in improving learning in an Alzheimer's Disease subject having Mild Cognitive Impairment (MCI). In some embodiments, provided herein is a pharmaceutically acceptable salt of Compound 1 for use in improving learning in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning in an Alzheimer's Disease subject having mild dementia. In some embodiments, provided herein is Compound 1 for use in improving learning in an Alzheimer's Disease subject having mild dementia. In some embodiments, provided herein is a pharmaceutically acceptable salt of Compound 1 for use in improving learning in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning and working memory in a subject having Mild Cognitive Impairment (MCI) associated with Alzheimer's Disease. In some embodiments, provided herein is Compound 1 for use in improving learning and working memory in a subject having Mild Cognitive Impairment (MCI) associated with Alzheimer's Disease. In some embodiments, provided herein is a pharmaceutically acceptable salt of Compound 1 for use in improving learning and working memory in a subject having Mild Cognitive Impairment (MCI) associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning and working memory in a subject having mild dementia associated with Alzheimer's Disease. In some embodiments, provided herein is Compound 1 for use in improving learning and working memory in a subject having mild dementia associated with Alzheimer's Disease. In some embodiments, provided herein is a pharmaceutically acceptable salt of Compound 1 for use in improving learning and working memory in a subject having mild dementia associated with Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning and working memory in a subject having Mild Cognitive Impairment (MCI) resulting from Alzheimer's Disease. In some embodiments, provided herein is Compound 1 for use in improving learning and working memory in a subject having Mild Cognitive Impairment (MCI) resulting from Alzheimer's Disease. In some embodiments, provided herein is a pharmaceutically acceptable salt of Compound 1 for use in improving learning and working memory in a subject having Mild Cognitive Impairment (MCI) resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning and working memory in a subject having mild dementia resulting from Alzheimer's Disease. In some embodiments, provided herein is Compound 1 for use in improving learning and working memory in a subject having mild dementia resulting from Alzheimer's Disease. In some embodiments, provided herein is a pharmaceutically acceptable salt of Compound 1 for use in improving learning and working memory in a subject having mild dementia resulting from Alzheimer's Disease. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning and working memory in an Alzheimer's Disease subject having Mild Cognitive Impairment (MCI). In some embodiments, provided herein is Compound 1 for use in improving learning and working memory in an Alzheimer's Disease subject having Mild Cognitive Impairment (MCI). In some embodiments, provided herein is a pharmaceutically acceptable salt of Compound 1 for use in improving learning and working memory in an Alzheimer's Disease subject having Mild Cognitive Impairment (MCI). In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, provided herein is Compound 1, or a pharmaceutically acceptable salt thereof for use in improving learning and working memory in an Alzheimer's Disease subject having mild dementia. In some embodiments, provided herein is Compound 1 for use in improving learning and working memory in an Alzheimer's Disease subject having mild dementia. In some embodiments, provided herein is a pharmaceutically acceptable salt of Compound 1 for use in improving learning and working memory in an Alzheimer's Disease subject having mild dementia. In some embodiments, Compound 1 is for administration to the subject daily. In some embodiments, a pharmaceutically acceptable salt of Compound 1 is for administration to the subject daily. In some embodiments, Compound 1 or a pharmaceutically acceptable salt thereof is for administration to the subject at a dose of about 0.3-6 mg/day. In some embodiments, Compound 1 is for administration to the subject at a dose of about 0.3-6 mg/day. In some embodiments, a pharmaceutically acceptable salt of Compound 1 is for administration to the subject at a dose of about 0.3-6 mg/day. In some embodiments, Compound 1 or a pharmaceutically acceptable salt thereof is for administration to the subject at a dose of about 3 mg/day. In some embodiments, Compound 1 is for administration to the subject at a dose of about 3 mg/day. In some embodiments, a pharmaceutically acceptable salt of Compound 1 is for administration to the subject at a dose of about 3 mg/day. In some embodiments, Compound 1 or a pharmaceutically acceptable salt thereof is for administration to the subject at a dose of 0.3-6 mg/day. In some embodiments, Compound 1 is for administration to the subject at a dose of 0.3-6 mg/day. In some embodiments, a pharmaceutically acceptable salt of Compound 1 is for administration to the subject at a dose of 0.3-6 mg/day. In some embodiments, Compound 1 or a pharmaceutically acceptable salt thereof is for administration to the subject at a dose of 3 mg/day. In some embodiments, Compound 1 is for administration to the subject at a dose of 3 mg/day. In some embodiments, a pharmaceutically acceptable salt of Compound 1 is for administration to the subject at a dose of 3 mg/day. In some embodiments, Compound 1 is for administration orally. In some embodiments, a pharmaceutically acceptable salt of Compound 1 is for administration orally.

In one aspect, the present disclosure provides a method for treating or preventing Alzheimer's disease, comprising administering to a subject in need thereof (a) an NMDA receptor positive allosteric modulator, and (b) an anti-amyloid beta antibody or antigen-binding fragment thereof. In some embodiments, the disclosure provides a provides a method for treating Alzheimer's disease, comprising administering to a subject in need thereof (a) an NMDA receptor positive allosteric modulator, and (b) an anti-amyloid beta antibody or antigen-binding fragment thereof. In another embodiment, the disclosure provides a method for preventing Alzheimer's disease, comprising administering to a subject in need thereof (a) an NMDA receptor positive allosteric modulator, and (b) an anti-amyloid beta antibody or antigen-binding fragment thereof.

In one aspect, the present disclosure provides a method for treating or preventing cognitive impairment in a subject having Alzheimer's disease, comprising administering to the subject (a) an NMDA receptor positive allosteric modulator, and (b) an anti-amyloid beta antibody or antigen-binding fragment thereof. In some embodiments, the disclosure provides a method for treating cognitive impairment in a subject having Alzheimer's disease, comprising administering to the subject (a) an NMDA receptor positive allosteric modulator, and (b) an anti-amyloid beta antibody or antigen-binding fragment thereof. In some embodiments, the disclosure provides a method for preventing cognitive impairment in a subject having Alzheimer's disease, comprising administering to the subject (a) an NMDA receptor positive allosteric modulator, and (b) an anti-amyloid beta antibody or antigen-binding fragment thereof.

In one aspect, the present disclosure provides a method for treating or preventing cognitive impairment due to Alzheimer's disease in a subject, comprising administering to the subject (a) an NMDA receptor positive allosteric modulator, and (b) an anti-amyloid beta antibody or antigen-binding fragment thereof. In some embodiments, the disclosure provides a method for treating cognitive impairment due to Alzheimer's disease in a subject, comprising administering to the subject (a) an NMDA receptor positive allosteric modulator, and (b) an anti-amyloid beta antibody or antigen-binding fragment thereof. In some embodiments, the disclosure provides a method for preventing cognitive impairment due to Alzheimer's disease in a subject, comprising administering to the subject (a) an NMDA receptor positive allosteric modulator, and (b) an anti-amyloid beta antibody or antigen-binding fragment thereof.

In some embodiments, the cognitive impairment is mild cognitive impairment.

In one aspect, the present disclosure provides a method for improving cognition in a subject having Alzheimer's disease, comprising administering to the subject (a) an NMDA receptor positive allosteric modulator, and (b) an anti-amyloid beta antibody or antigen-binding fragment thereof.

In one aspect, the present disclosure provides a method for slowing cognitive decline due to Alzheimer's disease in a subject, comprising administering to the subject (a) an NMDA receptor positive allosteric modulator, and (b) an anti-amyloid beta antibody or antigen-binding fragment thereof.

In one aspect, the present disclosure provides a method for decreasing amyloid beta plaque accumulation in the brain of a subject comprising administering to the subject (a) an NMDA receptor positive allosteric modulator, and (b) an anti-amyloid beta antibody or antigen-binding fragment thereof.

In one aspect, the present disclosure provides a method for decreasing amyloid beta plaques in the brain of a subject having Alzheimer's disease comprising administering to the subject (a) an NMDA receptor positive allosteric modulator, and (b) an anti-amyloid beta antibody or antigen-binding fragment thereof.

In one aspect, the present disclosure provides a method for decreasing the deposition of amyloid beta plaques in the brain of a subject having Alzheimer's disease, comprising administering to the subject (a) an NMDA receptor positive allosteric modulator, and (b) an anti-amyloid beta antibody or antigen-binding fragment thereof.

In one aspect, the disclosure provides a method of treating including Mild Cognitive Impairment (MCI) associated with Alzheimer's Disease in a subject, the method comprising administering to the subject (a) an NMDA receptor positive allosteric modulator, and (b) an anti-amyloid beta antibody or antigen-binding fragment thereof.

In one aspect, the disclosure provides a method of improving executive function in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving working memory in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning and working memory in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of treating mild dementia associated with Alzheimer's Disease in a subject, the method comprising administering to the subject (a) an NMDA receptor positive allosteric modulator, and (b) an anti-amyloid beta antibody or antigen-binding fragment thereof.

In one aspect, the disclosure provides a method of improving executive function in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving working memory in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning in a subject having mild dementia associated with Alzheimer's Disease, the method comprising administering to the subject (a) an NMDA receptor positive allosteric modulator, and (b) an anti-amyloid beta antibody or antigen-binding fragment thereof.

In one aspect, the disclosure provides a method of improving learning and working memory in a subject having mild dementia associated with Alzheimer's Disease, the method comprising administering to the subject (a) an NMDA receptor positive allosteric modulator, and (b) an anti-amyloid beta antibody or antigen-binding fragment thereof.

In some embodiments, the NMDA receptor positive allosteric modulator and the anti-amyloid beta antibody or antigen binding fragment thereof are administered simultaneously. In some embodiments, the NMDA receptor positive allosteric modulator and the anti-amyloid beta antibody or antigen binding fragment thereof are administered sequentially. In some embodiments, the NMDA receptor positive allosteric modulator and the anti-amyloid beta antibody or antigen binding fragment thereof are administered separately. In some embodiments, the anti-amyloid beta antibody or antigen binding fragment thereof is administered before the NMDA receptor positive allosteric modulator. In some embodiments, the NMDA receptor positive allosteric modulator is administered before the anti-amyloid beta antibody or antigen binding fragment thereof. In some embodiments, the frequency of administration of the anti-amyloid beta antibody or antigen binding fragment thereof and the NMDA receptor positive allosteric modulator is the same. In some embodiments, the frequency of administration of the anti-amyloid beta antibody or antigen binding fragment thereof and the NMDA receptor positive allosteric modulator is different.

In one aspect, the present disclosure provides a method for treating or preventing Alzheimer's disease, comprising administering to a subject in need thereof (a) a CYP46A1 inhibitor, and (b) an anti-amyloid beta antibody or antigen-binding fragment thereof. In some embodiments, the present disclosure provides a method for treating Alzheimer's disease, comprising administering to a subject in need thereof (a) a CYP46A1 inhibitor, and (b) an anti-amyloid beta antibody or antigen-binding fragment thereof. In some embodiments, the present disclosure provides a method for preventing Alzheimer's disease, comprising administering to a subject in need thereof (a) a CYP46A1 inhibitor, and (b) an anti-amyloid beta antibody or antigen-binding fragment thereof.

In one aspect, the present disclosure provides a method for treating or preventing cognitive impairment in a subject having Alzheimer's disease, comprising administering to the subject (a) a CYP46A1 inhibitor, and (b) an anti-amyloid beta antibody or antigen-binding fragment thereof. In some embodiments, the present disclosure provides a method for treating cognitive impairment in a subject having Alzheimer's disease, comprising administering to the subject (a) a CYP46A1 inhibitor, and (b) an anti-amyloid beta antibody or antigen-binding fragment thereof. In some embodiments, the present disclosure provides a method for preventing cognitive impairment in a subject having Alzheimer's disease, comprising administering to the subject (a) a CYP46A1 inhibitor, and (b) an anti-amyloid beta antibody or antigen-binding fragment thereof.

In one aspect, the present disclosure provides a method for treating or preventing cognitive impairment due to Alzheimer's disease in a subject, comprising administering to the subject (a) a CYP46A1 inhibitor, and (b) an anti-amyloid beta antibody or antigen-binding fragment thereof. In some embodiments, the present disclosure provides a method for treating cognitive impairment due to Alzheimer's disease in a subject, comprising administering to the subject (a) a CYP46A1 inhibitor, and (b) an anti-amyloid beta antibody or antigen-binding fragment thereof. In some embodiments, the present disclosure provides a method for preventing cognitive impairment due to Alzheimer's disease in a subject, comprising administering to the subject (a) a CYP46A1 inhibitor, and (b) an anti-amyloid beta antibody or antigen-binding fragment thereof.

In some embodiments, the cognitive impairment is mild cognitive impairment.

In one aspect, the present disclosure provides a method for improving cognition in a subject having Alzheimer's disease, comprising administering to the subject (a) a CYP46A1 inhibitor, and (b) an anti-amyloid beta antibody or antigen-binding fragment thereof.

In one aspect, the present disclosure provides a method for slowing cognitive decline due to Alzheimer's disease in a subject, comprising administering to the subject (a) a CYP46A1 inhibitor, and (b) an anti-amyloid beta antibody or antigen-binding fragment thereof.

In one aspect, the present disclosure provides a method for decreasing amyloid beta plaque accumulation in the brain of a subject comprising administering to the subject (a) a CYP46A1 inhibitor, and (b) an anti-amyloid beta antibody or antigen-binding fragment thereof.

In one aspect, the present disclosure provides a method for decreasing amyloid beta plaques in the brain of a subject having Alzheimer's disease comprising administering to the subject (a) a CYP46A1 inhibitor, and (b) an anti-amyloid beta antibody or antigen-binding fragment thereof.

In one aspect, the present disclosure provides a method for decreasing the deposition of amyloid beta plaques in the brain of a subject having Alzheimer's disease, comprising administering to the subject (a) a CYP46A1 inhibitor, and (b) an anti-amyloid beta antibody or antigen-binding fragment thereof.

In one aspect, the disclosure provides a method of treating Mild Cognitive Impairment (MCI) associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving executive function in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving working memory in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving learning in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving learning and working memory in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of treating mild dementia associated with Alzheimer's Disease in a subject, the method comprising administering to the subject (a) a CYP46A1 inhibitor, and (b) an anti-amyloid beta antibody or antigen-binding fragment thereof.

In one aspect, the disclosure provides a method of improving executive function in a subject having mild dementia associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving working memory in a subject having mild dementia associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving learning in a subject having mild dementia associated with Alzheimer's Disease, the method comprising administering to the subject (a) a CYP46A1 inhibitor, and (b) an anti-amyloid beta antibody or antigen-binding fragment thereof.

In one aspect, the disclosure provides a method of improving learning and working memory in a subject having mild dementia associated with Alzheimer's Disease, the method comprising administering to the subject (a) a CYP46A1 inhibitor, and (b) an anti-amyloid beta antibody or antigen-binding fragment thereof.

In some embodiments, the CYP46A1 inhibitor and the anti-amyloid beta antibody or antigen binding fragment thereof are administered simultaneously. In some embodiments, the CYP46A1 inhibitor and the anti-amyloid beta antibody or antigen binding fragment thereof are administered sequentially. In some embodiments, the CYP46A1 inhibitor and the anti-amyloid beta antibody or antigen binding fragment thereof are administered separately. In some embodiments, the anti-amyloid beta antibody or antigen binding fragment thereof is administered before the CYP46A1 inhibitor. In some embodiments, the CYP46A1 inhibitor is administered before the anti-amyloid beta antibody or antigen binding fragment thereof. In some embodiments, the frequency of administration of the anti-amyloid beta antibody or antigen binding fragment thereof and the CYP46A1 inhibitor is the same. In some embodiments, the frequency of administration of the anti-amyloid beta antibody or antigen binding fragment thereof and the CYP46A1 inhibitor is different.

In one aspect, the present disclosure provides (a) an NMDA receptor positive allosteric modulator, and (b) an anti-amyloid beta antibody or antigen-binding fragment thereof, for use in treating or preventing Alzheimer's disease. In some embodiments, the disclosure provides (a) an NMDA receptor positive allosteric modulator, and (b) an anti-amyloid beta antibody or antigen-binding fragment thereof for use in treating Alzheimer's disease. In another embodiment, the present disclosure provides (a) an NMDA receptor positive allosteric modulator, and (b) an anti-amyloid beta antibody or antigen-binding fragment thereof, for use in preventing Alzheimer's disease.

In one aspect, the present disclosure provides (a) an NMDA receptor positive allosteric modulator, and (b) an anti-amyloid beta antibody or antigen-binding fragment thereof, for use in treating or preventing cognitive impairment in a subject having Alzheimer's disease. In some embodiments, the disclosure provides (a) an NMDA receptor positive allosteric modulator, and (b) an anti-amyloid beta antibody or antigen-binding fragment thereof, for use in treating cognitive impairment in a subject having Alzheimer's disease. In some embodiments, the disclosure provides (a) an NMDA receptor positive allosteric modulator, and (b) an anti-amyloid beta antibody or antigen-binding fragment thereof, for use in preventing cognitive impairment in a subject having Alzheimer's disease.

In one aspect, the present disclosure provides (a) an NMDA receptor positive allosteric modulator, and (b) an anti-amyloid beta antibody or antigen-binding fragment thereof, for use in treating or preventing cognitive impairment due to Alzheimer's disease in a subject. In some embodiments, the disclosure provides (a) an NMDA receptor positive allosteric modulator, and (b) an anti-amyloid beta antibody or antigen-binding fragment thereof, for use in treating cognitive impairment due to Alzheimer's disease in a subject. In some embodiments, the disclosure provides (a) an NMDA receptor positive allosteric modulator, and (b) an anti-amyloid beta antibody or antigen-binding fragment thereof, for use in preventing cognitive impairment due to Alzheimer's disease in a subject.

In some embodiments, the cognitive impairment is mild cognitive impairment.

In one aspect, the present disclosure provides (a) an NMDA receptor positive allosteric modulator, and (b) an anti-amyloid beta antibody or antigen-binding fragment thereof, for use in improving cognition in a subject having Alzheimer's disease.

In one aspect, the present disclosure provides (a) an NMDA receptor positive allosteric modulator, and (b) an anti-amyloid beta antibody or antigen-binding fragment thereof, for use in slowing cognitive decline due to Alzheimer's disease in a subject.

In one aspect, the present disclosure provides (a) an NMDA receptor positive allosteric modulator, and (b) an anti-amyloid beta antibody or antigen-binding fragment thereof, for use in decreasing amyloid beta plaque accumulation in the brain of a subject.

In one aspect, the present disclosure provides (a) an NMDA receptor positive allosteric modulator, and (b) an anti-amyloid beta antibody or antigen-binding fragment thereof, for use in decreasing amyloid beta plaques in the brain of a subject having Alzheimer's disease.

In one aspect, the present disclosure provides (a) an NMDA receptor positive allosteric modulator, and (b) an anti-amyloid beta antibody or antigen-binding fragment thereof, for use in decreasing the deposition of amyloid beta plaques in the brain of a subject having Alzheimer's disease.

In one aspect, the present disclosure provides (a) a CYP46A1 inhibitor, and (b) an anti-amyloid beta antibody or antigen-binding fragment thereof for use in treating or preventing Alzheimer's disease, comprising administering to a subject in need thereof. In some embodiments, the present disclosure provides (a) a CYP46A1 inhibitor, and (b) an anti-amyloid beta antibody or antigen-binding fragment thereof for use in treating Alzheimer's disease. In some embodiments, the present disclosure provides (a) a CYP46A1 inhibitor, and (b) an anti-amyloid beta antibody or antigen-binding fragment thereof for use in preventing Alzheimer's disease.

In one aspect, the present disclosure provides (a) a CYP46A1 inhibitor, and (b) an anti-amyloid beta antibody or antigen-binding fragment thereof for use in treating or preventing cognitive impairment in a subject having Alzheimer's disease. In some embodiments, the present disclosure provides (a) a CYP46A1 inhibitor, and (b) an anti-amyloid beta antibody or antigen-binding fragment thereof for use in treating cognitive impairment in a subject having Alzheimer's disease. In some embodiments, the present disclosure provides (a) a CYP46A1 inhibitor, and (b) an anti-amyloid beta antibody or antigen-binding fragment thereof for use in preventing cognitive impairment in a subject having Alzheimer's disease.

In one aspect, the present disclosure provides (a) a CYP46A1 inhibitor, and (b) an anti-amyloid beta antibody or antigen-binding fragment thereof for use in treating or preventing cognitive impairment due to Alzheimer's disease in a subject. In some embodiments, the present disclosure provides (a) a CYP46A1 inhibitor, and (b) an anti-amyloid beta antibody or antigen-binding fragment thereof for use in treating cognitive impairment due to Alzheimer's disease in a subject. In some embodiments, the present disclosure provides (a) a CYP46A1 inhibitor, and (b) an anti-amyloid beta antibody or antigen-binding fragment thereof for use in preventing cognitive impairment due to Alzheimer's disease in a subject.

In some embodiments, the cognitive impairment is mild cognitive impairment.

In one aspect, the present disclosure provides (a) a CYP46A1 inhibitor, and (b) an anti-amyloid beta antibody or antigen-binding fragment thereof for use in improving cognition in a subject having Alzheimer's disease.

In one aspect, the present disclosure provides (a) a CYP46A1 inhibitor, and (b) an anti-amyloid beta antibody or antigen-binding fragment thereof for use in slowing cognitive decline due to Alzheimer's disease in a subject.

In one aspect, the present disclosure provides (a) a CYP46A1 inhibitor, and (b) an anti-amyloid beta antibody or antigen-binding fragment thereof for use in decreasing amyloid beta plaque accumulation in the brain of a subject.

In one aspect, the present disclosure provides (a) a CYP46A1 inhibitor, and (b) an anti-amyloid beta antibody or antigen-binding fragment thereof for use in decreasing amyloid beta plaques in the brain of a subject having Alzheimer's disease.

In one aspect, the present disclosure provides (a) a CYP46A1 inhibitor, and (b) an anti-amyloid beta antibody or antigen-binding fragment thereof for use in decreasing the deposition of amyloid beta plaques in the brain of a subject having Alzheimer's disease.

In one aspect, the present disclosure provides the use of an (a) an NMDA receptor positive allosteric modulator, and (b) an anti-amyloid beta antibody or antigen-binding fragment thereof, for the manufacture of a medicament for treating or preventing Alzheimer's disease. In some embodiments, the disclosure provides the use of an (a) an NMDA receptor positive allosteric modulator, and (b) an anti-amyloid beta antibody or antigen-binding fragment thereof, for the manufacture of a medicament for treating Alzheimer's disease. In another embodiment, the present disclosure provides the use of an (a) an NMDA receptor positive allosteric modulator, and (b) an anti-amyloid beta antibody or antigen-binding fragment thereof, for the manufacture of a medicament for preventing Alzheimer's disease.

In one aspect, the present disclosure provides the use of an (a) an NMDA receptor positive allosteric modulator, and (b) an anti-amyloid beta antibody or antigen-binding fragment thereof, for the manufacture of a medicament for treating or preventing cognitive impairment in a subject having Alzheimer's disease. In some embodiments, the disclosure provides the use of an (a) an NMDA receptor positive allosteric modulator, and (b) an anti-amyloid beta antibody or antigen-binding fragment thereof, for the manufacture of a medicament for treating cognitive impairment in a subject having Alzheimer's disease. In some embodiments, the disclosure provides the use of an (a) an NMDA receptor positive allosteric modulator, and (b) an anti-amyloid beta antibody or antigen-binding fragment thereof, for the manufacture of a medicament for preventing cognitive impairment in a subject having Alzheimer's disease.

In one aspect, the present disclosure provides the use of an (a) an NMDA receptor positive allosteric modulator, and (b) an anti-amyloid beta antibody or antigen-binding fragment thereof, for the manufacture of a medicament for treating or preventing cognitive impairment due to Alzheimer's disease in a subject. In some embodiments, the disclosure provides the use of an (a) an NMDA receptor positive allosteric modulator, and (b) an anti-amyloid beta antibody or antigen-binding fragment thereof, for the manufacture of a medicament for treating cognitive impairment due to Alzheimer's disease in a subject. In some embodiments, the disclosure provides the use of an (a) an NMDA receptor positive allosteric modulator, and (b) an anti-amyloid beta antibody or antigen-binding fragment thereof, for the manufacture of a medicament for preventing cognitive impairment due to Alzheimer's disease in a subject.

In some embodiments, the cognitive impairment is mild cognitive impairment.

In one aspect, the present disclosure provides the use of an (a) an NMDA receptor positive allosteric modulator, and (b) an anti-amyloid beta antibody or antigen-binding fragment thereof, for the manufacture of a medicament for improving cognition in a subject having Alzheimer's disease.

In one aspect, the present disclosure provides the use of an (a) an NMDA receptor positive allosteric modulator, and (b) an anti-amyloid beta antibody or antigen-binding fragment thereof, for the manufacture of a medicament for slowing cognitive decline due to Alzheimer's disease in a subject.

In one aspect, the present disclosure provides the use of an (a) an NMDA receptor positive allosteric modulator, and (b) an anti-amyloid beta antibody or antigen-binding fragment thereof, for the manufacture of a medicament for decreasing amyloid beta plaque accumulation in the brain of a subject.

In one aspect, the present disclosure provides the use of an (a) an NMDA receptor positive allosteric modulator, and (b) an anti-amyloid beta antibody or antigen-binding fragment thereof, for the manufacture of a medicament for decreasing amyloid beta plaques in the brain of a subject having Alzheimer's disease.

In one aspect, the present disclosure provides the use of an (a) an NMDA receptor positive allosteric modulator, and (b) an anti-amyloid beta antibody or antigen-binding fragment thereof, for the manufacture of a medicament for decreasing the deposition of amyloid beta plaques in the brain of a subject having Alzheimer's disease.

In one aspect, the present disclosure provides the use of (a) a CYP46A1 inhibitor, and (b) an anti-amyloid beta antibody or antigen-binding fragment thereof for the manufacture of a medicament for treating or preventing Alzheimer's disease, comprising administering to a subject in need thereof. In some embodiments, the present disclosure provides the use of (a) a CYP46A1 inhibitor, and (b) an anti-amyloid beta antibody or antigen-binding fragment thereof for the manufacture of a medicament for treating Alzheimer's disease. In some embodiments, the present disclosure provides the use of (a) a CYP46A1 inhibitor, and (b) an anti-amyloid beta antibody or antigen-binding fragment thereof for the manufacture of a medicament for preventing Alzheimer's disease.

In one aspect, the present disclosure provides the use of (a) a CYP46A1 inhibitor, and (b) an anti-amyloid beta antibody or antigen-binding fragment thereof for the manufacture of a medicament for treating or preventing cognitive impairment in a subject having Alzheimer's disease. In some embodiments, the present disclosure provides the use of (a) a CYP46A1 inhibitor, and (b) an anti-amyloid beta antibody or antigen-binding fragment thereof for the manufacture of a medicament for treating cognitive impairment in a subject having Alzheimer's disease. In some embodiments, the present disclosure provides the use of (a) a CYP46A1 inhibitor, and (b) an anti-amyloid beta antibody or antigen-binding fragment thereof for the manufacture of a medicament for preventing cognitive impairment in a subject having Alzheimer's disease.

In one aspect, the present disclosure provides the use of (a) a CYP46A1 inhibitor, and (b) an anti-amyloid beta antibody or antigen-binding fragment thereof for the manufacture of a medicament for treating or preventing cognitive impairment due to Alzheimer's disease in a subject. In some embodiments, the present disclosure provides the use of (a) a CYP46A1 inhibitor, and (b) an anti-amyloid beta antibody or antigen-binding fragment thereof for the manufacture of a medicament for treating cognitive impairment due to Alzheimer's disease in a subject. In some embodiments, the present disclosure provides the use of (a) a CYP46A1 inhibitor, and (b) an anti-amyloid beta antibody or antigen-binding fragment thereof for the manufacture of a medicament for preventing cognitive impairment due to Alzheimer's disease in a subject.

In some embodiments, the cognitive impairment is mild cognitive impairment.

In one aspect, the present disclosure provides the use of (a) a CYP46A1 inhibitor, and (b) an anti-amyloid beta antibody or antigen-binding fragment thereof for the manufacture of a medicament for improving cognition in a subject having Alzheimer's disease.

In one aspect, the present disclosure provides the use of (a) a CYP46A1 inhibitor, and (b) an anti-amyloid beta antibody or antigen-binding fragment thereof for the manufacture of a medicament for slowing cognitive decline due to Alzheimer's disease in a subject.

In one aspect, the present disclosure provides the use of (a) a CYP46A1 inhibitor, and (b) an anti-amyloid beta antibody or antigen-binding fragment thereof for the manufacture of a medicament for decreasing amyloid beta plaque accumulation in the brain of a subject.

In one aspect, the present disclosure provides the use of (a) a CYP46A1 inhibitor, and (b) an anti-amyloid beta antibody or antigen-binding fragment thereof for the manufacture of a medicament for decreasing amyloid beta plaques in the brain of a subject having Alzheimer's disease.

In one aspect, the present disclosure provides the use of (a) a CYP46A1 inhibitor, and (b) an anti-amyloid beta antibody or antigen-binding fragment thereof for the manufacture of a medicament for decreasing the deposition of amyloid beta plaques in the brain of a subject having Alzheimer's disease.

### Variations and Modifications

Variations, modifications, and other implementations of what is described herein will be readily apparent to those of ordinary skill in the art without departing from the spirit and scope of the invention. Accordingly, the invention is not be limited to the preceding description or the following examples.

### Exemplification

With aspects of the invention now being generally described, these will be more readily understood by reference to the following examples, which are included merely for purposes of illustration of certain features and embodiments of the invention and are not intended to be limiting.

### Example 1: Effect of Compound 1 on Mild Cognitive Impairment due to Alzheimer's Disease

The safety and tolerability of Compound 1 and its effects on cognitive, neuropsychiatric, and motor symptoms in patients suffering from Mild Cognitive Impairment due to Alzheimer's Disease (AD-MCI) was evaluated in participants 50 to 80 years old with MCI or mild dementia due to AD in an open label study. Patients were required to meet criteria for AD-MCI (NIH on Aging-Alzheimer's Association 2011 diagnostic guidelines) or mild dementia and have a score of 15-24 on the Montreal Cognitive Assessment (MoCA).

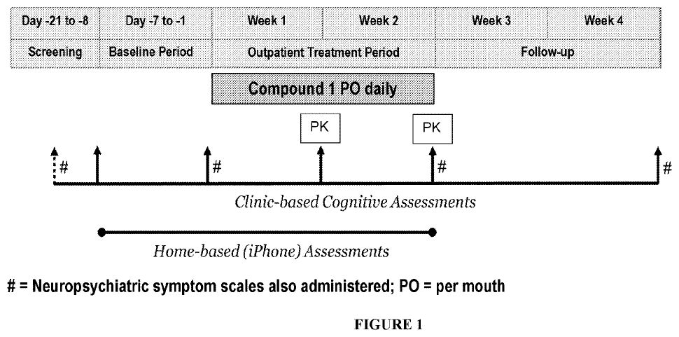

The Study comprised a 2-week screening period, a 1-week baseline period, a 2-week open-label treatment period, and a 2-week follow-up period. Patients were administered 3 mg of Compound 1 once daily in the morning for 14 days (FIG. 1). During the 1-week pre-treatment baseline period, patients underwent repeated cognitive testing using a comprehensive battery of tests to establish a robust cognitive baseline across multiple domains of cognitive function.

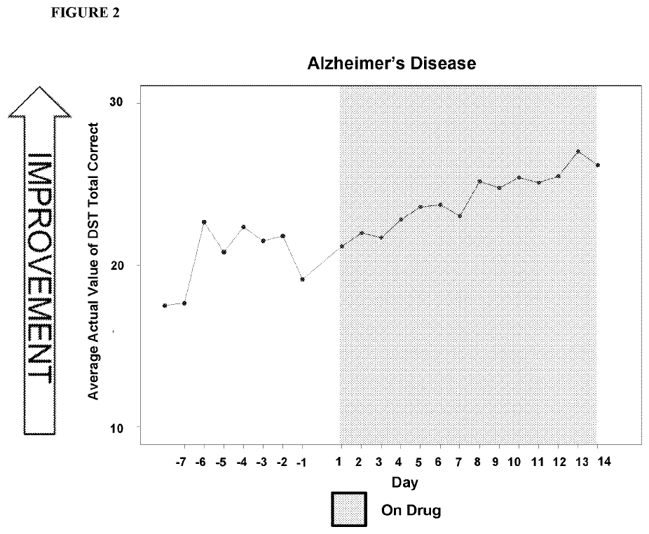

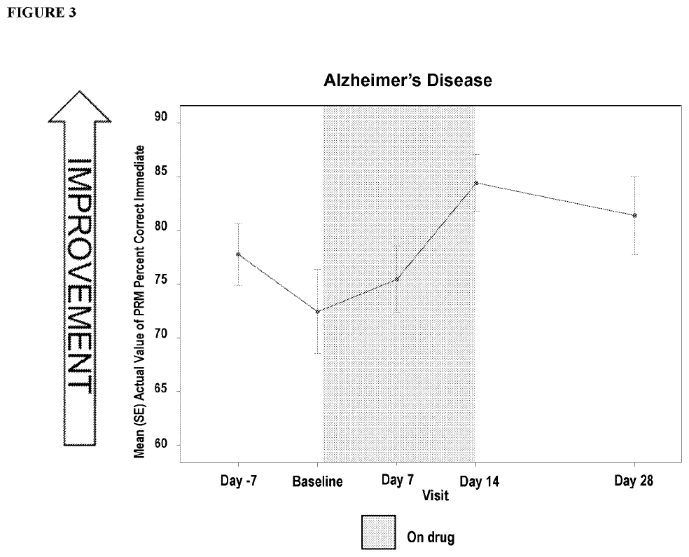

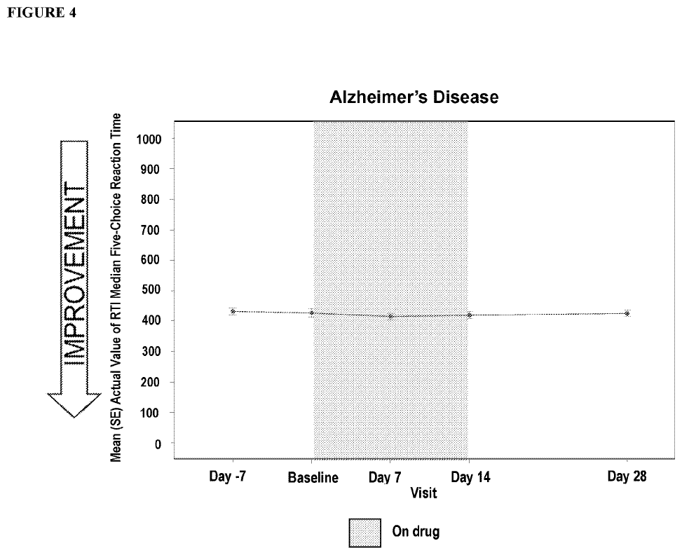

59 patients were screened and 26 were enrolled in the study. The majority of screen failures did not meet the inclusion criteria for the MoCA (required score of 15-24), with scores either too high (11) or too low (2/11). The study population was representative of patients with MCI or mild dementia due to AD in the United States. AD patients demonstrated relatively greater impairment in learning and memory across verbal and visual modalities. See FIGS. 2-4.

The baseline characteristics of the 26 subjects who were enrolled in the study as follows:

| Baseline characteristics | N = 26 | |
|---|---|---|
| Age, mean (SD, range), years | 67.0 | (9.1, 50-80) |
| Female, n (%) | 18 | (69.2) |
| Race, n (%) | 21 (80.8); | |
| White, black or African American; other | 4 (15.4); 1(3.8) | |
| MoCA score, mean (SD, range) | 20.7 | (2.6, 15-24) |

| | |
|---|---|
| Global CDR score, n (%) | 0; 23 (88.5); |
| 0; 0.5; 1; 2; 3 | 3 (11.5); 0; 0 |
| Amyloid PET status, n (%) | 3 (11.5); 1 (3.8); |
| BAPL1; BAPL2; BAPL3; N/A | 3 (11.5); 19 (73.1) |

Beginning on Day 1 and continuing through Day 14, participants self-administered 3.0 mg of Compound 1 (as six 0.5-mg oral tablets), once per day in the morning within 1 hour after initiating a meal containing approximately 30 g of fat, in accordance with the guidelines provided in a nutritional information brochure.

The key inclusion criteria for the subjects were as follows:
Be between the ages of 50 and 80 years, inclusive, at Screening
Meet the following criteria for MCI or mild dementia due to AD at Screening:
A memory complaint reported by the participant or his/her study partner
A CDR score of 0.5 to 1.0 (inclusive) with a memory box score ≥0.5
Essentially preserved activities of daily living, in the opinion of the investigator.
Brain MRI report, obtained within the 2 years preceding the Baseline Period, that is consistent with the diagnosis of AD-MCI with no clinically significant findings of non-AD pathology that could account for the observed cognitive impairment
Have a score of 15 to 24 (inclusive) on the MoCA at Screening
Have an estimated premorbid IQ≥85 (as assessed by the Test of Premorbid Functioning, performed at Screening

The key exclusion criteria for the subjects were as follows:
Have any medical or neurological condition (other than AD) that might be contributing to the participant's cognitive impairment or history of cognitive decline;
Have a history of brain surgery, deep brain stimulation, a significant head injury causing loss of consciousness greater than 30 minutes, or hospitalization due to a brain injury;
Have a history, presence, and/or current evidence of a clinically significant intracranial abnormality (e.g., stroke, hemorrhage, space-occupying lesion, or other non-AD pathology) that is likely to call into question a primary clinical diagnosis of AD;
Have a history of possible or probable cerebral amyloid angiopathy, according to the Boston Criteria;
Have a history of treatment with an anti-amyloid therapy (including biologics) without subsequent MRI demonstrating the absence of amyloid-related imaging abnormalities;
Have a condition that precludes undergoing an MRI, in accordance with standard operating procedures at the imaging facility (e.g., ferromagnetic metal in the body, claustrophobia), in a participant requiring MRI during Screening;
Be receiving any of prohibited medications, as outlined in the protocol, including medications with known effects at the NMDA receptor.

The Primary objective of the study was to assess (1) the incidence of Treatment-Emergent Adverse Event (TEAE).

The secondary objectives of the study were to assess:
the percentage of Participants With Clinically Significant Changes in Vital Sign Measurements, clinical laboratory analytes, electrocardiograms (ECGs), and responses on the Columbia-Suicide Severity Rating Scale (C-SSRS).

Additional objectives of the study were to assess the following:
PK profile of Compound 1 following administration of multiple doses as assessed by periodic PK sampling
Change from baseline on tests of executive function.
Change from baseline on tests of learning and memory.
Verbal Recognition Memory
Paired Associates Learning
Pattern Recognition Memory
Spatial Span
Change from baseline on tests of social cognition.
Emotion Recognition Task
Emotion Bias Task
Change from baseline on tests of psychomotor function.
Change from baseline on assessments of neuropsychiatric symptoms:
Change from baseline in performance on the Montreal Cognitive Assessment
Change from baseline in Clinical Global Impressions-Severity scores
Overall scores on Clinical Global Impressions-Improvement scales
Change from baseline in Amsterdam Instrumental Activities of Daily Living
Change from baseline in exploratory peripheral biomarkers
Amyloid PET Scan

The tests of Executive Functioning included (1) Multitasking Test (MTT), which assesses how well the subject manages conflicting information; (2) One Touch Stockings of Cambridge (OTS), which assesses how well the subject plans, holds onto, and manipulates information to solve a puzzle; (3) Spatial Working Memory (SWM), which assesses how well the subject develops and uses a strategy to find hidden information; (4) Digit Symbol Substitution Task (DSST), which assesses how efficiently can the subject complete a demanding task (daily measure); and (5) Two-Back Test (NBX), which asks is the symbol the same as that shown two symbols ago.

The tests of Learning and Memory included Paired Associates Learning (PAL), which assesses how well the subject remembers the location of hidden objects; Pattern Recognition Memory (PRM), which assesses how well the subject remembers a pattern; and Verbal Recognition Memory (PRM), which assesses how well the subject stores and retrieves verbal information.

The tests of Attention and Psychomotor Performance included Reaction Time (RTI), which assesses how fast the subject can respond to a stimulus (weekly measure); Psychomotor Vigilance Task (PVT), which assesses how fast can the subject respond to a stimulus (daily measure).

The tests of Social Cognition include Emotion Recognition Task (ERT), which assesses how quickly and accurately can the subject differentiate across a range of emotions; and Emotion Bias Task (EBT), which assesses how quickly and consistently can the subject identify an “ambiguous” emotion.

### Results:

### Primary Objectives:

No severe TEAEs, SAEs, or deaths were reported.
No TEAEs resulted in study drug discontinuation or withdrawal from the study.
   Of the 26 enrolled subjects, 7 experienced 8 TEAEs, 6 of which were considered related to study treatment. Of these, 4 (50%) were mild and 4 (50%) were moderate.
No clinically significant laboratory abnormalities were reported.
No clinically significant vital sign or ECG abnormalities were reported.
Pharmacokinetic data suggest that Day 7 and Day 14 plasma concentrations were consistent with the predicted range.

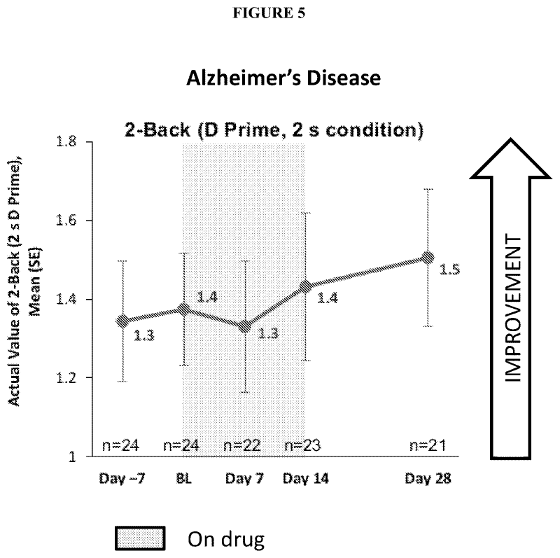

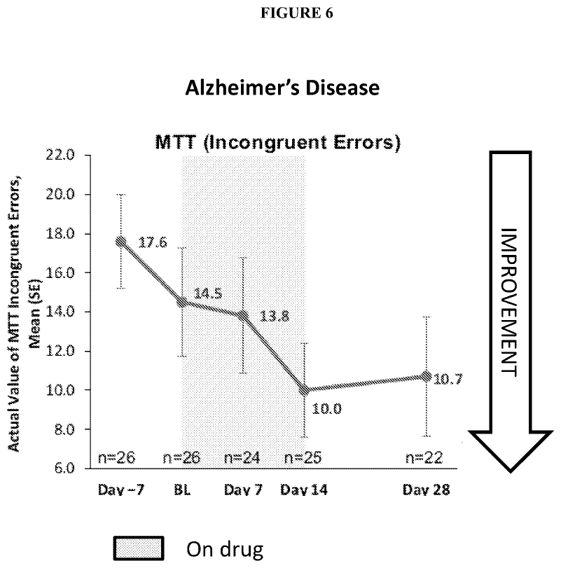

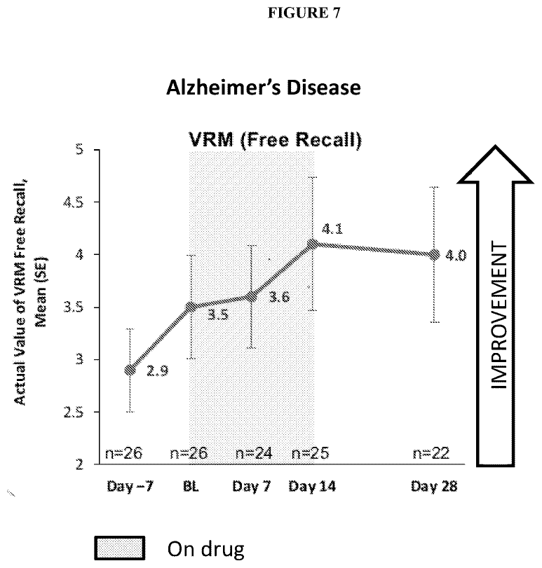

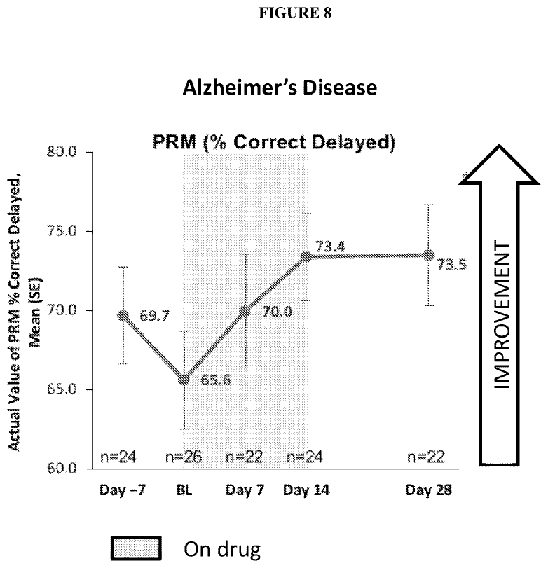

Secondary/Additional objectives: Compound 1 was associated with improved performance from baseline at Day 14 on all tests of executive functioning, with additional signals suggesting improved performance on tests of learning and memory (See Table 10). The study observed numerical improvement on all tasks assessing executive function (Multitasking, One Touch Stockings, Spatial working Memory, Digit Symbol Substitution, and 2-Back Test). No changes in attention or psychomotor processing were observed. For the learning and memory domain, numerical improvement was noted on pattern recognition memory, but no change (or slight worsening) was noted on paired associates learning and spatial span. An improvement was demonstrated in global cognition, assessed via MoCA, that was statistically significant (+2.3 pts) (see Table 11, see also FIGS. 5-8). The most consistent treatment signals were noted in tests of executive functioning, with additional signals in tests of learning and memory. No changes in attention or psychomotor processing were observed.

A summary of the cognitive findings is provided in Table 10:

TABLE 10

| | | Setting/ | Directional |
|---|---|---|---|
| Domain | Task | Frequency | Trend |
| Executive | Multitasking | Clinic/Weekly | Improvement |
| Functioning | One Touch Stockings | Clinic/Weekly | Improvement |
| | Spatial working | Clinic/Weekly | Improvement |
| | Memory | | |
| | Digit Symbol | Mobile/Daily | Improvement |
| | Substitution | | |
| | 2-Back Test | Clinic/Weekly | Improvement |
| Learning | Paired Associates | Clinic/Weekly | Mixed |
| & Memory | Learning | | |
| | Pattern Recognition | Clinic/Weekly | Improvement |
| | Memory | | |
| | Verbal Recognition | Clinic/Weekly | Improvement |
| | Memory | | |
| | Spatial Span | Clinic/Weekly | Mixed |
| Attention & | Reaction Time | Clinic/Weekly | No Change |
| Psychomotor | Psychomotor | Mobile/Daily | No Change |
| Speed | Vigilance Task | | |
| Global | MoCA | Monthly | Improvement |

TABLE 11

| | n | MoCA score, mean (SD) | |
|---|---|---|---|
| Baseline | 20 | 20.5 | (2.6) |
| Day 28 | 20 | (22.8 | (4.6) |
| CFB at Day 28 | 20 | 2.3 | (2.9) |

### Example 2: Mouse Model of AD

There are numerous mouse models of AD known in the art. Any such model (including but not limited to TG2576, APP/PS1, 5XFAD, APP23, PDAPP, TgCRND8, 3×Tg-AD, APOE, TREM2 or other AD mouse lines associated with excessive Amyloid beta production) would be useful to assess the pharmacological effect of the combination therapies (e.g., NMDA PAM+anti-amyloid beta antibody; or CYP46A1 inhibitor+anti-amyloid beta antibody) disclosed herein. Rodent models are evaluated using behavioral tests such as the Morris water maze (MWM), the radial maze, the Y-maze, the T-maze, fear conditioning (FC), and novel object recognition (NOR) tests. These tests play a crucial role as indicators of learning, memory, and cognitive functions, which correspond to the late phases of cognitive deficits in AD patients (Nakai et al., Alzheimer's Disease Animal Models: Elucidation of Biomarker and Therapeutic Approaches for Cognitive Impairment; Int. J. Mol. Sci., 22:5549 (2021).

Briefly, AD model transgenic mice are administered a vehicle alone (placebo), an NMDA PAM alone, an anti-amyloid beta antibody alone, or a combination of an NMDA PAM and an anti-amyloid beta antibody. Alternatively, the mice are administered a vehicle alone (placebo), a CYP46A1 inhibitor alone, an anti-amyloid beta antibody alone, or a combination of a CYP46A1 inhibitor and an anti-amyloid beta antibody. The mice will then be assessed using various classical behavioral tasks to evaluate cognitive and memory function. Additionally, biomarkers associated with AD will also be assessed.

### EQUIVALENTS

Those skilled in the art, will recognize, or be able to ascertain using no more than routine experimentation, numerous equivalents to the compounds, compositions, and methods of use thereof described herein. Such equivalents are considered to be within the scope of the invention.

### Numbered Embodiments

Certain embodiments of the disclosure are set forth in the following numbered paragraphs:

1. A method of treating Mild Cognitive Impairment (MCI) associated with Alzheimer's Disease in a subject, comprising administering to the subject a compound having the formula

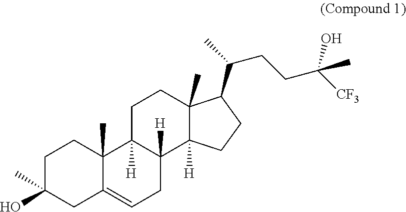

or a pharmaceutically acceptable salt thereof.
2. The method of embodiment 1, wherein Compound 1 or a pharmaceutically acceptable salt thereof is administered to the subject daily.
3 The method of embodiment 1 or 2, wherein Compound 1 or a pharmaceutically acceptable salt thereof is administered to the subject at a dose of about 3 mg/day.
4. The method of embodiment 3, wherein about 3 mg daily of Compound 1 is administered to the subject.
5. The method of any one of embodiments 1-4, wherein Compound 1 or a pharmaceutically acceptable salt thereof is administered orally.
6. A method of improving executive function in a subject having Mild Cognitive Impairment (MCI) associated with Alzheimer's Disease, the method comprising administering to the subject a compound having the formula

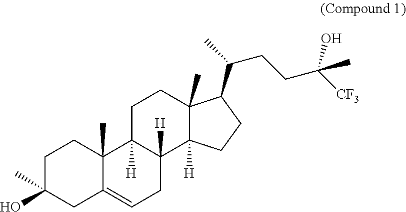

or a pharmaceutically acceptable salt thereof.
7. The method of embodiment 6, wherein Compound 1 or a pharmaceutically acceptable salt thereof is administered to the subject daily.
8. The method of embodiment 6 or 7, wherein Compound 1 or a pharmaceutically acceptable salt thereof is administered to the subject at a dose of about 3 mg/day.
9. The method of embodiment 8, wherein about 3 mg daily of Compound 1 is administered to the subject.
10. The method of any one of embodiments 6-9, wherein Compound 1 or a pharmaceutically acceptable salt thereof is administered orally.
11. A method of improving working memory in a subject having Mild Cognitive Impairment (MCI) associated with Alzheimer's Disease, the method comprising administering to the subject a compound having the formula

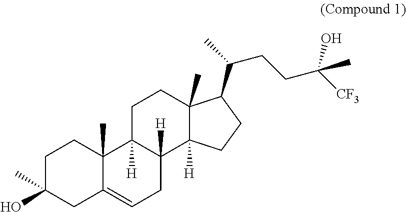

or a pharmaceutically acceptable salt thereof.
12. The method of embodiment 11, wherein Compound 1 or a pharmaceutically acceptable salt thereof is administered to the subject daily.
13. The method of embodiment 11 or 12, wherein Compound 1 or a pharmaceutically acceptable salt thereof is administered to the subject at a dose of about 3 mg/day.
14. The method of embodiment 13, wherein about 3 mg daily of Compound 1 is administered to the subject.
15. The method of any one of embodiments 11-14, wherein Compound 1 or a pharmaceutically acceptable salt thereof is administered orally.
16. A method of improving learning in a subject having Mild Cognitive Impairment (MCI) associated with Alzheimer's Disease, the method comprising administering to the subject a compound having the formula

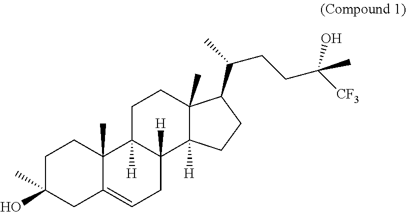

or a pharmaceutically acceptable salt thereof.
17. The method of embodiment 16, wherein Compound 1 or a pharmaceutically acceptable salt thereof is administered to the subject daily.
18. The method of embodiment 16 or 17, wherein Compound 1 or a pharmaceutically acceptable salt thereof is administered to the subject at a dose of about 3 mg/day.
19. The method of embodiment 18, wherein about 3 mg daily of Compound 1 is administered to the subject.
20. The method of any one of embodiments 16-19, wherein Compound 1 or a pharmaceutically acceptable salt thereof is administered orally.
21. A method of improving learning and working memory in a subject having Mild Cognitive Impairment (MCI) associated with Alzheimer's Disease, the method comprising administering to the subject a compound having the formula

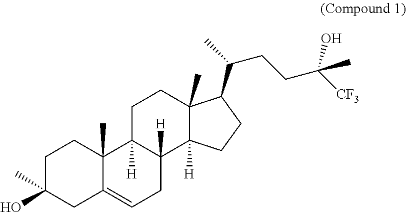

or a pharmaceutically acceptable salt thereof.
22. The method of embodiment 21, wherein Compound 1 or a pharmaceutically acceptable salt thereof is administered to the subject daily.
23. The method of embodiment 21 or 22, wherein Compound 1 or a pharmaceutically acceptable salt thereof is administered to the subject at a dose of about 3 mg/day.
24. The method of embodiment 23, wherein about 3 mg daily of Compound 1 is administered to the subject.
25. The method of any one of embodiments 21-24, wherein Compound 1 or a pharmaceutically acceptable salt thereof is administered orally.
26. The method according to any one of embodiments 1-25, wherein the subject is an adult.
27. A compound having the formula

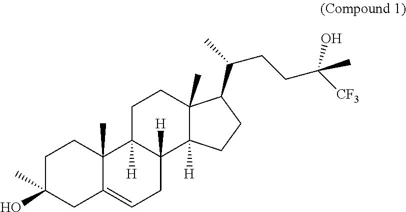

or a pharmaceutically acceptable salt thereof, for use in treating Mild Cognitive Impairment (MCI) associated with Alzheimer's Disease in a subject.
28. The compound for use according to embodiment 27, wherein Compound 1 or a pharmaceutically acceptable salt thereof is administered to the subject daily.
29. The compound for use according to embodiment 27 or 28, wherein Compound 1 or a pharmaceutically acceptable salt thereof is administered to the subject at a dose of about 3 mg/day.
30. The compound for use according to embodiment 29 wherein about 3 mg daily of Compound 1 is administered to the subject.
31. The compound for use according to any one of embodiments 27-30, wherein Compound 1 or a pharmaceutically acceptable salt thereof is administered orally.
32. A compound having the formula

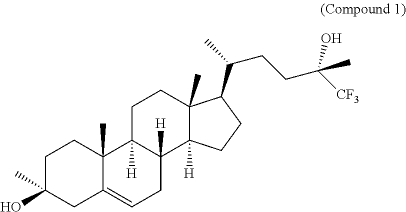

or a pharmaceutically acceptable salt thereof, for use in improving executive function in a subject having Mild Cognitive Impairment (MCI) associated with Alzheimer's Disease.
33. The compound for use according to embodiment 32, wherein Compound 1 or a pharmaceutically acceptable salt thereof is administered to the subject daily.
34. The compound for use according to embodiment 32 or 33, wherein Compound 1 or a pharmaceutically acceptable salt thereof is administered to the subject at a dose of about 3 mg/day.
35. The compound for use according to embodiment 34, wherein about 3 mg daily of Compound 1 is administered to the subject.
36. The compound for use according to any one of embodiments 32-35, wherein Compound 1 or a pharmaceutically acceptable salt thereof is administered orally.
37. A compound having the formula

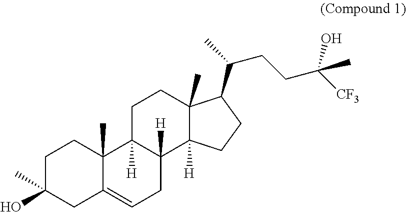

or a pharmaceutically acceptable salt thereof, for use in improving working memory in a subject having Mild Cognitive Impairment (MCI) associated with Alzheimer's Disease.
38. The compound for use according to embodiment 37, wherein Compound 1 or a pharmaceutically acceptable salt thereof is administered to the subject daily.
39. The compound for use according to embodiment 37 or 38, wherein Compound 1 or a pharmaceutically acceptable salt thereof is administered to the subject at a dose of about 3 mg/day.
40. The compound for use according to embodiment 39, wherein about 3 mg daily of Compound 1 is administered to the subject.
41. The compound for use according to embodiment 37-40, wherein Compound 1 or a pharmaceutically acceptable salt thereof is administered orally.
42. A compound having the formula

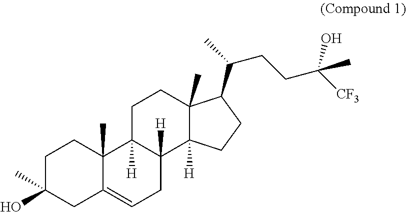

or a pharmaceutically acceptable salt thereof, for use in improving learning in a subject having Mild Cognitive Impairment (MCI) associated with Alzheimer's Disease.
43. The compound for use according to embodiment 42, wherein Compound 1 or a pharmaceutically acceptable salt thereof is administered to the subject daily.
44. The compound for use according to embodiment 42 or 43, wherein Compound 1 or a pharmaceutically acceptable salt thereof is administered to the subject at a dose of about 3 mg/day.
45. The compound for use according to embodiment 44, wherein about 3 mg daily of Compound 1 is administered to the subject.
46. The compound for use according to any one of embodiments 42-45, wherein Compound 1 or a pharmaceutically acceptable salt thereof is administered orally.
47. A compound having the formula

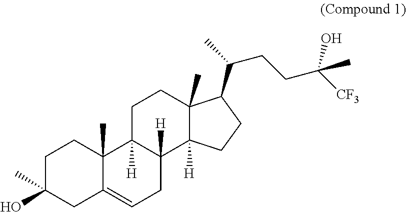

or a pharmaceutically acceptable salt thereof, for use in improving learning and working memory in a subject having Mild Cognitive Impairment (MCI) associated with Alzheimer's Disease.
48. The compound for use according to embodiment 47, wherein Compound 1 or a pharmaceutically acceptable salt thereof is administered to the subject daily.
49. The compound for use according to embodiment 47 or 48, wherein Compound 1 or a pharmaceutically acceptable salt thereof is administered to the subject at a dose of about 3 mg/day.
50. The compound for use according to embodiment 49, wherein about 3 mg daily of Compound 1 is administered to the subject.
51. The compound for use according to any one of embodiments 47-50, wherein Compound 1 or a pharmaceutically acceptable salt thereof is administered orally.
52. The compound for use according to any one of embodiments 27-51, wherein the subject is an adult.
53. Use of a compound having the formula

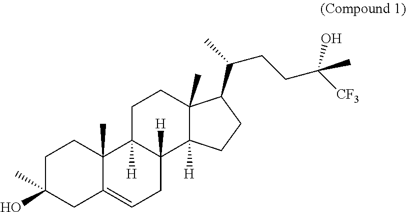

or a pharmaceutically acceptable salt thereof, for the manufacture of a medicament for treating Mild Cognitive Impairment (MCI) associated with Alzheimer's Disease in a subject.
54. The use according to embodiment 53, wherein Compound 1 or a pharmaceutically acceptable salt thereof is administered to the subject daily.
55. The use according to embodiment 53 or 54, wherein Compound 1 or a pharmaceutically acceptable salt thereof is administered to the subject at a dose of about 3 mg/day.
56. The use according to embodiment 55, wherein about 3 mg daily of Compound 1 is administered to the subject.
57. The use according to any one of embodiments 53-56, wherein Compound 1 or a pharmaceutically acceptable salt thereof is administered orally.
58. Use of a compound having the formula

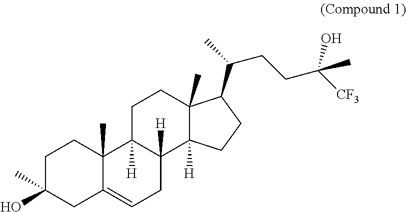

or a pharmaceutically acceptable salt thereof, for the manufacture of a medicament for improving executive function in a subject having Mild Cognitive Impairment (MCI) associated with Alzheimer's Disease.
59. The use according to embodiment 58, wherein Compound 1 or a pharmaceutically acceptable salt thereof is administered to the subject daily.
60. The use according to embodiment 58 or 59, wherein Compound 1 or a pharmaceutically acceptable salt thereof is administered to the subject at a dose of about 3 mg/day.
61. The use according to embodiment 60, wherein about 3 mg daily of Compound 1 is administered to the subject.
62. The use according to any one of embodiments 58-61, wherein Compound 1 or a pharmaceutically acceptable salt thereof is administered orally.
63. Use of a compound having the formula

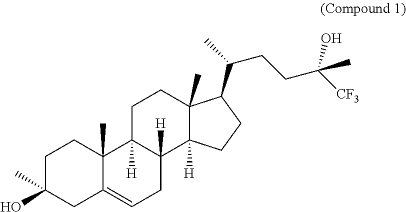

or a pharmaceutically acceptable salt thereof, for the manufacture of a medicament for improving working memory in a subject having Mild Cognitive Impairment (MCI) associated with Alzheimer's Disease.
64. The use according to embodiment 63, wherein Compound 1 or a pharmaceutically acceptable salt thereof is administered to the subject daily.
65. The use according to embodiment 63 or 64, wherein Compound 1 or a pharmaceutically acceptable salt thereof is administered to the subject at a dose of about 3 mg/day.
66. The use according to embodiment 65, wherein about 3 mg daily of Compound 1 is administered to the subject.
67. The use according to embodiments 63-66, wherein Compound 1 or a pharmaceutically acceptable salt thereof is administered orally.
68. Use of a compound having the formula

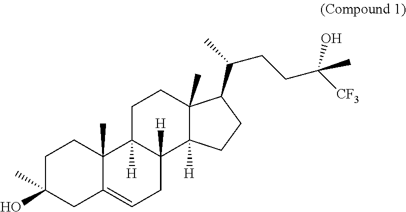

or a pharmaceutically acceptable salt thereof, for the manufacture of a medicament for improving learning in a subject having Mild Cognitive Impairment (MCI) associated with Alzheimer's Disease.
69. The use according to embodiment 68, wherein Compound 1 or a pharmaceutically acceptable salt thereof is administered to the subject daily.
70. The use according to embodiment 68 or 69, wherein Compound 1 or a pharmaceutically acceptable salt thereof is administered to the subject at a dose of about 3 mg/day.
71. The use according to embodiment 70, wherein about 3 mg daily of Compound 1 is administered to the subject.
72. The use according to any one of embodiments 68-71, wherein Compound 1 or a pharmaceutically acceptable salt thereof is administered orally.
73. Use of a compound having the formula

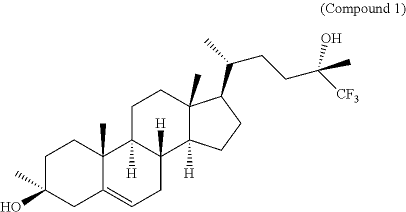

or a pharmaceutically acceptable salt thereof, for the manufacture of a medicament for improving learning and working memory in a subject having Mild Cognitive Impairment (MCI) associated with Alzheimer's Disease.
74. The use according to embodiment 73, wherein Compound 1 or a pharmaceutically acceptable salt thereof is administered to the subject daily.
75. The use according to embodiment 73 or 74, wherein Compound 1 or a pharmaceutically acceptable salt thereof is administered to the subject at a dose of about 3 mg/day.
76. The use according to embodiment 75, wherein about 3 mg daily of Compound 1 is administered to the subject.
77. The use according to any one of embodiments 73-76, wherein Compound 1 or a pharmaceutically acceptable salt thereof is administered orally.
78. The use according to any one of embodiments 53-77, wherein the subject is an adult.
79. A method of treating mild dementia associated with Alzheimer's Disease in a subject, comprising administering to the subject a compound having the formula

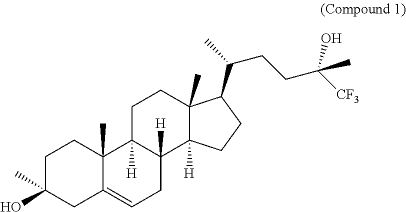

or a pharmaceutically acceptable salt thereof.
80. The method of embodiment 79, wherein Compound 1 or a pharmaceutically acceptable salt thereof is administered to the subject daily.
81. The method of embodiment 79 or 80, wherein Compound 1 or a pharmaceutically acceptable salt thereof is administered to the subject at a dose of about 3 mg/day.
82. The method of embodiment 81 wherein about 3 mg daily of Compound 1 is administered to the subject.
83. The method of any one of embodiments 79-82, wherein Compound 1 or a pharmaceutically acceptable salt thereof is administered orally.
84. A method of improving executive function in a subject having mild dementia associated with Alzheimer's Disease, the method comprising administering to the subject a compound having the formula

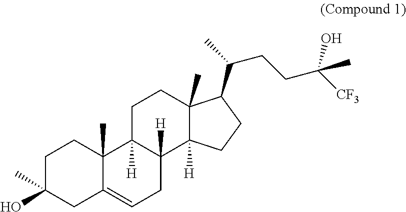

or a pharmaceutically acceptable salt thereof.
85. The method of embodiment 84, wherein Compound 1 or a pharmaceutically acceptable salt thereof is administered to the subject daily.
86. The method of embodiment 84 or 85, wherein Compound 1 or a pharmaceutically acceptable salt thereof is administered to the subject at a dose of about 3 mg/day.
87. The method of embodiment 86, wherein about 3 mg daily of Compound 1 is administered to the subject.
88. The method of any one of embodiments 84-87, wherein Compound 1 or a pharmaceutically acceptable salt thereof is administered orally.
89. A method of improving working memory in a subject having mild dementia associated with Alzheimer's Disease, the method comprising administering to the subject a compound having the formula

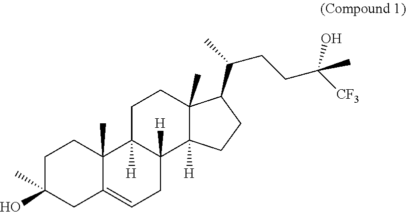

or a pharmaceutically acceptable salt thereof.
90. The method of embodiment 89, wherein Compound 1 or a pharmaceutically acceptable salt thereof is administered to the subject daily.
91. The method of embodiment 89 or 90, wherein Compound 1 or a pharmaceutically acceptable salt thereof is administered to the subject at a dose of about 3 mg/day.
92. The method of embodiment 91, wherein about 3 mg daily of Compound 1 is administered to the subject.
93. The method of any one of embodiments 89-92, wherein Compound 1 or a pharmaceutically acceptable salt thereof is administered orally.
94. A method of improving learning in a subject having mild dementia associated with Alzheimer's Disease, the method comprising administering to the subject a compound having the formula

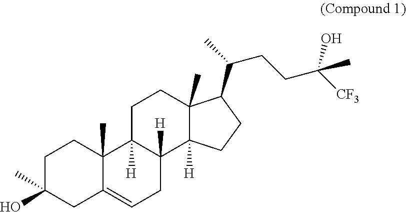

or a pharmaceutically acceptable salt thereof.
95. The method of embodiment 94, wherein Compound 1 or a pharmaceutically acceptable salt thereof is administered to the subject daily.
96. The method of embodiment 94 or 95, wherein Compound 1 or a pharmaceutically acceptable salt thereof is administered to the subject at a dose of about 3 mg/day.
97. The method of embodiment 96, wherein about 3 mg daily of Compound 1 is administered to the subject.
98. The method of any one of embodiments 94-97, wherein Compound 1 or a pharmaceutically acceptable salt thereof is administered orally.
99. A method of improving learning and working memory in a subject having mild dementia associated with Alzheimer's Disease, the method comprising administering to the subject a compound having the formula

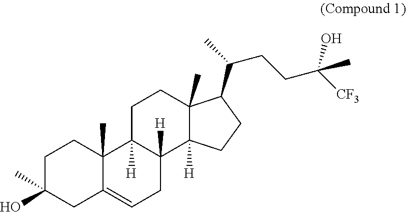

or a pharmaceutically acceptable salt thereof.
100. The method of embodiment 99 wherein Compound 1 or a pharmaceutically acceptable salt thereof is administered to the subject daily.
101. The method of embodiment 99 or 100, wherein Compound 1 or a pharmaceutically acceptable salt thereof is administered to the subject at a dose of about 3 mg/day.
102. The method of embodiment 101, wherein about 3 mg daily of Compound 1 is administered to the subject.
103. The method of any one of embodiments 99-102, wherein Compound 1 or a pharmaceutically acceptable salt thereof is administered orally.
104. The method of any one of embodiments 79-103, wherein the subject is an adult.
105. A compound having the formula

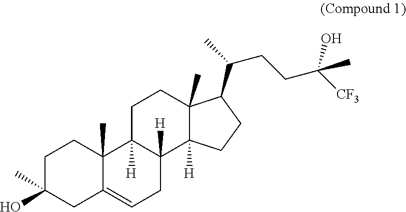

or a pharmaceutically acceptable salt thereof, for use in treating mild dementia associated with Alzheimer's Disease in a subject.
106. The compound for use according to embodiment 105, wherein Compound 1 or a pharmaceutically acceptable salt thereof is administered to the subject daily.
107. The compound for use according to embodiment 105 or 106, wherein Compound 1 or a pharmaceutically acceptable salt thereof is administered to the subject at a dose of about 3 mg/day.
108. The compound for use according to embodiment 107, wherein about 3 mg daily of Compound 1 is administered to the subject.
109. The compound for use according to any one of embodiments 105-108, wherein Compound 1 or a pharmaceutically acceptable salt thereof is administered orally.
110. A compound having the formula

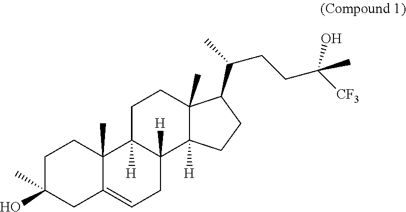

or a pharmaceutically acceptable salt thereof, for use in improving executive function in a subject having mild dementia associated with Alzheimer's Disease.
111. The compound for use according to embodiment 110, wherein Compound 1 or a pharmaceutically acceptable salt thereof is administered to the subject daily.
112. The compound for use according to embodiment 110 or 111, wherein Compound 1 or a pharmaceutically acceptable salt thereof is administered to the subject at a dose of about 3 mg/day.
113. The compound for use according to embodiment 112, wherein about 3 mg daily of Compound 1 is administered to the subject.
114. The compound for use according to any one of embodiments 110-113, wherein Compound 1 or a pharmaceutically acceptable salt thereof is administered orally.
115. A compound having the formula

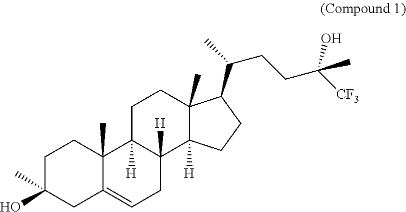

or a pharmaceutically acceptable salt thereof, for use in improving working memory in a subject having mild dementia associated with Alzheimer's Disease.
116. The compound for use according to embodiment 115, wherein Compound 1 or a pharmaceutically acceptable salt thereof is administered to the subject daily.
117. The compound for use according to embodiment 115 or 116, wherein Compound 1 or a pharmaceutically acceptable salt thereof is administered to the subject at a dose of about 3 mg/day.
118. The compound for use according to embodiment 117, wherein about 3 mg daily of Compound 1 is administered to the subject.
119. The compound for use according to embodiment 115-118, wherein Compound 1 or a pharmaceutically acceptable salt thereof is administered orally.
120. A compound having the formula

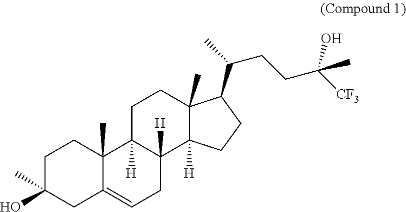

or a pharmaceutically acceptable salt thereof, for use in improving learning in a subject having mild dementia associated with Alzheimer's Disease.
121. The compound for use according to embodiment 120, wherein Compound 1 or a pharmaceutically acceptable salt thereof is administered to the subject daily.
122. The compound for use according to embodiment 120 or 121, wherein Compound 1 or a pharmaceutically acceptable salt thereof is administered to the subject at a dose of about 3 mg/day.
123. The compound for use according to embodiment 122, wherein about 3 mg daily of Compound 1 is administered to the subject.
124. The compound for use according to any one of embodiments 120-123, wherein Compound 1 or a pharmaceutically acceptable salt thereof is administered orally.
125. A compound having the formula

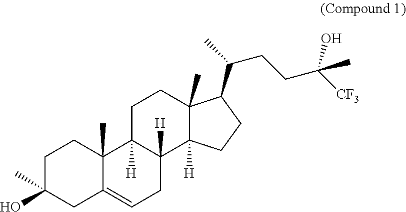

or a pharmaceutically acceptable salt thereof, for use in improving learning and working memory in a subject having mild dementia associated with Alzheimer's Disease.
126. The compound for use according to embodiment 125, wherein Compound 1 or a pharmaceutically acceptable salt thereof is administered to the subject daily.
127. The compound for use according to embodiment 125 or 126, wherein Compound 1 or a pharmaceutically acceptable salt thereof is administered to the subject at a dose of about 3 mg/day.
128. The compound for use according to embodiment 127, wherein about 3 mg daily of Compound 1 is administered to the subject.
129. The compound for use according to any one of embodiments 125-128, wherein Compound 1 or a pharmaceutically acceptable salt thereof is administered orally.
130. The compound for use according to any one of embodiments 105-129, wherein the subject is an adult.
131. Use of a compound having the formula

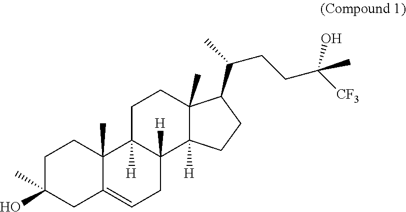

or a pharmaceutically acceptable salt thereof, for the manufacture of a medicament for treating mild dementia associated with Alzheimer's Disease in a subject.
132. The use according to embodiment 131, wherein Compound 1 or a pharmaceutically acceptable salt thereof is administered to the subject daily.
133. The use according to embodiment 131 or 132, wherein Compound 1 or a pharmaceutically acceptable salt thereof is administered to the subject at a dose of about 3 mg/day.
134. The use according to embodiment 133, wherein about 3 mg daily of Compound 1 is administered to the subject.
135. The use according to any one of embodiments 131-134, wherein Compound 1 or a pharmaceutically acceptable salt thereof is administered orally.
136. Use of a compound having the formula

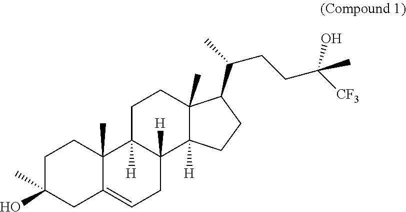

or a pharmaceutically acceptable salt thereof, for the manufacture of a medicament for improving executive function in a subject having mild dementia associated with Alzheimer's Disease.
137. The use according to embodiment 136, wherein Compound 1 or a pharmaceutically acceptable salt thereof is administered to the subject daily.
138. The use according to embodiment 136 or 137, wherein Compound 1 or a pharmaceutically acceptable salt thereof is administered to the subject at a dose of about 3 mg/day.
139. The use according to embodiment 138, wherein about 3 mg daily of Compound 1 is administered to the subject.
140. The use according to any one of embodiments 136-139, wherein Compound 1 or a pharmaceutically acceptable salt thereof is administered orally.
141. Use of a compound having the formula

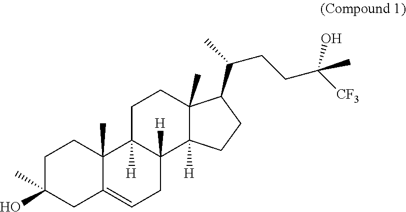

or a pharmaceutically acceptable salt thereof, for the manufacture of a medicament for improving working memory in a subject having mild dementia associated with Alzheimer's Disease.
142. The use according to embodiment 141, wherein Compound 1 or a pharmaceutically acceptable salt thereof is administered to the subject daily.
143. The use according to embodiment 141 or 142, wherein Compound 1 or a pharmaceutically acceptable salt thereof is administered to the subject at a dose of about 3 mg/day.
144. The use according to embodiment 143, wherein about 3 mg daily of Compound 1 is administered to the subject.
145. The use according to embodiments 141-144 wherein Compound 1 or a pharmaceutically acceptable salt thereof is administered orally.
146. Use of a compound having the formula

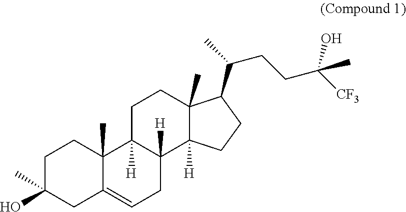

or a pharmaceutically acceptable salt thereof, for the manufacture of a medicament for improving learning in a subject having mild dementia associated with Alzheimer's Disease.
147. The use according to embodiment 146, wherein Compound 1 or a pharmaceutically acceptable salt thereof is administered to the subject daily.
148. The use according to embodiment 146 or 147, wherein Compound 1 or a pharmaceutically acceptable salt thereof is administered to the subject at a dose of about 3 mg/day.
149. The use according to embodiment 148, wherein about 3 mg daily of Compound 1 is administered to the subject.
150. The use according to any one of embodiments 146-149, wherein Compound 1 or a pharmaceutically acceptable salt thereof is administered orally.
151. Use of a compound having the formula

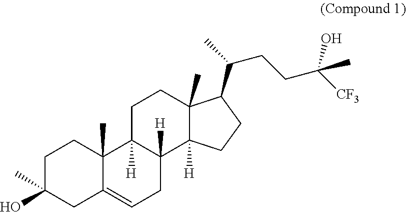

or a pharmaceutically acceptable salt thereof, for the manufacture of a medicament for improving learning and working memory in a subject having mild dementia associated with Alzheimer's Disease.
152. The use according to embodiment 151, wherein Compound 1 or a pharmaceutically acceptable salt thereof is administered to the subject daily.
153. The use according to embodiment 151 or 152, wherein Compound 1 or a pharmaceutically acceptable salt thereof is administered to the subject at a dose of about 3 mg/day.
154. The use according to embodiment 153, wherein about 3 mg daily of Compound 1 is administered to the subject.
155. The use according to any one of embodiments 151-154, wherein Compound 1 or a pharmaceutically acceptable salt thereof is administered orally.
156. The use according to any one of embodiments 131-156, wherein the subject is an adult.
157. A pharmaceutical composition comprising:
(a) an NMDA receptor positive allosteric modulator;
(b) an anti-amyloid beta antibody or antigen-binding fragment thereof; and
(c) a pharmaceutically acceptable carrier.
   158. The pharmaceutical composition according to embodiment 157, wherein the NMDA receptor positive allosteric modulator is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl) (6,7-dimethoxy-1-[(4-methoxyphenoxy)methyl]-3,4-dihydroisoquinolin-2 (1H)-yl)methanone (CIQ), NYX-458, and plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one), and pharmaceutically acceptable salts thereof.
   159. The pharmaceutical composition according to embodiment 157, wherein the NMDA receptor positive allosteric modulator is a neuroactive steroid, or a pharmaceutically acceptable salt thereof.
   160. The pharmaceutical composition according to embodiment 159, wherein the neuroactive steroid is selected from any one of compounds B1-B543; and pharmaceutically acceptable salts thereof.
   161. The pharmaceutical composition according to embodiment 160, wherein the neuroactive steroid is selected from any one of compounds B1-B140, and pharmaceutically acceptable salts thereof.
   162. The pharmaceutical composition according to embodiment 160, wherein the neuroactive steroid is selected from any one of compounds B150-B245, and pharmaceutically acceptable salts thereof.
   163. The pharmaceutical composition according to embodiment 160, wherein the neuroactive steroid is selected from any one of compounds B273-B349, and pharmaceutically acceptable salts thereof.
   164. The pharmaceutical composition according to embodiment 160, wherein the neuroactive steroid is selected from any one of compounds B395-B432, and pharmaceutically acceptable salts thereof.
   164A. The pharmaceutical composition according to embodiment 160, wherein the neuroactive steroid is selected from any one of compounds B433-B443 and pharmaceutically acceptable salts thereof and pharmaceutically acceptable salts thereof.
   164B. The pharmaceutical composition according to embodiment 160, wherein the neuroactive steroid is selected from any one of compounds B444-B451 and pharmaceutically acceptable salts thereof and pharmaceutically acceptable salts thereof.
   164C. The pharmaceutical composition according to embodiment 160, wherein the neuroactive steroid is selected from any one of compounds B452-B477 and pharmaceutically acceptable salts thereof and pharmaceutically acceptable salts thereof.
   164D. The pharmaceutical composition according to embodiment 160, wherein the neuroactive steroid is selected from any one of compounds B478-B530 and pharmaceutically acceptable salts thereof and pharmaceutically acceptable salts thereof.
   164E. The pharmaceutical composition according to embodiment 160, wherein the neuroactive steroid is selected from any one of compounds B531-B543 and pharmaceutically acceptable salts thereof and pharmaceutically acceptable salts thereof.
   165. The pharmaceutical composition according to any one of embodiments 157-164E, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   166. The pharmaceutical composition according to any one of embodiments 157-164E, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   167. The pharmaceutical composition according to embodiment 166, wherein the heavy chain variable domain (VH) comprises the amino acid sequence of SEQ ID NO: 7.
   168. The pharmaceutical composition according to embodiment 166, wherein the light chain variable domain (VL) comprises the amino acid sequence of SEQ ID NO: 8.
   169. The pharmaceutical composition according to embodiment 166, wherein the heavy chain variable domain (VH) comprises the amino acid sequence of SEQ ID NO: 7; and the light chain variable domain (VL) comprises the amino acid sequence of SEQ ID NO: 8.
   170. The pharmaceutical composition according to any one of embodiments 166-169, wherein said anti-amyloid beta antibody or antigen-binding fragment thereof comprises a human IgG1, IgG2, IgG3, or IgG4 constant domain.
   171. The pharmaceutical composition according to embodiment 170, wherein the anti-amyloid beta antibody or antigen-binding fragment thereof comprises a human IgG1 constant region.
   172. The pharmaceutical composition according to any one of embodiments 157-171, wherein the anti-amyloid beta antibody or antigen-binding fragment thereof is a human anti-amyloid beta antibody or antigen-binding fragment thereof.
   173. The pharmaceutical composition according to any one of embodiments 157-172, wherein the antigen-binding fragment is a single chain antibody, Fv, Fab, Fab′, F(ab′) 2, Fd, single chain Fv molecule (scFv), bispecific single chain Fv dimer, diabody, domain-deleted antibody or single domain antibody (dAb).
   174. The pharmaceutical composition according to any one of embodiments 157-173, wherein the heavy chain comprises the amino acid sequence of SEQ ID NO: 9.
   175. The pharmaceutical composition according to any one of embodiments 157-174, wherein the light chain comprises the amino acid sequence of SEQ ID NO: 10.
   176. The pharmaceutical composition according to any one of embodiments 157-175, wherein the anti-amyloid beta antibody is aducanumab.
   177. The pharmaceutical composition according to any one of embodiments 157-175, wherein the anti-amyloid beta antibody or antigen binding fragment thereof binds to the same conformational epitope of amyloid beta to which aducanumab binds.
   178. The pharmaceutical composition according to any one of embodiments 157-175, wherein the anti-amyloid beta antibody or antigen binding fragment thereof competitively inhibits binding of aducanumab to amyloid beta.
   179. The pharmaceutical composition according to any one of embodiments 157-178, wherein the anti-amyloid beta antibody or antigen binding fragment thereof binds to amyloid beta plaques, parenchymal amyloid, cerebrovascular amyloid, or diffuse amyloid beta deposits.
   180. A pharmaceutical composition comprising:
(a) a CYP46A1 inhibitor;
(b) an anti-amyloid beta antibody or antigen-binding fragment thereof; and
(c) a pharmaceutically acceptable carrier.
   181. The pharmaceutical composition according to embodiment 180, wherein the CYP46A1 inhibitor is selected from the group consisting of TAK-935, any one of compounds A1-A182, and pharmaceutically acceptable salts thereof.
   182. The pharmaceutical composition according to embodiment 181, wherein the CYP46A1 inhibitor is TAK-935, or a pharmaceutically acceptable salt thereof.
   183. The pharmaceutical composition according to embodiment 181, wherein the CYP46A1 inhibitor is selected from the group consisting of any one of compounds A1-A182, and pharmaceutically acceptable salts thereof.
   184. The pharmaceutical composition according to any one of embodiments 180-183, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   185. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   186. The pharmaceutical composition according to embodiment 185, wherein the heavy chain variable domain (VH) comprises the amino acid sequence of SEQ ID NO: 7.
   187. The pharmaceutical composition according to embodiment 185, wherein the light chain variable domain (VL) comprises the amino acid sequence of SEQ ID NO: 8.
   188. The pharmaceutical composition according to embodiment 185, wherein the heavy chain variable domain (VH) comprises the amino acid sequence of SEQ ID NO: 7; and the light chain variable domain (VL) comprises the amino acid sequence of SEQ ID NO: 8.
   189. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and
(b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16.
   190. The pharmaceutical composition according to embodiment 189, wherein the heavy chain variable domain (VH) comprises the amino acid sequence of SEQ ID NO: 17.
   191. The pharmaceutical composition according to embodiment 189, wherein the light chain variable domain (VL) comprises the amino acid sequence of SEQ ID NO: 18.
   192. The pharmaceutical composition according to embodiment 189, wherein the heavy chain variable domain (VH) comprises the amino acid sequence of SEQ ID NO: 17; and the light chain variable domain (VL) comprises the amino acid sequence of SEQ ID NO: 18.
   193. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and
(b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.
   194. The pharmaceutical composition according to embodiment 193, wherein the heavy chain variable domain (VH) comprises the amino acid sequence of SEQ ID NO: 27.
   195. The pharmaceutical composition according to embodiment 193, wherein the light chain variable domain (VL) comprises the amino acid sequence of SEQ ID NO: 28.
   196. The pharmaceutical composition according to embodiment 193, wherein the heavy chain variable domain (VH) comprises the amino acid sequence of SEQ ID NO: 27; and the light chain variable domain (VL) comprises the amino acid sequence of SEQ ID NO: 28.
   197. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and
(b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.
   198. The pharmaceutical composition according to embodiment 197, wherein the heavy chain variable domain (VH) comprises the amino acid sequence of SEQ ID NO: 37.
   199. The pharmaceutical composition according to embodiment 197, wherein the light chain variable domain (VL) comprises the amino acid sequence of SEQ ID NO: 38.
   200. The pharmaceutical composition according to embodiment 197, wherein the heavy chain variable domain (VH) comprises the amino acid sequence of SEQ ID NO: 37; and the light chain variable domain (VL) comprises the amino acid sequence of SEQ ID NO: 38.
   201. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and
(b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.
   202. The pharmaceutical composition according to embodiment 201, wherein the heavy chain variable domain (VH) comprises the amino acid sequence of SEQ ID NO: 47.
   203. The pharmaceutical composition according to embodiment 201, wherein the light chain variable domain (VL) comprises the amino acid sequence of SEQ ID NO: 48.
   204. The pharmaceutical composition according to embodiment 201, wherein the heavy chain variable domain (VH) comprises the amino acid sequence of SEQ ID NO: 47; and the light chain variable domain (VL) comprises the amino acid sequence of SEQ ID NO: 48.
   205. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and
(b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.
   206. The pharmaceutical composition according to embodiment 205, wherein the heavy chain variable domain (VH) comprises the amino acid sequence of SEQ ID NO: 57.
   207. The pharmaceutical composition according to embodiment 205, wherein the light chain variable domain (VL) comprises the amino acid sequence of SEQ ID NO: 58.
   208. The pharmaceutical composition according to embodiment 205, wherein the heavy chain variable domain (VH) comprises the amino acid sequence of SEQ ID NO: 57; and the light chain variable domain (VL) comprises the amino acid sequence of SEQ ID NO: 58.
   209. The pharmaceutical composition according to any one of embodiments 157-164E or 180-184, wherein the anti-amyloid beta antibody or antigen-binding fragment thereof comprises:
(a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and
(b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.
   210. The pharmaceutical composition according to embodiment 209, wherein the heavy chain variable domain (VH) comprises the amino acid sequence of SEQ ID NO: 67.
   211. The pharmaceutical composition according to embodiment 209, wherein the light chain variable domain (VL) comprises the amino acid sequence of SEQ ID NO: 68.
   212. The pharmaceutical composition according to embodiment 209, wherein the heavy chain variable domain (VH) comprises the amino acid sequence of SEQ ID NO: 67; and the light chain variable domain (VL) comprises the amino acid sequence of SEQ ID NO: 68.
   213. The pharmaceutical composition according to any one of embodiments 180-212, wherein said anti-amyloid beta antibody or antigen-binding fragment thereof comprises a human IgG1, IgG2, IgG3, or IgG4 constant domain.
   214. The pharmaceutical composition according to embodiment 213, wherein the anti-amyloid beta antibody or antigen-binding fragment thereof comprises a human IgG1 constant region.
   215. The pharmaceutical composition according to any one of embodiments 157-214, wherein the anti-amyloid beta antibody or antigen-binding fragment thereof is a human anti-amyloid beta antibody or antigen-binding fragment thereof.
   216. The pharmaceutical composition according to any one of embodiments 157-215 wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   217. The pharmaceutical composition according to any one of embodiments 157-216, wherein the heavy chain comprises the amino acid sequence of SEQ ID NO: 9.
   218. The pharmaceutical composition according to any one of embodiments 157-217, wherein the light chain comprises the amino acid sequence of SEQ ID NO: 10.
   219. The pharmaceutical composition according to any one of embodiments 157-218, wherein the anti-amyloid beta antibody is aducanumab.
   220. The pharmaceutical composition according to any one of embodiments 157-219, wherein the anti-amyloid beta antibody or antigen binding fragment thereof binds to the same conformational epitope of amyloid beta to which aducanumab binds.
   221. The pharmaceutical composition according to any one of embodiments 157-219, wherein the anti-amyloid beta antibody or antigen binding fragment thereof competitively inhibits binding of aducanumab to amyloid beta.
   222. The pharmaceutical composition according to any one of embodiments 157-216, wherein the heavy chain comprises the amino acid sequence of SEQ ID NO: 19.
   223. The pharmaceutical composition according to any one of embodiments 157-216 or 222, wherein the light chain comprises the amino acid sequence of SEQ ID NO: 20.
   224. The pharmaceutical composition according to any one of embodiments 157-216 or 222-223, wherein the anti-amyloid beta antibody is solazenumab.
   225. The pharmaceutical composition according to any one of embodiments 157-216 or 222-224, wherein the anti-amyloid beta antibody or antigen binding fragment thereof binds to the same conformational epitope of amyloid beta to which solazenumab binds.
   226. The pharmaceutical composition according to any one of embodiments 157-216 or 222-224, wherein the anti-amyloid beta antibody or antigen binding fragment thereof competitively inhibits binding of solazenumab to amyloid beta.
   227. The pharmaceutical composition according to any one of embodiments 157-216, wherein the heavy chain comprises the amino acid sequence of SEQ ID NO: 29.
   228. The pharmaceutical composition according to any one of embodiments 157-216 or 227, wherein the light chain comprises the amino acid sequence of SEQ ID NO: 30.
   229. The pharmaceutical composition according to any one of embodiments 157-216 or 227-228, wherein the anti-amyloid beta antibody is crenezumab.
   230. The pharmaceutical composition according to any one of embodiments 157-216 or 227-230, wherein the anti-amyloid beta antibody or antigen binding fragment thereof binds to the same conformational epitope of amyloid beta to which crenezumab binds.
   231. The pharmaceutical composition according to any one of embodiments 157-216 or 227-230, wherein the anti-amyloid beta antibody or antigen binding fragment thereof competitively inhibits binding of crenezumab to amyloid beta.
   232. The pharmaceutical composition according to any one of embodiments 157-216, wherein the heavy chain comprises the amino acid sequence of SEQ ID NO: 39.
   233. The pharmaceutical composition according to any one of embodiments 157-216 or 232, wherein the light chain comprises the amino acid sequence of SEQ ID NO: 40.
   234. The pharmaceutical composition according to any one of embodiments 157-216 or 232-233, wherein the anti-amyloid beta antibody is gantenerumab.
   235. The pharmaceutical composition according to any one of embodiments 157-216 or 232-234, wherein the anti-amyloid beta antibody or antigen binding fragment thereof binds to the same conformational epitope of amyloid beta to which gantenerumab binds.
   236. The pharmaceutical composition according to any one of embodiments 157-216 or 232-234, wherein the anti-amyloid beta antibody or antigen binding fragment thereof competitively inhibits binding of gantenerumab to amyloid beta.
   237. The pharmaceutical composition according to any one of embodiments 157-216, wherein the heavy chain comprises the amino acid sequence of SEQ ID NO: 49.
   238. The pharmaceutical composition according to any one of embodiments 157-216 or 237, wherein the light chain comprises the amino acid sequence of SEQ ID NO: 50.
   239. The pharmaceutical composition according to any one of embodiments 157-216 or 237-238, wherein the anti-amyloid beta antibody is ponezumab.
   240. The pharmaceutical composition according to any one of embodiments 157-216 or 237-239, wherein the anti-amyloid beta antibody or antigen binding fragment thereof binds to the same conformational epitope of amyloid beta to which ponezumab binds.
   241. The pharmaceutical composition according to any one of embodiments 157-216 or 237-239, wherein the anti-amyloid beta antibody or antigen binding fragment thereof competitively inhibits binding of ponezumab to amyloid beta.
   242. The pharmaceutical composition according to any one of embodiments 157-216, wherein the heavy chain comprises the amino acid sequence of SEQ ID NO: 59.
   243. The pharmaceutical composition according to any one of embodiments 157-216 or 242, wherein the light chain comprises the amino acid sequence of SEQ ID NO: 60.
   244. The pharmaceutical composition according to any one of embodiments 157-216 or 242-244, wherein the anti-amyloid beta antibody is bapineuzumab.
   245. The pharmaceutical composition according to any one of embodiments 157-216 or 242-244, wherein the anti-amyloid beta antibody or antigen binding fragment thereof binds to the same conformational epitope of amyloid beta to which bapineuzumab binds.
   246. The pharmaceutical composition according to any one of embodiments 157-216 or 242-244, wherein the anti-amyloid beta antibody or antigen binding fragment thereof competitively inhibits binding of bapineuzumab to amyloid beta.
   247. The pharmaceutical composition according to any one of embodiments 157-216, wherein the heavy chain comprises the amino acid sequence of SEQ ID NO: 69.
   248. The pharmaceutical composition according to any one of embodiments 157-216 or 247, wherein the light chain comprises the amino acid sequence of SEQ ID NO: 70.
   249. The pharmaceutical composition according to any one of embodiments 157-216 or 247-244, wherein the anti-amyloid beta antibody is lecanumab.
   250. The pharmaceutical composition according to any one of embodiments 157-216 or 247-249, wherein the anti-amyloid beta antibody or antigen binding fragment thereof binds to the same conformational epitope of amyloid beta to which lecanumab binds.
   251. The pharmaceutical composition according to any one of embodiments 157-216 or 247-249, wherein the anti-amyloid beta antibody or antigen binding fragment thereof competitively inhibits binding of lecanumab to amyloid beta.
   252. The pharmaceutical composition according to any one of embodiments 157-251, wherein the anti-amyloid beta antibody or antigen binding fragment thereof binds to amyloid beta plaques, parenchymal amyloid, cerebrovascular amyloid, or diffuse amyloid beta deposits.
   253. A kit comprising a first container, a second container and a package insert, wherein:
the first container comprises a composition comprising an NMDA receptor positive allosteric modulator;
the second container comprises composition comprising an anti-amyloid beta antibody or antigen-binding fragment thereof; and
the package insert comprises instructions for treating Alzheimer's disease in a subject.
   254. The kit according to embodiment 253, wherein the NMDA receptor positive allosteric modulator is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl) (6,7-dimethoxy-1-[(4-methoxyphenoxy)methyl]-3,4-dihydroisoquinolin-2 (1H)-yl)methanone (CIQ), NYX-458, and plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one), and pharmaceutically acceptable salts thereof.
   255. The kit according to embodiment 253, wherein the NMDA receptor positive allosteric modulator is a neuroactive steroid, or a pharmaceutically acceptable salt thereof.
   256. The kit according to embodiment 255, wherein the neuroactive steroid is selected from any one of compounds B1-B543; and pharmaceutically acceptable salts thereof.
   257. The kit according to embodiment 255, wherein the neuroactive steroid is selected from any one of compounds B1-B140, and pharmaceutically acceptable salts thereof.
   258. The kit according to embodiment 255 wherein the neuroactive steroid is selected from any one of compounds B150-B245, and pharmaceutically acceptable salts thereof.
   259. The kit according to embodiment 255, wherein the neuroactive steroid is selected from any one of compounds B273-B349, and pharmaceutically acceptable salts thereof.
   260. The kit according to embodiment 255, wherein the neuroactive steroid is selected from any one of compounds B395-B432, and pharmaceutically acceptable salts thereof.
   260A. The pharmaceutical composition according to embodiment 255, wherein the neuroactive steroid is selected from any one of compounds B433-B443 and pharmaceutically acceptable salts thereof and pharmaceutically acceptable salts thereof.
   260B. The pharmaceutical composition according to embodiment 255 wherein the neuroactive steroid is selected from any one of compounds B444-B451 and pharmaceutically acceptable salts thereof and pharmaceutically acceptable salts thereof.
   260C. The pharmaceutical composition according to embodiment 255, wherein the neuroactive steroid is selected from any one of compounds B452-B477 and pharmaceutically acceptable salts thereof and pharmaceutically acceptable salts thereof.
   260D. The pharmaceutical composition according to embodiment 255, wherein the neuroactive steroid is selected from any one of compounds B478-B530 and pharmaceutically acceptable salts thereof and pharmaceutically acceptable salts thereof.
   260E. The pharmaceutical composition according to embodiment 255, wherein the neuroactive steroid is selected from any one of compounds B531-B543 and pharmaceutically acceptable salts thereof and pharmaceutically acceptable salts thereof.
   261. The kit according to any one of embodiments 253-260E, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   262. The kit according to any one of embodiments 253-260E, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   263. The kit according to embodiment 262, wherein the heavy chain variable domain (VH) comprises the amino acid sequence of SEQ ID NO: 7.
   264. The kit according to embodiment 262, wherein the light chain variable domain (VL) comprises the amino acid sequence of SEQ ID NO: 8.
   265. The kit according to embodiment 262, wherein the heavy chain variable domain (VH) comprises the amino acid sequence of SEQ ID NO: 7; and the light chain variable domain (VL) comprises the amino acid sequence of SEQ ID NO: 8.
   266. The kit according to any one of embodiments 253-265, wherein said anti-amyloid beta antibody or antigen-binding fragment thereof comprises a human IgG1, IgG2, IgG3, or IgG4 constant domain.
   267. The kit according to embodiment 266, wherein the anti-amyloid beta antibody or antigen-binding fragment thereof comprises a human IgG1 constant region.
   268. The kit according to any one of embodiments 253-267, wherein the anti-amyloid beta antibody or antigen-binding fragment thereof is a human anti-amyloid beta antibody or antigen-binding fragment thereof.
   269. The kit according to any one of embodiments 253-268, wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   270. The kit according to any one of embodiments 253-269, wherein the heavy chain comprises the amino acid sequence of SEQ ID NO: 9.
   271. The kit according to any one of embodiments 253-270, wherein the light chain comprises the amino acid sequence of SEQ ID NO: 10.
   272. The kit according to any one of embodiments 253-271, wherein the anti-amyloid beta antibody is aducanumab.
   273. The kit according to any one of embodiments 253-272, wherein the anti-amyloid beta antibody or antigen binding fragment thereof binds to the same conformational epitope of amyloid beta to which aducanumab binds.
   274. The kit according to any one of embodiments 253-272, wherein the anti-amyloid beta antibody or antigen binding fragment thereof competitively inhibits binding of aducanumab to amyloid beta.
   275. The kit according to any one of embodiments 253-274, wherein the anti-amyloid beta antibody or antigen binding fragment thereof binds to amyloid beta plaques, parenchymal amyloid, cerebrovascular amyloid, or diffuse amyloid beta deposits.
   276. A kit comprising a first container, a second container and a package insert, wherein:
the first container comprises a composition comprising CYP46A1 inhibitor;
the second container comprises composition comprising an anti-amyloid beta antibody or antigen-binding fragment thereof; and
the package insert comprises instructions for treating Alzheimer's disease in a subject.
   277. The kit according to embodiment 276, wherein the CYP46A1 inhibitor is selected from the group consisting of TAK-935, any one of compounds A1-A182, and pharmaceutically acceptable salts thereof.
   278. The kit according to embodiment 277, wherein the CYP46A1 inhibitor is TAK-935, or a pharmaceutically acceptable salt thereof.
   279. The kit according to embodiment 278, wherein the CYP46A1 inhibitor is selected from the group consisting of any one of compounds A1-A182, and pharmaceutically acceptable salts thereof.
   280. The kit according to any one of embodiments 276-279, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   281. The kit according to any one of embodiments 276-279, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   282. The kit according to embodiment 281, wherein the heavy chain variable domain (VH) comprises the amino acid sequence of SEQ ID NO: 7.
   283. The kit according to embodiment 281, wherein the light chain variable domain (VL) comprises the amino acid sequence of SEQ ID NO: 8.
   284. The kit according to embodiment 281, wherein the heavy chain variable domain (VH) comprises the amino acid sequence of SEQ ID NO: 7; and the light chain variable domain (VL) comprises the amino acid sequence of SEQ ID NO: 8.
   285. The kit according to any one of embodiments 253-279, wherein the anti-amyloid beta antibody or antigen-binding fragment thereof comprises:
(a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and
(b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16.
   286. The kit according to embodiment 285, wherein the heavy chain variable domain (VH) comprises the amino acid sequence of SEQ ID NO: 17.
   287. The kit according to embodiment 285, wherein the light chain variable domain (VL) comprises the amino acid sequence of SEQ ID NO: 18.
   288. The kit according to embodiment 285, wherein the heavy chain variable domain (VH) comprises the amino acid sequence of SEQ ID NO: 17; and the light chain variable domain (VL) comprises the amino acid sequence of SEQ ID NO: 18.
   289. The kit according to any one of embodiments 253-279, wherein the anti-amyloid beta antibody or antigen-binding fragment thereof comprises:
(a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and
(b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.
   290. The kit according to embodiment 289, wherein the heavy chain variable domain (VH) comprises the amino acid sequence of SEQ ID NO: 27.
   291. The kit according to embodiment 289, wherein the light chain variable domain (VL) comprises the amino acid sequence of SEQ ID NO: 28.
   292. The kit according to embodiment 289, wherein the heavy chain variable domain (VH) comprises the amino acid sequence of SEQ ID NO: 27; and the light chain variable domain (VL) comprises the amino acid sequence of SEQ ID NO: 28.
   293. The kit according to any one of embodiments 253-279, wherein the anti-amyloid beta antibody or antigen-binding fragment thereof comprises:
(a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and
(b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.
   294. The kit according to embodiment 293, wherein the heavy chain variable domain (VH) comprises the amino acid sequence of SEQ ID NO: 37.
   295. The kit according to embodiment 293, wherein the light chain variable domain (VL) comprises the amino acid sequence of SEQ ID NO: 38.
   296. The kit according to embodiment 293, wherein the heavy chain variable domain (VH) comprises the amino acid sequence of SEQ ID NO: 37; and the light chain variable domain (VL) comprises the amino acid sequence of SEQ ID NO: 38.
   297. The kit according to any one of embodiments 253-279, wherein the anti-amyloid beta antibody or antigen-binding fragment thereof comprises:
(a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and
(b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.
   298. The kit according to embodiment 297, wherein the heavy chain variable domain (VH) comprises the amino acid sequence of SEQ ID NO: 47.
   299. The kit according to embodiment 297, wherein the light chain variable domain (VL) comprises the amino acid sequence of SEQ ID NO: 48.
   300. The kit according to embodiment 297, wherein the heavy chain variable domain (VH) comprises the amino acid sequence of SEQ ID NO: 47; and the light chain variable domain (VL) comprises the amino acid sequence of SEQ ID NO: 48.
   301. The kit according to any one of embodiments 253-279, wherein the anti-amyloid beta antibody or antigen-binding fragment thereof comprises:
(a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and
(b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.
   302. The kit according to embodiment 301, wherein the heavy chain variable domain (VH) comprises the amino acid sequence of SEQ ID NO: 57.
   303. The kit according to embodiment 301, wherein the light chain variable domain (VL) comprises the amino acid sequence of SEQ ID NO: 58.
   304. The kit according to embodiment 301, wherein the heavy chain variable domain (VH) comprises the amino acid sequence of SEQ ID NO: 57; and the light chain variable domain (VL) comprises the amino acid sequence of SEQ ID NO: 58.
   305. The kit according to any one of embodiments 253-279, wherein the anti-amyloid beta antibody or antigen-binding fragment thereof comprises:
(a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and
(b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.
   306. The kit according to embodiment 305, wherein the heavy chain variable domain (VH) comprises the amino acid sequence of SEQ ID NO: 67.
   307. The kit according to embodiment 305, wherein the light chain variable domain (VL) comprises the amino acid sequence of SEQ ID NO: 68.
   308. The kit according to embodiment 305, wherein the heavy chain variable domain (VH) comprises the amino acid sequence of SEQ ID NO: 67; and the light chain variable domain (VL) comprises the amino acid sequence of SEQ ID NO: 68.
   309. The kit according to any one of embodiments 253-308, wherein said anti-amyloid beta antibody or antigen-binding fragment thereof comprises a human IgG1, IgG2, IgG3, or IgG4 constant domain.
   310. The kit according to embodiment 309, wherein the anti-amyloid beta antibody or antigen-binding fragment thereof comprises a human IgG1 constant region.
   311. The kit according to any one of embodiments 253-310, wherein the anti-amyloid beta antibody or antigen-binding fragment thereof is a human anti-amyloid beta antibody or antigen-binding fragment thereof.
   312. The kit according to any one of embodiments 253-311, wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   313. The kit according to any one of embodiments 253-312, wherein the heavy chain comprises the amino acid sequence of SEQ ID NO: 9.
   314. The kit according to any one of embodiments 253-313, wherein the light chain comprises the amino acid sequence of SEQ ID NO: 10.
   315. The kit according to any one of embodiments 253-314, wherein the anti-amyloid beta antibody is aducanumab.
   316. The kit according to any one of embodiments 253-315, wherein the anti-amyloid beta antibody or antigen binding fragment thereof binds to the same conformational epitope of amyloid beta to which aducanumab binds.
   317. The kit according to any one of embodiments 253-316, wherein the anti-amyloid beta antibody or antigen binding fragment thereof competitively inhibits binding of aducanumab to amyloid beta.
   318. The kit according to any one of embodiments 253-312, wherein the heavy chain comprises the amino acid sequence of SEQ ID NO: 19.
   319. The kit according to any one of embodiments 253-312 or 318, wherein the light chain comprises the amino acid sequence of SEQ ID NO: 20.
   320. The kit according to any one of embodiments 253-312 or 318-319, wherein the anti-amyloid beta antibody is solazenumab.
   321. The kit according to any one of embodiments 253-312 or 318-320, wherein the anti-amyloid beta antibody or antigen binding fragment thereof binds to the same conformational epitope of amyloid beta to which solazenumab binds.
   322. The kit according to any one of embodiments 253-312 or 318-320, wherein the anti-amyloid beta antibody or antigen binding fragment thereof competitively inhibits binding of solazenumab to amyloid beta.
   323. The kit according to any one of embodiments 253-312, wherein the heavy chain comprises the amino acid sequence of SEQ ID NO: 29.
   324. The kit according to any one of embodiments 253-312 or 323, wherein the light chain comprises the amino acid sequence of SEQ ID NO: 30.
   325. The kit according to any one of embodiments 253-312 or 323-324, wherein the anti-amyloid beta antibody is crenezumab.
   326. The kit according to any one of embodiments 253-312 or 323-325, wherein the anti-amyloid beta antibody or antigen binding fragment thereof binds to the same conformational epitope of amyloid beta to which crenezumab binds.
   327. The kit according to any one of embodiments 253-312 or 323-325, wherein the anti-amyloid beta antibody or antigen binding fragment thereof competitively inhibits binding of crenezumab to amyloid beta.
   328. The kit according to any one of embodiments 253-312, wherein the heavy chain comprises the amino acid sequence of SEQ ID NO: 39.
   329. The kit according to any one of embodiments 253-312 or 328, wherein the light chain comprises the amino acid sequence of SEQ ID NO: 40.
   330. The kit according to any one of embodiments 253-312 or 328-329, wherein the anti-amyloid beta antibody is gantenerumab.
   331. The kit according to any one of embodiments 253-312 or 328-330, wherein the anti-amyloid beta antibody or antigen binding fragment thereof binds to the same conformational epitope of amyloid beta to which gantenerumab binds.
   332. The kit according to any one of embodiments 253-312 and 328-330, wherein the anti-amyloid beta antibody or antigen binding fragment thereof competitively inhibits binding of gantenerumab to amyloid beta.
   333. The kit according to any one of embodiments 253-312, wherein the heavy chain comprises the amino acid sequence of SEQ ID NO: 49.
   334. The kit according to any one of embodiments 253-312 or 333, wherein the light chain comprises the amino acid sequence of SEQ ID NO: 50.
   335. The kit according to any one of embodiments 253-312 or 333-334, wherein the anti-amyloid beta antibody is ponezumab.
   336. The kit according to any one of embodiments 253-312 or 333-335, wherein the anti-amyloid beta antibody or antigen binding fragment thereof binds to the same conformational epitope of amyloid beta to which ponezumab binds.
   337. The kit according to any one of embodiments 253-312 or 333-335, wherein the anti-amyloid beta antibody or antigen binding fragment thereof competitively inhibits binding of ponezumab to amyloid beta.
   338. The kit according to any one of embodiments 253-312, wherein the heavy chain comprises the amino acid sequence of SEQ ID NO: 59.
   339. The kit according to any one of embodiments 253-312 or 338, wherein the light chain comprises the amino acid sequence of SEQ ID NO: 60.
   340. The kit according to any one of embodiments 253-312 or 338-339, wherein the anti-amyloid beta antibody is bapineuzumab.
   341. The kit according to any one of embodiments 253-312 or 338-340, wherein the anti-amyloid beta antibody or antigen binding fragment thereof binds to the same conformational epitope of amyloid beta to which bapineuzumab binds.
   342. The kit according to any one of embodiments 253-312 or 338-340, wherein the anti-amyloid beta antibody or antigen binding fragment thereof competitively inhibits binding of bapineuzumab to amyloid beta.
   343. The kit according to any one of embodiments 253-312, wherein the heavy chain comprises the amino acid sequence of SEQ ID NO: 69.
   344. The kit according to any one of embodiments 253-312 or 343, wherein the light chain comprises the amino acid sequence of SEQ ID NO: 70.
   345. The kit according to any one of embodiments 253-312 or 343-344, wherein the anti-amyloid beta antibody is lecanumab.
   346. The kit according to any one of embodiments 253-312 or 343-345, wherein the anti-amyloid beta antibody or antigen binding fragment thereof binds to the same conformational epitope of amyloid beta to which lecanumab binds.
   347. The kit according to any one of embodiments 253-312 or 343-345, wherein the anti-amyloid beta antibody or antigen binding fragment thereof competitively inhibits binding of lecanumab to amyloid beta.
   348. The kit according to any one of embodiment 253-347, wherein the anti-amyloid beta antibody or antigen binding fragment thereof binds to amyloid beta plaques, parenchymal amyloid, cerebrovascular amyloid, or diffuse amyloid beta deposits.
   349. A method for treating or preventing Alzheimer's disease, comprising administering to a subject in need thereof (a) an NMDA receptor positive allosteric modulator, and (b) an anti-amyloid beta antibody or antigen-binding fragment thereof.
   350. A method for treating or preventing cognitive impairment in a subject having Alzheimer's disease, comprising administering to the subject (a) an NMDA receptor positive allosteric modulator, and (b) an anti-amyloid beta antibody or antigen-binding fragment thereof.
   351. A method for treating or preventing cognitive impairment due to Alzheimer's disease in a subject, comprising administering to the subject (a) an NMDA receptor positive allosteric modulator, and (b) an anti-amyloid beta antibody or antigen-binding fragment thereof.
   352. The method of embodiment 350 or 351, wherein the cognitive impairment is mild cognitive impairment.
   353. A method for improving cognition in a subject having Alzheimer's disease, comprising administering to the subject (a) an NMDA receptor positive allosteric modulator, and (b) an anti-amyloid beta antibody or antigen-binding fragment thereof.
   354. A method for slowing cognitive decline due to Alzheimer's disease in a subject, comprising administering to the subject (a) an NMDA receptor positive allosteric modulator, and (b) an anti-amyloid beta antibody or antigen-binding fragment thereof.
   355. A method of treating including Mild Cognitive Impairment (MCI) associated with Alzheimer's Disease in a subject, the method comprising administering to the subject (a) an NMDA receptor positive allosteric modulator, and (b) an anti-amyloid beta antibody or antigen-binding fragment thereof.
   356. A method of improving executive function in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.
   357. A method of improving working memory in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.
   358. A method of improving learning in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.
   359. A method of improving learning and working memory in a subject having Mild Cognitive Impairment (MCI) associated with Alzheimer's Disease, the method comprising administering to the subject (a) an NMDA receptor positive allosteric modulator, and (b) an anti-amyloid beta antibody or antigen-binding fragment thereof.
   360. A method of treating mild dementia associated with Alzheimer's Disease in a subject, the method comprising administering to the subject (a) an NMDA receptor positive allosteric modulator, and (b) an anti-amyloid beta antibody or antigen-binding fragment thereof.
   361. A method of improving executive function in a subject having mild dementia associated with Alzheimer's Disease in a subject, the method comprising administering to the subject (a) an NMDA receptor positive allosteric modulator, and (b) an anti-amyloid beta antibody or antigen-binding fragment thereof.
   362. A method of improving working memory in a subject having mild dementia associated with Alzheimer's Disease in a subject, the method comprising administering to the subject (a) an NMDA receptor positive allosteric modulator, and (b) an anti-amyloid beta antibody or antigen-binding fragment thereof.
   363. A method of improving learning in a subject having mild dementia associated with Alzheimer's Disease in a subject, the method comprising administering to the subject (a) an NMDA receptor positive allosteric modulator, and (b) an anti-amyloid beta antibody or antigen-binding fragment thereof.
   364. A method of improving learning and working memory in a subject having mild dementia associated with Alzheimer's Disease in a subject, the method comprising administering to the subject (a) an NMDA receptor positive allosteric modulator, and (b) an anti-amyloid beta antibody or antigen-binding fragment thereof.
   365. The method according to any one of embodiments 349-364, wherein the NMDA receptor positive allosteric modulator is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl) (6,7-dimethoxy-1-[(4-methoxyphenoxy)methyl]-3,4-dihydroisoquinolin-2 (1H)-yl)methanone (CIQ), NYX-458, and plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one) and pharmaceutically acceptable salts thereof.
   366. The method according to any one of embodiments 349-364, wherein the NMDA receptor positive allosteric modulator is a neuroactive steroid, or a pharmaceutically acceptable salt thereof.
   367. The method according to embodiment 366, wherein the neuroactive steroid is selected from any one of compounds B1-B543; and pharmaceutically acceptable salts thereof.
   368. The method according to embodiment 367, wherein the neuroactive steroid is selected from any one of compounds B1-B140, and pharmaceutically acceptable salts thereof.
   369. The method according to embodiment 367, wherein the neuroactive steroid is selected from any one of compounds B150-B245, and pharmaceutically acceptable salts thereof.
   370. The method according to embodiment 367, wherein the neuroactive steroid is selected from any one of compounds B273-B349, and pharmaceutically acceptable salts thereof.
   371A. The method according to embodiment 367, wherein the neuroactive steroid is selected from any one of compounds B395-B432, and pharmaceutically acceptable salts thereof.
   371B. The pharmaceutical composition according to embodiment 367, wherein the neuroactive steroid is selected from any one of compounds B433-B443 and pharmaceutically acceptable salts thereof and pharmaceutically acceptable salts thereof.
   371C. The pharmaceutical composition according to embodiment 367, wherein the neuroactive steroid is selected from any one of compounds B444-B451 and pharmaceutically acceptable salts thereof and pharmaceutically acceptable salts thereof.
   371D. The pharmaceutical composition according to embodiment 367, wherein the neuroactive steroid is selected from any one of compounds B452-B477 and pharmaceutically acceptable salts thereof and pharmaceutically acceptable salts thereof.
   371E. The pharmaceutical composition according to embodiment 367, wherein the neuroactive steroid is selected from any one of compounds B478-B530 and pharmaceutically acceptable salts thereof and pharmaceutically acceptable salts thereof.
   371F. The pharmaceutical composition according to embodiment 367, wherein the neuroactive steroid is selected from any one of compounds B531-B543 and pharmaceutically acceptable salts thereof and pharmaceutically acceptable salts thereof.
   371. The method according to any one of embodiments 349-371F, wherein the NMDA receptor positive allosteric modulator and the anti-amyloid beta antibody or antigen binding fragment thereof are administered simultaneously.
   372. The method according to any one of embodiments 349-371F, wherein the NMDA receptor positive allosteric modulator and the anti-amyloid beta antibody or antigen binding fragment thereof are administered sequentially.
   373. The method according to any one of embodiments 349-371F, wherein the NMDA receptor positive allosteric modulator and the anti-amyloid beta antibody or antigen binding fragment thereof are administered separately.
   374. The method according to embodiments 372 or 373, wherein the anti-amyloid beta antibody or antigen binding fragment thereof is administered before the NMDA receptor positive allosteric modulator.
   375. The method according to embodiments 372 or 373, wherein the NMDA receptor positive allosteric modulator is administered before the anti-amyloid beta antibody or antigen binding fragment thereof.
   376. The method according to any one of embodiments 349-375, wherein the frequency of administration of the anti-amyloid beta antibody or antigen binding fragment thereof and the NMDA receptor positive allosteric modulator is the same.
   377. The method according to any one of embodiments 349-375, wherein the frequency of administration of the anti-amyloid beta antibody or antigen binding fragment thereof and the NMDA receptor positive allosteric modulator is different.
   378. A method for treating or preventing Alzheimer's disease, comprising administering to a subject in need thereof (a) a CYP46A1 inhibitor, and (b) an anti-amyloid beta antibody or antigen-binding fragment thereof.
   379. A method for treating or preventing cognitive impairment in a subject having Alzheimer's disease, comprising administering to the subject (a) a CYP46A1 inhibitor, and (b) an anti-amyloid beta antibody or antigen-binding fragment thereof.
   380. A method for treating or preventing cognitive impairment due to Alzheimer's disease in a subject, comprising administering to the subject (a) a CYP46A1 inhibitor, and (b) an anti-amyloid beta antibody or antigen-binding fragment thereof.
   381. The method of embodiment 379 or 380, wherein the cognitive impairment is mild cognitive impairment.
   382. A method for improving cognition in a subject having Alzheimer's disease, comprising administering to the subject (a) a CYP46A1 inhibitor, and (b) an anti-amyloid beta antibody or antigen-binding fragment thereof.
   383. A method for slowing cognitive decline due to Alzheimer's disease in a subject, comprising administering to the subject (a) a CYP46A1 inhibitor, and (b) an anti-amyloid beta antibody or antigen-binding fragment thereof.
   384. A method of treating Mild Cognitive Impairment (MCI) associated with Alzheimer's Disease in a subject, the method comprising administering to the subject (a) a CYP46A1 inhibitor, and (b) an anti-amyloid beta antibody or antigen-binding fragment thereof.
   385. A method of improving executive function in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.
   386. A method of improving working memory in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.
   387. A method of improving learning in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.
   388. A method of improving learning and working memory in a subject having Mild Cognitive Impairment (MCI) associated with Alzheimer's Disease, the method comprising administering to the subject (a) a CYP46A1 inhibitor, and (b) an anti-amyloid beta antibody or antigen-binding fragment thereof.
   389. A method of treating mild dementia associated with Alzheimer's Disease in a subject, the method comprising administering to the subject (a) a CYP46A1 inhibitor, and (b) an anti-amyloid beta antibody or antigen-binding fragment thereof.
   390. A method of improving executive function in a subject having mild dementia associated with Alzheimer's Disease in a subject, the method comprising administering to the subject (a) a CYP46A1 inhibitor, and (b) an anti-amyloid beta antibody or antigen-binding fragment thereof.
   391. A method of improving working memory in a subject having mild dementia associated with Alzheimer's Disease in a subject, the method comprising administering to the subject (a) a CYP46A1 inhibitor, and (b) an anti-amyloid beta antibody or antigen-binding fragment thereof.
   392. A method of improving learning in a subject having mild dementia associated with Alzheimer's Disease in a subject, the method comprising administering to the subject (a) a CYP46A1 inhibitor, and (b) an anti-amyloid beta antibody or antigen-binding fragment thereof.
   393. A method of improving learning and working memory in a subject having mild dementia associated with Alzheimer's Disease in a subject, the method comprising administering to the subject (a) a CYP46A1 inhibitor, and (b) an anti-amyloid beta antibody or antigen-binding fragment thereof.
   394. The method according to any one of embodiments 378-393, wherein the CYP46A1 inhibitor is selected from the group consisting of TAK-935, any one of compounds A1-A182, and pharmaceutically acceptable salts thereof.
   395. The method according to embodiment 394, wherein the CYP46A1 inhibitor is TAK-935, or a pharmaceutically acceptable salt thereof.
   396. The method according to embodiment 394, wherein the CYP46A1 inhibitor is selected from the group consisting of any one of compounds A1-A182, and pharmaceutically acceptable salts thereof.
   397. The method according to any one of embodiments 349-396, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   398. The method according to any one of embodiments 349-396, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   399. The method according to embodiment 398, wherein the heavy chain variable domain (VH) comprises the amino acid sequence of SEQ ID NO: 7.
   400. The method according to embodiment 398, wherein the light chain variable domain (VL) comprises the amino acid sequence of SEQ ID NO: 8.
   401. The method according to embodiment 398, wherein the heavy chain variable domain (VH) comprises the amino acid sequence of SEQ ID NO: 7; and the light chain variable domain (VL) comprises the amino acid sequence of SEQ ID NO: 8.
   402. The method according to any one of embodiments 349-397, wherein the anti-amyloid beta antibody or antigen-binding fragment thereof comprises:
(a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and
(b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16.
   403. The method according to embodiment 402, wherein the heavy chain variable domain (VH) comprises the amino acid sequence of SEQ ID NO: 17.
   404. The method according to embodiment 402, wherein the light chain variable domain (VL) comprises the amino acid sequence of SEQ ID NO: 18.
   405. The method according to embodiment 402, wherein the heavy chain variable domain (VH) comprises the amino acid sequence of SEQ ID NO: 17; and the light chain variable domain (VL) comprises the amino acid sequence of SEQ ID NO: 18.
   406. The method according to any one of embodiments 349-396, wherein the anti-amyloid beta antibody or antigen-binding fragment thereof comprises:
(a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and
(b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.
   407. The method according to embodiment 406, wherein the heavy chain variable domain (VH) comprises the amino acid sequence of SEQ ID NO: 27.
   408. The method according to embodiment 406, wherein the light chain variable domain (VL) comprises the amino acid sequence of SEQ ID NO: 28.
   409. The method according to embodiment 406, wherein the heavy chain variable domain (VH) comprises the amino acid sequence of SEQ ID NO: 27; and the light chain variable domain (VL) comprises the amino acid sequence of SEQ ID NO: 28.
   410. The method according to any one of embodiments 349-396, wherein the anti-amyloid beta antibody or antigen-binding fragment thereof comprises:
(a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and
(b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.
   411. The method according to embodiment 410, wherein the heavy chain variable domain (VH) comprises the amino acid sequence of SEQ ID NO: 37.
   412. The method according to embodiment 410, wherein the light chain variable domain (VL) comprises the amino acid sequence of SEQ ID NO: 38.
   413. The method according to embodiment 410, wherein the heavy chain variable domain (VH) comprises the amino acid sequence of SEQ ID NO: 37; and the light chain variable domain (VL) comprises the amino acid sequence of SEQ ID NO: 38.
   414. The method according to any one of embodiments 349-396, wherein the anti-amyloid beta antibody or antigen-binding fragment thereof comprises:
(a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and
(b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.
   415. The method according to embodiment 414, wherein the heavy chain variable domain (VH) comprises the amino acid sequence of SEQ ID NO: 47.
   416. The method according to embodiment 414, wherein the light chain variable domain (VL) comprises the amino acid sequence of SEQ ID NO: 48.
   417. The method according to embodiment 414, wherein the heavy chain variable domain (VH) comprises the amino acid sequence of SEQ ID NO: 47; and the light chain variable domain (VL) comprises the amino acid sequence of SEQ ID NO: 48.
   418. The method according to any one of embodiments 349-396, wherein the anti-amyloid beta antibody or antigen-binding fragment thereof comprises:
(a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and
(b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.
   419. The method according to embodiment 418, wherein the heavy chain variable domain (VH) comprises the amino acid sequence of SEQ ID NO: 57.
   420. The method according to embodiment 418, wherein the light chain variable domain (VL) comprises the amino acid sequence of SEQ ID NO: 58.
   421. The method according to embodiment 418, wherein the heavy chain variable domain (VH) comprises the amino acid sequence of SEQ ID NO: 57; and the light chain variable domain (VL) comprises the amino acid sequence of SEQ ID NO: 58.
   422. The method according to any one of embodiments 349-396, wherein the anti-amyloid beta antibody or antigen-binding fragment thereof comprises:
(a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and
(b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.
   423. The method according to embodiment 422, wherein the heavy chain variable domain (VH) comprises the amino acid sequence of SEQ ID NO: 67.
   424. The method according to embodiment 422, wherein the light chain variable domain (VL) comprises the amino acid sequence of SEQ ID NO: 68.
   425. The method according to embodiment 422, wherein the heavy chain variable domain (VH) comprises the amino acid sequence of SEQ ID NO: 67; and the light chain variable domain (VL) comprises the amino acid sequence of SEQ ID NO: 68.
   426. The method according to any one of embodiments 349-425, wherein said anti-amyloid beta antibody or antigen-binding fragment thereof comprises a human IgG1, IgG2, IgG3, or IgG4 constant domain.
   427. The method according to embodiment 426, wherein the anti-amyloid beta antibody or antigen-binding fragment thereof comprises a human IgG1 constant region.
   428. The method according to any one of embodiments 349-427, wherein the anti-amyloid beta antibody or antigen-binding fragment thereof is a human anti-amyloid beta antibody or antigen-binding fragment thereof.
   429. The method according to any one of embodiments 349-428, wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   430. The method according to any one of embodiments 349-429, wherein the heavy chain comprises the amino acid sequence of SEQ ID NO: 9.
   431. The method according to any one of embodiments 349-430, wherein the light chain comprises the amino acid sequence of SEQ ID NO: 10.
   432. The method according to any one of embodiments 349-431, wherein the anti-amyloid beta antibody is aducanumab.
   433. The method according to any one of embodiments 349-432, wherein the anti-amyloid beta antibody or antigen binding fragment thereof binds to the same conformational epitope of amyloid beta to which aducanumab binds.
   434. The method according to any one of embodiments 349-432, wherein the anti-amyloid beta antibody or antigen binding fragment thereof competitively inhibits binding of aducanumab to amyloid beta.
   435. The method according to any one of embodiments 349-429, wherein the heavy chain comprises the amino acid sequence of SEQ ID NO: 19.
   436. The method according to any one of embodiments 349-429 or 435, wherein the light chain comprises the amino acid sequence of SEQ ID NO: 20.
   437. The method according to any one of embodiments 349-429 or 435-436, wherein the anti-amyloid beta antibody is solazenumab.
   438. The method according to any one of embodiments 349-429 or 435-437, wherein the anti-amyloid beta antibody or antigen binding fragment thereof binds to the same conformational epitope of amyloid beta to which solazenumab binds.
   439. The method according to any one of embodiments 349-429 or 435-437, wherein the anti-amyloid beta antibody or antigen binding fragment thereof competitively inhibits binding of solazenumab to amyloid beta.
   440. The method according to any one of embodiments 349-429, wherein the heavy chain comprises the amino acid sequence of SEQ ID NO: 29.
   441. The method according to any one of embodiments 349-429 or 440, wherein the light chain comprises the amino acid sequence of SEQ ID NO: 30.
   442. The method according to any one of embodiments 349-429 or 440-441, wherein the anti-amyloid beta antibody is crenezumab.
   443. The method according to any one of embodiments 349-429 or 440-441, wherein the anti-amyloid beta antibody or antigen binding fragment thereof binds to the same conformational epitope of amyloid beta to which crenezumab binds.
   444. The method according to any one of embodiments 349-429 or 440-442, wherein the anti-amyloid beta antibody or antigen binding fragment thereof competitively inhibits binding of crenezumab to amyloid beta.
   445. The method according to any one of embodiments 349-429, wherein the heavy chain comprises the amino acid sequence of SEQ ID NO: 39.
   446. The method according to any one of embodiments 349-429 or 445, wherein the light chain comprises the amino acid sequence of SEQ ID NO: 40.
   447. The method according to any one of embodiments 349-429 or 445-446, wherein the anti-amyloid beta antibody is gantenerumab.
   448. The method according to any one of embodiments 349-429 or 5447, wherein the anti-amyloid beta antibody or antigen binding fragment thereof binds to the same conformational epitope of amyloid beta to which gantenerumab binds.
   449. The method according to any one of embodiments 349-429 and 445-447, wherein the anti-amyloid beta antibody or antigen binding fragment thereof competitively inhibits binding of gantenerumab to amyloid beta.
   450. The method according to any one of embodiments 349-429, wherein the heavy chain comprises the amino acid sequence of SEQ ID NO: 49.
   451. The method according to any one of embodiments 349-429 or 450, wherein the light chain comprises the amino acid sequence of SEQ ID NO: 50.
   452. The method according to any one of embodiments 349-429 or 450-451, wherein the anti-amyloid beta antibody is ponezumab.
   453. The method according to any one of embodiments 349-429 or 450-452, wherein the anti-amyloid beta antibody or antigen binding fragment thereof binds to the same conformational epitope of amyloid beta to which ponezumab binds.
   454. The method according to any one of embodiments 349-429 or 450-452, wherein the anti-amyloid beta antibody or antigen binding fragment thereof competitively inhibits binding of ponezumab to amyloid beta.
   455. The method according to any one of embodiments 349-429, wherein the heavy chain comprises the amino acid sequence of SEQ ID NO: 59.
   456. The method according to any one of embodiments 349-429 or 455, wherein the light chain comprises the amino acid sequence of SEQ ID NO: 60.
   457. The method according to any one of embodiments 349-429 or 455-456, wherein the anti-amyloid beta antibody is bapineuzumab.
   458. The method according to any one of embodiments 349-429 or 455-457, wherein the anti-amyloid beta antibody or antigen binding fragment thereof binds to the same conformational epitope of amyloid beta to which bapineuzumab binds.
   459. The method according to any one of embodiments 349-429 or 455-457, wherein the anti-amyloid beta antibody or antigen binding fragment thereof competitively inhibits binding of bapineuzumab to amyloid beta.
   460. The method according to any one of embodiments 349-429, wherein the heavy chain comprises the amino acid sequence of SEQ ID NO: 69.
   461. The method according to any one of embodiments 349-429 or 460, wherein the light chain comprises the amino acid sequence of SEQ ID NO: 70.
   462. The method according to any one of embodiments 349-429 or 460-461, wherein the anti-amyloid beta antibody is lecanumab.
   463. The method according to any one of embodiments 349-429 or 460-462, wherein the anti-amyloid beta antibody or antigen binding fragment thereof binds to the same conformational epitope of amyloid beta to which lecanumab binds.
   464. The method according to any one of embodiments 349-429 or 460-462, wherein the anti-amyloid beta antibody or antigen binding fragment thereof competitively inhibits binding of lecanumab to amyloid beta.
   465. The method according to any one of embodiments 349-464, wherein the anti-amyloid beta antibody or antigen binding fragment thereof binds to amyloid beta plaques, parenchymal amyloid, cerebrovascular amyloid, or diffuse amyloid beta deposits.
   466. The method according to any one of embodiments 349-465, wherein the CYP46A1 inhibitor and the anti-amyloid beta antibody or antigen binding fragment thereof are administered simultaneously.
   467. The method according to any one of embodiments 349-465, wherein the CYP46A1 inhibitor and the anti-amyloid beta antibody or antigen binding fragment thereof are administered sequentially.
   468. The method according to any one of embodiments 349-465, wherein the CYP46A1 inhibitor and the anti-amyloid beta antibody or antigen binding fragment thereof are administered separately.
   469. The method according to embodiments 467 or 468, wherein the anti-amyloid beta antibody or antigen binding fragment thereof is administered before the CYP46A1 inhibitor.
   470. The method according to embodiments 467 or 468, wherein the CYP46A1 inhibitor is administered before the anti-amyloid beta antibody or antigen binding fragment thereof.
   471. The method according to any one of embodiments 349-470, wherein the frequency of administration of the anti-amyloid beta antibody or antigen binding fragment thereof and the CYP46A1 inhibitor is the same.
   472. The method according to any one of embodiments 349-470, wherein the frequency of administration of the anti-amyloid beta antibody or antigen binding fragment thereof and the CYP46A1 inhibitor is different.
   473. Use of (a) an NMDA receptor positive allosteric modulator, and (b) an anti-amyloid beta antibody or antigen-binding fragment thereof, for the manufacture of a medicament for treating or preventing Alzheimer's disease in a subject.
   474. Use of (a) an NMDA receptor positive allosteric modulator, and (b) an anti-amyloid beta antibody or antigen-binding fragment thereof, for the manufacture of a medicament for treating or preventing cognitive impairment in a subject having Alzheimer's disease.
   475. Use of (a) an NMDA receptor positive allosteric modulator, and (b) an anti-amyloid beta antibody or antigen-binding fragment thereof, for the manufacture of a medicament for treating or preventing cognitive impairment due to Alzheimer's disease in a subject.
   476. The use of embodiment 474 or 475, wherein the cognitive impairment is mild cognitive impairment.
   477. Use of (a) an NMDA receptor positive allosteric modulator, and (b) an anti-amyloid beta antibody or antigen-binding fragment thereof, for the manufacture of a medicament for improving cognition in a subject having Alzheimer's disease.
   478. Use of (a) an NMDA receptor positive allosteric modulator, and (b) an anti-amyloid beta antibody or antigen-binding fragment thereof, for the manufacture of a medicament for slowing cognitive decline due to Alzheimer's disease in a subject.
   479. Use of (a) a CYP46A1 inhibitor, and (b) an anti-amyloid beta antibody or antigen-binding fragment thereof, for the manufacture of a medicament treating or preventing Alzheimer's disease in a subject.
   480. Use of (a) a CYP46A1 inhibitor, and (b) an anti-amyloid beta antibody or antigen-binding fragment thereof, for the manufacture of a medicament for treating or preventing cognitive impairment in a subject having Alzheimer's disease.
   481. Use of (a) a CYP46A1 inhibitor, and (b) an anti-amyloid beta antibody or antigen-binding fragment thereof, for the manufacture of a medicament for treating or preventing cognitive impairment due to Alzheimer's disease in a subject.
   482. The use embodiment 480 or 481, wherein the cognitive impairment is mild cognitive impairment.
   483. Use of (a) a CYP46A1 inhibitor, and (b) an anti-amyloid beta antibody or antigen-binding fragment thereof, for the manufacture of a medicament for improving cognition in a subject having Alzheimer's disease.
   484. Use of (a) a CYP46A1 inhibitor, and (b) an anti-amyloid beta antibody or antigen-binding fragment thereof, for the manufacture of a medicament for slowing cognitive decline due to Alzheimer's disease in a subject.
   485. The use according to any one of embodiments 473-484, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   486. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   487. The use according to embodiment 486, wherein the heavy chain variable domain (VH) comprises the amino acid sequence of SEQ ID NO: 7.
   488. The use according to embodiment 486, wherein the light chain variable domain (VL) comprises the amino acid sequence of SEQ ID NO: 8.
   489. The use according to embodiment 486, wherein the heavy chain variable domain (VH) comprises the amino acid sequence of SEQ ID NO: 7; and the light chain variable domain (VL) comprises the amino acid sequence of SEQ ID NO: 8.
   490. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and
(b) a light chain variable domain (VL) comprising (i) LCDR1 comprising the amino acid sequence of SEQ ID NO: 14, (ii) LCDR2 comprising the amino acid sequence of SEQ ID NO: 15, and (iii) LCDR3 comprising the amino acid sequence of SEQ ID NO: 16.
   491. The use according to embodiment 490, wherein the heavy chain variable domain (VH) comprises the amino acid sequence of SEQ ID NO: 17.
   492. The use according to embodiment 490, wherein the light chain variable domain (VL) comprises the amino acid sequence of SEQ ID NO: 18.
   493. The use according to embodiment 490, wherein the heavy chain variable domain (VH) comprises the amino acid sequence of SEQ ID NO: 17; and the light chain variable domain (VL) comprises the amino acid sequence of SEQ ID NO: 18.
   494. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 21, (ii) HCDR2 comprising the amino acid sequence of SEQ ID NO: 22, and (iii) HCDR3 comprising the amino acid sequence of SEQ ID NO: 23; and
(b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.
   495. The use according to embodiment 494, wherein the heavy chain variable domain (VH) comprises the amino acid sequence of SEQ ID NO: 27.
   496. The use according to embodiment 494, wherein the light chain variable domain (VL) comprises the amino acid sequence of SEQ ID NO: 28.
   497. The use according to embodiment 494, wherein the heavy chain variable domain (VH) comprises the amino acid sequence of SEQ ID NO: 27; and the light chain variable domain (VL) comprises the amino acid sequence of SEQ ID NO: 28.
   498. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and
(b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.
   499. The use according to embodiment 498, wherein the heavy chain variable domain (VH) comprises the amino acid sequence of SEQ ID NO: 37.
   500. The use according to embodiment 498, wherein the light chain variable domain (VL) comprises the amino acid sequence of SEQ ID NO: 38.
   501. The use according to embodiment 498, wherein the heavy chain variable domain (VH) comprises the amino acid sequence of SEQ ID NO: 37; and the light chain variable domain (VL) comprises the amino acid sequence of SEQ ID NO: 38.
   502. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and
(b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.
   503. The use according to embodiment 502, wherein the heavy chain variable domain (VH) comprises the amino acid sequence of SEQ ID NO: 47.
   504. The use according to embodiment 502, wherein the light chain variable domain (VL) comprises the amino acid sequence of SEQ ID NO: 48.
   505. The use according to embodiment 502, wherein the heavy chain variable domain (VH) comprises the amino acid sequence of SEQ ID NO: 47; and the light chain variable domain (VL) comprises the amino acid sequence of SEQ ID NO: 48.
   506. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and
(b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.
   507. The use according to embodiment 506, wherein the heavy chain variable domain (VH) comprises the amino acid sequence of SEQ ID NO: 57.
   508. The use according to embodiment 506, wherein the light chain variable domain (VL) comprises the amino acid sequence of SEQ ID NO: 58.
   509. The use according to embodiment 506, wherein the heavy chain variable domain (VH) comprises the amino acid sequence of SEQ ID NO: 57; and the light chain variable domain (VL) comprises the amino acid sequence of SEQ ID NO: 58.
   510. The use according to any one of embodiments 473-484, wherein the anti-amyloid beta antibody or antigen-binding fragment thereof comprises:
(a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and
(b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.
   511. The use according to embodiment 510, wherein the heavy chain variable domain (VH) comprises the amino acid sequence of SEQ ID NO: 67.
   512. The use according to embodiment 510, wherein the light chain variable domain (VL) comprises the amino acid sequence of SEQ ID NO: 68.
   513. The use according to embodiment 510, wherein the heavy chain variable domain (VH) comprises the amino acid sequence of SEQ ID NO: 67; and the light chain variable domain (VL) comprises the amino acid sequence of SEQ ID NO: 68.
   514. The use according to any one of embodiments 473-513, wherein said anti-amyloid beta antibody or antigen-binding fragment thereof comprises a human IgG1, IgG2, IgG3, or IgG4 constant domain.
   515. The use according to embodiment 514, wherein the anti-amyloid beta antibody or antigen-binding fragment thereof comprises a human IgG1 constant region.
   516. The use according to any one of embodiments 473-515, wherein the anti-amyloid beta antibody or antigen-binding fragment thereof is a human anti-amyloid beta antibody or antigen-binding fragment thereof.
   517. The use according to any one of embodiments 473-516, wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   518. The use according to any one of embodiments 473-517, wherein the heavy chain comprises the amino acid sequence of SEQ ID NO: 9.
   519. The use according to any one of embodiments 473-518, wherein the light chain comprises the amino acid sequence of SEQ ID NO: 10.
   520. The use according to any one of embodiments 473-519, wherein the anti-amyloid beta antibody is aducanumab.
   521. The use according to any one of embodiments 473-520, wherein the anti-amyloid beta antibody or antigen binding fragment thereof binds to the same conformational epitope of amyloid beta to which aducanumab binds.
   522. The use according to any one of embodiments 473-521, wherein the anti-amyloid beta antibody or antigen binding fragment thereof competitively inhibits binding of aducanumab to amyloid beta.
   523. The use according to any one of embodiments 473-517, wherein the heavy chain comprises the amino acid sequence of SEQ ID NO: 19.
   524. The use according to any one of embodiments 473-517 or 523, wherein the light chain comprises the amino acid sequence of SEQ ID NO: 20.
   525. The use according to any one of embodiments 473-517 or 523-524, wherein the anti-amyloid beta antibody is solazenumab.
   526. The use according to any one of embodiments 473-517 or 523-525, wherein the anti-amyloid beta antibody or antigen binding fragment thereof binds to the same conformational epitope of amyloid beta to which solazenumab binds.
   527. The use according to any one of embodiments 473-517 or 523-525, wherein the anti-amyloid beta antibody or antigen binding fragment thereof competitively inhibits binding of solazenumab to amyloid beta.
   528. The use according to any one of embodiments 473-517, wherein the heavy chain comprises the amino acid sequence of SEQ ID NO: 29.
   529. The use according to any one of embodiments 473-517 or 528, wherein the light chain comprises the amino acid sequence of SEQ ID NO: 30.
   530. The use according to any one of embodiments 473-517 or 528-529, wherein the anti-amyloid beta antibody is crenezumab.
   531. The use according to any one of embodiments 473-517 or 528-530, wherein the anti-amyloid beta antibody or antigen binding fragment thereof binds to the same conformational epitope of amyloid beta to which crenezumab binds.
   532. The use according to any one of embodiments 473-517 or 528-530, wherein the anti-amyloid beta antibody or antigen binding fragment thereof competitively inhibits binding of crenezumab to amyloid beta.
   533. The use according to any one of embodiments 473-517, wherein the heavy chain comprises the amino acid sequence of SEQ ID NO: 39.
   534. The use according to any one of embodiments 473-517 or 533, wherein the light chain comprises the amino acid sequence of SEQ ID NO: 40.
   535. The use according to any one of embodiments 473-517 or 533-534, wherein the anti-amyloid beta antibody is gantenerumab.
   536. The use according to any one of embodiments 473-517 or 533-535, wherein the anti-amyloid beta antibody or antigen binding fragment thereof binds to the same conformational epitope of amyloid beta to which gantenerumab binds.
   537. The use according to any one of embodiments 473-517 and 533-535, wherein the anti-amyloid beta antibody or antigen binding fragment thereof competitively inhibits binding of gantenerumab to amyloid beta.
   538. The use according to any one of embodiments 473-517, wherein the heavy chain comprises the amino acid sequence of SEQ ID NO: 49.
   539. The use according to any one of embodiments 473-517 or 538, wherein the light chain comprises the amino acid sequence of SEQ ID NO: 50.
   540. The use according to any one of embodiments 473-517 or 538-539, wherein the anti-amyloid beta antibody is ponezumab.
   541. The use according to any one of embodiments 473-517 or 538-540, wherein the anti-amyloid beta antibody or antigen binding fragment thereof binds to the same conformational epitope of amyloid beta to which ponezumab binds.
   542. The use according to any one of embodiments 473-517 or 538-540, wherein the anti-amyloid beta antibody or antigen binding fragment thereof competitively inhibits binding of ponezumab to amyloid beta.
   543. The use according to any one of embodiments 473-517, wherein the heavy chain comprises the amino acid sequence of SEQ ID NO: 59.
   544. The use according to any one of embodiments 473-517 or 543, wherein the light chain comprises the amino acid sequence of SEQ ID NO: 60.
   545. The use according to any one of embodiments 473-517 or 543-544, wherein the anti-amyloid beta antibody is bapineuzumab.
   546. The use according to any one of embodiments 473-517 or 543-545, wherein the anti-amyloid beta antibody or antigen binding fragment thereof binds to the same conformational epitope of amyloid beta to which bapineuzumab binds.
   547. The use according to any one of embodiments 473-517 or 543-545, wherein the anti-amyloid beta antibody or antigen binding fragment thereof competitively inhibits binding of bapineuzumab to amyloid beta.
   548. The use according to any one of embodiments 473-517, wherein the heavy chain comprises the amino acid sequence of SEQ ID NO: 69.
   549. The use according to any one of embodiments 473-517 or 548, wherein the light chain comprises the amino acid sequence of SEQ ID NO: 70.
   550. The use according to any one of embodiments 473-517 or 548-549, wherein the anti-amyloid beta antibody is lecanumab.
   551. The use according to any one of embodiments 473-517 or 548-550, wherein the anti-amyloid beta antibody or antigen binding fragment thereof binds to the same conformational epitope of amyloid beta to which lecanumab binds.
   552. The use according to any one of embodiments 473-517 or 548-550, wherein the anti-amyloid beta antibody or antigen binding fragment thereof competitively inhibits binding of lecanumab to amyloid beta.
   553. The use according to any one of embodiments 473-552, wherein the anti-amyloid beta antibody or antigen binding fragment thereof binds to amyloid beta plaques, parenchymal amyloid, cerebrovascular amyloid, or diffuse amyloid beta deposits.
   554. The use according to any one of embodiments 473-478 or 485-553, wherein the NMDA receptor positive allosteric modulator is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl) (6,7-dimethoxy-1-[(4-methoxyphenoxy)methyl]-3,4-dihydroisoquinolin-2 (1H)-yl)methanone (CIQ), NYX-458, and plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one) and pharmaceutically acceptable salts thereof.
   555. The use according to any one of embodiments 473-478 or 485-553, wherein the NMDA receptor positive allosteric modulator is a neuroactive steroid, or a pharmaceutically acceptable salt thereof.
   556. The use according to embodiment 555, wherein the neuroactive steroid is selected from any one of compounds B1-B543; and pharmaceutically acceptable salts thereof.
   557. The use according to embodiment 555, wherein the neuroactive steroid is selected from any one of compounds B1-B140, and pharmaceutically acceptable salts thereof.
   558. The use according to embodiment 555, wherein the neuroactive steroid is selected from any one of compounds B150-B245, and pharmaceutically acceptable salts thereof.
   559. The use according to embodiment 555, wherein the neuroactive steroid is selected from any one of compounds B273-B349, and pharmaceutically acceptable salts thereof.
   560. The use according to embodiment 555, wherein the neuroactive steroid is selected from any one of compounds B395-B432, and pharmaceutically acceptable salts thereof.
   560A. The pharmaceutical composition according to embodiment 555, wherein the neuroactive steroid is selected from any one of compounds B433-B443 and pharmaceutically acceptable salts thereof and pharmaceutically acceptable salts thereof.
   560B. The pharmaceutical composition according to embodiment 555, wherein the neuroactive steroid is selected from any one of compounds B444-B451 and pharmaceutically acceptable salts thereof and pharmaceutically acceptable salts thereof.
   560C. The pharmaceutical composition according to embodiment 555, wherein the neuroactive steroid is selected from any one of compounds B452-B477 and pharmaceutically acceptable salts thereof and pharmaceutically acceptable salts thereof.
   560D. The pharmaceutical composition according to embodiment 555, wherein the neuroactive steroid is selected from any one of compounds B478-B530 and pharmaceutically acceptable salts thereof and pharmaceutically acceptable salts thereof.
   560E. The pharmaceutical composition according to embodiment 555, wherein the neuroactive steroid is selected from any one of compounds B531-B543 and pharmaceutically acceptable salts thereof and pharmaceutically acceptable salts thereof.
   561. The use according to any one of embodiments 473-478 or 485-560E, wherein the NMDA receptor positive allosteric modulator and the anti-amyloid beta antibody or antigen binding fragment thereof are administered simultaneously.
   562. The use according to any one of embodiments 473-478 or 485-560E, wherein the NMDA receptor positive allosteric modulator and the anti-amyloid beta antibody or antigen binding fragment thereof are administered sequentially.
   563. The use according to any one of embodiments 473-478 or 485-560E, wherein the NMDA receptor positive allosteric modulator and the anti-amyloid beta antibody or antigen binding fragment thereof are administered separately.
   564. The use according to embodiments 562 or 563 wherein the anti-amyloid beta antibody or antigen binding fragment thereof is administered before the NMDA receptor positive allosteric modulator.
   565. The use according to embodiments 563 or 564, wherein the NMDA receptor positive allosteric modulator is administered before the anti-amyloid beta antibody or antigen binding fragment thereof.
   566. The use according to any one of embodiments 473-478 or 485-565, wherein the frequency of administration of the anti-amyloid beta antibody or antigen binding fragment thereof and the NMDA receptor positive allosteric modulator is the same.
   567. The use according to any one of embodiments 473-473 or 485-565, wherein the frequency of administration of the anti-amyloid beta antibody or antigen binding fragment thereof and the NMDA receptor positive allosteric modulator is different.
   568. The use according to any one of embodiments 479-5534, wherein the CYP46A1 inhibitor is selected from the group consisting of TAK-935, any one of compounds A1-A182, and pharmaceutically acceptable salts thereof.
   569. The use according to embodiment 568, wherein the CYP46A1 inhibitor is TAK-935, or a pharmaceutically acceptable salt thereof.
   570. The use according to embodiment 568, wherein the CYP46A1 inhibitor is selected from the group consisting of any one of compounds A1-A182, and pharmaceutically acceptable salts thereof.
   571. The use according to any one of embodiments 479-553 or 568-570, wherein the CYP46A1 inhibitor and the anti-amyloid beta antibody or antigen binding fragment thereof are administered simultaneously.
   572. The use according to any one of embodiments 479-553 or 568-570, wherein the CYP46A1 inhibitor and the anti-amyloid beta antibody or antigen binding fragment thereof are administered sequentially.
   573. The use according to any one of embodiments 479-553 or 568-570, wherein the CYP46A1 inhibitor and the anti-amyloid beta antibody or antigen binding fragment thereof are administered separately.
   574. The use according to embodiments 572 or 573, wherein the anti-amyloid beta antibody or antigen binding fragment thereof is administered before the CYP46A1 inhibitor.
   575. The use according to embodiments 572 or 573, wherein the CYP46A1 inhibitor is administered before the anti-amyloid beta antibody or antigen binding fragment thereof.
   576. The use according to any one of embodiments 479-553 or 568-575, wherein the frequency of administration of the anti-amyloid beta antibody or antigen binding fragment thereof and the CYP46A1 inhibitor is the same.
   577. The use according to any one of embodiments 479-553 or 569-575, wherein the frequency of administration of the anti-amyloid beta antibody or antigen binding fragment thereof and the CYP46A1 inhibitor is different.
   578. An NMDA receptor positive allosteric modulator, and the anti-amyloid beta antibody or antigen-binding fragment thereof, for use in treating or preventing Alzheimer's disease in a subject.
   579. An NMDA receptor positive allosteric modulator, and the anti-amyloid beta antibody or antigen-binding fragment thereof, for use in treating or preventing cognitive impairment in a subject having Alzheimer's disease.
   580. An NMDA receptor positive allosteric modulator, and the anti-amyloid beta antibody or antigen-binding fragment thereof, for use in treating or preventing cognitive impairment due to Alzheimer's disease in a subject.
   581. The NMDA receptor positive allosteric modulator, and the anti-amyloid beta antibody or antigen-binding fragment thereof for use according to embodiment 579 or 580, wherein the cognitive impairment is mild cognitive impairment.
   582. An NMDA receptor positive allosteric modulator, and the anti-amyloid beta antibody or antigen-binding fragment thereof, for use in improving cognition in a subject having Alzheimer's disease.
   583. An NMDA receptor positive allosteric modulator, and the anti-amyloid beta antibody or antigen-binding fragment thereof, for use in slowing cognitive decline due to Alzheimer's disease in a subject.
   584. The NMDA receptor positive allosteric modulator, and the anti-amyloid beta antibody or antigen-binding fragment thereof, for use according to any one of embodiments 578-583, wherein the NMDA receptor positive allosteric modulator is selected from the group consisting of 9-iodophenanthrene-3-carboxylic acid (UBP512), 6-(4-methylpentyl) naphthalene-2-carboxylic acid (UBP684), 4-Cyclohexyl-N-(7-hydroxy-5-methyl-2-propyl[1,2,4]triazolo[1,5-a]pyrimidin-6-yl)benzenesulfonamide (GNE-9278), 2-butyl-7-((ethyl(phenyl)amino)methyl)-5H-[1,3,4]thiadiazolo[3,2-a]pyrimidin-5-one (GNE-3476), methyl 4-(4-hydroxy-1-(2-(6-methyl-1H-indol-3-yl)ethyl)-3-nicotinoyl-5-oxo-2,5-dihydro-1H-pyrrol-2-yl)benzoate (PYD-111), methyl 4-(3-acetyl-4-hydroxy-1-(2-(2-methyl-1H-indol-3-yl)ethyl)-5-oxo-2,5-dihydro-1H-pyrrol-2-yl)benzoate (PYD-106), 3-chlorophenyl) (6,7-dimethoxy-1-[(4-methoxyphenoxy)methyl]-3,4-dihydroisoquinolin-2 (1H)-yl)methanone (CIQ), NYX-458, and plazinemdor (5-(3-chloro-4-fluorophenyl)-7-cyclopropyl-3-(2-(3-fluoro-3-methylazetidin-1-yl)-2-oxoethyl)-3,7-dihydro-4H-pyrrolo[2,3-d]pyrimidin-4-one) and pharmaceutically acceptable salts thereof.
   585. The NMDA receptor positive allosteric modulator, and the anti-amyloid beta antibody or antigen-binding fragment thereof, for use according to any one of embodiments
   578-583, wherein the NMDA receptor positive allosteric modulator is a neuroactive steroid, or a pharmaceutically acceptable salt thereof.
   586. The NMDA receptor positive allosteric modulator, and the anti-amyloid beta antibody or antigen-binding fragment thereof, for use according to embodiment 585, wherein the neuroactive steroid is selected from any one of compounds B1-B543; and pharmaceutically acceptable salts thereof.
   587. The NMDA receptor positive allosteric modulator, and the anti-amyloid beta antibody or antigen-binding fragment thereof, for use according to embodiment 585, wherein the neuroactive steroid is selected from any one of compounds B1-B140, and pharmaceutically acceptable salts thereof.
   588. The NMDA receptor positive allosteric modulator, and the anti-amyloid beta antibody or antigen-binding fragment thereof, for use according to embodiment 585, wherein the neuroactive steroid is selected from any one of compounds B150-B245, and pharmaceutically acceptable salts thereof.
   589. The NMDA receptor positive allosteric modulator, and the anti-amyloid beta antibody or antigen-binding fragment thereof, for use according to embodiment 585, wherein the neuroactive steroid is selected from any one of compounds B273-B349, and pharmaceutically acceptable salts thereof.
   590. The NMDA receptor positive allosteric modulator, and the anti-amyloid beta antibody or antigen-binding fragment thereof, for use according to embodiment 585, wherein the neuroactive steroid is selected from any one of compounds B395-B432, and pharmaceutically acceptable salts thereof.
   590A. The pharmaceutical composition according to embodiment 585 wherein the neuroactive steroid is selected from any one of compounds B433-B443 and pharmaceutically acceptable salts thereof and pharmaceutically acceptable salts thereof.
   590B. The pharmaceutical composition according to embodiment 585, wherein the neuroactive steroid is selected from any one of compounds B444-B451 and pharmaceutically acceptable salts thereof and pharmaceutically acceptable salts thereof.
   590C. The pharmaceutical composition according to embodiment 585, wherein the neuroactive steroid is selected from any one of compounds B452-B477 and pharmaceutically acceptable salts thereof and pharmaceutically acceptable salts thereof.
   590D. The pharmaceutical composition according to embodiment 585, wherein the neuroactive steroid is selected from any one of compounds B478-B530 and pharmaceutically acceptable salts thereof and pharmaceutically acceptable salts thereof.
   5910E. The pharmaceutical composition according to embodiment 585, wherein the neuroactive steroid is selected from any one of compounds B531-B543 and pharmaceutically acceptable salts thereof and pharmaceutically acceptable salts thereof.
   591. The NMDA receptor positive allosteric modulator, and the anti-amyloid beta antibody or antigen-binding fragment thereof, for use according to any one of embodiments 578-590E, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   592. The NMDA receptor positive allosteric modulator, and the anti-amyloid beta antibody or antigen-binding fragment thereof, for use according to any one of embodiments 578-590E, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   593. The NMDA receptor positive allosteric modulator, and the anti-amyloid beta antibody or antigen-binding fragment thereof, for use according to embodiment 592, wherein the heavy chain variable domain (VH) comprises the amino acid sequence of SEQ ID NO: 7.
   594. The NMDA receptor positive allosteric modulator, and the anti-amyloid beta antibody or antigen-binding fragment thereof, for use according to embodiment 592, wherein the light chain variable domain (VL) comprises the amino acid sequence of SEQ ID NO: 8.
   595. The NMDA receptor positive allosteric modulator, and the anti-amyloid beta antibody or antigen-binding fragment thereof, for use according to embodiment 592, wherein the heavy chain variable domain (VH) comprises the amino acid sequence of SEQ ID NO: 7; and the light chain variable domain (VL) comprises the amino acid sequence of SEQ ID NO: 8.
   596. The NMDA receptor positive allosteric modulator, and the anti-amyloid beta antibody or antigen-binding fragment thereof, for use according to any one of embodiments 578-590, wherein the anti-amyloid beta antibody or antigen-binding fragment thereof comprises:
(a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and
(b) a light chain variable domain (VL) comprising (i) LCDR1 comprising the amino acid sequence of SEQ ID NO: 4, (ii) LCDR2 comprising the amino acid sequence of SEQ ID NO: 15, and (iii) LCDR3 comprising the amino acid sequence of SEQ ID NO: 16.
   597. The NMDA receptor positive allosteric modulator, and the anti-amyloid beta antibody or antigen-binding fragment thereof, for use according to embodiment 596, wherein the heavy chain variable domain (VH) comprises the amino acid sequence of SEQ ID NO: 17.
   598. The NMDA receptor positive allosteric modulator, and the anti-amyloid beta antibody or antigen-binding fragment thereof, for use according to embodiment 596, wherein the light chain variable domain (VL) comprises the amino acid sequence of SEQ ID NO: 18.
   599. The NMDA receptor positive allosteric modulator, and the anti-amyloid beta antibody or antigen-binding fragment thereof, for use according to embodiment 596, wherein the heavy chain variable domain (VH) comprises the amino acid sequence of SEQ ID NO: 17; and the light chain variable domain (VL) comprises the amino acid sequence of SEQ ID NO: 18.
   600. The NMDA receptor positive allosteric modulator, and the anti-amyloid beta antibody or antigen-binding fragment thereof, for use according to any one of embodiments 578-590, wherein the anti-amyloid beta antibody or antigen-binding fragment thereof comprises:
(a) a heavy chain variable domain (VH) comprising (i) HCDR1 comprising the amino acid sequence of SEQ ID NO: 221, (ii) HCDR2 comprising the amino acid sequence of SEQ ID NO: 2, and (iii) HCDR3 comprising the amino acid sequence of SEQ ID NO: 23; and
(b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.
   601. The NMDA receptor positive allosteric modulator, and the anti-amyloid beta antibody or antigen-binding fragment thereof, for use according to embodiment 600, wherein the heavy chain variable domain (VH) comprises the amino acid sequence of SEQ ID NO: 27.
   602. The NMDA receptor positive allosteric modulator, and the anti-amyloid beta antibody or antigen-binding fragment thereof, for use according to embodiment 600, wherein the light chain variable domain (VL) comprises the amino acid sequence of SEQ ID NO: 28.
   603. The NMDA receptor positive allosteric modulator, and the anti-amyloid beta antibody or antigen-binding fragment thereof, for use according to embodiment 600, wherein the heavy chain variable domain (VH) comprises the amino acid sequence of SEQ ID NO: 27; and the light chain variable domain (VL) comprises the amino acid sequence of SEQ ID NO: 28.
   604. The NMDA receptor positive allosteric modulator, and the anti-amyloid beta antibody or antigen-binding fragment thereof, for use according to any one of embodiments 578-590, wherein the anti-amyloid beta antibody or antigen-binding fragment thereof comprises:
(a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and
(b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.
   605. The NMDA receptor positive allosteric modulator, and the anti-amyloid beta antibody or antigen-binding fragment thereof, for use according to embodiment 604, wherein the heavy chain variable domain (VH) comprises the amino acid sequence of SEQ ID NO: 37.
   606. The NMDA receptor positive allosteric modulator, and the anti-amyloid beta antibody or antigen-binding fragment thereof, for use according to embodiment 604, wherein the light chain variable domain (VL) comprises the amino acid sequence of SEQ ID NO: 38.
   607. The NMDA receptor positive allosteric modulator, and the anti-amyloid beta antibody or antigen-binding fragment thereof, for use according to embodiment 604, wherein the heavy chain variable domain (VH) comprises the amino acid sequence of SEQ ID NO: 37; and the light chain variable domain (VL) comprises the amino acid sequence of SEQ ID NO: 38.
   608. The NMDA receptor positive allosteric modulator, and the anti-amyloid beta antibody or antigen-binding fragment thereof, for use according to any one of embodiments 578-590, wherein the anti-amyloid beta antibody or antigen-binding fragment thereof comprises:
(a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and
(b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.
   609. The NMDA receptor positive allosteric modulator, and the anti-amyloid beta antibody or antigen-binding fragment thereof, for use according to embodiment 609, wherein the heavy chain variable domain (VH) comprises the amino acid sequence of SEQ ID NO: 47.
   610. The NMDA receptor positive allosteric modulator, and the anti-amyloid beta antibody or antigen-binding fragment thereof, for use according to embodiment 609, wherein the light chain variable domain (VL) comprises the amino acid sequence of SEQ ID NO: 48.
   611. The NMDA receptor positive allosteric modulator, and the anti-amyloid beta antibody or antigen-binding fragment thereof, for use according to embodiment 609, wherein the heavy chain variable domain (VH) comprises the amino acid sequence of SEQ ID NO: 47; and the light chain variable domain (VL) comprises the amino acid sequence of SEQ ID NO: 48.
   612. The NMDA receptor positive allosteric modulator, and the anti-amyloid beta antibody or antigen-binding fragment thereof, for use according to any one of embodiments 578-590, wherein the anti-amyloid beta antibody or antigen-binding fragment thereof comprises:
(a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and
(b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.
   613. The NMDA receptor positive allosteric modulator, and the anti-amyloid beta antibody or antigen-binding fragment thereof, for use according to embodiment 612, wherein the heavy chain variable domain (VH) comprises the amino acid sequence of SEQ ID NO: 57.
   614. The NMDA receptor positive allosteric modulator, and the anti-amyloid beta antibody or antigen-binding fragment thereof, for use according to embodiment 612, wherein the light chain variable domain (VL) comprises the amino acid sequence of SEQ ID NO: 58.
   615. The NMDA receptor positive allosteric modulator, and the anti-amyloid beta antibody or antigen-binding fragment thereof, for use according to embodiment 612, wherein the heavy chain variable domain (VH) comprises the amino acid sequence of SEQ ID NO: 57; and the light chain variable domain (VL) comprises the amino acid sequence of SEQ ID NO: 58.
   616. The NMDA receptor positive allosteric modulator, and the anti-amyloid beta antibody or antigen-binding fragment thereof, for use according to any one of embodiments 578-590, wherein the anti-amyloid beta antibody or antigen-binding fragment thereof comprises:
(a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and
(b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.
   617. The NMDA receptor positive allosteric modulator, and the anti-amyloid beta antibody or antigen-binding fragment thereof, for use according to embodiment 616, wherein the heavy chain variable domain (VH) comprises the amino acid sequence of SEQ ID NO: 67.
   618. The NMDA receptor positive allosteric modulator, and the anti-amyloid beta antibody or antigen-binding fragment thereof, for use according to embodiment 616, wherein the light chain variable domain (VL) comprises the amino acid sequence of SEQ ID NO: 68.
   619. The NMDA receptor positive allosteric modulator, and the anti-amyloid beta antibody or antigen-binding fragment thereof, for use according to embodiment 616, wherein the heavy chain variable domain (VH) comprises the amino acid sequence of SEQ ID NO: 67; and the light chain variable domain (VL) comprises the amino acid sequence of SEQ ID NO: 68.
   620. The NMDA receptor positive allosteric modulator, and the anti-amyloid beta antibody or antigen-binding fragment thereof, for use according to any one of embodiments 578-619, wherein said anti-amyloid beta antibody or antigen-binding fragment thereof comprises a human IgG1, IgG2, IgG3, or IgG4 constant domain.
   621. The NMDA receptor positive allosteric modulator, and the anti-amyloid beta antibody or antigen-binding fragment thereof, for use according to embodiment 620, wherein the anti-amyloid beta antibody or antigen-binding fragment thereof comprises a human IgG1 constant region.
   622. The NMDA receptor positive allosteric modulator, and the anti-amyloid beta antibody or antigen-binding fragment thereof, for use according to any one of embodiments 578-621, wherein the anti-amyloid beta antibody or antigen-binding fragment thereof is a human anti-amyloid beta antibody or antigen-binding fragment thereof.
   623. The NMDA receptor positive allosteric modulator, and the anti-amyloid beta antibody or antigen-binding fragment thereof, for use according to any one of embodiments 578-622, wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   624. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 9.
   625. The NMDA receptor positive allosteric modulator, and the anti-amyloid beta antibody or antigen-binding fragment thereof, for use according to any one of embodiments 578-624, wherein the light chain comprises the amino acid sequence of SEQ ID NO: 10.
   626. The NMDA receptor positive allosteric modulator, and the anti-amyloid beta antibody or antigen-binding fragment thereof, for use according to any one of embodiments 578-625, wherein the anti-amyloid beta antibody is aducanumab.
   627. The NMDA receptor positive allosteric modulator, and the anti-amyloid beta antibody or antigen-binding fragment thereof, for use according to any one of embodiments 578-626, wherein the anti-amyloid beta antibody or antigen binding fragment thereof binds to the same conformational epitope of amyloid beta to which aducanumab binds.
   628. The NMDA receptor positive allosteric modulator, and the anti-amyloid beta antibody or antigen-binding fragment thereof, for use according to any one of embodiments 578-627, wherein the anti-amyloid beta antibody or antigen binding fragment thereof competitively inhibits binding of aducanumab to amyloid beta.
   629. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 19.
   630. The NMDA receptor positive allosteric modulator, and the anti-amyloid beta antibody or antigen-binding fragment thereof, for use according to any one of embodiments 578-623 or 629, wherein the light chain comprises the amino acid sequence of SEQ ID NO: 20.
   631. The NMDA receptor positive allosteric modulator, and the anti-amyloid beta antibody or antigen-binding fragment thereof, for use according to any one of embodiments 578-623 or 629-630, wherein the anti-amyloid beta antibody is solazenumab.
   632. The NMDA receptor positive allosteric modulator, and the anti-amyloid beta antibody or antigen-binding fragment thereof, for use according to any one of embodiments 578-623 or 629-631, wherein the anti-amyloid beta antibody or antigen binding fragment thereof binds to the same conformational epitope of amyloid beta to which solazenumab binds.
   633. The NMDA receptor positive allosteric modulator, and the anti-amyloid beta antibody or antigen-binding fragment thereof, for use according to any one of embodiments 578-623 or 629-631, wherein the anti-amyloid beta antibody or antigen binding fragment thereof competitively inhibits binding of solazenumab to amyloid beta.
   634. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 29.
   635. The NMDA receptor positive allosteric modulator, and the anti-amyloid beta antibody or antigen-binding fragment thereof, for use according to any one of embodiments 578-623 or 634, wherein the light chain comprises the amino acid sequence of SEQ ID NO: 30.
   636. The NMDA receptor positive allosteric modulator, and the anti-amyloid beta antibody or antigen-binding fragment thereof, for use according to any one of embodiments 578-623 or 634-635, wherein the anti-amyloid beta antibody is crenezumab.
   637. The NMDA receptor positive allosteric modulator, and the anti-amyloid beta antibody or antigen-binding fragment thereof, for use according to any one of embodiments 578-623 or 634-636, wherein the anti-amyloid beta antibody or antigen binding fragment thereof binds to the same conformational epitope of amyloid beta to which crenezumab binds.
   638. The NMDA receptor positive allosteric modulator, and the anti-amyloid beta antibody or antigen-binding fragment thereof, for use according to any one of embodiments 578-623 or 634-636, wherein the anti-amyloid beta antibody or antigen binding fragment thereof competitively inhibits binding of crenezumab to amyloid beta.
   639. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 39.
   640. The NMDA receptor positive allosteric modulator, and the anti-amyloid beta antibody or antigen-binding fragment thereof, for use according to any one of embodiments 578-623 or 639, wherein the light chain comprises the amino acid sequence of SEQ ID NO: 40.
   641. The NMDA receptor positive allosteric modulator, and the anti-amyloid beta antibody or antigen-binding fragment thereof, for use according to any one of embodiments 578-623 or 639-640, wherein the anti-amyloid beta antibody is gantenerumab.
   642. The NMDA receptor positive allosteric modulator, and the anti-amyloid beta antibody or antigen-binding fragment thereof, for use according to any one of embodiments 578-623 or 639-641, wherein the anti-amyloid beta antibody or antigen binding fragment thereof binds to the same conformational epitope of amyloid beta to which gantenerumab binds.
   643. The NMDA receptor positive allosteric modulator, and the anti-amyloid beta antibody or antigen-binding fragment thereof, for use according to any one of embodiments 578-623 or 639-641, wherein the anti-amyloid beta antibody or antigen binding fragment thereof competitively inhibits binding of gantenerumab to amyloid beta.
   644. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 49.
   645. The NMDA receptor positive allosteric modulator, and the anti-amyloid beta antibody or antigen-binding fragment thereof, for use according to any one of embodiments 578-623 or 644, wherein the light chain comprises the amino acid sequence of SEQ ID NO: 50.
   646. The NMDA receptor positive allosteric modulator, and the anti-amyloid beta antibody or antigen-binding fragment thereof, for use according to any one of embodiments 578-623 or 644-645, wherein the anti-amyloid beta antibody is ponezumab.
   647. The NMDA receptor positive allosteric modulator, and the anti-amyloid beta antibody or antigen-binding fragment thereof, for use according to any one of embodiments 578-623 or 644-646, wherein the anti-amyloid beta antibody or antigen binding fragment thereof binds to the same conformational epitope of amyloid beta to which ponezumab binds.
   648. The NMDA receptor positive allosteric modulator, and the anti-amyloid beta antibody or antigen-binding fragment thereof, for use according to any one of embodiments 578-623 or 644-646, wherein the anti-amyloid beta antibody or antigen binding fragment thereof competitively inhibits binding of ponezumab to amyloid beta.
   649. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 59.
   650. The NMDA receptor positive allosteric modulator, and the anti-amyloid beta antibody or antigen-binding fragment thereof, for use according to any one of embodiments 578-623 or 649, wherein the light chain comprises the amino acid sequence of SEQ ID NO: 60.
   651. The NMDA receptor positive allosteric modulator, and the anti-amyloid beta antibody or antigen-binding fragment thereof, for use according to any one of embodiments 578-623 or 649-650, wherein the anti-amyloid beta antibody is bapineuzumab.
   652. The NMDA receptor positive allosteric modulator, and the anti-amyloid beta antibody or antigen-binding fragment thereof, for use according to any one of embodiments 578-623 or 649-651, wherein the anti-amyloid beta antibody or antigen binding fragment thereof binds to the same conformational epitope of amyloid beta to which a bapineuzumab binds.
   653. The NMDA receptor positive allosteric modulator, and the anti-amyloid beta antibody or antigen-binding fragment thereof, for use according to any one of embodiments 578-623 or 649-651, wherein the anti-amyloid beta antibody or antigen binding fragment thereof competitively inhibits binding of bapineuzumab to amyloid beta.
   654. The NMDA receptor positive allosteric modulator, and the anti-amyloid beta antibody or antigen-binding fragment thereof, for use according to any one of embodiments 578-623, wherein the heavy chain comprises the amino acid sequence of SEQ ID NO: 69.
   655. The NMDA receptor positive allosteric modulator, and the anti-amyloid beta antibody or antigen-binding fragment thereof, for use according to any one of embodiments 578-623 or 654, wherein the light chain comprises the amino acid sequence of SEQ ID NO: 70.
   656. The NMDA receptor positive allosteric modulator, and the anti-amyloid beta antibody or antigen-binding fragment thereof, for use according to any one of embodiments 578-623 or 654-655, wherein the anti-amyloid beta antibody is lecanumab.
   657. The NMDA receptor positive allosteric modulator, and the anti-amyloid beta antibody or antigen-binding fragment thereof, for use according to any one of embodiments 578-623 or 654-656, wherein the anti-amyloid beta antibody or antigen binding fragment thereof binds to the same conformational epitope of amyloid beta to which lecanumab binds.
   658. The NMDA receptor positive allosteric modulator, and the anti-amyloid beta antibody or antigen-binding fragment thereof, for use according to any one of embodiments 578-623 or 654-656, wherein the anti-amyloid beta antibody or antigen binding fragment thereof competitively inhibits binding of lecanumab to amyloid beta.
   659. The NMDA receptor positive allosteric modulator, and the anti-amyloid beta antibody or antigen-binding fragment thereof, for use according to any one of embodiments 578-658, wherein the anti-amyloid beta antibody or antigen binding fragment thereof binds to amyloid beta plaques, parenchymal amyloid, cerebrovascular amyloid, or diffuse amyloid beta deposits.
   660. The NMDA receptor positive allosteric modulator, and the anti-amyloid beta antibody or antigen-binding fragment thereof, for use according to any one of embodiments 578-659, wherein the NMDA receptor positive allosteric modulator and the anti-amyloid beta antibody or antigen binding fragment thereof are administered simultaneously.
   661. The NMDA receptor positive allosteric modulator, and the anti-amyloid beta antibody or antigen-binding fragment thereof, for use according to any one of embodiments 578-660, wherein the NMDA receptor positive allosteric modulator and the anti-amyloid beta antibody or antigen binding fragment thereof are administered sequentially.
   662. The NMDA receptor positive allosteric modulator, and the anti-amyloid beta antibody or antigen-binding fragment thereof, for use according to any one of embodiments 578-660, wherein the NMDA receptor positive allosteric modulator and the anti-amyloid beta antibody or antigen binding fragment thereof are administered separately.
   663. The NMDA receptor positive allosteric modulator, and the anti-amyloid beta antibody or antigen-binding fragment thereof, for use according to any one of embodiments 578-660, wherein the anti-amyloid beta antibody or antigen binding fragment thereof is administered before the NMDA receptor positive allosteric modulator.
   664. The NMDA receptor positive allosteric modulator, and the anti-amyloid beta antibody or antigen-binding fragment thereof, for use according to any one of embodiments 578-663, wherein the NMDA receptor positive allosteric modulator is administered before the anti-amyloid beta antibody or antigen binding fragment thereof.
   665. The NMDA receptor positive allosteric modulator, and the anti-amyloid beta antibody or antigen-binding fragment thereof, for use according to any one of embodiments 578-664, wherein the frequency of administration of the anti-amyloid beta antibody or antigen binding fragment thereof and the NMDA receptor positive allosteric modulator is the same.
   666. The NMDA receptor positive allosteric modulator, and the anti-amyloid beta antibody or antigen-binding fragment thereof, for use according to any one of embodiments 578-665, wherein the frequency of administration of the anti-amyloid beta antibody or antigen binding fragment thereof and the NMDA receptor positive allosteric modulator is different.
   667. A CYP46A1 inhibitor, and an anti-amyloid beta antibody or antigen-binding fragment thereof, for use in treating or preventing Alzheimer's disease in a subject.
   668. A CYP46A1 inhibitor, and an anti-amyloid beta antibody or antigen-binding fragment thereof, for use in treating or preventing cognitive impairment in a subject having Alzheimer's disease.
   669. A CYP46A1 inhibitor, and an anti-amyloid beta antibody or antigen-binding fragment thereof, for use in treating or preventing cognitive impairment due to Alzheimer's disease in a subject.
   670. The CYP46A1 inhibitor, and the anti-amyloid beta antibody or antigen-binding fragment thereof for use according to embodiment 668 or 669, wherein the cognitive impairment is mild cognitive impairment.
   671. A CYP46A1 inhibitor, and an anti-amyloid beta antibody or antigen-binding fragment thereof, for use in improving cognition in a subject having Alzheimer's disease.
   672. A CYP46A1 inhibitor, and an anti-amyloid beta antibody or antigen-binding fragment thereof, for use in slowing cognitive decline due to Alzheimer's disease in a subject.
   673. The CYP46A1 inhibitor, and the anti-amyloid beta antibody or antigen-binding fragment thereof for use according to any one of embodiment 667-672, wherein the CYP46A1 inhibitor is selected from the group consisting of TAK-935, any one of compounds A1-A182, and pharmaceutically acceptable salts thereof.
   674. The CYP46A1 inhibitor, and the anti-amyloid beta antibody or antigen-binding fragment thereof for use according to embodiment 673, wherein the CYP46A1 inhibitor is TAK-935, or a pharmaceutically acceptable salt thereof.
   675. The CYP46A1 inhibitor, and the anti-amyloid beta antibody or antigen-binding fragment thereof for use according to embodiment 673, wherein the CYP46A1 inhibitor is selected from the group consisting of any one of compounds A1-A182, and pharmaceutically acceptable salts thereof.
   676. The CYP46A1 inhibitor, and the anti-amyloid beta antibody or antigen-binding fragment thereof for use according to any one of embodiments 667-675, wherein the anti-amyloid beta antibody or antigen-binding fragment thereof is selected from the group consisting of bapineuzumab, solanezumab, gantenerumab, crenezumab, ponezumab, lecanemab (BAN2401), aducanumab, and antigen-binding fragments thereof.
   677. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 1, (ii) HCDR2 comprising the amino acid sequence of SEQ ID NO: 2, and (iii) HCDR3 comprising the amino acid sequence of SEQ ID NO: 3; and
(b) a light chain variable domain (VL) comprising (i) LCDR1 comprising the amino acid sequence of SEQ ID NO: 4, (ii) LCDR2 comprising the amino acid sequence of SEQ ID NO: 5, and (iii) LCDR3 comprising the amino acid sequence of SEQ ID NO: 6.
   678. The CYP46A1 inhibitor, and the anti-amyloid beta antibody or antigen-binding fragment thereof for use according to embodiment 677, wherein the heavy chain variable domain (VH) comprises the amino acid sequence of SEQ ID NO: 7.
   679. The CYP46A1 inhibitor, and the anti-amyloid beta antibody or antigen-binding fragment thereof for use according to embodiment 677, wherein the light chain variable domain (VL) comprises the amino acid sequence of SEQ ID NO: 8.
   680. The CYP46A1 inhibitor, and the anti-amyloid beta antibody or antigen-binding fragment thereof for use according to embodiment 677, wherein the heavy chain variable domain (VH) comprises the amino acid sequence of SEQ ID NO: 7; and the light chain variable domain (VL) comprises the amino acid sequence of SEQ ID NO: 8.
   681. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 11, (ii) HCDR2 comprising the amino acid sequence of SEQ ID NO: 12, and (iii) HCDR3 comprising the amino acid sequence of SEQ ID NO: 13; and
(b) a light chain variable domain (VL) comprising (i) LCDR1 comprising the amino acid sequence of SEQ ID NO: 4, (ii) LCDR2 comprising the amino acid sequence of SEQ ID NO: 15, and (iii) LCDR3 comprising the amino acid sequence of SEQ ID NO: 16.
   682. The CYP46A1 inhibitor, and the anti-amyloid beta antibody or antigen-binding fragment thereof for use according to embodiment 681, wherein the heavy chain variable domain (VH) comprises the amino acid sequence of SEQ ID NO: 17.
   683. The CYP46A1 inhibitor, and the anti-amyloid beta antibody or antigen-binding fragment thereof for use according to embodiment 681, wherein the light chain variable domain (VL) comprises the amino acid sequence of SEQ ID NO: 18.
   684. The CYP46A1 inhibitor, and the anti-amyloid beta antibody or antigen-binding fragment thereof for use according to embodiment 681, wherein the heavy chain variable domain (VH) comprises the amino acid sequence of SEQ ID NO: 17; and the light chain variable domain (VL) comprises the amino acid sequence of SEQ ID NO: 18.
   685. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 221, (ii) HCDR2 comprising the amino acid sequence of SEQ ID NO: 2, and (iii) HCDR3 comprising the amino acid sequence of SEQ ID NO: 23; and
(b) a light chain variable domain (VL) comprising (i) LCDR1 comprising the amino acid sequence of SEQ ID NO: 24, (ii) LCDR2 comprising the amino acid sequence of SEQ ID NO: 25, and (iii) LCDR3 comprising the amino acid sequence of SEQ ID NO: 26.
   686. The CYP46A1 inhibitor, and the anti-amyloid beta antibody or antigen-binding fragment thereof for use according to embodiment 685, wherein the heavy chain variable domain (VH) comprises the amino acid sequence of SEQ ID NO: 27.
   687. The CYP46A1 inhibitor, and the anti-amyloid beta antibody or antigen-binding fragment thereof for use according to embodiment 685, wherein the light chain variable domain (VL) comprises the amino acid sequence of SEQ ID NO: 28.
   688. The CYP46A1 inhibitor, and the anti-amyloid beta antibody or antigen-binding fragment thereof for use according to embodiment 685, wherein the heavy chain variable domain (VH) comprises the amino acid sequence of SEQ ID NO: 27; and the light chain variable domain (VL) comprises the amino acid sequence of SEQ ID NO: 28.
   689. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 31, (ii) HCDR2 comprising the amino acid sequence of SEQ ID NO: 32, and (iii) HCDR3 comprising the amino acid sequence of SEQ ID NO: 33; and
(b) a light chain variable domain (VL) comprising (i) LCDR1 comprising the amino acid sequence of SEQ ID NO: 34, (ii) LCDR2 comprising the amino acid sequence of SEQ ID NO: 35, and (iii) LCDR3 comprising the amino acid sequence of SEQ ID NO: 36.
   690. The CYP46A1 inhibitor, and the anti-amyloid beta antibody or antigen-binding fragment thereof for use according to embodiment 689, wherein the heavy chain variable domain (VH) comprises the amino acid sequence of SEQ ID NO: 37.
   691. The CYP46A1 inhibitor, and the anti-amyloid beta antibody or antigen-binding fragment thereof for use according to embodiment 689, wherein the light chain variable domain (VL) comprises the amino acid sequence of SEQ ID NO: 38.
   692. The CYP46A1 inhibitor, and the anti-amyloid beta antibody or antigen-binding fragment thereof for use according to embodiment 689, wherein the heavy chain variable domain (VH) comprises the amino acid sequence of SEQ ID NO: 37; and the light chain variable domain (VL) comprises the amino acid sequence of SEQ ID NO: 38.
   693. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 41, (ii) HCDR2 comprising the amino acid sequence of SEQ ID NO: 42, and (iii) HCDR3 comprising the amino acid sequence of SEQ ID NO: 43; and
(b) a light chain variable domain (VL) comprising (i) LCDR1 comprising the amino acid sequence of SEQ ID NO: 44, (ii) LCDR2 comprising the amino acid sequence of SEQ ID NO: 45, and (iii) LCDR3 comprising the amino acid sequence of SEQ ID NO: 46.
   694. The CYP46A1 inhibitor, and the anti-amyloid beta antibody or antigen-binding fragment thereof for use according to embodiment 693, wherein the heavy chain variable domain (VH) comprises the amino acid sequence of SEQ ID NO: 47.
   695. The CYP46A1 inhibitor, and the anti-amyloid beta antibody or antigen-binding fragment thereof for use according to embodiment 693, wherein the light chain variable domain (VL) comprises the amino acid sequence of SEQ ID NO: 48.
   696. The CYP46A1 inhibitor, and the anti-amyloid beta antibody or antigen-binding fragment thereof for use according to embodiment 693, wherein the heavy chain variable domain (VH) comprises the amino acid sequence of SEQ ID NO: 47; and the light chain variable domain (VL) comprises the amino acid sequence of SEQ ID NO: 48.
   697. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 51, (ii) HCDR2 comprising the amino acid sequence of SEQ ID NO: 52, and (iii) HCDR3 comprising the amino acid sequence of SEQ ID NO: 53; and
(b) a light chain variable domain (VL) comprising (i) LCDR1 comprising the amino acid sequence of SEQ ID NO: 54, (ii) LCDR2 comprising the amino acid sequence of SEQ ID NO: 55, and (iii) LCDR3 comprising the amino acid sequence of SEQ ID NO: 56.
   698. The CYP46A1 inhibitor, and the anti-amyloid beta antibody or antigen-binding fragment thereof for use according to embodiment 697, wherein the heavy chain variable domain (VH) comprises the amino acid sequence of SEQ ID NO: 57.
   699. The CYP46A1 inhibitor, and the anti-amyloid beta antibody or antigen-binding fragment thereof for use according to embodiment 697, wherein the light chain variable domain (VL) comprises the amino acid sequence of SEQ ID NO: 58.
   700. The CYP46A1 inhibitor, and the anti-amyloid beta antibody or antigen-binding fragment thereof for use according to embodiment 697, wherein the heavy chain variable domain (VH) comprises the amino acid sequence of SEQ ID NO: 57; and the light chain variable domain (VL) comprises the amino acid sequence of SEQ ID NO: 58.
   701. The CYP46A1 inhibitor, and the anti-amyloid beta antibody or antigen-binding fragment thereof for use according to any one of embodiments 667-675, wherein the anti-amyloid beta antibody or antigen-binding fragment thereof comprises:
(a) a heavy chain variable domain (VH) comprising (i) HCDR1 comprising the amino acid sequence of SEQ ID NO: 61, (ii) HCDR2 comprising the amino acid sequence of SEQ ID NO: 62, and (iii) HCDR3 comprising the amino acid sequence of SEQ ID NO: 63; and
(b) a light chain variable domain (VL) comprising (i) LCDR1 comprising the amino acid sequence of SEQ ID NO: 64, (ii) LCDR2 comprising the amino acid sequence of SEQ ID NO: 65, and (iii) LCDR3 comprising the amino acid sequence of SEQ ID NO: 66.
   702. The CYP46A1 inhibitor, and the anti-amyloid beta antibody or antigen-binding fragment thereof for use according to embodiment 701, wherein the heavy chain variable domain (VH) comprises the amino acid sequence of SEQ ID NO: 67.
   703. The CYP46A1 inhibitor, and the anti-amyloid beta antibody or antigen-binding fragment thereof for use according to embodiment 701, wherein the light chain variable domain (VL) comprises the amino acid sequence of SEQ ID NO: 68.
   704. The CYP46A1 inhibitor, and the anti-amyloid beta antibody or antigen-binding fragment thereof for use according to embodiment 701, wherein the heavy chain variable domain (VH) comprises the amino acid sequence of SEQ ID NO: 67; and the light chain variable domain (VL) comprises the amino acid sequence of SEQ ID NO: 68.
   705. The CYP46A1 inhibitor, and the anti-amyloid beta antibody or antigen-binding fragment thereof for use according to any one of embodiments 667-704, wherein said anti-amyloid beta antibody or antigen-binding fragment thereof comprises a human IgG1, IgG2, IgG3, or IgG4 constant domain.
   706. The CYP46A1 inhibitor, and the anti-amyloid beta antibody or antigen-binding fragment thereof for use according to embodiment 705, wherein the anti-amyloid beta antibody or antigen-binding fragment thereof comprises a human IgG1 constant region.
   707. The CYP46A1 inhibitor, and the anti-amyloid beta antibody or antigen-binding fragment thereof for use according to any one of embodiments 667-706, wherein the anti-amyloid beta antibody or antigen-binding fragment thereof is a human anti-amyloid beta antibody or antigen-binding fragment thereof.
   708. The CYP46A1 inhibitor, and the anti-amyloid beta antibody or antigen-binding fragment thereof for use according to any one of embodiments 667-707, wherein the antigen-binding fragment is a single chain antibody, Fv, Fab, Fab′, F(ab′)₂, Fd, single chain Fv molecule (scFv), bispecific single chain Fv dimer, diabody, domain-deleted antibody or single domain antibody (dAb).
   709. The CYP46A1 inhibitor, and the anti-amyloid beta antibody or antigen-binding fragment thereof for use according to any one of embodiments 667-708, wherein the heavy chain comprises the amino acid sequence of SEQ ID NO: 9.
   710. The CYP46A1 inhibitor, and the anti-amyloid beta antibody or antigen-binding fragment thereof for use according to any one of embodiments 667-709, wherein the light chain comprises the amino acid sequence of SEQ ID NO: 10.
   711. The CYP46A1 inhibitor, and the anti-amyloid beta antibody or antigen-binding fragment thereof for use according to any one of embodiments 667-710, wherein the anti-amyloid beta antibody is aducanumab.
   712. The CYP46A1 inhibitor, and the anti-amyloid beta antibody or antigen-binding fragment thereof for use according to any one of embodiments 667-711, wherein the anti-amyloid beta antibody or antigen binding fragment thereof binds to the same conformational epitope of amyloid beta to which aducanumab binds.
   713. The CYP46A1 inhibitor, and the anti-amyloid beta antibody or antigen-binding fragment thereof for use according to any one of embodiments 667-712, wherein the anti-amyloid beta antibody or antigen binding fragment thereof competitively inhibits binding of aducanumab to amyloid beta.
   714. The CYP46A1 inhibitor, and the anti-amyloid beta antibody or antigen-binding fragment thereof, for use according to any one of embodiments 667-708, wherein the heavy chain comprises the amino acid sequence of SEQ ID NO: 19.
   715. The CYP46A1 inhibitor, and the anti-amyloid beta antibody or antigen-binding fragment thereof, for use according to any one of embodiments 667-708 or 714, wherein the light chain comprises the amino acid sequence of SEQ ID NO: 20.
   716. The CYP46A1 inhibitor, and the anti-amyloid beta antibody or antigen-binding fragment thereof, for use according to any one of embodiments 667-708 or 714-715, wherein the anti-amyloid beta antibody is solazenumab.
   717. The CYP46A1 inhibitor, and the anti-amyloid beta antibody or antigen-binding fragment thereof, for use according to any one of embodiments 667-708 or 714-716, wherein the anti-amyloid beta antibody or antigen binding fragment thereof binds to the same conformational epitope of amyloid beta to which solazenumab binds.
   718. The CYP46A1 inhibitor, and the anti-amyloid beta antibody or antigen-binding fragment thereof, for use according to any one of embodiments 667-708 or 714-716, wherein the anti-amyloid beta antibody or antigen binding fragment thereof competitively inhibits binding of solazenumab to amyloid beta.
   719. The CYP46A1 inhibitor, and the anti-amyloid beta antibody or antigen-binding fragment thereof, for use according to any one of embodiments 667-708, wherein the heavy chain comprises the amino acid sequence of SEQ ID NO: 29.
   720. The CYP46A1 inhibitor, and the anti-amyloid beta antibody or antigen-binding fragment thereof, for use according to any one of embodiments 667-708 or 719, wherein the light chain comprises the amino acid sequence of SEQ ID NO: 30.
   721. The CYP46A1 inhibitor, and the anti-amyloid beta antibody or antigen-binding fragment thereof, for use according to any one of embodiments 667-708 or 719-720, wherein the anti-amyloid beta antibody is crenezumab.
   722. The CYP46A1 inhibitor, and the anti-amyloid beta antibody or antigen-binding fragment thereof, for use according to any one of embodiments 667-708 or 719-721, wherein conformational epitope of amyloid beta to which crenezumab binds.
   723. The CYP46A1 inhibitor, and the anti-amyloid beta antibody or antigen-binding fragment thereof, for use according to any one of embodiments 667-708 or 719-721, wherein the anti-amyloid beta antibody or antigen binding fragment thereof competitively inhibits binding of crenezumab to amyloid beta.
   724. The CYP46A1 inhibitor, and the anti-amyloid beta antibody or antigen-binding fragment thereof, for use according to any one of embodiments 667-708, wherein the heavy chain comprises the amino acid sequence of SEQ ID NO: 39.
   725. The CYP46A1 inhibitor, and the anti-amyloid beta antibody or antigen-binding fragment thereof, for use according to any one of embodiments 667-708 or 724, wherein the light chain comprises the amino acid sequence of SEQ ID NO: 40.
   726. The CYP46A1 inhibitor, and the anti-amyloid beta antibody or antigen-binding fragment thereof, for use according to any one of embodiments 667-708 or 724-725, wherein the anti-amyloid beta antibody is gantenerumab.
   727. The CYP46A1 inhibitor, and the anti-amyloid beta antibody or antigen-binding fragment thereof, for use according to any one of embodiments 667-708 or 724-726, wherein the anti-amyloid beta antibody or antigen binding fragment thereof binds to the same conformational epitope of amyloid beta to which gantenerumab binds.
   728. The CYP46A1 inhibitor, and the anti-amyloid beta antibody or antigen-binding fragment thereof, for use according to any one of embodiments 667-708 or 724-726, wherein the anti-amyloid beta antibody or antigen binding fragment thereof competitively inhibits binding of gantenerumab to amyloid beta.
   729. The CYP46A1 inhibitor, and the anti-amyloid beta antibody or antigen-binding fragment thereof, for use according to any one of embodiments 667-708, wherein the heavy chain comprises the amino acid sequence of SEQ ID NO: 49.
   730. The CYP46A1 inhibitor, and the anti-amyloid beta antibody or antigen-binding fragment thereof, for use according to any one of embodiments 667-708 or 729, wherein the light chain comprises the amino acid sequence of SEQ ID NO: 50.
   731. The CYP46A1 inhibitor, and the anti-amyloid beta antibody or antigen-binding fragment thereof, for use according to any one of embodiments 667-708 or 729-730, wherein the anti-amyloid beta antibody is ponezumab.
   732. The CYP46A1 inhibitor, and the anti-amyloid beta antibody or antigen-binding fragment thereof, for use according to any one of embodiments 667-708 or 729-731, wherein conformational epitope of amyloid beta to which ponezumab binds.
   733. The CYP46A1 inhibitor, and the anti-amyloid beta antibody or antigen-binding fragment thereof, for use according to any one of embodiments 667-708 or 729-731, wherein the anti-amyloid beta antibody or antigen binding fragment thereof competitively inhibits binding of ponezumab to amyloid beta.
   734. The CYP46A1 inhibitor, and the anti-amyloid beta antibody or antigen-binding fragment thereof, for use according to any one of embodiments 667-708, wherein the heavy chain comprises the amino acid sequence of SEQ ID NO: 59.
   735. The CYP46A1 inhibitor, and the anti-amyloid beta antibody or antigen-binding fragment thereof, for use according to any one of embodiments 667-708 or 734, wherein the light chain comprises the amino acid sequence of SEQ ID NO: 60.
   736. The CYP46A1 inhibitor, and the anti-amyloid beta antibody or antigen-binding fragment thereof, for use according to any one of embodiments 667-708 or 734-735, wherein the anti-amyloid beta antibody is bapineuzumab.
   737. The CYP46A1 inhibitor, and the anti-amyloid beta antibody or antigen-binding fragment thereof, for use according to any one of embodiments 667-708 or 734-736, wherein the anti-amyloid beta antibody or antigen binding fragment thereof binds to the same conformational epitope of amyloid beta to which a bapineuzumab binds.
   738. The CYP46A1 inhibitor, and the anti-amyloid beta antibody or antigen-binding fragment thereof, for use according to any one of embodiments 667-708 or 734-736, wherein the anti-amyloid beta antibody or antigen binding fragment thereof competitively inhibits binding of bapineuzumab to amyloid beta.
   739. The CYP46A1 inhibitor, and the anti-amyloid beta antibody or antigen-binding fragment thereof, for use according to any one of embodiments 667-708, wherein the heavy chain comprises the amino acid sequence of SEQ ID NO: 69.
   740. The CYP46A1 inhibitor, and the anti-amyloid beta antibody or antigen-binding fragment thereof, for use according to any one of embodiments 667-708 or 739, wherein the light chain comprises the amino acid sequence of SEQ ID NO: 70.
   741. The CYP46A1 inhibitor, and the anti-amyloid beta antibody or antigen-binding fragment thereof, for use according to any one of embodiments 667-708 or 739-740, wherein the anti-amyloid beta antibody is lecanumab.
   742. The CYP46A1 inhibitor, and the anti-amyloid beta antibody or antigen-binding fragment thereof, for use according to any one of embodiments 667-708 or 739-741, wherein conformational epitope of amyloid beta to which lecanumab binds.
   743. The CYP46A1 inhibitor, and the anti-amyloid beta antibody or antigen-binding fragment thereof, for use according to any one of embodiments 667-708 or 739-741, wherein the anti-amyloid beta antibody or antigen binding fragment thereof competitively inhibits binding of lecanumab to amyloid beta.
   744. The CYP46A1 inhibitor, and the anti-amyloid beta antibody or antigen-binding fragment thereof for use according to any one of embodiments 667-743, wherein the anti-amyloid beta antibody or antigen binding fragment thereof binds to amyloid beta plaques, parenchymal amyloid, cerebrovascular amyloid, or diffuse amyloid beta deposits.
   745. The CYP46A1 inhibitor, and the anti-amyloid beta antibody or antigen-binding fragment thereof for use according to any one of embodiments 667-744, wherein the CYP46A1 inhibitor and the anti-amyloid beta antibody or antigen binding fragment thereof are administered simultaneously.
   746. The CYP46A1 inhibitor, and the anti-amyloid beta antibody or antigen-binding fragment thereof for use according to any one of embodiments 667-744, wherein the CYP46A1 inhibitor and the anti-amyloid beta antibody or antigen binding fragment thereof are administered sequentially.
   747. The CYP46A1 inhibitor, and the anti-amyloid beta antibody or antigen-binding fragment thereof for use according to any one of embodiments 667-744, wherein the CYP46A1 inhibitor and the anti-amyloid beta antibody or antigen binding fragment thereof are administered separately.
   748. The CYP46A1 inhibitor, and the anti-amyloid beta antibody or antigen-binding fragment thereof for use according to any one of embodiments 746 or 747, wherein the anti-amyloid beta antibody or antigen binding fragment thereof is administered before the CYP46A1 inhibitor.
   749. The CYP46A1 inhibitor, and the anti-amyloid beta antibody or antigen-binding fragment thereof for use according to any one of embodiments 746 or 747, wherein the CYP46A1 inhibitor is administered before the anti-amyloid beta antibody or antigen binding fragment thereof.
   750. The CYP46A1 inhibitor, and the anti-amyloid beta antibody or antigen-binding fragment thereof for use according to any one of embodiments 667-749, wherein the frequency of administration of the anti-amyloid beta antibody or antigen binding fragment thereof and the CYP46A1 inhibitor is the same.
   751. The CYP46A1 inhibitor, and the anti-amyloid beta antibody or antigen-binding fragment thereof for use according to any one of embodiments 667-749, wherein the frequency of administration of the anti-amyloid beta antibody or antigen binding fragment thereof and the CYP46A1 inhibitor is different.

1. A method of:
a) treating Mild Cognitive Impairment (MCI) associated with Alzheimer's Disease in a subject;
b) improving executive function in a subject having MCI associated with Alzheimer's Disease;
c) improving working memory in a subject having MCI associated with Alzheimer's Disease;
d) improving learning in a subject having MCI associated with Alzheimer's Disease;
comprising administering to the subject a compound having the formula 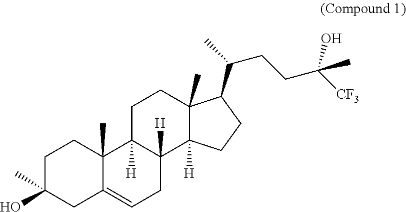
or a pharmaceutically acceptable salt thereof.

2.-5. (canceled)

6. A method of:
a) treating mild dementia associated with Alzheimer's Disease in a subject;
b) improving executive function in a subject having mild dementia associated with Alzheimer's Disease;
c) improving working memory in a subject having mild dementia associated with Alzheimer's Disease;
d) improving learning in a subject having mild dementia associated with Alzheimer's Disease;
comprising administering to the subject a compound having the formula 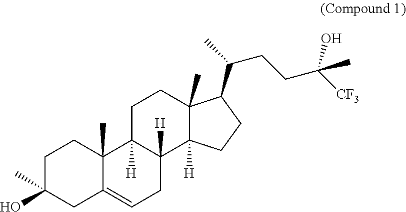
or a pharmaceutically acceptable salt thereof.

7.-12. (canceled)

13. The method of claim 1, wherein about 3 mg daily of Compound 1 is administered to the subject.

14.-36. (canceled)

37. A pharmaceutical composition comprising:
(1) (a) an NMDA receptor positive allosteric modulator;
(b) an anti-amyloid beta antibody or antigen-binding fragment thereof, and
(c) a pharmaceutically acceptable carrier; or
(2) (a) a CYP46A1 inhibitor;
(b) an anti-amyloid beta antibody or antigen-binding fragment thereof; and
(c) a pharmaceutically acceptable carrier.

38.-50. (canceled)

51. A kit comprising
(1) a first container, a second container and a package insert, wherein:
the first container comprises a composition comprising an NMDA receptor positive allosteric modulator;
the second container comprises composition comprising an anti-amyloid beta antibody or antigen-binding fragment thereof; and
the package insert comprises instructions for treating Alzheimer's disease in a subject; or
(2) a first container, a second container and a package insert, wherein:
the first container comprises a composition comprising CYP46A1 inhibitor;
the second container comprises composition comprising an anti-amyloid beta antibody or antigen-binding fragment thereof; and
the package insert comprises instructions for treating Alzheimer's disease in a subject.

52. (canceled)

53. A method for treating or preventing Alzheimer's disease, comprising administering to a subject in need thereof a pharmaceutical composition according to claim 37.

54. A method for treating or preventing cognitive impairment in a subject having Alzheimer's disease, comprising administering to the subject a pharmaceutical composition according to claim 37.

55.-100. (canceled)


SEQUENCE LISTING (70 sequences)

SEQ ID NO: 1 - protein, 5 aa, Artificial Sequence
  Description of Artificial Sequence Synthetic peptide
SYGMH

SEQ ID NO: 2 - protein, 16 aa, Artificial Sequence
  Description of Artificial Sequence Synthetic peptide
VIWEDGMKYYTDSVKG

SEQ ID NO: 3 - protein, 15 aa, Artificial Sequence
  Description of Artificial Sequence Synthetic peptide
DRGIGARRGPYYMDV

SEQ ID NO: 4 - protein, 11 aa, Artificial Sequence
  Description of Artificial Sequence Synthetic peptide
RASQSISSYLN

SEQ ID NO: 5 - protein, 7 aa, Artificial Sequence
  Description of Artificial Sequence Synthetic peptide
AASSLQS

SEQ ID NO: 6 - protein, 9 aa, Artificial Sequence
  Description of Artificial Sequence Synthetic peptide
QQSYSTPLI

SEQ ID NO: 7 - protein, 124 aa, Artificial Sequence
  Description of Artificial Sequence Synthetic polypeptide
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVYVSS

SEQ ID NO: 8 - protein, 108 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKR

SEQ ID NO: 9 - protein, 453 aa, Artificial Sequence
  Description of Artificial Sequence Synthetic polypeptide
QVQLVESGGGVVQPGRSLRLSCAASGFAFSSYGMHWVRQAPGKGLEWVAVIWFDGTKKYYTDSVKGRFTISRDNSKNTLYLQMNTLRAEDTAVYYCARDRGIGARRGPYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 10 - protein, 214 aa, Artificial Sequence
  Description of Artificial Sequence Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 8 aa, Artificial Sequence
  Description of Artificial Sequence Synthetic peptide
GFTFSRYS

SEQ ID NO: 12 - protein, 8 aa, Artificial Sequence
  Description of Artificial Sequence Synthetic peptide
INSVGNST

SEQ ID NO: 13 - protein, 5 aa, Artificial Sequence
  Description of Artificial Sequence Synthetic peptide
ASGDY

SEQ ID NO: 14 - protein, 11 aa, Artificial Sequence
  Description of Artificial Sequence Synthetic peptide
QSLIYSDGNAY

SEQ ID NO: 15 - protein, 3 aa, Artificial Sequence
  Description of Artificial Sequence Synthetic peptide
KVS

SEQ ID NO: 16 - protein, 9 aa, Artificial Sequence
  Description of Artificial Sequence Synthetic peptide
SQSTHVPWT

SEQ ID NO: 17 - protein, 97 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCA

SEQ ID NO: 18 - protein, 100 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVP

SEQ ID NO: 19 - protein, 442 aa, Artificial Sequence
  Description of Artificial Sequence Synthetic polypeptide
EVQLVESGGGLVQPGGSLRLSCAASGFTFSRYSMSWVRQAPGKGLELVAQINSVGNSTYYPDTVKGRFTISRDNAKNTLYLQMNSLRAEDTAVYYCASGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 219 aa, Artificial Sequence
  Description of Artificial Sequence Synthetic polypeptide
DVVMTQSPLSLPVTLGQPASISCRSSQSLIYSDGNAYLHWFLQKPGQSPRLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, Artificial Sequence
  Description of Artificial Sequence Synthetic peptide
GFTFSSYG

SEQ ID NO: 22 - protein, 8 aa, Artificial Sequence
  Description of Artificial Sequence Synthetic peptide
INSNGGST

SEQ ID NO: 23 - protein, 5 aa, Artificial Sequence
  Description of Artificial Sequence Synthetic peptide
ASGDY

SEQ ID NO: 24 - protein, 11 aa, Artificial Sequence
  Description of Artificial Sequence Synthetic peptide
QSLVYSNGDTY

SEQ ID NO: 25 - protein, 3 aa, Artificial Sequence
  Description of Artificial Sequence Synthetic peptide
KVS

SEQ ID NO: 26 - protein, 9 aa, Artificial Sequence
  Description of Artificial Sequence Synthetic peptide
SQSTHVPWT

SEQ ID NO: 27 - protein, 97 aa, Artificial Sequence
  Description of Artificial Sequence Synthetic polypeptide
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCA

SEQ ID NO: 28 - protein, 99 aa, Artificial Sequence
  Description of Artificial Sequence Synthetic polypeptide
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHV

SEQ ID NO: 29 - protein, 440 aa, Artificial Sequence
  Description of Artificial Sequence Synthetic polypeptide
EVQLVESGGGLVQPGGSLRLSCAASGFTFSSYGMSWVRQAPGKGLELVASINSNGGSTYYPDSVKGRFTISRDNAKNSLYLQMNSLRAEDTAVYYCASGDYWGQGTTVTVSSASTKGPSVFPLAPCSRSTSESTAALGCLVKDYFPEPVTVSWNSGALTSGVHTFPAVLQSSGLYSLSSVCHVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 219 aa, Artificial Sequence
  Description of Artificial Sequence Synthetic polypeptide
DIVMTQSPLSLPVTPGEPASISCRSSQSLVYSNGDTYLHWYLQKPGQSPQLLIYKVSNRFSGVPDRFSGSGSGTDFTLKISRVEAEDVGVYYCSQSTHVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 8 aa, Artificial Sequence
  Description of Artificial Sequence Synthetic peptide
GFTFSSYA

SEQ ID NO: 32 - protein, 8 aa, Artificial Sequence
  Description of Artificial Sequence Synthetic peptide
INASGTRT

SEQ ID NO: 33 - protein, 19 aa, Artificial Sequence
  Description of Artificial Sequence Synthetic peptide
ARGKGNTHKPYGYVRYFDV

SEQ ID NO: 34 - protein, 7 aa, Artificial Sequence
  Description of Artificial Sequence Synthetic peptide
QSVSSSY

SEQ ID NO: 35 - protein, 3 aa, Artificial Sequence
  Description of Artificial Sequence Synthetic peptide
GAS

SEQ ID NO: 36 - protein, 9 aa, Artificial Sequence
  Description of Artificial Sequence Synthetic peptide
LQIYNMPIT

SEQ ID NO: 37 - protein, 93 aa, Artificial Sequence
  Description of Artificial Sequence Synthetic polypeptide
QVELVESGGGLVQPGGSLRLSCAASGFTFSSYAMSWVRQAPGKGLEWVSAINASGTRTYYADSVKGRFTISRDNSKNTLYLQMNSLRAEDTAV

SEQ ID NO: 38 - protein, 95 aa, Artificial Sequence
  Description of Artificial Sequence Synthetic polypeptide
IVLTQSPATLSLSPGERATLSCRASQSVSSSYLAWYQQKPGQAPRLLIYGASSRATGVPARFSGSGSGTDFTLTISSLEPEDFATYYCLQIYNMP

SEQ ID NO: 39 - protein, 456 aa, Artificial Sequence
  Description of Artificial Sequence Synthetic polypeptide
QVELVESGGGLVQPGGSLRLSCAASGFTFSSYAMSWVRQAPGKGLEWVSAINASGTRTYYADSVKGRFTISRDNSKNTLYLQMNSLRAEDTAVYYCARGKGNTHKPYGYVRYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 215 aa, Artificial Sequence
  Description of Artificial Sequence Synthetic polypeptide
DIVLTQSPATLSLSPGERATLSCRASQSVSSSYLAWYQQKPGQAPRLLIYGASSRATGVPARFSGSGSGTDFTLTISSLEPEDFATYYCLQIYNMPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 8 aa, Artificial Sequence
  Description of Artificial Sequence Synthetic peptide
GYYTEAYY

SEQ ID NO: 42 - protein, 8 aa, Artificial Sequence
  Description of Artificial Sequence Synthetic peptide
IDPATGNT

SEQ ID NO: 43 - protein, 9 aa, Artificial Sequence
  Description of Artificial Sequence Synthetic peptide
ASLYSLPVY

SEQ ID NO: 44 - protein, 11 aa, Artificial Sequence
  Description of Artificial Sequence Synthetic peptide
QSLLYSDAKTY

SEQ ID NO: 45 - protein, 3 aa, Artificial Sequence
  Description of Artificial Sequence Synthetic peptide
QIS

SEQ ID NO: 46 - protein, 9 aa, Artificial Sequence
  Description of Artificial Sequence Synthetic peptide
LQGTHYPVL

SEQ ID NO: 47 - protein, 97 aa, Artificial Sequence
  Description of Artificial Sequence Synthetic polypeptide
QVQLVQSGAEVKKPGASVKVSCKASGYYTEAYYIHWVRQAPGQGLEWMGRIDPATGNTKYAPRLQDRVTMTRDTSTSTVYMELSSLRSEDTAVYYCA

SEQ ID NO: 48 - protein, 100 aa, Artificial Sequence
  Description of Artificial Sequence Synthetic polypeptide
DVVMTQSPLSLPVTLGQPASISCKSSQSLLYSDAKTYLNWFQQRPGQSPRRLIYQISRLDPGVPDRFSGSGSGTDFTLKISRVEAEDVGVYYCLQGTHYP

SEQ ID NO: 49 - protein, 442 aa, Artificial Sequence
  Description of Artificial Sequence Synthetic polypeptide
QVQLVQSGAEVKKPGASVKVSCKASGYYTEAYYIHWVRQAPGQGLEWMGRIDPATGNTKYAPRLQDRVTMTRDTSTSTVYMELSSLRSEDTAVYYCASLYSLPVYWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPSSIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 50 - protein, 219 aa, Artificial Sequence
  Description of Artificial Sequence Synthetic polypeptide
DVVMTQSPLSLPVTLGQPASISCKSSQSLLYSDAKTYLNWFQQRPGQSPRRLIYQISRLDPGVPDRFSGSGSGTDFTLKISRVEAEDVGVYYCLQGTHYPVLFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 8 aa, Artificial Sequence
  Description of Artificial Sequence Synthetic peptide
GFTFSNYG

SEQ ID NO: 52 - protein, 10 aa, Artificial Sequence
  Description of Artificial Sequence Synthetic peptide
ASIRSGGGRT

SEQ ID NO: 53 - protein, 12 aa, Artificial Sequence
  Description of Artificial Sequence Synthetic peptide
VRYDHYSGSSDY

SEQ ID NO: 54 - protein, 11 aa, Artificial Sequence
  Description of Artificial Sequence Synthetic peptide
QSLLDSDGKTY

SEQ ID NO: 55 - protein, 3 aa, Artificial Sequence
  Description of Artificial Sequence Synthetic peptide
LVS

SEQ ID NO: 56 - protein, 9 aa, Artificial Sequence
  Description of Artificial Sequence Synthetic peptide
WQGTHFPRT

SEQ ID NO: 57 - protein, 98 aa, Artificial Sequence
  Description of Artificial Sequence Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVR

SEQ ID NO: 58 - protein, 100 aa, Artificial Sequence
  Description of Artificial Sequence Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFP

SEQ ID NO: 59 - protein, 447 aa, Artificial Sequence
  Description of Artificial Sequence Synthetic polypeptide
EVQLLESGGGLVQPGGSLRLSCAASGFTFSNYGMSWVRQAPGKGLEWVASIRSGGGRTYYSDNVKGRFTISRDNSKNTLYLQMNSLRAEDTAVYYCVRYDHYSGSSDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNKYTQKSLSLSPG

SEQ ID NO: 60 - protein, 180 aa, Artificial Sequence
  Description of Artificial Sequence Synthetic polypeptide
DVVMTQSPLSLPVTPGEPASISCKSSQSLLDSDGKTYLNWLLQKPGQSPQRLIYLVSKLDSGVPDRFSGSGSGTDFTLKISRVEAEDVGVYYCWQGTHFPRTFGQGTKVEIKRTVAAPSVFIFPPSDEQLKSGTASVVCLLNNFYPREAKVQWKVDNALQSGNSQESVTEQDSKDSTYSL

SEQ ID NO: 61 - protein, 8 aa, Artificial Sequence
  Description of Artificial Sequence Synthetic peptide
GFTFSSFG

SEQ ID NO: 62 - protein, 8 aa, Artificial Sequence
  Description of Artificial Sequence Synthetic peptide
ISSGSSTI

SEQ ID NO: 63 - protein, 17 aa, Artificial Sequence
  Description of Artificial Sequence Synthetic peptide
AREGGYYYGRSYYTMDY

SEQ ID NO: 64 - protein, 11 aa, Artificial Sequence
  Description of Artificial Sequence Synthetic peptide
QSIVHSNGNTY

SEQ ID NO: 65 - protein, 3 aa, Artificial Sequence
  Description of Artificial Sequence Synthetic peptide
KVS

SEQ ID NO: 66 - protein, 9 aa, Artificial Sequence
  Description of Artificial Sequence Synthetic peptide
FQGSHVPPT

SEQ ID NO: 67 - protein, 98 aa, Artificial Sequence
  Description of Artificial Sequence Synthetic polypeptide
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAR

SEQ ID NO: 68 - protein, 100 aa, Artificial Sequence
  Description of Artificial Sequence Synthetic polypeptide
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVP

SEQ ID NO: 69 - protein, 454 aa, Artificial Sequence
  Description of Artificial Sequence Synthetic polypeptide
EVQLVESGGGLVQPGGSLRLSCSASGFTFSSFGMHWVRQAPGKGLEWVAYISSGSSTIYYGDTVKGRFTISRDNAKNSLFLQMSSLRAEDTAVYYCAREGGYYYGRSYYTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 219 aa, Artificial Sequence
  Description of Artificial Sequence Synthetic polypeptide
DVVMTQSPLSLPVTPGAPASISCRSSQSIVHSNGNTYLEWYLQKPGQSPKLLIYKVSNRFSGVPDRFSGSGSGTDFTLRISRVEAEDVGIYYCFQGSHVPPTFGPGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC